SEQUENCE LISTING (12 sequences)

SEQ ID NO: 1 - DNA, 37830 bp, unidentified
  source 1..37830: mol_type = unassigned DNA; note = Chimpanzee adenovirus
catcatcaataatataccttattttggattgaagccaatatgataatgagatgggcggcgcggggcgggaggcgggtccgggggcgggccggcgggcggggcggtgtggcggaagtggactttgtaagtgtggcggatgtgacttgctagtgccgggcgcggtaaaagtgacgttttccgtgcgcgacaacgcccacgggaagtgacatttttcccgcggtttttaccggatgttgtagtgaatttgggcgtaaccaagtaagatttggccattttcgcgggaaaactgaaacggggaagtgaaatctgattaatttcgcgttagtcataccgcgtaatatttgtcgagggccgagggactttggccgattacgtggaggactcgcccaggtgttttttgaggtgaatttccgcgttccgggtcaaagtctccgttttattattatagtcagctgacgcggagtgtatttataccctctgatctcgtcaagtggccactcttgagtgccagcgagtagagttttctcctctgccgctctccgctccgctccgctcggctctgacaccggggaaaaaatgagacatttcacctacgatggcggtgtgctcaccggccagctggctgctgaagtcctggacaccctgatcgaggaggtattggccgataattatcctccctcgactccttttgagccacctacacttcacgaactctacgatctggatgtggtggggcccagcgatccgaacgagcaggcggtttccagtttttttccagagtccatgttgttggccagccaggagggggtcgaacttgagacccctcctccgatcgtggattcccccgatccgccgcagctgactaggcagcccgagcgctgtgcgggacctgagactatgccccagctgctacctgaggtgatcgatctcacctgtaatgagtctggttttccacccagcgaggatgaggacgaagagggtgagcagtttgtgttagattctgtggaacaacccgggcgaggatgcaggtcttgtcaatatcaccggaaaaacacaggagactcccagattatgtgttctctgtgttatatgaagatgacctgtatgtttatttacagtaagtttatcatctgtgggcaggtgggctatagtgtgggtggtggtctttggggggttttttaatatatgtcaggggttatgctgaagacttttttattgtgatttttaaaggtccagtgtctgagcccgagcaagaacctgaaccggagcctgagccttctcgccccaggagaaagcctgtaatcttaactagacccagcgcaccggtagcgagaggcctcagcagcgcggagaccaccgactccggtgcttcctcatcacccccggagattcaccccctggtgcccctgtgtcccgttaagcccgttgccgtgagagtcagtgggcggcggtctgctgtggagtgcattgaggacttgctttttgattcacaggaacctttggacttgagcttgaaacgccccaggcattaaacctggtcacctggactgaatgagttgacgcctatgtttgcttttgaatgacttaatgtgtatagataataaagagtgagataatgttttaattgcatggtgtgtttaacttgggcggagtctgctgggtatataagcttccctgggctaaacttggttacacttgacctcatggaggcctgggagtgtttggagaactttgccggagttcgtgccttgctggacgagagctctaacaatacctcttggtggtggaggtatttgtggggctctccccagggcaagttagtttgtagaatcaaggaggattacaagtgggaatttgaagagcttttgaaatcctgtggtgagctattggattctttgaatctaggccaccaggctctcttccaggagaaggtcatcaggactttggatttttccacaccggggcgcattgcagccgcggttgcttttctagcttttttgaaggatagatggagcgaagagacccacttgagttcgggctacgtcctggattttctggccatgcaactgtggagagcatggatcagacacaagaacaggctgcaactgttgtcttccgtccgcccgttgctgattccggcggaggagcaacaggccgggtcagaggaccgggcccgtcgggatccggaggagagggcaccgaggccgggcgagaggagcgcgctgaacctgggaaccgggctgagcggccatccacatcgggagtgaatgtcgggcaggtggtggatctttttccagaactgcggcggattttgactattagggaggatgggcaatttgttaagggtcttaagagggagaggggggcttctgagcataacgaggaggccagtaatttagcttttagcttgatgaccagacaccgtccagagtgcatcacttttcagcagattaaggacaattgtgccaatgagttggatctgttgggtcagaagtatagcatagagcagctgaccacttactggctgcagccgggtgatgatctggaggaagctattagggtgtatgctaaggtggccctgcggcccgattgcaagtacaagctcaaggggctggtgaatatcaggaattgttgctacatttctggcaacggggcggaggtggagatagagaccgaagacagggtggctttcagatgcagcatgatgaatatgtggccgggggtgctgggcatggacggggtggtgattatgaatgtgaggttcacggggcccaactttaacggcacggtgtttttggggaacaccaacctggtcctgcacggggtgagcttctatgggtttaacaacacctgtgtggaggcctggaccgatgtgaaggtccgcggttgcgccttttatggatgttggaaggccatagtgagccgccctaagagcaggagttccattaagaaatgcttgtttgagaggtgcaccttggggatcctggccgagggcaactgcagggtgcgccacaatgtggcctccgagtgcggttgcttcatgctagtcaagagcgtggcggtaatcaagcataatatggtgtgcggcaacagcgaggacaaggcctcacagatgctgacctgcacggatggcaactgccacttgctgaagaccatccatgtaaccagccacagccggaaggcctggcccgtgttcgagcacaacttgctgacccgctgctccttgcatctgggcaacaggcggggggtgttcctgccctatcaatgcaactttagtcacaccaagatcttgctagagcccgagagcatgtccaaggtgaacttgaacggggtgtttgacatgaccatgaagatctggaaggtgctgaggtacgacgagaccaggtcccggtgcagaccctgcgagtgcgggggcaagcatatgaggaaccagcccgtgatgctggatgtgaccgaggagctgaggacagaccacttggttctggcctgcaccagggccgagtttggttctagcgatgaagacacagattgaggtgggtgagtgggcgtggcctggggtggtcatgaaaatatataagttgggggtcttagggtctctttatttgtgttgcagagaccgccggagccatgagcgggagcagcagcagcagcagtagcagcagcgccttggatggcagcatcgtgagcccttatttgacgacgcggatgccccactgggccggggtgcgtcagaatgtgatgggctccagcatcgacggccgacccgtcctgcccgcaaattccgccacgctgacctatgcgaccgtcgcggggacgccgttggacgccaccgccgccgccgccgccaccgcagccgcctcggccgtgcgcagcctggccacggactttgcattcctgggaccactggcgacaggggctacttctcgggccgctgctgccgccgttcgcgatgacaagctgaccgccctgctggcgcagttggatgcgcttactcgggaactgggtgacctttctcagcaggtcatggccctgcgccagcaggtctcctccctgcaagctggcgggaatgcttctcccacaaatgccgtttaagataaataaaaccagactctgtttggattaaagaaaagtagcaagtgcattgctctctttatttcataattttccgcgcgcgataggccctagaccagcgttctcggtcgttgagggtgcggtgtatcttctccaggacgtggtagaggtggctctggacgttgagatacatgggcatgagcccgtcccgggggtggaggtagcaccactgcagagcttcatgctccggggtggtgttgtagatgatccagtcgtagcaggagcgctgggcatggtgcctaaaaatgtccttcagcagcaggccgatggccagggggaggcccttggtgtaagtgtttacaaaacggttaagttgggaagggtgcattcggggagagatgatgtgcatcttggactgtatttttagattggcgatgtttccgcccagatcccttctgggattcatgttgtgcaggaccaccagtacagtgtatccggtgcacttggggaatttgtcatgcagcttagagggaaaagcgtggaagaacttggagacgcctttgtggcctcccagattttccatgcattcgtccatgatgatggcaatgggcccgcgggaggcagcttgggcaaagatatttctggggtcgctgacgtcgtagttgtgttccagggtgaggtcgtcataggccatttttacaaagcgcgggcggagggtgcccgactgggggatgatggtcccctctggccctggggcgtagttgccctcgcagatctgcatttcccaggccttaatctcggaggggggaatcatatccacctgcggggcgatgaagaaaacggtttccggagccggggagattaactgggatgagagcaggtttctaagcagctgtgattttccacaaccggtgggcccataaataacacctataaccggttgcagctggtagtttagagagctgcagctgccgtcgtcccggaggaggggggccacctcgttgagcatgtccctgacgcgcatgttctccccgaccagatccgccagaaggcgctcgccgcccagggacagcagctcttgcaaggaagcaaagtttttcagcggcttgaggccgtccgccgtgggcatgtttttcagggtctggctcagcagctccaggcggtcccagagctcggtgacgtgctctacggcatctctatccagcatatctcctcgtttcgcgggttggggcgactttcgctgtagggcaccaagcggtggtcgtccagcggggccagagtcatgtccttccatgggcgcagggtcctcgtcagggtggtctgggtcacggtgaaggggtgcgctccgggctgagcgcttgccaaggtgcgcttgaggctggttctgctggtgctgaagcgctgccggtcttcgccctgcgcgtcggccaggtagcatttgaccatggtgtcatagtccagcccctccgcggcgtgtcccttggcgcgcagcttgcccttggaggtggcgccgcacgaggggcagagcaggctcttgagcgcgtagagcttgggggcgaggaagaccgattcgggggagtaggcgtccgcgccgcagaccccgcacacggtctcgcactccaccagccaggtgagctcggggcgcgccgggtcaaaaaccaggtttcccccatgctttttgatgcgtttcttacctcgggtctccatgaggtggtgtccccgctcggtgacgaagaggctgtccgtgtctccgtagaccgacttgaggggtcttttctccaggggggtccctcggtcttcctcgtagaggaactcggaccactctgagacgaaggcccgcgtccaggccaggacgaaggaggctatgtgggaggggtagcggtcgttgtccactagggggtccaccttctccaaggtgtgaagacacatgtcgccttcctcggcgtccaggaaggtgattggcttgtaggtgtaggccacgtgaccgggggttcctgacgggggggtataaaagggggtgggggcgcgctcgtcgtcactctcttccgcatcgctgtctgcgagggccagctgctggggtgagtattccctctcgaaggcgggcatgacctccgcgctgaggttgtcagtttccaaaaacgaggaggatttgatgttcacctgtcccgaggtgatacctttgagggtacccgcgtccatctggtcagaaaacacgatctttttattgtccagcttggtggcgaacgacccgtagagggcgttggagagcagcttggcgatggagcgcagggtctggttcttgtccctgtcggcgcgctccttggccgcgatgttgagctgcacgtactcgcgcgcgacgcagcgccactcggggaagacggtggtgcgctcgtcgggcaccaggcgcacgcgccagccgcggttgtgcagggtgaccaggtccacgctggtggcgacctcgccgcgcaggcgctcgttggtccagcagagacggccgcccttgcgcgagcagaaggggggcagggggtcgagctgggtctcgtccggggggtccgcgtccacggtgaaaaccccggggcgcaggcgcgcgtcgaagtagtctatcttgcaaccttgcatgtccagcgcctgctgccagtcgcgggcggcgagcgcgcgctcgtaggggttgagcggcgggccccagggcatggggtgggtgagtgcggaggcgtacatgccgcagatgtcatagacgtagaggggctcccgcaggaccccgatgtaggtggggtagcagcggccgccgcggatgctggcgcgcacgtagtcatacagctcgtgcgagggggcgaggaggtcggggcccaggttggtgcgggcggggcgctccgcgcggaagacgatctgcctgaagatggcatgcgagttggaagagatggtggggcgctggaagacgttgaagctggcgtcctgcaggccgacggcgtcgcgcacgaaggaggcgtaggagtcgcgcagcttgtgtaccagctcggcggtgacctgcacgtcgagcgcgcagtagtcgagggtctcgcggatgatgtcatatttagcctgccccttctttttccacagctcgcggttgaggacaaactcttcgcggtctttccagtactcttggatcgggaaaccgtccggttccgaacggtaagagcctagcatgtagaactggttgacggcctggtaggcgcagcagcccttctccacggggagggcgtaggcctgcgcggccttgcggagcgaggtgtgggtcagggcgaaggtgtccctgaccatgactttgaggtactggtgcttgaagtcggagtcgtcgcagccgccccgctcccagagcgagaagtcggtgcgcttcttggagcgggggttgggcagagcgaaggtgacatcgttgaagaggattttgcccgcgcggggcatgaagttgcgggtgatgcggaagggccccggcacttcagagcggttgttgatgacctgggcggcgagcacgatctcgtcgaagccgttgatgttgtggcccacgatgtagagttccaggaagcggggccggccctttacggtgggcagcttctttagctcttcgtaggtgagctcctcgggcgaggcgaggccgtgctcggccagggcccagtccgcgaggtgcgggttgtctctgaggaaggacttccagaggtcgcgggccaggagggtctgcaggcggtctctgaaggtcctgaactggcggcccacggccattttttcgggggtgatgcagtagaaggtgagggggtcttgctgccagcggtcccagtcgagctgcagggcgaggtcgcgcgcggcggtgaccaggcgctcgtcgcccccgaatttcatgaccagcatgaagggcacgagctgctttccgaaggcccccatccaagtgtaggtctctacatcgtaggtgacaaagaggcgctccgtgcgaggatgcgagccgatcgggaagaactggatctcccgccaccagttggaggagtggctgttgatgtggtggaagtagaagtcccgtcgccgggccgaacactcgtgctggcttttgtaaaagcgagcgcagtactggcagcgctgcacgggctgtacctcatgcacgagatgcacctttcgcccgcgcacgaggaagccgaggggaaatctgagccccccgcctggctcgcggcatggctggttctcttctactttggatgcgtgtccgtctccgtctggctcctcgaggggtgttacggtggagcggaccaccacgccgcgcgagccgcaggtccagatatcggcgcgcggcggtcggagtttgatgacgacatcgcgcagctgggagctgtccatggtctggagctcccgcggcggcggcaggtcagccgggagttcttgcaggttcacctcgcagagtcgggccagggcgcggggcaggtctaggtggtacctgatctctaggggcgtgttggtggcggcgtcgatggcttgcaggagcccgcagccccggggggcgacgacggtgccccgcggggtggtggtggtggtggcggtgcagctcagaagcggtgccgcgggcgggcccccggaggtagggggggctccggtcccgcgggcaggggcggcagcggcacgtcggcgtggagcgcgggcaggagttggtgctgtgcccggaggttgctggcgaaggcgacgacgcggcggttgatctcctggatctggcgcctctgcgtgaagacgacgggcccggtgagcttgaacctgaaagagagttcgacagaatcaatctcggtgtcattgaccgcggcctggcgcaggatctcctgcacgtctcccgagttgtcttggtaggcgatctcggccatgaactgctcgatctcttcctcctggaggtctccgcgtccggcgcgttccacggtggccgccaggtcgttggagatgcgccccatgagctgcgagaaggcgttgagtccgccctcgttccagactcggctgtagaccacgcccccctggtcatcgcgggcgcgcatgaccacctgcgcgaggttgagctccacgtgccgcgcgaagacggcgtagttgcgcagacgctggaagaggtagttgagggtggtggcggtgtgctcggccacgaagaagttcatgacccagcggcgcaacgtggattcgttgatgtcccccaaggcctccagccgttccatggcctcgtagaagtccacggcgaagttgaaaaactgggagttgcgcgccgacacggtcaactcctcctccagaagacggatgagctcggcgacggtgtcgcgcacctcgcgctcgaaggctatggggatctcttcctccgctagcatcaccacctcctcctcttcctcctcttctggcacttccatgatggcttcctcctcttcggggggtggcggcggcggcggtgggggagggggcgctctgcgccggcggcggcgcaccgggaggcggtccacgaagcgcgcgatcatctccccgcggcggcggcgcatggtctcggtgacggcgcggccgttctcccgggggcgcagttggaagacgccgccggacatctggtgctggggcgggtggccgtgaggcagcgagacggcgctgacgatgcatctcaacaattgctgcgtaggtacgccgccgagggacctgagggagtccatatccaccggatccgaaaacctttcgaggaaggcgtctaaccagtcgcagtcgcaaggtaggctgagcaccgtggcgggcggcggggggtggggggagtgtctggcggaggtgctgctgatgatgtaattgaagtaggcggacttgacacggcggatggtcgacaggagcaccatgtccttgggtccggcctgctggatgcggaggcggtcggctatgccccaggcttcgttctggcatcggcgcaggtccttgtagtagtcttgcatgagcctttccaccggcacctcttctccttcctcttctgcttcttccatgtctgcttcggccctggggcggcgccgcgcccccctgccccccatgcgcgtgaccccgaaccccctgagcggttggagcagggccaggtcggcgacgacgcgctcggccaggatggcctgctgcacctgcgtgagggtggtttggaagtcatccaagtccacgaagcggtggtaggcgcccgtgttgatggtgtaggtgcagttggccatgacggaccagttgacggtctggtggcccggttgcgacatctcggtgtacctgagtcgcgagtaggcgcgggagtcgaagacgtagtcgttgcaagtccgcaccaggtactggtagcccaccaggaagtgcggcggcggctggcggtagaggggccagcgcagggtggcgggggctccgggggccaggtcttccagcatgaggcggtggtaggcgtagatgtacctggacatccaggtgatacccgcggcggtggtggaggcgcgcgggaagtcgcgcacccggttccagatgttgcgcaggggcagaaagtgctccatggtaggcgtgctctgtccagtcagacgcgcgcagtcgttgatactctagaccagggaaaacgaaagccggtcagcgggcactcttccgtggtctggtgaatagatcgcaagggtatcatggcggagggcctcggttcgagccccgggtccgggccggacggtccgccatgatccacgcggttaccgcccgcgtgtcgaacccaggtgtgcgacgtcagacaacggtggagtgttccttttggcgtttttctggccgggcgccggcgccgcgtaagagactaagccgcgaaagcgaaagcagtaagtggctcgctccccgtagccggagggatccttgctaagggttgcgttgcggcgaaccccggttcgaatcccgtactcgggccggccggacccgcggctaaggtgttggattggcctccccctcgtataaagaccccgcttgcggattgactccggacacggggacgagccccttttatttttgctttccccagatgcatccggtgctgcggcagatgcgccccccgccccagcagcagcaacaacaccagcaagagcggcagcaacagcagcgggagtcatgcagggccccctcacccaccctcggcgggccggccacctcggcgtccgcggccgtgtctggcgcctgcggcggcggcggggggccggctgacgaccccgaggagcccccgcggcgcagggccagacactacctggacctggaggagggcgagggcctggcgcggctgggggcgccgtctcccgagcgccacccgcgggtgcagctgaagcgcgactcgcgcgaggcgtacgtgcctcggcagaacctgttcagggaccgcgcgggcgaggagcccgaggagatgcgggacaggaggttcagcgcagggcgggagctgcggcaggggctgaaccgcgagcggctgctgcgcgaggaggactttgagcccgacgcgcggacggggatcagccccgcgcgcgcgcacgtggcggccgccgacctggtgacggcgtacgagcagacggtgaaccaggagatcaacttccaaaagagtttcaacaaccacgtgcgcacgctggtggcgcgcgaggaggtgaccatcgggctgatgcacctgtgggactttgtaagcgcgctggtgcagaaccccaacagcaagcctctgacggcgcagctgttcctgatagtgcagcacagcagggacaacgaggcgtttagggacgcgctgctgaacatcaccgagcccgagggtcggtggctgctggacctgattaacatcctgcagagcatagtggtgcaggagcgcagcctgagcctggccgacaaggtggcggccatcaactactcgatgctgagcctgggcaagttttacgcgcgcaagatctaccagacgccgtacgtgcccatagacaaggaggtgaagatcgacggtttttacatgcgcatggcgctgaaggtgctcaccctgagcgacgacctgggcgtgtaccgcaacgagcgcatccacaaggccgtgagcgtgagccggcggcgcgagctgagcgaccgcgagctgatgcacagcctgcagcgggcgctggcgggcgccggcagcggcgacagggaggcggagtcctacttcgatgcgggggcggacctgcgctgggcgcccagccggcgggccctggaggccgcgggggtccgcgaggactatgacgaggacggcgaggaggatgaggagtacgagctagaggagggcgagtacctggactaaaccgcgggtggtgtttccggtagatgcaagacccgaacgtggtggacccggcgctgcgggcggctctgcagagccagccgtccggccttaactcctcagacgactggcgacaggtcatggaccgcatcatgtcgctgacggcgcgtaacccggacgcgttccggcagcagccgcaggccaacaggctctccgccatcctggaggcggtggtgcctgcgcgctcgaaccccacgcacgagaaggtgctggccatagtgaacgcgctggccgagaacagggccatccgcccggacgaggccgggctggtgtacgacgcgctgctgcagcgcgtggcccgctacaacagcggcaacgtgcagaccaacctggaccggctggtgggggacgtgcgcgaggcggtggcgcagcgcgagcgcgcggatcggcagggcaacctgggctccatggtggcgctgaatgccttcctgagcacgcagccggccaacgtgccgcgggggcaggaagactacaccaactttgtgagcgcgctgcggctgatggtgaccgagaccccccagagcgaggtgtaccagtcgggcccggactacttcttccagaccagcagacagggcctgcagacggtgaacctgagccaggctttcaagaacctgcgggggctgtggggcgtgaaggcgcccaccggcgaccgggcgacggtgtccagcctgctgacgcccaactcgcgcctgctgctgctgctgatcgcgccgttcacggacagcggcagcgtgtcccgggacacctacctggggcacctgctgaccctgtaccgcgaggccatcgggcaggcgcaggtggacgagcacaccttccaggagatcaccagcgtgagccgcgcgctggggcaggaggacacgagcagcctggaggcgactctgaactacctgctgaccaaccggcggcagaagattccctcgctgcacagcctgacctccgaggaggagcgcatcttgcgctacgtgcagcagagcgtgagcctgaacctgatgcgcgacggggtgacgcccagcgtggcgctggacatgaccgcgcgcaacatggaaccgggcatgtacgccgcgcaccggccttacatcaaccgcctgatggactacctgcatcgcgcggcggccgtgaaccccgagtactttaccaacgccatcctgaacccgcactggctcccgccgcccgggttctacagcgggggcttcgaggtcccggagaccaacgatggcttcctgtgggacgacatggacgacagcgtgttctccccgcggccgcaggcgctggcggaagcgtccctgctgcgtcccaagaaggaggaggaggaggaggcgagtcgccgccgcggcagcagcggcgtggcttctctgtccgagctgggggcggcagccgccgcgcgccccgggtccctgggcggcagcccctttccgagcctggtggggtctctgcacagcgagcgcaccacccgccctcggctgctgggcgaggacgagtacctgaataactccctgctgcagccggtgcgggagaaaaacctgcctcccgccttccccaacaacgggatagagagcctggtggacaagatgagcagatggaagacctatgcgcaggagcacagggacgcgcctgcgctccggccgcccacgcggcgccagcgccacgaccggcagcgggggctggtgtgggatgacgaggactccgcggacgatagcagcgtgctggacctgggagggagcggcaacccgttcgcgcacctgcgcccccgcctggggaggatgttttaaaaaaaaaaaaaaaaagcaagaagcatgatgcaaaaattaaataaaactcaccaaggccatggcgaccgagcgttggtttcttgtgttcccttcagtatgcggcgcgcggcgatgtaccaggagggacctcctccctcttacgagagcgtggtgggcgcggcggcggcggcgccctcttctccctttgcgtcgcagctgctggagccgccgtacgtgcctccgcgctacctgcggcctacgggggggagaaacagcatccgttactcggagctggcgcccctgttcgacaccacccgggtgtacctggtggacaacaagtcggcggacgtggcctccctgaactaccagaacgaccacagcaattttttgaccacggtcatccagaacaatgactacagcccgagcgaggccagcacccagaccatcaatctggatgaccggtcgcactggggcggcgacctgaaaaccatcctgcacaccaacatgcccaacgtgaacgagttcatgttcaccaataagttcaaggcgcgggtgatggtgtcgcgctcgcacaccaaggaagaccgggtggagctgaagtacgagtgggtggagttcgagctgccagagggcaactactccgagaccatgaccattgacctgatgaacaacgcgatcgtggagcactatctgaaagtgggcaggcagaacggggtcctggagagcgacatcggggtcaagttcgacaccaggaacttccgcctggggctggaccccgtgaccgggctggttatgcccggggtgtacaccaacgaggccttccatcccgacatcatcctgctgcccggctgcggggtggacttcacttacagccgcctgagcaacctcctgggcatccgcaagcggcagcccttccaggagggcttcaggatcacctacgaggacctggaggggggcaacatccccgcgctcctcgatgtggaggcctaccaggatagcttgaaggaaaatgaggcgggacaggaggataccgcccccgccgcctccgccgccgccgagcagggcgaggatgctgctgacaccgcggccgcggacggggcagaggccgaccccgctatggtggtggaggctcccgagcaggaggaggacatgaatgacagtgcggtgcgcggagacaccttcgtcacccggggggaggaaaagcaagcggaggccgaggccgcggccgaggaaaagcaactggcggcagcagcggcggcggcggcgttggccgcggcggaggctgagtctgaggggaccaagcccgccaaggagcccgtgattaagcccctgaccgaagatagcaagaagcgcagttacaacctgctcaaggacagcaccaacaccgcgtaccgcagctggtacctggcctacaactacggcgacccgtcgacgggggtgcgctcctggaccctgctgtgcacgccggacgtgacctgcggctcggagcaggtgtactggtcgctgcccgacatgatgcaagaccccgtgaccttccgctccacgcggcaggtcagcaacttcccggtggtgggcgccgagctgctgcccgtgcactccaagagcttctacaacgaccaggccgtctactcccagctcatccgccagttcacctctctgacccacgtgttcaatcgctttcctgagaaccagattctggcgcgcccgcccgcccccaccatcaccaccgtcagtgaaaacgttcctgctctcacagatcacgggacgctaccgctgcgcaacagcatcggaggagtccagcgagtgaccgttactgacgccagacgccgcacctgcccctacgtttacaaggccttgggcatagtctcgccgcgcgtcctttccagccgcactttttgagcaacaccaccatcatgtccatcctgatctcacccagcaataactccggctggggactgctgcgcgcgcccagcaagatgttcggaggggcgaggaagcgttccgagcagcaccccgtgcgcgtgcgcgggcacttccgcgccccctggggagcgcacaaacgcggccgcgcggggcgcaccaccgtggacgacgccatcgactcggtggtggagcaggcgcgcaactacaggcccgcggtctctaccgtggacgcggccatccagaccgtggtgcggggcgcgcggcggtacgccaagctgaagagccgccggaagcgcgtggcccgccgccaccgccgccgacccggggccgccgccaaacgcgccgccgcggccctgcttcgccgggccaagcgcacgggccgccgcgccgccatgagggccgcgcgccgcttggccgccggcatcaccgccgccaccatggccccccgtacccgaagacgcgcggccgccgccgccgccgccgccatcagtgacatggccagcaggcgccggggcaacgtgtactgggtgcgcgactcggtgaccggcacgcgcgtgcccgtgcgcttccgccccccgcggacttgagatgatgtgaaaaaacaacactgagtctcctgctgttgtgtgtatcccagcggcggcggcgcgcgcagcgtcatgtccaagcgcaaaatcaaagaagagatgctccaggtcgtcgcgccggagatctatgggcccccgaagaaggaagagcaggattcgaagccccgcaagataaagcgggtcaaaaagaaaaagaaagatgatgacgatgccgatggggaggtggagttcctgcgcgccacggcgcccaggcgcccggtgcagtggaagggccggcgcgtaaagcgcgtcctgcgccccggcaccgcggtggtcttcacgcccggcgagcgctccacccggactttcaagcgcgtctatgacgaggtgtacggcgacgaagacctgctggagcaggccaacgagcgcttcggagagtttgcttacgggaagcgtcagcgggcgctggggaaggaggacctgctggcgctgccgctggaccagggcaaccccacccccagtctgaagcccgtgaccctgcagcaggtgctgccgagcagcgcaccctccgaggcgaagcggggtctgaagcgcgagggcggcgacctggcgcccaccgtgcagctcatggtgcccaagcggcagaggctggaggatgtgctggagaaaatgaaagtagaccccggtctgcagccggacatcagggtccgccccatcaagcaggtggcgccgggcctcggcgtgcagaccgtggacgtggtcatccccaccggcaactcccccgccgccgccaccactaccgctgcctccacggacatggagacacagaccgatcccgccgcagccgcagccgcagccgccgccgcgacctcctcggcggaggtgcagacggacccctggctgccgccggcgatgtcagctccccgcgcgcgtcgcgggcgcaggaagtacggcgccgccaacgcgctcctgcccgagtacgccttgcatccttccatcgcgcccacccccggctaccgaggctatacctaccgcccgcgaagagccaagggttccacccgccgtccccgccgacgcgccgccgccaccacccgccgccgccgccgcagacgccagcccgcactggctccagtctccgtgaggaaagtggcgcgcgacggacacaccctggtgctgcccagggcgcgctaccaccccagcatcgtttaaaagcctgttgtggttcttgcagatatggccctcacttgccgcctccgtttcccggtgccgggataccgaggaggaagatcgcgccgcaggaggggtctggccggccgcggcctgagcggaggcagccgccgcgcgcaccggcggcgacgcgccaccagccgacgcatgcgcggcggggtgctgcccctgttaatccccctgatcgccgcggcgatcggcgccgtgcccgggatcgcctccgtggccttgcaagcgtcccagaggcattgacagacttgcaaacttgcaaatatggaaaaaaaaaccccaataaaaaagtctagactctcacgctcgcttggtcctgtgactattttgtagaatggaagacatcaactttgcgtcgctggccccgcgtcacggctcgcgcccgttcctgggacactggaacgatatcggcaccagcaacatgagcggtggcgccttcagttggggctctctgtggagcggcattaaaagtatcgggtctgccgttaaaaattacggctcccgggcctggaacagcagcacgggccagatgttgagagacaagttgaaagagcagaacttccagcagaaggtggtggagggcctggcctccggcatcaacggggtggtggacctggccaaccaggccgtgcagaataagatcaacagcagactggacccccggccgccggtggaggaggtgccgccggcgctggagacggtgtcccccgatgggcgtggcgagaagcgcccgcggcccgatagggaagagaccactctggtcacgcagaccgatgagccgcccccgtatgaggaggccctgaagcaaggtctgcccaccacgcggcccatcgcgcccatggccaccggggtggtgggccgccacacccccgccacgctggacttgcctccgcccgccgatgtgccgcagcagcagaaggcggcacagccgggcccgcccgcgaccgcctcccgttcctccgccggtcctctgcgccgcgcggccagcggcccccgcgggggggtcgcgaggcacggcaactggcagagcacgctgaacagcatcgtgggtctgggggtgcggtccgtgaagcgccgccgatgctactgaatagcttagctaacgtgttgtatgtgtgtatgcgccctatgtcgccgccagaggagctgctgagtcgccgccgttcgcgcgcccaccaccaccgccactccgcccctcaagatggcgaccccatcgatgatgccgcagtggtcgtacatgcacatctcgggccaggacgcctcggagtacctgagccccgggctggtgcagttcgcccgcgccaccgagagctacttcagcctgagtaacaagtttaggaaccccacggtggcgcccacgcacgatgtgaccaccgaccggtctcagcgcctgacgctgcggttcattcccgtggaccgcgaggacaccgcgtactcgtacaaggcgcggttcaccctggccgtgggcgacaaccgcgtgctggacatggcctccacctactttgacatccgcggggtgctggaccggggtcccactttcaagccctactctggcaccgcctacaactccctggcccccaagggcgctcccaactcctgcgagtgggagcaagaggaaactcaggcagttgaagaagcagcagaagaggaagaagaagatgctgacggtcaagctgaggaagagcaagcagctaccaaaaagactcatgtatatgctcaggctcccctttctggcgaaaaaattagtaaagatggtctgcaaataggaacggacgctacagctacagaacaaaaacctatttatgcagaccctacattccagcccgaaccccaaatcggggagtcccagtggaatgaggcagatgctacagtcgccggcggtagagtgctaaagaaatctactcccatgaaaccatgctatggttcctatgcaagacccacaaatgctaatggaggtcagggtgtactaacggcaaatgcccagggacagctagaatctcaggttgaaatgcaattcttttcaacttctgaaaacgcccgtaacgaggctaacaacattcagcccaaattggtgctgtatagtgaggatgtgcacatggagaccccggatacgcacctttcttacaagcccgcaaaaagcgatgacaattcaaaaatcatgctgggtcagcagtccatgcccaacagacctaattacatcggcttcagagacaactttatcggcctcatgtattacaatagcactggcaacatgggagtgcttgcaggtcaggcctctcagttgaatgcagtggtggacttgcaagacagaaacacagaactgtcctaccagctcttgcttgattccatgggtgacagaaccagatacttttccatgtggaatcaggcagtggacagttatgacccagatgttagaattattgaaaatcatggaactgaagacgagctccccaactattgtttccctctgggtggcataggggtaactgacacttaccaggctgttaaaaccaacaatggcaataacgggggccaggtgacttggacaaaagatgaaacttttgcagatcgcaatgaaataggggtgggaaacaatttcgctatggagatcaacctcagtgccaacctgtggagaaacttcctgtactccaacgtggcgctgtacctaccagacaagcttaagtacaacccctccaatgtggacatctctgacaaccccaacacctacgattacatgaacaagcgagtggtggccccggggctggtggactgctacatcaacctgggcgcgcgctggtcgctggactacatggacaacgtcaaccccttcaaccaccaccgcaatgcgggcctgcgctaccgctccatgctcctgggcaacgggcgctacgtgcccttccacatccaggtgccccagaagttctttgccatcaagaacctcctcctcctgccgggctcctacacctacgagtggaacttcaggaaggatgtcaacatggtcctccagagctctctgggtaacgatctcagggtggacggggccagcatcaagttcgagagcatctgcctctacgccaccttcttccccatggcccacaacacggcctccacgctcgaggccatgctcaggaacgacaccaacgaccagtccttcaatgactacctctccgccgccaacatgctctaccccatacccgccaacgccaccaacgtccccatctccatcccctcgcgcaactgggcggccttccgcggctgggccttcacccgcctcaagaccaaggagaccccctccctgggctcgggattcgacccctactacacctactcgggctccattccctacctggacggcaccttctacctcaaccacactttcaagaaggtctcggtcaccttcgactcctcggtcagctggccgggcaacgaccgtctgctcacccccaacgagttcgagatcaagcgctcggtcgacggggagggctacaacgtggcccagtgcaacatgaccaaggactggttcctggtccagatgctggccaactacaacatcggctaccagggcttctacatcccagagagctacaaggacaggatgtactccttcttcaggaacttccagcccatgagccggcaggtggtggaccagaccaagtacaaggactaccaggaggtgggcatcatccaccagcacaacaactcgggcttcgtgggctacctcgcccccaccatgcgcgagggacaggcctaccccgccaacttcccctatccgctcataggcaagaccgcggtcgacagcatcacccagaaaaagttcctctgcgaccgcaccctctggcgcatccccttctccagcaacttcatgtccatgggtgcgctctcggacctgggccagaacttgctctacgccaactccgcccacgccctcgacatgaccttcgaggtcgaccccatggacgagcccacccttctctatgttctgttcgaagtctttgacgtggtccgggtccaccagccgcaccgcggcgtcatcgagaccgtgtacctgcgtacgcccttctcggccggcaacgccaccacctaaagaagcaagccgcagtcatcgccgcctgcatgccgtcgggttccaccgagcaagagctcagggccatcgtcagagacctgggatgcgggccctattttttgggcaccttcgacaagcgcttccctggctttgtctccccacacaagctggcctgcgccatcgtcaacacggccggccgcgagaccgggggcgtgcactggctggccttcgcctggaacccgcgctccaaaacatgcttcctctttgaccccttcggcttttcggaccagcggctcaagcaaatctacgagttcgagtacgagggcttgctgcgtcgcagcgccatcgcctcctcgcccgaccgctgcgtcaccctcgaaaagtccacccagaccgtgcaggggcccgactcggccgcctgcggtctcttctgctgcatgtttctgcacgcctttgtgcactggcctcagagtcccatggaccgcaaccccaccatgaacttgctgacgggggtgcccaactccatgctccagagcccccaggtcgagcccaccctgcgccgcaaccaggagcagctctacagcttcctggagcgccactcgccttacttccgccgccacagcgcacagatcaggagggccacctccttctgccacttgcaagagatgcaagaagggtaataacgatgtacacactttttttctcaataaatggcatctttttatttatacaagctctctggggtattcatttcccaccaccacccgccgttgtcgccatctggctctatttagaaatcgaaagggttctgccgggagtcgccgtgcgccacgggcagggacacgttgcgatactggtagcgggtgccccacttgaactcgggcaccaccaggcgaggcagctcggggaagttttcgctccacaggctgcgggtcagcaccagcgcgttcatcaggtcgggcgccgagatcttgaagtcgcagttggggccgccgccctgcgcgcgcgagttgcggtacaccgggttgcagcactggaacaccaacagcgccgggtgcttcacgctggccagcacgctgcggtcggagatcagctcggcgtccaggtcctccgcgttgctcagcgcgaacggggtcatcttgggcacttgccgccccaggaagggcgcgtgccccggtttcgagttgcagtcgcagcgcagcgggatcagcaggtgcccgtgcccggactcggcgttggggtacagcgcgcgcatgaaggcctgcatctggcggaaggccatctgggccttggcgccctccgagaagaacatgccgcaggacttgcccgagaactggtttgcggggcagctggcgtcgtgcaggcagcagcgcgcgtcggtgttggcgatctgcaccacgttgcgcccccaccggttcttcacgatcttggccttggacgattgctccttcagcgcgcgctgcccgttctcgctggtcacatccatctcgatcacatgttccttgttcaccatgctgctgccgtgcagacacttcagctcgccctccgtctcggtgcagcggtgctgccacagcgcgcagcccgtgggctcgaaagacttgtaggtcacctccgcgaaggactgcaggtacccctgcaaaaagcggcccatcatggtcacgaaggtcttgttgctgctgaaggtcagctgcagcccgcggtgctcctcgttcagccaggtcttgcacacggccgccagcgcctccacctggtcgggcagcatcttgaagttcaccttcagctcattctccacgtggtacttgtccatcagcgtgcgcgccgcctccatgcccttctcccaggccgacaccagcggcaggctcacggggttcttcaccatcaccgtggccgccgcctccgccgcgctttcgctttccgccccgctgttctcttcctcttcctcctcttcctcgccgccgcccactcgcagcccccgcaccacggggtcgtcttcctgcaggcgctgcaccttgcgcttgccgttgcgcccctgcttgatgcgcacgggcgggttgctgaagcccaccatcaccagcgcggcctcttcttgctcgtcctcgctgtccagaatgacctccggggagggggggttggtcatcctcagtaccgaggcacgcttctttttcttcctgggggcgttcgccagctccgcggctgcggccgctgccgaggtcgaaggccgagggctgggcgtgcgcggcaccagcgcgtcctgcgagccgtcctcgtcctcctcggactcgagacggaggcgggcccgcttcttcgggggcgcgcggggcggcggaggcggcggcggcgacggagacggggacgagacatcgtccagggtgggtggacggcgggccgcgccgcgtccgcgctcgggggtggtctcgcgctggtcctcttcccgactggccatctcccactgctccttctcctataggcagaaagagatcatggagtctctcatgcgagtcgagaaggaggaggacagcctaaccgccccctctgagccctccaccaccgccgccaccaccgccaatgccgccgcggacgacgcgcccaccgagaccaccgccagtaccaccctccccagcgacgcacccccgctcgagaatgaagtgctgatcgagcaggacccgggttttgtgagcggagaggaggatgaggtggatgagaaggagaaggaggaggtcgccgcctcagtgccaaaagaggataaaaagcaagaccaggacgacgcagataaggatgagacagcagtcgggcgggggaacggaagccatgatgctgatgacggctacctagacgtgggagacgacgtgctgcttaagcacctgcaccgccagtgcgtcatcgtctgcgacgcgctgcaggagcgctgcgaagtgcccctggacgtggcggaggtcagccgcgcctacgagcggcacctcttcgcgccgcacgtgccccccaagcgccgggagaacggcacctgcgagcccaacccgcgtctcaacttctacccggtcttcgcggtacccgaggtgctggccacctaccacatctttttccaaaactgcaagatccccctctcctgccgcgccaaccgcacccgcgccgacaaaaccctgaccctgcggcagggcgcccacatacctgatatcgcctctctggaggaagtgcccaagatcttcgagggtctcggtcgcgacgagaaacgggcggcgaacgctctgcacggagacagcgaaaacgagagtcactcgggggtgctggtggagctcgagggcgacaacgcgcgcctggccgtactcaagcgcagcatagaggtcacccactttgcctacccggcgctcaacctgccccccaaggtcatgagtgtggtcatgggcgagctcatcatgcgccgcgcccagcccctggccgcggatgcaaacttgcaagagtcctccgaggaaggcctgcccgcggtcagcgacgagcagctggcgcgctggctggagacccgcgaccccgcgcagctggaggagcggcgcaagctcatgatggccgcggtgctggtcaccgtggagctcgagtgtctgcagcgcttcttcgcggaccccgagatgcagcgcaagctcgaggagaccctgcactacaccttccgccagggctacgtgcgccaggcctgcaagatctccaacgtggagctctgcaacctggtctcctacctgggcatcctgcacgagaaccgcctcgggcagaacgtcctgcactccaccctcaaaggggaggcgcgccgcgactacatccgcgactgcgcctacctcttcctctgctacacctggcagacggccatgggggtctggcagcagtgcctggaggagcgcaacctcaaggagctggaaaagctcctcaagcgcaccctcagggacctctggacgggcttcaacgagcgctcggtggccgccgcgctggcggacatcatctttcccgagcgcctgctcaagaccctgcagcagggcctgcccgacttcaccagccagagcatgctgcagaacttcaggactttcatcctggagcgctcgggcatcctgccggccacttgctgcgcgctgcccagcgacttcgtgcccatcaagtacagggagtgcccgccgccgctctggggccactgctacctcttccagctggccaactacctcgcctaccactcggacctcatggaagacgtgagcggcgagggcctgctcgagtgccactgccgctgcaacctctgcacgccccaccgctctctagtctgcaacccgcagctgctcagcgagagtcagattatcggtaccttcgagctgcagggtccctcgcctgacgagaagtccgcggctccagggctgaaactcactccggggctgtggacttccgcctacctacgcaaatttgtacctgaggactaccacgcccacgagatcaggttctacgaagaccaatcccgcccgcccaaggcggagctcaccgcctgcgtcatcacccaggggcacatcctgggccaattgcaagccatcaacaaagcccgccgagagttcttgctgaaaaagggtcggggggtgtacctggacccccagtccggcgaggagctaaacccgctacccccgccgccgccccagcagcgggaccttgcttcccaggatggcacccagaaagaagcagcagccgccgccgccgccgcagccatacatgcttctggaggaagaggaggaggactgggacagtcaggcagaggaggtttcggacgaggagcaggaggagatgatggaagactgggaggaggacagcagcctagacgaggaagcttcagaggccgaagaggtggcagacgcaacaccatcgccctcggtcgcagccccctcgccggggcccctgaaatcctccgaacccagcaccagcgctataacctccgctcctccggcgccggcgccacccgcccgcagacccaaccgtagatgggacaccacaggaaccggggtcggtaagtccaagtgcccgccgccgccaccgcagcagcagcagcagcagcgccagggctaccgctcgtggcgcgggcacaagaacgccatagtcgcctgcttgcaagactgcgggggcaacatctctttcgcccgccgcttcctgctattccaccacggggtcgcctttccccgcaatgtcctgcattactaccgtcatctctacagcccctactgcagcggcgacccagaggcggcagcggcagccacagcggcgaccaccacctaggaagatatcctccgcgggcaagacagcggcagcagcggccaggagacccgcggcagcagcggcgggagcggtgggcgcactgcgcctctcgcccaacgaacccctctcgacccgggagctcagacacaggatcttccccactttgtatgccatcttccaacagagcagaggccaggagcaggagctgaaaataaaaaacagatctctgcgctccctcacccgcagctgtctgtatcacaaaagcgaagatcagcttcggcgcacgctggaggacgcggaggcactcttcagcaaatactgcgcgctcactcttaaagactagctccgcgcccttctcgaatttaggcgggagaaaactacgtcatcgccggccgccgcccagcccgcccagccgagatgagcaaagagattcccacgccatacatgtggagctaccagccgcagatgggactcgcggcgggagcggcccaggactactccacccgcatgaactacatgagcgcgggaccccacatgatctcacaggtcaacgggatccgcgcccagcgaaaccaaatactgctggaacaggcggccatcaccgccacgccccgccataatctcaacccccgaaattggcccgccgccctcgtgtaccaggaaaccccctccgccaccaccgtactacttccgcgtgacgcccaggccgaagtccagatgactaactcaggggcgcagctcgcgggcggctttcgtcacggggcgcggccgctccgaccaggtataagacacctgatgatcagaggccgaggtatccagctcaacgacgagtcggtgagctcttcgctcggtctccgtccggacggaactttccagctcgccggatccggccgctcttcgttcacgccccgccaggcgtacctgactctgcagacctcgtcctcggagccccgctccggcggcatcggaaccctccagttcgtggaggagttcgtgccctcggtctacttcaaccccttctcgggacctcccggacgctaccccgaccagttcattccgaactttgacgcggtgaaggactcggcggacggctacgactgaatgtcaggtgtcgaggcagagcagcttcgcctgagacacctcgagcactgccgccgccacaagtgcttcgcccgcggttctggtgagttctgctactttcagctacccgaggagcataccgaggggccggcgcacggcgtccgcctgaccacccagggcgaggttacctgttccctcatccgggagtttaccctccgtcccctgctagtggagcgggagcggggtccctgtgtcctaactatcgcctgcaactgccctaaccctggattacatcaagatctttgctgtcatctctgtgctgagtttaataaacgctgagatcagaatctactggggctcctgtcgccatcctgtgaacgccaccgtcttcacccaccccgaccaggcccaggcgaacctcacctgcggtctgcatcggagggccaagaagtacctcacctggtacttcaacggcaccccctttgtggtttacaacagcttcgacggggacggagtctccctgaaagaccagctctccggtctcagctactccatccacaagaacaccaccctccaactcttccctccctacctgccgggaacctacgagtgcgtcaccggccgctgcacccacctcacccgcctgatcgtaaaccagagctttccgggaacagataactccctcttccccagaacaggaggtgagctcaggaaactccccggggaccagggcggagacgtaccttcgacccttgtggggttaggattttttattaccgggttgctggctcttttaatcaaagtttccttgagatttgttctttccttctacgtgtatgaacacctcaacctccaataactctaccctttcttcggaatcaggtgacttctctgaaatcgggcttggtgtgctgcttactctgttgatttttttccttatcatactcagccttctgtgcctcaggctcgccgcctgctgcgcacacatctatatctactgctggttgctcaagtgcaggggtcgccacccaagatgaacaggtacatggtcctatcgatcctaggcctgctggccctggcggcctgcagcgccgccaaaaaagagattacctttgaggagcccgcttgcaatgtaactttcaagcccgagggtgaccaatgcaccaccctcgtcaaatgcgttaccaatcatgagaggctgcgcatcgactacaaaaacaaaactggccagtttgcggtctatagtgtgtttacgcccggagacccctctaactactctgtcaccgtcttccagggcggacagtctaagatattcaattacactttccctttttatgagttatgcgatgcggtcatgtacatgtcaaaacagtacaacctgtggcctccctctccccaggcgtgtgtggaaaatactgggtcttactgctgtatggctttcgcaatcactacgctcgctctaatctgcacggtgctatacataaaattcaggcagaggcgaatctttatcgatgaaaagaaaatgccttgatcgctaacaccggctttctatctgcagaatgaatgcaatcacctccctactaatcaccaccaccctccttgcgattgcccatgggttgacacgaatcgaagtgccagtggggtccaatgtcaccatggtgggccccgccggcaattccaccctcatgtgggaaaaatttgtccgcaatcaatgggttcatttctgctctaaccgaatcagtatcaagcccagagccatctgcgatgggcaaaatctaactctgatcaatgtgcaaatgatggatgctgggtactattacgggcagcggggagaaatcattaattactggcgaccccacaaggactacatgctgcatgtagtcgaggcacttcccactaccacccccactaccacctctcccaccaccaccaccactactactactactactactactactactactaccactaccgctgcccgccatacccgcaaaagcaccatgattagcacaaagccccctcgtgctcactcccacgccggcgggcccatcggtgcgacctcagaaaccaccgagctttgcttctgccaatgcactaacgccagcgctcatgaactgttcgacctggagaatgaggatgtccagcagagctccgcttgcctgacccaggaggctgtggagcccgttgccctgaagcagatcggtgattcaataattgactcttcttcttttgccactcccgaataccctcccgattctactttccacatcacgggtaccaaagaccctaacctctctttctacctgatgctgctgctctgtatctctgtggtctcttccgcgctgatgttactggggatgttctgctgcctgatctgccgcagaaagagaaaagctcgctctcagggccaaccactgatgcccttcccctaccccccggattttgcagataacaagatatgagctcgctgctgacactaaccgctttactagcctgcgctctaacccttgtcgcttgcgactcgagattccacaatgtcacagctgtggcaggagaaaatgttactttcaactccacggccgatacccagtggtcgtggagtggctcaggtagctacttaactatctgcaatagctccacttcccccggcatatccccaaccaagtaccaatgcaatgccagcctgttcaccctcatcaacgcttccaccctggacaatggactctatgtaggctatgtaccctttggtgggcaaggaaagacccacgcttacaacctggaagttcgccagcccagaaccactacccaagcttctcccaccaccaccaccaccaccaccatcaccagcagcagcagcagcagcagccacagcagcagcagcagattattgactttggttttggccagctcatctgccgctacccaggccatctacagctctgtgcccgaaaccactcagatccaccgcccagaaacgaccaccgccaccaccctacacacctccagcgatcagatgccgaccaacatcacccccttggctcttcaaatgggacttacaagccccactccaaaaccagtggatgcggccgaggtctccgccctcgtcaatgactgggcggggctgggaatgtggtggttcgccataggcatgatggcgctctgcctgcttctgctctggctcatctgctgcctccaccgcaggcgagccagaccccccatctatagacccatcattgtcctgaaccccgataatgatgggatccatagattggatggcctgaaaaacctacttttttcttttacagtatgataaattgagacatgcctcgcattttcttgtacatgttccttctcccaccttttctggggtgttctacgctggccgctgtgtctcacctggaggtagactgcctctcacccttcactgtctacctgctttacggattggtcaccctcactctcatctgcagcctaatcacagtaatcatcgccttcatccagtgcattgattacatctgtgtgcgcctcgcatacttcagacaccacccgcagtaccgagacaggaacattgcccaacttctaagactgctctaatcatgcataagactgtgatctgccttctgatcctctgcatcctgcccaccctcacctcctgccagtacaccacaaaatctccgcgcaaaagacatgcctcctgccgcttcacccaactgtggaatatacccaaatgctacaacgaaaagagcgagctctccgaagcttggctgtatggggtcatctgtgtcttagttttctgcagcactgtctttgccctcataatctacccctactttgatttgggatggaacgcgatcgatgccatgaattaccccacctttcccgcacccgagataattccactgcgacaagttgtacccgttgtcgttaatcaacgccccccatcccctacgcccactgaaatcagctactttaacctaacaggcggagatgactgacgccctagatctagaaatggacggcatcagtaccgagcagcgtctcctagagaggcgcaggcaggcggctgagcaagagcgcctcaatcaggagctccgagatctcgttaacctgcaccagtgcaaaagaggcatcttttgtctggtaaagcaggccaaagtcacctacgagaagaccggcaacagccaccgcctcagttacaaattgcccacccagcgccagaagctggtgctcatggtgggtgagaatcccatcaccgtcacccagcactcggtagagaccgaggggtgtctgcactccccctgtcggggtccagaagacctctgcaccctggtaaagaccctgtgcggtctcagagatttagtcccctttaactaatcaaacactggaatcaataaaaagaatcacttacttaaaatcagacagcaggtctctgtccagtttattcagcagcacctccttcccctcctcccaactctggtactccaaacgccttctggcggcaaacttcctccacaccctgaagggaatgtcagattcttgctcctgtccctccgcacccactatcttcatgttgttgcagatgaagcgcaccaaaacgtctgacgagagcttcaaccccgtgtacccctatgacacggaaagcggccctccctccgtccctttcctcacccctcccttcgtgtctcccgatggattccaagaaagtccccccggggtcctgtctctgaacctggccgagcccctggtcacttcccacggcatgctcgccctgaaaatgggaagtggcctctccctggacgacgctggcaacctcacctctcaagatatcaccaccgctagccctcccctcaaaaaaaccaagaccaacctcagcctagaaacctcatcccccctaactgtgagcacctcaggcgccctcaccgtagcagccgccgctcccctggcggtggccggcacctccctcaccatgcaatcagaggcccccctgacagtacaggatgcaaaactcaccctggccaccaaaggccccctgaccgtgtctgaaggcaaactggccttgcaaacatcggccccgctgacggccgctgacagcagcaccctcacagtcagtgccacaccaccccttagcacaagcaatggcagcttgggtattgacatgcaagcccccatttacaccaccaatggaaaactaggacttaactttggcgctcccctgcatgtggtagacagcctaaatgcactgactgtagttactggccaaggtcttacgataaacggaacagccctacaaactagagtctcaggtgccctcaactatgacacatcaggaaacctagaattgagagctgcagggggtatgcgagttgatgcaaatggtcaacttatccttgatgtagcttacccatttgatgcacaaaacaatctcagccttaggcttggacagggacccctgtttgttaactctgcccacaacttggatgttaactacaacagaggcctctacctgttcacatctggaaataccaaaaagctagaagttaatatcaaaacagccaagggtctcatttatgatgacactgctatagcaatcaatgcgggtgatgggctacagtttgactcaggctcagatacaaatccattaaaaactaaacttggattaggactggattatgactccagcagagccataattgctaaactgggaactggcctaagctttgacaacacaggtgccatcacagtaggcaacaaaaatgatgacaagcttaccttgtggaccacaccagacccatcccctaactgtagaatctattcagagaaagatgctaaattcacacttgttttgactaaatgcggcagtcaggtgttggccagcgtttctgttttatctgtaaaaggtagccttgcgcccatcagtggcacagtaactagtgctcagattgtcctcagatttgatgaaaatggagttctactaagcaattcttcccttgaccctcaatactggaactacagaaaaggtgaccttacagagggcactgcatataccaacgcagtgggatttatgcccaacctcacagcatacccaaaaacacagagccaaactgctaaaagcaacattgtaagtcaggtttacttgaatggggacaaatccaaacccatgaccctcaccattaccctcaatggaactaatgaaacaggagatgccacagtaagcacttactccatgtcattctcatggaactggaatggaagtaattacattaatgaaacgttccaaaccaactccttcaccttctcctacatcgcccaagaataaaaagcatgacgctgttgatttgattcaatgtgtttctgttttattttcaagcacaacaaaatcattcaagtcattcttccatcttagcttaatagacacagtagcttaatagacccagtagtgcaaagccccattctagcttatagatcagacagtgataattaaccaccaccaccaccataccttttgattcaggaaatcatgatcatcacaggatcctagtcttcaggccgccccctccctcccaagacacagaatacacagtcctctccccccgactggctttaaataacaccatctggttggtcacagacatgttcttaggggttatattccacacggtctcctgccgcgccaggcgctcgtcggtgatgttgataaactctcccggcagctcgctcaagttcacgtcgctgtccagcggctgaacctccggctgacgcgataactgtgcgaccggctgctggacgaacggaggccgcgcctacaagggggtagagtcataatcctcggtcaggatagggcggtgatgcagcagcagcgagcgaaacatctgctgccgccgccgctccgtccggcaggaaaacaacacgccggtggtctcctccgcgataatccgcaccgcccgcagcatcagcttcctcgttctccgcgcgcagcacctcacccttatctcgctcaaatcggcgcagtaggtacagcacagcaccacgatgttattcatgatcccacagtgcagggcgctgtatccaaagctcatgccgggaaccaccgcccccacgtggccatcgtaccacaagcgcacgtaaatcaagtgtcgacccctcatgaacgcgctggacacaaacattacttccttgggcatgttgtaattcaccacctcccggtaccagataaacctctggttgaacagggcaccttccaccaccatcctgaaccaagaggccagaacctgcccaccggctatgcactgcagggaacccgggttggaacaatgacaatgcagactccaaggctcgtaaccgtggatcatccggctgctgaaggcatcgatgttggcacaacacagacacacgtgcatgcactttctcatgattagcagctcttccctcgtcaggatcatatcccaaggaataacccattcttgaatcaacgtaaaacccacacagcagggaaggcctcgcacataactcacgttgtgcatggtcagcgtgttgcattccggaaacagcggatgatcctccagtatcgaggcgcgggtctccttctcacagggaggtaaagggtccctgctgtacggactgcgccgggacgaccgagatcgtgttgagcgtagtgtcatggaaaagggaacgccggacgtggtcatacttcttgaagcagaaccaggttcgcgcgtggcaggcctccttgcgtctgcggtctcgccgtctagctcgctccgtgtgatagttgtagtacagccactcccgcagagcgtcgaggcgcaccctggcttccggatctatgtagactccgtcttgcaccgcggccctgataatatccaccaccgtagaataagcaacacccagccaagcaatacactcgctctgcgagcggcagacaggaggagcgggcagagatgggagaaccatgataaaaaactttttttaaagaatattttccaattcttcgaaagtaagatctatcaagtggcagcgctcccctccactggcgcggtcaaactctacggccaaagcacagacaacggcatttctaagatgttccttaatggcgtccaaaagacacaccgctctcaagttgcagtaaactatgaatgaaaacccatccggctgattttccaatatagacgcgccggcagcgtccaccaaacccagataattttcttctctccagcggtttacgatctgtctaagcaaatcccttatatcaagtccgaccatgccaaaaatctgctcaagagcgccctccaccttcatgtacaagcagcgcatcatgattgcaaaaattcaggttcttcagagacctgtataagattcaaaatgggaacattaacaaaaattcctctgtcgcgcagatcccttcgcagggcaagctgaacataatcagacaggtccgaacggaccagtgaggccaaatccccaccaggaaccagatccagagaccctatactgattatgacgcgcatactcggggctatgctgaccagcgtagcgccgatgtaggcgtgctgcatgggcggcgagataaaatgcaaagtgctggttaaaaaatcaggcaaagcctcgcgcaaaaaagctaacacatcataatcatgctcatgcaggtagttgcaggtaagctcaggaaccaaaacggaataacacacgattttcctctcaaacatgacttcgcggatactgcgtaaaacaaaaaattataaataaaaaattaattaaataacttaaacattggaagcctgtctcacaacaggaaaaaccactttaatcaacataagacgggccacgggcatgccggcatagccgtaaaaaaattggtccccgtgattaacaagtaccacagacagctccccggtcatgtcgggggtcatcatgtgagactctgtatacacgtctggattgtgaacatcagacaaacaaagaaatcgagccacgtagcccggaggtataatcacccgcaggcggaggtacagcaaaacgacccccataggaggaatcacaaaattagtaggagaaaaaaatacataaacaccagaaaaaccctgttgctgaggcaaaatagcgccctcccgatccaaaacaacataaagcgcttccacaggagcagccataacaaagacccgagtcttaccagtaaaagaaaaaagatctctcaacgcagcaccagcaccaacacttcgcagtgtaaaaggccaagtgccgagagagtatatataggaataaaaagtgacgtaaacgggcaaagtccaaaaaacgcccagaaaaaccgcacgcgaacctacgccccgaaacgaaagccaaaaaacactagacactcccttccggcgtcaacttccgctttcccacgctacgtcacttcccccggtcaaacaaactacatatcccgaacttccaagtcgccacgcccaaaacaccgcctacacctccccgcccgccggcccgcccccggacccgcctcccgccccgcgccgcccatctcattatcatattggcttcaatccaaaataaggtatattattgatgatg

SEQ ID NO: 2 - DNA, 36571 bp, unidentified
  source 1..36571: mol_type = unassigned DNA; note = Chimpanzee adenovirus
catcatcaataatatacctcaaacttttggtgcgcgttaatatgcaaatgagctgtttgaatttggggatgcggggcgctgattggctgcgggagcggcgaccgttaggggcggggcgggtgacgttttgatgacgtggccgtgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacgtaggcgtcagctgatcgccagggtatttaaacctgcgctcactagtcaagaggccactcttgagtgccagcgagtagagttttctcctccgcgccgcgagtcagatctacactttgaaagatgaggcacttgagagacctgcccggtaatgttttcctggctactgggaacgagattctggaattggtggtggacgccatgatgggtgacgaccctcccgagccccctaccccatttgaggcgccttcgctgtacgatttgtatgatctggaggtggatgtgcccgagaacgaccccaacgaggaggcggtgaatgatttgtttagcgatgccgcgctgctggctgccgagcaggctaatacggactttggctcagacagcgattcttctctccataccccgagacccggcagaggtgagaaaaagatccccgagcttaaaggggaagagctcgacctgcgctgctatgaggaatgcttgcctccgagcgatgatgaggaggacgaggaggcgattcgagctgcagcgaaccagggagtgaaagctgcgggcgaaagctttagcctggactgtcctactctgcccggacacggctgtaagtcttgtgaatttcatcgcatgaatactggagataagaatgtgatgtgtgccctgtgctatatgagagcttacaaccattgtgtttacagtaagtgtgattaactttagttgggaaggcagagggtgactgggtgctgactggtttatttatgtatatgttttttatgtgtaggtcccgtctctgacgcagatgagacccccacttcagagtgcatttcatcacccccagaaattggcgaggaaccgcccgaagatattattcatagaccagttgcagtgagagtcaccgggcggagagcagctgtggagagtttggatgacttgctacagggtggggatgaacctttggacttgtgtacccggaaacgccccaggcactaagtgccacacatgtgtgtttacttaaggtgatgtcagtatttatagggtgtggagtgcaataaaatccgtgttgactttaagtgcgtggtttatgactcaggggtggggactgtgggtatataagcaggtgcagacctgtgtggtcagttcagagcaggactcatggagatctggacggtcttggaagactttcaccagactagacagctgctagagaactcatcggagggagtctcttacctgtggagattctgcttcggtgggcctctagctaagctagtctatagggccaagcaggattataaggatcaatttgaggatattttgagagagtgtcctggtatttttgactctctcaacttgggccatcagtctcactttaaccagagtattctgagagcccttgacttttccactcctggcagaactaccgccgcggtagccttttttgcctttatccttgacaaatggagtcaagaaacccatttcagcagggattaccgtctggactgcttagcagtagctttgtggagaacatggaggtgccagcgcctgaatgcaatctccggctacttgccagtacagccggtagacacgctgaggatcctgagtctccagtcaccccaggaacaccaacgccgccagcagccgcagcaggagcagcagcaagaggaggaccgagaagagaacccgagagccggtctggaccctccggtggcggaggaggaggagtagctgacttgtttcccgagctgcgccgggtgctgactaggtcttccagtggacgggagagggggattaagcgggagaggcatgaggagactagtcacagaactgaactgactgtcagtctgatgagccgcaggcgcccagaatcggtgtggtggcatgaggtgcagtcgcaggggatagatgaggtctcggtgatgcatgagaaatattccctagaacaagtcaagacttgttggttggagcctgaggatgattgggaggtagccatcaggaattatgccaagctagctctgaagccagacaagaagtacaagattaccaaactgattaatatcagaaattcctgctacatttcagggaatggggccgaggtggagatcagtacccaggagagggtggccttcagatgctgcatgatgaatatgtacccgggggtggtgggcatggagggagtcacctttatgaacgcgaggttcaggggcgatgggtataatggggtggtctttatggccaacaccaagctgacagtgcacggatgctccttctttggcttcaataacatgtgcatcgaggcctggggcagtgtttcagtgaggggatgcagtttttcagccaactggatgggggtcgtgggcagaaccaagagcaaggtgtcagtgaagaaatgcctgttcgagaggtgccacctgggggtgatgagcgagggcgaagccaaagtcaaacactgcgcctctactgagacgggctgctttgtgctgatcaagggcaatgcccaagtcaagcataacatgatctgtggggcctcggatgagcgcggctaccagatgctgacctgcgccggtgggaacagccatatgctggccaccgtgcatgtgacctcgcacccccgcaagacatggcccgagttcgagcacaacgtcatgacccgctgcaatgtgcacctgggctcccgccgaggcatgttcatgccctaccagtgcaacatgcaatttgtgaaggtgctgctggagcccgatgccatgtccagagtgagcctgacgggggtgtttgacatgaatgtggagatgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttgggggaggtggagggcctggatgaggggcagaatgactaaaatctgtgtttttctgcgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcgaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgctgctgctgatggcccagctcgaggccctgacccagcgcctgggcgagctgacccagcaggttgctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgctgcacgatgtccttgaggaggagactgatggccacgggcagccccttggtgtaggtgttgacgaacctgttgagctgggagggatgcatgcggggggagatgagatgcatcttggcctggatcttgagattggcgatgttcccgcccagatcccgccgggggttcatgttgtgcaggaccaccagcacggtgtatccggtgcacttggggaatttgtcatgcaacttggaagggaaggcgtgaaagaatttggagacgcccttgtggccgcccaggttttccatgcactcatccatgatgatggcgatgggcccgtgggcggcggcctgggcaaagacgtttcgggggtcggacacatcgtagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgcgccgaaagcaggttccggagcagctgggacttgccgcagccggtggggccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagcccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccagggtcgcagggtccgcgtcagcgtggtctccgtcacggtgaaggggtgcgcgccgggctgggcgcttgcgagggtgcgcttcaggctcatccggctggtcgagaaccgctcccggtcggcgccctgtgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcggggcggtcggggtcaaaaacgaggtttcctccgtgctttttgatgcgtttcttacctctggtctccatgagctcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagagcagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagatcgtccagcgccgcttgccagtcgcgcacggccagcgcgcgctcgtaggggctgaggggcgtgccccagggcatggggtgcgtgagcgcggaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgtgccgaggttggagcgttgcggcttttcggcgcggtagacgatctggcggaagatggcgtgggagttggaggagatggtgggcctctggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgactttgaggaactggtgcttgaagtcgaggtcgtcgcagccgccctgctcccagagttggaagtccgtgcgcttcttgtaggcggggttgggcaaagcgaaagtaacatcgttgaagaggatcttgcccgcgcggggcatgaagttgcgagtgatgcggaaaggctggggcacctcggcccggttgttgatgacctgggcggcgaggacgatctcgtcgaagccgttgatgttgtgcccgacgatgtagagttccacgaatcgcgggcagcccttgacgtggggcagcttcttgagctcgtcgtaggtgagctcggcggggtcgctgagcccgtgctgctcgagggcccagtcggcgacgtgggggttggcgctgaggaaggaagtccagagatccacggccagggcggtctgcaagcggtcccggtactgacggaactgctggcccacggccattttttcgggggtgacgcagtagaaggtgcgggggtcgccgtgccagcggtcccacttgagttggagggcgaggtcgtgggcgagctcgacgagcggcgggtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccagttggaggaatggctgttgatgtgatggaagtagaaatgccgacggcgcgccgagcactcgtgcttgtgtttatacaagcgtccgcagtgctcgcaacgctgcacgggatgcacgtgctgcacgagctgtacctgggttcctttgacgaggaatttcagtgggcagtggagcgctggcggctgcatctggtgctgtactacgtcctggccatcggcgtggccatcgtctgcctcgatggtggtcatgctgacgagcccgcgcgggaggcaggtccagacctcggctcggacgggtcggagagcgaggacgagggcgcgcaggccggagctgtccagggtcctgagacgctgcggagtcaggtcagtgggcagcggcggcgcgcggttgacttgcaggagcttttccagggcgcgcgggaggtccagatggtacttgatctccacggcgccgttggtggcgacgtccacggcttgcagggtcccgtgcccctggggcgccaccaccgtgccccgtttcttcttgggcggcggcggctccatgcttagaagcggcggcgaggacgcgcgccgggcggcaggggcggctcggggcccggaggcaggggcggcaggggcacgtcggcgccgcgcgcgggcaggttctggtactgcgcccggagaagactggcgtgagcgacgacgcgacggttgacgtcctggatctgacgcctctgggtgaaggccacgggacccgtgagtttgaacctgaaagagagttcgacagaatcaatttcggtatcgttgacggcggcctgccgcaggatctcttgcacgtcgcccgagttgtcctggtaggcgatctcggtcatgaactgctcgatctcctcctcctgaaggtctccgcggccggcgcgctcgacggtggccgcgaggtcgttggagatgcggcccatgagctgcgagaaggcgttcatgccggcctcgttccagacgcggctgtagaccacggctccgttggggtcgcgcgcgcgcatgaccacctgggcgaggttaagctcgacgtggcgcgtgaagaccgcgtagttgcagaggcgctggtagaggtagttgagcgtggtggcgatgtgctcggtgacgaagaagtacatgatccagcggcggagcggcatctcgctgacgtcgcccagggcttccaagcgctccatggtctcgtagaagtccacggcgaagttgaaaaactgggagttgcgcgccgagacggtcaactcctcctccagaagacggatgagctcggcgatggtggcgcgcacctcgcgctcgaaggccccggggggctcctcttcttccatctcctcctcctcttcctcctccactaacatctcttctacttcctcctcaggaggcggcggcgggggaggggccctgcgtcgccggcggcgcacgggcagacggtcgatgaagcgctcgatggtctccccgcgccggcgacgcatggtctcggtgacggcgcgcccgtcctcgcggggccgcagcgtgaagacgccgccgcgcatctccaggtggccgccgggggggtctccgttgggcagggagagggcgctgacgatgcatcttatcaattggcccgtagggactccgcgcaaggacctgagcgtctcgagatccacgggatccgaaaaccgctgaacgaaggcttcgagccagtcgcagtcgcaaggtaggctgagcccggtttcttgttcttcgggtatttggtcgggaggcgggcgggcgatgctgctggtgatgaagttgaagtaggcggtcctgagacggcggatggtggcgaggagcaccaggtccttgggcccggcttgctggatgcgcagacggtcggccatgccccaggcgtggtcctgacacctggcgaggtccttgtagtagtcctgcatgagccgctctacgggcacgtcctcctcgcccgcgcggccgtgcatgcgcgtgagcccgaacccgcgctgcggctggacgagcgccaggtcggcgacgacgcgctcggcgaggatggcctgctggatctgggtgagggtggtctggaagtcgtcgaagtcgacgaagcggtggtaggctccggtgttgatggtgtaggagcagttggccatgacggaccagttgacggtctggtggccggggcgcacgagctcgtggtacttgaggcgcgagtaggcgcgcgtgtcgaagatgtagtcgttgcaggtgcgcacgaggtactggtatccgacgaggaagtgcggcggcggctggcggtagagcggccatcgctcggtggcgggggcgccgggcgcgaggtcctcgagcatgaggcggtggtagccgtagatgtacctggacatccaggtgatgccggcggcggtggtggaggcgcgcgggaactcgcggacgcggttccagatgttgcgcagcggcaggaagtagttcatggtggccgcggtctggcccgtgaggcgcgcgcagtcgtggatgctctagacatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgctaacgaaacctccaggatacggaggcgggtcgttttttggccttggtcgctggtcatgaaaaactagtaagcgcggaaagcggccgcccgcgatggctcgctgccgtagtctggagaaagaatcgccagggttgcgttgcggtgtgccccggttcgagcctcagcgctcggtgccggccggattccgcggctaacgtgggcgtggctgccccgtcgtttccaagaccccttagccagccgacttctccagttacggagcgagcccctctttttcttgtgtttttgccagatgcatcccgtactgcggcagatgcgcccccaccctccaccacaaccgcccctaccgcagcagcagcaacagccggcgcttctgcccccgccccagcagcagcagccagccactaccgcggcggccgccgtgagcggagccggcgttcagtatgacctggccttggaagagggcgaggggctggcgcggctgggggcgtcgtcgccggagcggcacccgcgcgtgcagatgaaaagggacgctcgcgaggcctacgtgcccaagcagaacctgttcagagacaggagcggcgaggagcccgaggagatgcgcgcctcccgcttccacgcggggcgggagctgcggcgcggcctggaccgaaagcgggtgctgagggacgaggatttcgaggcggacgagctgacggggatcagccccgcgcgcgcgcacgtggccgcggccaacctggtcacggcgtacgagcagaccgtgaaggaggagagcaactttcaaaaatccttcaacaaccacgtgcgcacgctgatcgcgcgcgaggaggtgaccctgggcctgatgcacctgtgggacctgctggaggccatcgtgcagaaccccacgagcaagccgctgacggcgcagctgtttctggtggtgcagcacagtcgggacaacgagacgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagcctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagccgccggcgcgagctgagcgaccaggagctgatgcacagcctgcagcgggccctgaccggggccgggaccgagggggagagctactttgacatgggcgcggacctgcgctggcagcccagccgccgggccttggaagctgccggcggcgtgccctacgtggaggaggtggacgatgaggaggaggagggcgagtacctggaagactgatggcgcgaccgtatttttgctagatgcagcaacagccaccgccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagacgaacctggaccgcatggtgaccgacgtgcgcgaggcggtgtcgcagcgcgagcggttccaccgcgagtcgaacctgggctccatggtggcgctgaacgccttcctgagcacgcagcccgccaacgtgccccggggccaggaggactacaccaacttcatcagcgcgctgcggctgatggtggccgaggtgccccagagcgaggtgtaccagtcggggccggactacttcttccagaccagtcgccagggcttgcagaccgtgaacctgagccaggctttcaagaacttgcagggactgtggggcgtgcaggccccggtcggggaccgcgcgacggtgtcgagcctgctgacgccgaactcgcgcctgctgctgctgctggtggcgcccttcacggacagcggcagcgtgagccgcgactcgtacctgggctacctgcttaacctgtaccgcgaggccatcgggcaggcgcacgtggacgagcagacctaccaggagatcacccacgtgagccgcgcgctgggccaggaggacccgggcaacctggaggccaccctgaacttcctgctgaccaaccggtcgcagaagatcccgccccagtacgcgctgagcaccgaggaggagcgcatcctgcgctacgtgcagcagagcgtggggctgttcctgatgcaggagggggccacgcccagcgccgcgctcgacatgaccgcgcgcaacatggagcccagcatgtacgcccgcaaccgcccgttcatcaataagctgatggactacttgcatcgggcggccgccatgaactcggactactttaccaacgccatcttgaacccgcactggctcccgccgcccgggttctacacgggcgagtacgacatgcccgaccccaacgacgggttcctgtgggatgacgtggacagcagcgtgttctcgccgcgtcccaccaccaccgtgtggaagaaagagggcggggaccggcggccgtcctcggcgctgtccggtcgcgcgggtgctgccgcggcggtgcccgaggccgccagcccctttccgagcctgcccttttcgctgaacagcgtgcgcagcagcgagctgggtcggctgacgcggccgcgcctgctgggcgaggaggagtacctgaacgactccttgttgaggcccgagcgcgaaaagaacttccccaataacgggatagagagcctggtggacaagatgagccgctggaagacgtacgcgcacgagcacagggacgagccccgagctagcagcgcaggcacccgtagacgccagcggcacgacaggcagcggggtctggtgtgggacgatgaggattccgccgacgacagcagcgtgttggacttgggtgggagtggtggtggtaacccgttcgctcacttgcgcccccgtatcgggcgcctgatgtaagaatctgaaaaataaaaaacggtactcaccaaggccatggcgaccagcgtgcgttcttctctgttgtttgtagtagtatgatgaggcgcgtgtacccggagggtcctcctccctcgtacgagagcgtgatgcagcaggcggtggcggcggcgatgcagcccccgctggaggcgccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaacgatttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcgcgcaagacccccaacggggtcacagtaacagatggtagtcaggacgagctgacctacgagtgggtggagtttgagctgcccgagggcaacttctcggtgaccatgaccatcgatctgatgaacaacgccatcatcgacaactacttggcggtggggcggcagaacggggtgctggagagcgacatcggcgtgaagttcgacacgcgcaacttccggctgggctgggaccccgtgaccgagctggtgatgccgggcgtgtacaccaacgaggccttccaccccgacatcgtcctgctgcccggctgcggcgtggacttcaccgagagccgcctcagcaacctgctgggcatccgcaagcggcagcccttccaggagggcttccagatcctgtacgaggacctggaggggggcaacatccccgcgctcttggatgtcgaagcctacgagaaaagcaaggaggatagcaccgccgtggctaccgccgcgactgtggcagatgccactgtcaccaggggcgatacattcgccacccaggcggaggaagcagccgccctagcggcgaccgatgatagtgaaagtaagatagttatcaagccggtggagaaggacagcaaggacaggagctacaacgttctatcggatggaaagaacaccgcctaccgcagctggtacctggcctacaactacggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgtcactgacgccagacgccgcacctgcccctacgtctacaaggccctgggcgtagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggtcgcgtgcgctcgcgcaccaccgtcgacgacgtgatcgaccaggtggtggccgacgcgcgcaactacacgcccgccgccgcgcccgcctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcctccggcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggcggcggtgaaggaggaaagaaagccccgcaaactgaagcgggtcaaaaaggacaaaaaggaggaggaagatgtggacggactggtggagtttgtgcgcgagttcgccccccggcggcgcgtgcagtggcgcgggcggaaagtgaaaccggtgctgcggcccggcaccacggtggtcttcacgcccggcgagcgttccggctccgcctccaagcgctcctacgacgaggtgtacggggacgaggacatcctcgagcaggcggccgagcgtctgggcgagtttgcttacggcaagcgcagccgccccgcgcccttgaaagaggaggcggtgtccatcccgctggaccacggcaaccccacgccgagcctgaagccggtgaccctgcagcaggtgctgccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccggcgccggcttccaccaccactcgccgaagacgcaagtacggcgcggccagcctgctgatgcccaactacgcgctgcatccttccatcatccccacgccgggctaccgcggcacgcgcttctaccgcggctacagcagccgccgcaagaccaccacccgccgccgccgtcgccgcacccgccgcagcaccaccgcgacttccgccgccgccttggtgcggagagtgtaccgcagcgggcgtgagcctctgaccctgccgcgcgcgcgctaccacccgagcatcgccatttaactctgccgtcgcctccttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacagcttggaaaatttgtaataaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagcagccagcccgcgaccctggacttgcctccacctcgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtatatgtatgtccgccgaccagaaggaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcacccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccccagcttcaaaccctactccggcaccgcctacaacagcctggctcccaagggagcgcccaacacctcacagtggataaccaaagacaatggaactgataagacatacagttttggaaatgctccagtcagaggattggacattacagaagagggtctccaaataggaaccgatgagtcagggggtgaaagcaagaaaatttttgcagacaaaacctatcagcctgaacctcagcttggagatgaggaatggcatgatactattggagctgaagacaagtatggaggcagagcgcttaaacctgccaccaacatgaaaccctgctatgggtctttcgccaagccaactaatgctaagggaggtcaggctaaaagcagaaccaaggacgatggcactactgagcctgatattgacatggccttctttgacgatcgcagtcagcaagctagtttcagtccagaacttgttttgtatactgagaatgtcgatctggacaccccggatacccacattatttacaaacctggcactgatgaaacaagttcttctttcaacttgggtcagcagtccatgcccaacagacccaactacattggcttcagagacaactttatcgggctcatgtactacaacagcactggcaatatgggtgtactggccggtcaggcctcccagctgaatgctgtggtggacttgcaggacagaaacactgaactgtcctaccagctcttgcttgactctctgggtgacagaaccaggtatttcagtatgtggaatcaggcggtggacagctatgaccccgatgtgcgcattattgaaaatcacggtgtggaggatgaactccccaactattgcttccctttgaatggtgtgggctttacagatacattccagggaattaaggttaaaactacaaataacggaacagcaaatgctacagagtgggaatctgatacctctgtcaataatgctaatgagattgccaagggcaatcctttcgccatggagatcaacatccaggccaacctgtggcggaacttcctctacgcgaacgtggcgctgtacctgcccgactcctacaagtacacgccggccaacatcacgctgcccaccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagaggggtacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacgcccttctcggccggcaacgccaccacctaagcctcttgcttcttgcaagatgacggcctgtggctccggcgagcaggagctcagggccatcctccgcgacctgggctgcgggccctacttcctgggcaccttcgacaagcgcttcccgggattcatggccccgcacaagctggcctgcgccatcgtcaacacggccggccgcgagaccgggggcgagcactggctggccttcgcctggaacccgcgcacccacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgcctactttcgctcccaccgcgcgcgcatcgagaaggccaccgccttcgaccgcatgaatcaagacatgtaaactgtgtgtatgtgaatgctttattcataataaacagcacatgtttatgccaccttctctgaggctctgactttatttagaaatcgaaggggttctgccggctctcggcgtgccccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggatatcttgaaatcacagttgggacccgcgttctgcgcgcgagagttgcggtacacggggttgcagcactggaacaccatcagggccgggtgcttcacgctcgccagcaccgtcgcgtcggtgatgccctccacgtccagatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcaccgcagcttgccctcggcttcggtgcatccgtgcagccacagcgcgcagccggtgcactcccagttcttgtgggcgatctgggagtgcgagtgcacgaagccctgcaggaagcggcccatcatcgcggtcagggtcttgttgctggtgaaggtcagcgggatgccgcggtgctcctcgttcacatacaggtggcagatgcggcggtacacctcgccctgctcgggcatcagctggaaggcggacttcaggtcgctctccacgcggtaccgctccatcagcagcgtcatgacttccatgcccttctcccaggccgaaacgatcggcaggctcagggggttcttcaccgttgtcatcttagtcgccgccgccgaggtcagggggtcgttctcgtccagggtctcaaacactcgcttgccgtccttctcggtgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttcttcttggccgtcgtccgagaccacgcggcggtaggcatgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaagcatggagactcagccatcgtcgccaacatcgccatctgcccccgccgccgccgacgagaaccagcagcagcagaatgaaagcttaaccgccccgccgcccagccccacctccgacgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagagccaggctgggctcgagcatggcgactacctgagcggggcagaggacgtgctcatcaagcatctggcccgccaatgcatcatcgtcaaggatgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcgcctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtcctcaagcgcacggtcgagctgacccacttcgcctacccggcgctcaacctgccccccaaggtcatgagcgccgtcatggaccaggtgctcatcaagcgcgcctcgcccctctcggaggaggagatgcaggaccccgagagctcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctgaaggccctgtggaccgggttcgacgagcgcaccaccgcctcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcgctgccctcggacttcgtgccgctgaccttccgcgagtgccccccgccgctctggagccactgctacctgctgcgtctggccaactacctggcctaccactcggacgtgatcgaggacgtcagcggcgagggtctgctcgagtgccactgccgctgcaacctctgcacgccgcaccgctccctggcctgcaacccccagctgctgagcgagacccagatcatcggcaccttcgagttgcaaggccccggcgaggagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacttggacccccagaccggagaggagctcaaccccagcttcccccaggatgcccagaggaagcagcaagaagctgaaagtggagctgccgctgccgccggaggatttggaggaagactgggagagcagtcaggcagaggaggaggagatggaagactgggacagcactcaggcagaggaggacagcctgcaagacagtctggaagacgaggtggaggaggaggcagaggaagaagcagccgccgccagaccgtcgtcctcggcggagaaagcaagcagcacggataccatctccgctccgggtcggggtctcggcggccgggcccacagtaggtgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagaaaaccagcagcagctagaaaatccacagcggcggcggcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccgccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcggctgacctagctcggcttcgacacctggaccactgccgccgcttccgctgcttcgctcgggatctcgccgagtttgcctactttgagctgcccgaggagcaccctcagggcccggcccacggagtgcggatcatcgtcgaagggggcctcgactcccacctgcttcggatcttcagccagcgtccgatcctggtcgagcgcgagcaaggacagacccgtctgaccctgtactgcatctgcaaccaccccggcctgcatgaaagtctttgttgtctgctgtgtactgagtataataaaagctgagatcagcgactactccggacttccgtgtgttcctgaatccatcaaccagtccctgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgtcgctccccgctactaacaaccaaactacccaccaacgccaccgtcgcgacctttcctctgaatctaataccactaccggaggtgagctccgaggtcgaccaacctctgggatttactacggcccctgggaggtggtggggttaatagcgctaggcctagttgtgggtgggcttttggctctctgctacctatacctcccttgctgttcgtacttagtggtgctgtgttgctggtttaagaaatggggcagatcaccctagtgagctgcggtgtgctggtggcggtggtgctttcgattgtgggactgggcggcgcggctgtagtgaaggagaaggccgatccctgcttgcatttcaatcccgacaaatgccagctgagttttcagcccgatggcaatcggtgcgcggtgctgatcaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaataatactttcatttttgcgcacatgtgcgacacggtcatgtggatgagcaagcagtacgatatgtggccccccacgaaggagaacatcgtggtcttctccatcgcttacagcctgtgcacggtgctaatcaccgctatcgtgtgcctgagcattcacatgctcatcgctattcgccccagaaataatgccgaaaaagagaaacagccataacacgttttttcacacaccttgtttttacagacaatgcgtctgttaaattttttaaacattgtgctcagtattgcttatgcctctggctatgcaaacatacagaaaaccctctatgtaggatctgatgatacactagagggtacccaatcacaagctagggtttcatggtatttttataaaagctcagataatcctattactctttgcaaaggtgatcaggggcggacaacaaagccgcctatcacatttagctgtaccagaacaaatctcacgcttttctcaattacaaaacaatatgctggtatttattacagtacaaactttcatagtgggcaagataaatattatactgttaaggtagaaaatcctaccactcctagaactaccaccaccaccaccaccaccaccactactgcgaagcccactaaacctaaaactaccaagaaaaccactgtgaaaactacaactagaaccaccacaactacagaaaccaccaccagcacaacacttgctgcaactacacacacacacactgagctaaccttacagaccactaatgatttgatagccctgttgcaaaagggggataacagcaccacttccaatgaggagatacccaaatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggccttttagttttttctatcattacctctgctctatgcaattctgacaatgaggacgttactgtcgttgtcggatcaaattatacactgaaaggtccagcgaagggtatgctttcgtggtattgctggtttggaactgacactgatcaaactgagctttgcaatgcaatgaaaggtcaaataccaacctcaaaaattaaacataaatgcaatggtactgacttagtactactcaatatcacgaaatcatatgctggcagctattcatgccctggagatgatgctgagaacatgattttttacaaagtaactgttgttgatcccactactccaccacccaccaccacaactactcacaccacacacacagaacaaacaccagaggcagcagaagcagagttggccttccaggttcacggagattcctttgctgtcaatacccctacacccgatcatcggtgtccggggctgctagtcagcggcattgtcggtgtgctttcgggattagcagtcataatcatctgcatgttcatttttgcttgctgctatagaaggctttaccgacaaaaatcagacccactgctgaacctctatgtttaattttttccagagccatgaaggcagttagcgctctagttttttgttctttgattggcattgttttttgcaatcctattactagagttagctttattaaagatgtgaatgttactgaggggggcaatgtgacactggtaggtgtagagggtgctaaaaacaccacctggacaaaataccaccttgggtggaaagatatttgcaattggagtgtcactgtgtacacatgtgagggagttaatcttaccattgtcaatgccacctcagctcaaaatggtagaattcaaggacaaagtgttagtgtgaccagtgatgggtattttacccaacatacttttatctatgacgttaaagtcataccactgcctacgcctagcccacctagcaccactacacaaacaacccacactacacagacaaccacatacagtacatcaaatcagcctaccaccactacagcagcagaggttgccagctcgtctggagttcaagtggcatttttgttgttgcccccatctagcagtcccactgctattaccaatgagcagactactgcatttttgtccactgtcgagagccacaccacagctacctccagtgccttctctagcaccgccaatctctcctcgctttcctctacaccaatcagtcccgctactactactacccccgctattcttcccactcccctgaagcaaacagacggcggcatgcaatggcagatcaccctgctcattgtgatcgggttggtcatcctagccgtgttgctctactacatcttctgccgccgcattcccaacgcgcaccgcaagccggtctacaagcccatcattgtcgggcagccggagccgcttcaggtggaagggggtctaaggaatcttctcttctcttttacagtatggtgattgaactatgattcctagacaattcttgatcactattcttatctgcctcctccaagtctgtgccaccctcgctctggtggccaacgccagtccagactgtattgggcccttcgcctcctacgtgctctttgccttcatcacctgcatctgctgctgtagcatagtctgcctgcttatcaccttcttccagttcattgactggatctttgtgcgcatcgcctacctgcgccaccacccccagtaccgcgaccagcgagtggcgcagctgctcaggctcctctgataagcatgcgggctctgctacttctcgcgcttctgctgttagtgctcccccgtcccgttgacccccggccccccactcagtcccccgaggaggtccgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcaacctcaggcacacgcactaccaccaccacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggacggcatagccatccaccagtgcaagaaaggcatcttctgcctggtgaaacaggccaagatctcctacgaggtcacccagaccgaccatcgcctctcctacgagctcctgcagcagcgccagaagttcacctgcctggtcggagtcaaccccatcgtcatcacccagcagtcgggcgataccaaggggtgcatccactgctcctgcgactcccccgactgcgtccacactctgatcaagaccctctgcggcctccgcgacctcctccccatgaactaatcacccacttatccagtgaaataaaaaaataatcatttgatttgaaataaagatacaatcatattgatgatttgagtttaacaaaaataaagaatcacttacttgaaatctgataccaggtctctgtccatattttctgccaacaccacctcactcccctcttcccagctctggtactgcaggccccggcgggctgcaaacttcctccacacgctgaaggggatgtcaaattcctcctgcccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgactcctcgggaaaactcatctccaacacggccaccaaggccgctgcccctctcagtttttccaacaacaccatttcccttaacatggatcaccccttttacactaaagatggaaaattagccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgtcgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatatgtcaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggagctaactcttataccttctcctacatcgcccaagaatgaatactgtatcccaccctgcatgcccaaccctcccccacctctgtctatatggaaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacaggaccaccaggttgttcaacagtccatagttcaacacgctccagccgaaactcatcgcgggaaggatgctacccacgtggccgtcgtaccagatcctcaggtaaatcaagtggcgccccctccagaacacgctgcccatgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcacacgctcatgcatctcttcagcactctcagctcctcgggggtcaaaaccatatcccagggcacgggaaactcttgcaggacagcgaagcccgcagaacagggcaatcctcgcacataacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgaagcagaacctggtccgggcgctgcacaccgatcgccggcggcggtctcggcgcttggaacgctcggtgttgaagttgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagcacttcaaaatgaaggtcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcgggatatcaaaatctctgccgcgatccctgagctcctccctcagcaataactgtaagtactctttcatatcctctccgaaatttttagccataggacccccaggaataagagaagggcaagccacattacagataaaccgaagtcccccccagtgagcattgccaaatgtaagattgaaataagcatgctggctagacccggtgatatcttccagataactggacagaaaatcgggcaagcaatttttaagaaaatcaacaaaagaaaaatcttccaggtgcacgtttagggcctcgggaacaacgatggagtaagtgcaaggggtgcgttccagcatggttagttagctgatctgtaaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgagaagaagcatacacccccggaacattggagtccgtgagtgaaaaaaagcggccgaggaagcaatgaggcactacaacgctcactctcaagtccagcaaagcgatgccatgcggatgaagcacaaaattttcaggtgcgtaaaaaatgtaattactcccctcctgcacaggcagcgaagctcccgatccctccagatacacatacaaagcctcagcgtccatagcttaccgagcggcagcagcagcggcacacaacaggcgcaagagtcagagaaaagactgagctctaacctgtccgcccgctctctgctcaatatatagccccagatctacactgacgtaaaggccaaagtctaaaaatacccgccaaataatcacacacgcccagcacacgcccagaaaccggtgacacactcaaaaaaatacgcgcacttcctcaaacgcccaaactgccgtcatttccgggttcccacgctacgtcatcaaaacacgactttcaaattccgtcgaccgttaaaaacgtcacccgccccgcccctaacggtcgccgctcccgcagccaatcagcgccccgcatccccaaattcaaacagctcatttgcatattaacgcgcaccaaaagtttgaggtatattattgatgatg

SEQ ID NO: 3 - DNA, 1109 bp, synthetic construct
  misc_feature 1..1109: note = Description of Artificial Sequence: Synthetic polynucleotide
  source 1..1109: mol_type = other DNA
ggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgctccctatcagtgatagagatctccctatcagtgatagagatcgtcgacgagctcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 4 - DNA, 37559 bp, synthetic construct
  misc_feature 1..37559: note = Description of Artificial Sequence: Synthetic polynucleotide
  source 1..37559: mol_type = other DNA
catcatcaataatataccttattttggattgaagccaatatgataatgagatgggcggcgcggggcggggcgcggggcgggaggcgggtttgggggcgggccggcgggcggggcggtgtggcggaagtggactttgtaagtgtggcggatgtgacttgctagtgccgggcgcggtaaaagtgacgttttccgtgcgcgacaacgcccccgggaagtgacatttttcccgcggtttttaccggatgttgtagtgaatttgggcgtaaccaagtaagatttggccattttcgcgggaaaactgaaacggggaagtgaaatctgattaattttgcgttagtcataccgcgtaatatttgtctagggccgagggactttggccgattacgtggaggactcgcccaggtgttttttgaggtgaatttccgcgttccgggtcaaagtctgcgttttattattataggatatcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgagatcttccgtttatctaggtaccagatatcgccaccatggaactgctgatcctgaaggccaacgccatcaccaccatcctgaccgccgtgaccttctgcttcgccagcggccagaacatcaccgaggaattctaccagagcacctgtagcgccgtgagcaagggctacctgagcgccctgagaaccggctggtacaccagcgtgatcaccatcgagctgagcaacatcaaagaaaacaagtgcaacggcaccgacgccaaagtgaagctgatcaagcaggaactggacaagtacaagaacgccgtgaccgagctgcagctgctgatgcagagcacccccgccaccaacaaccgggccagacgggagctgccccggttcatgaactacaccctgaacaacgccaaaaagaccaacgtgaccctgagcaagaagcggaagcggcggttcctgggctttctgctgggcgtgggcagcgccattgccagcggcgtggccgtgtctaaggtgctgcacctggaaggcgaagtgaacaagatcaagagcgccctgctgagcaccaacaaggccgtggtgtccctgagcaacggcgtgagcgtgctgaccagcaaggtgctggatctgaagaactacatcgacaagcagctgctgcccatcgtgaacaagcagagctgcagcatcagcaacatcgagacagtgatcgagttccagcagaagaacaaccggctgctggaaatcacccgggagttcagcgtgaacgccggcgtgaccacccctgtgtccacctacatgctgaccaacagcgagctgctgagcctgatcaacgacatgcccatcaccaacgaccagaaaaagctgatgagcaacaacgtgcagatcgtgcggcagcagagctactccatcatgtccatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgacagaggctggtactgcgacaacgccggcagcgtgtcattctttccacaggccgagacatgcaaggtgcagagcaaccgggtgttctgcgacaccatgaacagcctgaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgacgagttcgacgccagcatcagccaggtgaacgagaagatcaaccagagcctggccttcatccggaagtccgacgagctgctgcacaatgtgaatgccggcaagtccaccaccaaccggaagcggagagcccctgtgaagcagaccctgaacttcgacctgctgaagctggccggcgacgtggagagcaatcccggccctatggccctgagcaaagtgaaactgaacgatacactgaacaaggaccagctgctgtccagcagcaagtacaccatccagcggagcaccggcgacagcatcgatacccccaactacgacgtgcagaagcacatcaacaagctgtgcggcatgctgctgatcacagaggacgccaaccacaagttcaccggcctgatcggcatgctgtacgccatgagccggctgggccgggaggacaccatcaagatcctgcgggacgccggctaccacgtgaaggccaatggcgtggacgtgaccacacaccggcaggacatcaacggcaaagaaatgaagttcgaggtgctgaccctggccagcctgaccaccgagatccagatcaatatcgagatcgagagccggaagtcctacaagaaaatgctgaaagaaatgggcgaggtggcccccgagtacagacacgacagccccgactgcggcatgatcatcctgtgtatcgccgccctggtgatcacaaagctggccgctggcgacagatctggcctgacagccgtgatcagacgggccaacaatgtgctgaagaacgagatgaagcggtacaagggcctgctgcccaaggacattgccaacagcttctacgaggtgttcgagaagtacccccacttcatcgacgtgttcgtgcacttcggcattgcccagagcagcaccagaggcggctccagagtggagggcatcttcgccggcctgttcatgaacgcctacggcgctggccaggtgatgctgagatggggcgtgctggccaagagcgtgaagaacatcatgctgggccacgccagcgtgcaggccgagatggaacaggtggtggaggtgtacgagtacgcccagaagctgggcggagaggccggcttctaccacatcctgaacaaccctaaggcctccctgctgtccctgacccagttcccccacttctccagcgtggtgctgggaaatgccgccggactgggcatcatgggcgagtaccggggcacccccagaaaccaggacctgtacgacgccgccaaggcctacgccgagcagctgaaagaaaacggcgtgatcaactacagcgtgctggacctgaccgctgaggaactggaagccatcaagcaccagctgaaccccaaggacaacgacgtggagctgggaggcggaggatctggcggcggaggcatgagcagacggaacccctgcaagttcgagatccggggccactgcctgaacggcaagcggtgccacttcagccacaactacttcgagtggccccctcatgctctgctggtgcggcagaacttcatgctgaaccggatcctgaagtccatggacaagagcatcgacaccctgagcgagatcagcggagccgccgagctggacagaaccgaggaatatgccctgggcgtggtgggagtgctggaaagctacatcggctccatcaacaacatcacaaagcagagcgcctgcgtggccatgagcaagctgctgacagagctgaacagcgacgacatcaagaagctgagggacaacgaggaactgaacagccccaagatccgggtgtacaacaccgtgatcagctacattgagagcaaccgcaagaacaacaagcagaccatccatctgctgaagcggctgcccgccgacgtgctgaaaaagaccatcaagaacaccctggacatccacaagtccatcaccatcaacaatcccaaagaaagcaccgtgtctgacaccaacgatcacgccaagaacaacgacaccacctgatgagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgatcagcgatcgctgaggtgggtgagtgggcgtggcctggggtggtcatgaaaatatataagttgggggtcttagggtctctttatttgtgttgcagagaccgccggagccatgagcgggagcagcagcagcagcagtagcagcagcgccttggatggcagcatcgtgagcccttatttgacgacgcggatgccccactgggccggggtgcgtcagaatgtgatgggctccagcatcgacggccgacccgtcctgcccgcaaattccgccacgctgacctatgcgaccgtcgcggggacgccgttggacgccaccgccgccgccgccgccaccgcagccgcctcggccgtgcgcagcctggccacggactttgcattcctgggaccactggcgacaggggctacttctcgggccgctgctgccgccgttcgcgatgacaagctgaccgccctgctggcgcagttggatgcgcttactcgggaactgggtgacctttctcagcaggtcatggccctgcgccagcaggtctcctccctgcaagctggcgggaatgcttctcccacaaatgccgtttaagataaataaaaccagactctgtttggattaaagaaaagtagcaagtgcattgctctctttatttcataattttccgcgcgcgataggccctagaccagcgttctcggtcgttgagggtgcggtgtatcttctccaggacgtggtagaggtggctctggacgttgagatacatgggcatgagcccgtcccgggggtggaggtagcaccactgcagagcttcatgctccggggtggtgttgtagatgatccagtcgtagcaggagcgctgggcatggtgcctaaaaatgtccttcagcagcaggccgatggccagggggaggcccttggtgtaagtgtttacaaaacggttaagttgggaagggtgcattcggggagagatgatgtgcatcttggactgtatttttagattggcgatgtttccgcccagatcccttctgggattcatgttgtgcaggaccaccagtacagtgtatccggtgcacttggggaatttgtcatgcagcttagagggaaaagcgtggaagaacttggagacgcctttgtggcctcccagattttccatgcattcgtccatgatgatggcaatgggcccgcgggaggcagcttgggcaaagatatttctggggtcgctgacgtcgtagttgtgttccagggtgaggtcgtcataggccatttttacaaagcgcgggcggagggtgcccgactgggggatgatggtcccctctggccctggggcgtagttgccctcgcagatctgcatttcccaggccttaatctcggaggggggaatcatatccacctgcggggcgatgaagaaaacggtttccggagccggggagattaactgggatgagagcaggtttctaagcagctgtgattttccacaaccggtgggcccataaataacacctataaccggttgcagctggtagtttagagagctgcagctgccgtcgtcccggaggaggggggccacctcgttgagcatgtccctgacgcgcatgttctccccgaccagatccgccagaaggcgctcgccgcccagggacagcagctcttgcaaggaagcaaagtttttcagcggcttgaggccgtccgccgtgggcatgtttttcagggtctggctcagcagctccaggcggtcccagagctcggtgacgtgctctacggcatctctatccagcatatctcctcgtttcgcgggttggggcgactttcgctgtagggcaccaagcggtggtcgtccagcggggccagagtcatgtccttccatgggcgcagggtcctcgtcagggtggtctgggtcacggtgaaggggtgcgctccgggctgagcgcttgccaaggtgcgcttgaggctggttctgctggtgctgaagcgctgccggtcttcgccctgcgcgtcggccaggtagcatttgaccatggtgtcatagtccagcccctccgcggcgtgtcccttggcgcgcagcttgcccttggaggtggcgccgcacgaggggcagagcaggctcttgagcgcgtagagcttgggggcgaggaagaccgattcgggggagtaggcgtccgcgccgcagaccccgcacacggtctcgcactccaccagccaggtgagctcggggcgcgccgggtcaaaaaccaggtttcccccatgctttttgatgcgtttcttacctcgggtctccatgaggtggtgtccccgctcggtgacgaagaggctgtccgtgtctccgtagaccgacttgaggggtcttttctccaggggggtccctcggtcttcctcgtagaggaactcggaccactctgagacgaaggcccgcgtccaggccaggacgaaggaggctatgtgggaggggtagcggtcgttgtccactagggggtccaccttctccaaggtgtgaagacacatgtcgccttcctcggcgtccaggaaggtgattggcttgtaggtgtaggccacgtgaccgggggttcctgacgggggggtataaaagggggtgggggcgcgctcgtcgtcactctcttccgcatcgctgtctgcgagggccagctgctggggtgagtattccctctcgaaggcgggcatgacctccgcgctgaggttgtcagtttccaaaaacgaggaggatttgatgttcacctgtcccgaggtgatacctttgagggtacccgcgtccatctggtcagaaaacacgatctttttattgtccagcttggtggcgaacgacccgtagagggcgttggagagcagcttggcgatggagcgcagggtctggttcttgtccctgtcggcgcgctccttggccgcgatgttgagctgcacgtactcgcgcgcgacgcagcgccactcggggaagacggtggtgcgctcgtcgggcaccaggcgcacgcgccagccgcggttgtgcagggtgaccaggtccacgctggtggcgacctcgccgcgcaggcgctcgttggtccagcagagacggccgcccttgcgcgagcagaaggggggcagggggtcgagctgggtctcgtccggggggtccgcgtccacggtgaaaaccccggggcgcaggcgcgcgtcgaagtagtctatcttgcaaccttgcatgtccagcgcctgctgccagtcgcgggcggcgagcgcgcgctcgtaggggttgagcggcgggccccagggcatggggtgggtgagtgcggaggcgtacatgccgcagatgtcatagacgtagaggggctcccgcaggaccccgatgtaggtggggtagcagcggccgccgcggatgctggcgcgcacgtagtcatacagctcgtgcgagggggcgaggaggtcggggcccaggttggtgcgggcggggcgctccgcgcggaagacgatctgcctgaagatggcatgcgagttggaagagatggtggggcgctggaagacgttgaagctggcgtcctgcaggccgacggcgtcgcgcacgaaggaggcgtaggagtcgcgcagcttgtgtaccagctcggcggtgacctgcacgtcgagcgcgcagtagtcgagggtctcgcggatgatgtcatatttagcctgccccttctttttccacagctcgcggttgaggacaaactcttcgcggtctttccagtactcttggatcgggaaaccgtccggttccgaacggtaagagcctagcatgtagaactggttgacggcctggtaggcgcagcagcccttctccacggggagggcgtaggcctgcgcggccttgcggagcgaggtgtgggtcagggcgaaggtgtccctgaccatgactttgaggtactggtgcttgaagtcggagtcgtcgcagccgccccgctcccagagcgagaagtcggtgcgcttcttggagcgggggttgggcagagcgaaggtgacatcgttgaagaggattttgcccgcgcggggcatgaagttgcgggtgatgcggaagggccccggcacttcagagcggttgttgatgacctgggcggcgagcacgatctcgtcgaagccgttgatgttgtggcccacgatgtagagttccaggaagcggggccggccctttacggtgggcagcttctttagctcttcgtaggtgagctcctcgggcgaggcgaggccgtgctcggccagggcccagtccgcgaggtgcgggttgtctctgaggaaggacttccagaggtcgcgggccaggagggtctgcaggcggtctctgaaggtcctgaactggcggcccacggccattttttcgggggtgatgcagtagaaggtgagggggtcttgctgccagcggtcccagtcgagctgcagggcgaggtcgcgcgcggcggtgaccaggcgctcgtcgcccccgaatttcatgaccagcatgaagggcacgagctgctttccgaaggcccccatccaagtgtaggtctctacatcgtaggtgacaaagaggcgctccgtgcgaggatgcgagccgatcgggaagaactggatctcccgccaccagttggaggagtggctgttgatgtggtggaagtagaagtcccgtcgccgggccgaacactcgtgctggcttttgtaaaagcgagcgcagtactggcagcgctgcacgggctgtacctcatgcacgagatgcacctttcgcccgcgcacgaggaagccgaggggaaatctgagccccccgcctggctcgcggcatggctggttctcttctactttggatgcgtgtccgtctccgtctggctcctcgaggggtgttacggtggagcggaccaccacgccgcgcgagccgcaggtccagatatcggcgcgcggcggtcggagtttgatgacgacatcgcgcagctgggagctgtccatggtctggagctcccgcggcggcggcaggtcagccgggagttcttgcaggttcacctcgcagagtcgggccagggcgcggggcaggtctaggtggtacctgatctctaggggcgtgttggtggcggcgtcgatggcttgcaggagcccgcagccccggggggcgacgacggtgccccgcggggtggtggtggtggtggcggtgcagctcagaagcggtgccgcgggcgggcccccggaggtagggggggctccggtcccgcgggcaggggcggcagcggcacgtcggcgtggagcgcgggcaggagttggtgctgtgcccggaggttgctggcgaaggcgacgacgcggcggttgatctcctggatctggcgcctctgcgtgaagacgacgggcccggtgagcttgaacctgaaagagagttcgacagaatcaatctcggtgtcattgaccgcggcctggcgcaggatctcctgcacgtctcccgagttgtcttggtaggcgatctcggccatgaactgctcgatctcttcctcctggaggtctccgcgtccggcgcgttccacggtggccgccaggtcgttggagatgcgccccatgagctgcgagaaggcgttgagtccgccctcgttccagactcggctgtagaccacgcccccctggtcatcgcgggcgcgcatgaccacctgcgcgaggttgagctccacgtgccgcgcgaagacggcgtagttgcgcagacgctggaagaggtagttgagggtggtggcggtgtgctcggccacgaagaagttcatgacccagcggcgcaacgtggattcgttgatgtcccccaaggcctccagccgttccatggcctcgtagaagtccacggcgaagttgaaaaactgggagttgcgcgccgacacggtcaactcctcctccagaagacggatgagctcggcgacggtgtcgcgcacctcgcgctcgaaggctatggggatctcttcctccgctagcatcaccacctcctcctcttcctcctcttctggcacttccatgatggcttcctcctcttcggggggtggcggcggcggcggtgggggagggggcgctctgcgccggcggcggcgcaccgggaggcggtccacgaagcgcgcgatcatctccccgcggcggcggcgcatggtctcggtgacggcgcggccgttctcccgggggcgcagttggaagacgccgccggacatctggtgctggggcgggtggccgtgaggcagcgagacggcgctgacgatgcatctcaacaattgctgcgtaggtacgccgccgagggacctgagggagtccatatccaccggatccgaaaacctttcgaggaaggcgtctaaccagtcgcagtcgcaaggtaggctgagcaccgtggcgggcggcggggggtggggggagtgtctggcggaggtgctgctgatgatgtaattgaagtaggcggacttgacacggcggatggtcgacaggagcaccatgtccttgggtccggcctgctggatgcggaggcggtcggctatgccccaggcttcgttctggcatcggcgcaggtccttgtagtagtcttgcatgagcctttccaccggcacctcttctccttcctcttctgcttcttccatgtctgcttcggccctggggcggcgccgcgcccccctgccccccatgcgcgtgaccccgaaccccctgagcggttggagcagggccaggtcggcgacgacgcgctcggccaggatggcctgctgcacctgcgtgagggtggtttggaagtcatccaagtccacgaagcggtggtaggcgcccgtgttgatggtgtaggtgcagttggccatgacggaccagttgacggtctggtggcccggttgcgacatctcggtgtacctgagtcgcgagtaggcgcgggagtcgaagacgtagtcgttgcaagtccgcaccaggtactggtagcccaccaggaagtgcggcggcggctggcggtagaggggccagcgcagggtggcgggggctccgggggccaggtcttccagcatgaggcggtggtaggcgtagatgtacctggacatccaggtgatacccgcggcggtggtggaggcgcgcgggaagtcgcgcacccggttccagatgttgcgcaggggcagaaagtgctccatggtaggcgtgctctgtccagtcagacgcgcgcagtcgttgatactctagaccagggaaaacgaaagccggtcagcgggcactcttccgtggtctggtgaatagatcgcaagggtatcatggcggagggcctcggttcgagccccgggtccgggccggacggtccgccatgatccacgcggttaccgcccgcgtgtcgaacccaggtgtgcgacgtcagacaacggtggagtgttccttttggcgtttttctggccgggcgccggcgccgcgtaagagactaagccgcgaaagcgaaagcagtaagtggctcgctccccgtagccggagggatccttgctaagggttgcgttgcggcgaaccccggttcgaatcccgtactcgggccggccggacccgcggctaaggtgttggattggcctccccctcgtataaagaccccgcttgcggattgactccggacacggggacgagccccttttatttttgctttccccagatgcatccggtgctgcggcagatgcgccccccgccccagcagcagcaacaacaccagcaagagcggcagcaacagcagcgggagtcatgcagggccccctcacccaccctcggcgggccggccacctcggcgtccgcggccgtgtctggcgcctgcggcggcggcggggggccggctgacgaccccgaggagcccccgcggcgcagggccagacactacctggacctggaggagggcgagggcctggcgcggctgggggcgccgtctcccgagcgccacccgcgggtgcagctgaagcgcgactcgcgcgaggcgtacgtgcctcggcagaacctgttcagggaccgcgcgggcgaggagcccgaggagatgcgggacaggaggttcagcgcagggcgggagctgcggcaggggctgaaccgcgagcggctgctgcgcgaggaggactttgagcccgacgcgcggacggggatcagccccgcgcgcgcgcacgtggcggccgccgacctggtgacggcgtacgagcagacggtgaaccaggagatcaacttccaaaagagtttcaacaaccacgtgcgcacgctggtggcgcgcgaggaggtgaccatcgggctgatgcacctgtgggactttgtaagcgcgctggtgcagaaccccaacagcaagcctctgacggcgcagctgttcctgatagtgcagcacagcagggacaacgaggcgtttagggacgcgctgctgaacatcaccgagcccgagggtcggtggctgctggacctgattaacatcctgcagagcatagtggtgcaggagcgcagcctgagcctggccgacaaggtggcggccatcaactactcgatgctgagcctgggcaagttttacgcgcgcaagatctaccagacgccgtacgtgcccatagacaaggaggtgaagatcgacggtttttacatgcgcatggcgctgaaggtgctcaccctgagcgacgacctgggcgtgtaccgcaacgagcgcatccacaaggccgtgagcgtgagccggcggcgcgagctgagcgaccgcgagctgatgcacagcctgcagcgggcgctggcgggcgccggcagcggcgacagggaggcggagtcctacttcgatgcgggggcggacctgcgctgggcgcccagccggcgggccctggaggccgcgggggtccgcgaggactatgacgaggacggcgaggaggatgaggagtacgagctagaggagggcgagtacctggactaaaccgcgggtggtgtttccggtagatgcaagacccgaacgtggtggacccggcgctgcgggcggctctgcagagccagccgtccggccttaactcctcagacgactggcgacaggtcatggaccgcatcatgtcgctgacggcgcgtaacccggacgcgttccggcagcagccgcaggccaacaggctctccgccatcctggaggcggtggtgcctgcgcgctcgaaccccacgcacgagaaggtgctggccatagtgaacgcgctggccgagaacagggccatccgcccggacgaggccgggctggtgtacgacgcgctgctgcagcgcgtggcccgctacaacagcggcaacgtgcagaccaacctggaccggctggtgggggacgtgcgcgaggcggtggcgcagcgcgagcgcgcggatcggcagggcaacctgggctccatggtggcgctgaatgccttcctgagcacgcagccggccaacgtgccgcgggggcaggaagactacaccaactttgtgagcgcgctgcggctgatggtgaccgagaccccccagagcgaggtgtaccagtcgggcccggactacttcttccagaccagcagacagggcctgcagacggtgaacctgagccaggctttcaagaacctgcgggggctgtggggcgtgaaggcgcccaccggcgaccgggcgacggtgtccagcctgctgacgcccaactcgcgcctgctgctgctgctgatcgcgccgttcacggacagcggcagcgtgtcccgggacacctacctggggcacctgctgaccctgtaccgcgaggccatcgggcaggcgcaggtggacgagcacaccttccaggagatcaccagcgtgagccgcgcgctggggcaggaggacacgagcagcctggaggcgactctgaactacctgctgaccaaccggcggcagaagattccctcgctgcacagcctgacctccgaggaggagcgcatcttgcgctacgtgcagcagagcgtgagcctgaacctgatgcgcgacggggtgacgcccagcgtggcgctggacatgaccgcgcgcaacatggaaccgggcatgtacgccgcgcaccggccttacatcaaccgcctgatggactacctgcatcgcgcggcggccgtgaaccccgagtactttaccaacgccatcctgaacccgcactggctcccgccgcccgggttctacagcgggggcttcgaggtcccggagaccaacgatggcttcctgtgggacgacatggacgacagcgtgttctccccgcggccgcaggcgctggcggaagcgtccctgctgcgtcccaagaaggaggaggaggaggaggcgagtcgccgccgcggcagcagcggcgtggcttctctgtccgagctgggggcggcagccgccgcgcgccccgggtccctgggcggcagcccctttccgagcctggtggggtctctgcacagcgagcgcaccacccgccctcggctgctgggcgaggacgagtacctgaataactccctgctgcagccggtgcgggagaaaaacctgcctcccgccttccccaacaacgggatagagagcctggtggacaagatgagcagatggaagacctatgcgcaggagcacagggacgcgcctgcgctccggccgcccacgcggcgccagcgccacgaccggcagcgggggctggtgtgggatgacgaggactccgcggacgatagcagcgtgctggacctgggagggagcggcaacccgttcgcgcacctgcgcccccgcctggggaggatgttttaaaaaaaaaaaaaaaaagcaagaagcatgatgcaaaaattaaataaaactcaccaaggccatggcgaccgagcgttggtttcttgtgttcccttcagtatgcggcgcgcggcgatgtaccaggagggacctcctccctcttacgagagcgtggtgggcgcggcggcggcggcgccctcttctccctttgcgtcgcagctgctggagccgccgtacgtgcctccgcgctacctgcggcctacgggggggagaaacagcatccgttactcggagctggcgcccctgttcgacaccacccgggtgtacctggtggacaacaagtcggcggacgtggcctccctgaactaccagaacgaccacagcaattttttgaccacggtcatccagaacaatgactacagcccgagcgaggccagcacccagaccatcaatctggatgaccggtcgcactggggcggcgacctgaaaaccatcctgcacaccaacatgcccaacgtgaacgagttcatgttcaccaataagttcaaggcgcgggtgatggtgtcgcgctcgcacaccaaggaagaccgggtggagctgaagtacgagtgggtggagttcgagctgccagagggcaactactccgagaccatgaccattgacctgatgaacaacgcgatcgtggagcactatctgaaagtgggcaggcagaacggggtcctggagagcgacatcggggtcaagttcgacaccaggaacttccgcctggggctggaccccgtgaccgggctggttatgcccggggtgtacaccaacgaggccttccatcccgacatcatcctgctgcccggctgcggggtggacttcacttacagccgcctgagcaacctcctgggcatccgcaagcggcagcccttccaggagggcttcaggatcacctacgaggacctggaggggggcaacatccccgcgctcctcgatgtggaggcctaccaggatagcttgaaggaaaatgaggcgggacaggaggataccgcccccgccgcctccgccgccgccgagcagggcgaggatgctgctgacaccgcggccgcggacggggcagaggccgaccccgctatggtggtggaggctcccgagcaggaggaggacatgaatgacagtgcggtgcgcggagacaccttcgtcacccggggggaggaaaagcaagcggaggccgaggccgcggccgaggaaaagcaactggcggcagcagcggcggcggcggcgttggccgcggcggaggctgagtctgaggggaccaagcccgccaaggagcccgtgattaagcccctgaccgaagatagcaagaagcgcagttacaacctgctcaaggacagcaccaacaccgcgtaccgcagctggtacctggcctacaactacggcgacccgtcgacgggggtgcgctcctggaccctgctgtgcacgccggacgtgacctgcggctcggagcaggtgtactggtcgctgcccgacatgatgcaagaccccgtgaccttccgctccacgcggcaggtcagcaacttcccggtggtgggcgccgagctgctgcccgtgcactccaagagcttctacaacgaccaggccgtctactcccagctcatccgccagttcacctctctgacccacgtgttcaatcgctttcctgagaaccagattctggcgcgcccgcccgcccccaccatcaccaccgtcagtgaaaacgttcctgctctcacagatcacgggacgctaccgctgcgcaacagcatcggaggagtccagcgagtgaccgttactgacgccagacgccgcacctgcccctacgtttacaaggccttgggcatagtctcgccgcgcgtcctttccagccgcactttttgagcaacaccaccatcatgtccatcctgatctcacccagcaataactccggctggggactgctgcgcgcgcccagcaagatgttcggaggggcgaggaagcgttccgagcagcaccccgtgcgcgtgcgcgggcacttccgcgccccctggggagcgcacaaacgcggccgcgcggggcgcaccaccgtggacgacgccatcgactcggtggtggagcaggcgcgcaactacaggcccgcggtctctaccgtggacgcggccatccagaccgtggtgcggggcgcgcggcggtacgccaagctgaagagccgccggaagcgcgtggcccgccgccaccgccgccgacccggggccgccgccaaacgcgccgccgcggccctgcttcgccgggccaagcgcacgggccgccgcgccgccatgagggccgcgcgccgcttggccgccggcatcaccgccgccaccatggccccccgtacccgaagacgcgcggccgccgccgccgccgccgccatcagtgacatggccagcaggcgccggggcaacgtgtactgggtgcgcgactcggtgaccggcacgcgcgtgcccgtgcgcttccgccccccgcggacttgagatgatgtgaaaaaacaacactgagtctcctgctgttgtgtgtatcccagcggcggcggcgcgcgcagcgtcatgtccaagcgcaaaatcaaagaagagatgctccaggtcgtcgcgccggagatctatgggcccccgaagaaggaagagcaggattcgaagccccgcaagataaagcgggtcaaaaagaaaaagaaagatgatgacgatgccgatggggaggtggagttcctgcgcgccacggcgcccaggcgcccggtgcagtggaagggccggcgcgtaaagcgcgtcctgcgccccggcaccgcggtggtcttcacgcccggcgagcgctccacccggactttcaagcgcgtctatgacgaggtgtacggcgacgaagacctgctggagcaggccaacgagcgcttcggagagtttgcttacgggaagcgtcagcgggcgctggggaaggaggacctgctggcgctgccgctggaccagggcaaccccacccccagtctgaagcccgtgaccctgcagcaggtgctgccgagcagcgcaccctccgaggcgaagcggggtctgaagcgcgagggcggcgacctggcgcccaccgtgcagctcatggtgcccaagcggcagaggctggaggatgtgctggagaaaatgaaagtagaccccggtctgcagccggacatcagggtccgccccatcaagcaggtggcgccgggcctcggcgtgcagaccgtggacgtggtcatccccaccggcaactcccccgccgccgccaccactaccgctgcctccacggacatggagacacagaccgatcccgccgcagccgcagccgcagccgccgccgcgacctcctcggcggaggtgcagacggacccctggctgccgccggcgatgtcagctccccgcgcgcgtcgcgggcgcaggaagtacggcgccgccaacgcgctcctgcccgagtacgccttgcatccttccatcgcgcccacccccggctaccgaggctatacctaccgcccgcgaagagccaagggttccacccgccgtccccgccgacgcgccgccgccaccacccgccgccgccgccgcagacgccagcccgcactggctccagtctccgtgaggaaagtggcgcgcgacggacacaccctggtgctgcccagggcgcgctaccaccccagcatcgtttaaaagcctgttgtggttcttgcagatatggccctcacttgccgcctccgtttcccggtgccgggataccgaggaggaagatcgcgccgcaggaggggtctggccggccgcggcctgagcggaggcagccgccgcgcgcaccggcggcgacgcgccaccagccgacgcatgcgcggcggggtgctgcccctgttaatccccctgatcgccgcggcgatcggcgccgtgcccgggatcgcctccgtggccttgcaagcgtcccagaggcattgacagacttgcaaacttgcaaatatggaaaaaaaaaccccaataaaaaagtctagactctcacgctcgcttggtcctgtgactattttgtagaatggaagacatcaactttgcgtcgctggccccgcgtcacggctcgcgcccgttcctgggacactggaacgatatcggcaccagcaacatgagcggtggcgccttcagttggggctctctgtggagcggcattaaaagtatcgggtctgccgttaaaaattacggctcccgggcctggaacagcagcacgggccagatgttgagagacaagttgaaagagcagaacttccagcagaaggtggtggagggcctggcctccggcatcaacggggtggtggacctggccaaccaggccgtgcagaataagatcaacagcagactggacccccggccgccggtggaggaggtgccgccggcgctggagacggtgtcccccgatgggcgtggcgagaagcgcccgcggcccgatagggaagagaccactctggtcacgcagaccgatgagccgcccccgtatgaggaggccctgaagcaaggtctgcccaccacgcggcccatcgcgcccatggccaccggggtggtgggccgccacacccccgccacgctggacttgcctccgcccgccgatgtgccgcagcagcagaaggcggcacagccgggcccgcccgcgaccgcctcccgttcctccgccggtcctctgcgccgcgcggccagcggcccccgcgggggggtcgcgaggcacggcaactggcagagcacgctgaacagcatcgtgggtctgggggtgcggtccgtgaagcgccgccgatgctactgaatagcttagctaacgtgttgtatgtgtgtatgcgccctatgtcgccgccagaggagctgctgagtcgccgccgttcgcgcgcccaccaccaccgccactccgcccctcaagatggcgaccccatcgatgatgccgcagtggtcgtacatgcacatctcgggccaggacgcctcggagtacctgagccccgggctggtgcagttcgcccgcgccaccgagagctacttcagcctgagtaacaagtttaggaaccccacggtggcgcccacgcacgatgtgaccaccgaccggtctcagcgcctgacgctgcggttcattcccgtggaccgcgaggacaccgcgtactcgtacaaggcgcggttcaccctggccgtgggcgacaaccgcgtgctggacatggcctccacctactttgacatccgcggggtgctggaccggggtcccactttcaagccctactctggcaccgcctacaactccctggcccccaagggcgctcccaactcctgcgagtgggagcaagaggaaactcaggcagttgaagaagcagcagaagaggaagaagaagatgctgacggtcaagctgaggaagagcaagcagctaccaaaaagactcatgtatatgctcaggctcccctttctggcgaaaaaattagtaaagatggtctgcaaataggaacggacgctacagctacagaacaaaaacctatttatgcagaccctacattccagcccgaaccccaaatcggggagtcccagtggaatgaggcagatgctacagtcgccggcggtagagtgctaaagaaatctactcccatgaaaccatgctatggttcctatgcaagacccacaaatgctaatggaggtcagggtgtactaacggcaaatgcccagggacagctagaatctcaggttgaaatgcaattcttttcaacttctgaaaacgcccgtaacgaggctaacaacattcagcccaaattggtgctgtatagtgaggatgtgcacatggagaccccggatacgcacctttcttacaagcccgcaaaaagcgatgacaattcaaaaatcatgctgggtcagcagtccatgcccaacagacctaattacatcggcttcagagacaactttatcggcctcatgtattacaatagcactggcaacatgggagtgcttgcaggtcaggcctctcagttgaatgcagtggtggacttgcaagacagaaacacagaactgtcctaccagctcttgcttgattccatgggtgacagaaccagatacttttccatgtggaatcaggcagtggacagttatgacccagatgttagaattattgaaaatcatggaactgaagacgagctccccaactattgtttccctctgggtggcataggggtaactgacacttaccaggctgttaaaaccaacaatggcaataacgggggccaggtgacttggacaaaagatgaaacttttgcagatcgcaatgaaataggggtgggaaacaatttcgctatggagatcaacctcagtgccaacctgtggagaaacttcctgtactccaacgtggcgctgtacctaccagacaagcttaagtacaacccctccaatgtggacatctctgacaaccccaacacctacgattacatgaacaagcgagtggtggccccggggctggtggactgctacatcaacctgggcgcgcgctggtcgctggactacatggacaacgtcaaccccttcaaccaccaccgcaatgcgggcctgcgctaccgctccatgctcctgggcaacgggcgctacgtgcccttccacatccaggtgccccagaagttctttgccatcaagaacctcctcctcctgccgggctcctacacctacgagtggaacttcaggaaggatgtcaacatggtcctccagagctctctgggtaacgatctcagggtggacggggccagcatcaagttcgagagcatctgcctctacgccaccttcttccccatggcccacaacacggcctccacgctcgaggccatgctcaggaacgacaccaacgaccagtccttcaatgactacctctccgccgccaacatgctctaccccatacccgccaacgccaccaacgtccccatctccatcccctcgcgcaactgggcggccttccgcggctgggccttcacccgcctcaagaccaaggagaccccctccctgggctcgggattcgacccctactacacctactcgggctccattccctacctggacggcaccttctacctcaaccacactttcaagaaggtctcggtcaccttcgactcctcggtcagctggccgggcaacgaccgtctgctcacccccaacgagttcgagatcaagcgctcggtcgacggggagggctacaacgtggcccagtgcaacatgaccaaggactggttcctggtccagatgctggccaactacaacatcggctaccagggcttctacatcccagagagctacaaggacaggatgtactccttcttcaggaacttccagcccatgagccggcaggtggtggaccagaccaagtacaaggactaccaggaggtgggcatcatccaccagcacaacaactcgggcttcgtgggctacctcgcccccaccatgcgcgagggacaggcctaccccgccaacttcccctatccgctcataggcaagaccgcggtcgacagcatcacccagaaaaagttcctctgcgaccgcaccctctggcgcatccccttctccagcaacttcatgtccatgggtgcgctctcggacctgggccagaacttgctctacgccaactccgcccacgccctcgacatgaccttcgaggtcgaccccatggacgagcccacccttctctatgttctgttcgaagtctttgacgtggtccgggtccaccagccgcaccgcggcgtcatcgagaccgtgtacctgcgtacgcccttctcggccggcaacgccaccacctaaagaagcaagccgcagtcatcgccgcctgcatgccgtcgggttccaccgagcaagagctcagggccatcgtcagagacctgggatgcgggccctattttttgggcaccttcgacaagcgcttccctggctttgtctccccacacaagctggcctgcgccatcgtcaacacggccggccgcgagaccgggggcgtgcactggctggccttcgcctggaacccgcgctccaaaacatgcttcctctttgaccccttcggcttttcggaccagcggctcaagcaaatctacgagttcgagtacgagggcttgctgcgtcgcagcgccatcgcctcctcgcccgaccgctgcgtcaccctcgaaaagtccacccagaccgtgcaggggcccgactcggccgcctgcggtctcttctgctgcatgtttctgcacgcctttgtgcactggcctcagagtcccatggaccgcaaccccaccatgaacttgctgacgggggtgcccaactccatgctccagagcccccaggtcgagcccaccctgcgccgcaaccaggagcagctctacagcttcctggagcgccactcgccttacttccgccgccacagcgcacagatcaggagggccacctccttctgccacttgcaagagatgcaagaagggtaataacgatgtacacactttttttctcaataaatggcatctttttatttatacaagctctctggggtattcatttcccaccaccacccgccgttgtcgccatctggctctatttagaaatcgaaagggttctgccgggagtcgccgtgcgccacgggcagggacacgttgcgatactggtagcgggtgccccacttgaactcgggcaccaccaggcgaggcagctcggggaagttttcgctccacaggctgcgggtcagcaccagcgcgttcatcaggtcgggcgccgagatcttgaagtcgcagttggggccgccgccctgcgcgcgcgagttgcggtacaccgggttgcagcactggaacaccaacagcgccgggtgcttcacgctggccagcacgctgcggtcggagatcagctcggcgtccaggtcctccgcgttgctcagcgcgaacggggtcatcttgggcacttgccgccccaggaagggcgcgtgccccggtttcgagttgcagtcgcagcgcagcgggatcagcaggtgcccgtgcccggactcggcgttggggtacagcgcgcgcatgaaggcctgcatctggcggaaggccatctgggccttggcgccctccgagaagaacatgccgcaggacttgcccgagaactggtttgcggggcagctggcgtcgtgcaggcagcagcgcgcgtcggtgttggcgatctgcaccacgttgcgcccccaccggttcttcacgatcttggccttggacgattgctccttcagcgcgcgctgcccgttctcgctggtcacatccatctcgatcacatgttccttgttcaccatgctgctgccgtgcagacacttcagctcgccctccgtctcggtgcagcggtgctgccacagcgcgcagcccgtgggctcgaaagacttgtaggtcacctccgcgaaggactgcaggtacccctgcaaaaagcggcccatcatggtcacgaaggtcttgttgctgctgaaggtcagctgcagcccgcggtgctcctcgttcagccaggtcttgcacacggccgccagcgcctccacctggtcgggcagcatcttgaagttcaccttcagctcattctccacgtggtacttgtccatcagcgtgcgcgccgcctccatgcccttctcccaggccgacaccagcggcaggctcacggggttcttcaccatcaccgtggccgccgcctccgccgcgctttcgctttccgccccgctgttctcttcctcttcctcctcttcctcgccgccgcccactcgcagcccccgcaccacggggtcgtcttcctgcaggcgctgcaccttgcgcttgccgttgcgcccctgcttgatgcgcacgggcgggttgctgaagcccaccatcaccagcgcggcctcttcttgctcgtcctcgctgtccagaatgacctccggggagggggggttggtcatcctcagtaccgaggcacgcttctttttcttcctgggggcgttcgccagctccgcggctgcggccgctgccgaggtcgaaggccgagggctgggcgtgcgcggcaccagcgcgtcctgcgagccgtcctcgtcctcctcggactcgagacggaggcgggcccgcttcttcgggggcgcgcggggcggcggaggcggcggcggcgacggagacggggacgagacatcgtccagggtgggtggacggcgggccgcgccgcgtccgcgctcgggggtggtctcgcgctggtcctcttcccgactggccatctcccactgctccttctcctataggcagaaagagatcatggagtctctcatgcgagtcgagaaggaggaggacagcctaaccgccccctctgagccctccaccaccgccgccaccaccgccaatgccgccgcggacgacgcgcccaccgagaccaccgccagtaccaccctccccagcgacgcacccccgctcgagaatgaagtgctgatcgagcaggacccgggttttgtgagcggagaggaggatgaggtggatgagaaggagaaggaggaggtcgccgcctcagtgccaaaagaggataaaaagcaagaccaggacgacgcagataaggatgagacagcagtcgggcgggggaacggaagccatgatgctgatgacggctacctagacgtgggagacgacgtgctgcttaagcacctgcaccgccagtgcgtcatcgtctgcgacgcgctgcaggagcgctgcgaagtgcccctggacgtggcggaggtcagccgcgcctacgagcggcacctcttcgcgccgcacgtgccccccaagcgccgggagaacggcacctgcgagcccaacccgcgtctcaacttctacccggtcttcgcggtacccgaggtgctggccacctaccacatctttttccaaaactgcaagatccccctctcctgccgcgccaaccgcacccgcgccgacaaaaccctgaccctgcggcagggcgcccacatacctgatatcgcctctctggaggaagtgcccaagatcttcgagggtctcggtcgcgacgagaaacgggcggcgaacgctctgcacggagacagcgaaaacgagagtcactcgggggtgctggtggagctcgagggcgacaacgcgcgcctggccgtactcaagcgcagcatagaggtcacccactttgcctacccggcgctcaacctgccccccaaggtcatgagtgtggtcatgggcgagctcatcatgcgccgcgcccagcccctggccgcggatgcaaacttgcaagagtcctccgaggaaggcctgcccgcggtcagcgacgagcagctggcgcgctggctggagacccgcgaccccgcgcagctggaggagcggcgcaagctcatgatggccgcggtgctggtcaccgtggagctcgagtgtctgcagcgcttcttcgcggaccccgagatgcagcgcaagctcgaggagaccctgcactacaccttccgccagggctacgtgcgccaggcctgcaagatctccaacgtggagctctgcaacctggtctcctacctgggcatcctgcacgagaaccgcctcgggcagaacgtcctgcactccaccctcaaaggggaggcgcgccgcgactacatccgcgactgcgcctacctcttcctctgctacacctggcagacggccatgggggtctggcagcagtgcctggaggagcgcaacctcaaggagctggaaaagctcctcaagcgcaccctcagggacctctggacgggcttcaacgagcgctcggtggccgccgcgctggcggacatcatctttcccgagcgcctgctcaagaccctgcagcagggcctgcccgacttcaccagccagagcatgctgcagaacttcaggactttcatcctggagcgctcgggcatcctgccggccacttgctgcgcgctgcccagcgacttcgtgcccatcaagtacagggagtgcccgccgccgctctggggccactgctacctcttccagctggccaactacctcgcctaccactcggacctcatggaagacgtgagcggcgagggcctgctcgagtgccactgccgctgcaacctctgcacgccccaccgctctctagtctgcaacccgcagctgctcagcgagagtcagattatcggtaccttcgagctgcagggtccctcgcctgacgagaagtccgcggctccagggctgaaactcactccggggctgtggacttccgcctacctacgcaaatttgtacctgaggactaccacgcccacgagatcaggttctacgaagaccaatcccgcccgcccaaggcggagctcaccgcctgcgtcatcacccaggggcacatcctgggccaattgcaagccatcaacaaagcccgccgagagttcttgctgaaaaagggtcggggggtgtacctggacccccagtccggcgaggagctaaacccgctacccccgccgccgccccagcagcgggaccttgcttcccaggatggcacccagaaagaagcagcagccgccgccgccgccgcagccatacatgcttctggaggaagaggaggaggactgggacagtcaggcagaggaggtttcggacgaggagcaggaggagatgatggaagactgggaggaggacagcagcctagacgaggaagcttcagaggccgaagaggtggcagacgcaacaccatcgccctcggtcgcagccccctcgccggggcccctgaaatcctccgaacccagcaccagcgctataacctccgctcctccggcgccggcgccacccgcccgcagacccaaccgtagatgggacaccacaggaaccggggtcggtaagtccaagtgcccgccgccgccaccgcagcagcagcagcagcagcgccagggctaccgctcgtggcgcgggcacaagaacgccatagtcgcctgcttgcaagactgcgggggcaacatctctttcgcccgccgcttcctgctattccaccacggggtcgcctttccccgcaatgtcctgcattactaccgtcatctctacagcccctactgcagcggcgacccagaggcggcagcggcagccacagcggcgaccaccacctaggaagatatcctccgcgggcaagacagcggcagcagcggccaggagacccgcggcagcagcggcgggagcggtgggcgcactgcgcctctcgcccaacgaacccctctcgacccgggagctcagacacaggatcttccccactttgtatgccatcttccaacagagcagaggccaggagcaggagctgaaaataaaaaacagatctctgcgctccctcacccgcagctgtctgtatcacaaaagcgaagatcagcttcggcgcacgctggaggacgcggaggcactcttcagcaaatactgcgcgctcactcttaaagactagctccgcgcccttctcgaatttaggcgggagaaaactacgtcatcgccggccgccgcccagcccgcccagccgagatgagcaaagagattcccacgccatacatgtggagctaccagccgcagatgggactcgcggcgggagcggcccaggactactccacccgcatgaactacatgagcgcgggaccccacatgatctcacaggtcaacgggatccgcgcccagcgaaaccaaatactgctggaacaggcggccatcaccgccacgccccgccataatctcaacccccgaaattggcccgccgccctcgtgtaccaggaaaccccctccgccaccaccgtactacttccgcgtgacgcccaggccgaagtccagatgactaactcaggggcgcagctcgcgggcggctttcgtcacggggcgcggccgctccgaccaggtataagacacctgatgatcagaggccgaggtatccagctcaacgacgagtcggtgagctcttcgctcggtctccgtccggacggaactttccagctcgccggatccggccgctcttcgttcacgccccgccaggcgtacctgactctgcagacctcgtcctcggagccccgctccggcggcatcggaaccctccagttcgtggaggagttcgtgccctcggtctacttcaaccccttctcgggacctcccggacgctaccccgaccagttcattccgaactttgacgcggtgaaggactcggcggacggctacgactgaatgtcaggtgtcgaggcagagcagcttcgcctgagacacctcgagcactgccgccgccacaagtgcttcgcccgcggttctggtgagttctgctactttcagctacccgaggagcataccgaggggccggcgcacggcgtccgcctgaccacccagggcgaggttacctgttccctcatccgggagtttaccctccgtcccctgctagtggagcgggagcggggtccctgtgtcctaactatcgcctgcaactgccctaaccctggattacatcaagatctttgctgtcatctctgtgctgagtttaataaacgctgagatcagaatctactggggctcctgtcgccatcctgtgaacgccaccgtcttcacccaccccgaccaggcccaggcgaacctcacctgcggtctgcatcggagggccaagaagtacctcacctggtacttcaacggcaccccctttgtggtttacaacagcttcgacggggacggagtctccctgaaagaccagctctccggtctcagctactccatccacaagaacaccaccctccaactcttccctccctacctgccgggaacctacgagtgcgtcaccggccgctgcacccacctcacccgcctgatcgtaaaccagagctttccgggaacagataactccctcttccccagaacaggaggtgagctcaggaaactccccggggaccagggcggagacgtaccttcgacccttgtggggttaggattttttattaccgggttgctggctcttttaatcaaagtttccttgagatttgttctttccttctacgtgtatgaacacctcaacctccaataactctaccctttcttcggaatcaggtgacttctctgaaatcgggcttggtgtgctgcttactctgttgatttttttccttatcatactcagccttctgtgcctcaggctcgccgcctgctgcgcacacatctatatctactgctggttgctcaagtgcaggggtcgccacccaagatgaacaggtacatggtcctatcgatcctaggcctgctggccctggcggcctgcagcgccgccaaaaaagagattacctttgaggagcccgcttgcaatgtaactttcaagcccgagggtgaccaatgcaccaccctcgtcaaatgcgttaccaatcatgagaggctgcgcatcgactacaaaaacaaaactggccagtttgcggtctatagtgtgtttacgcccggagacccctctaactactctgtcaccgtcttccagggcggacagtctaagatattcaattacactttccctttttatgagttatgcgatgcggtcatgtacatgtcaaaacagtacaacctgtggcctccctctccccaggcgtgtgtggaaaatactgggtcttactgctgtatggctttcgcaatcactacgctcgctctaatctgcacggtgctatacataaaattcaggcagaggcgaatctttatcgatgaaaagaaaatgccttgatcgctaacaccggctttctatctgcagaatgaatgcaatcacctccctactaatcaccaccaccctccttgcgattgcccatgggttgacacgaatcgaagtgccagtggggtccaatgtcaccatggtgggccccgccggcaattccaccctcatgtgggaaaaatttgtccgcaatcaatgggttcatttctgctctaaccgaatcagtatcaagcccagagccatctgcgatgggcaaaatctaactctgatcaatgtgcaaatgatggatgctgggtactattacgggcagcggggagaaatcattaattactggcgaccccacaaggactacatgctgcatgtagtcgaggcacttcccactaccacccccactaccacctctcccaccaccaccaccactactactactactactactactactactactaccactaccgctgcccgccatacccgcaaaagcaccatgattagcacaaagccccctcgtgctcactcccacgccggcgggcccatcggtgcgacctcagaaaccaccgagctttgcttctgccaatgcactaacgccagcgctcatgaactgttcgacctggagaatgaggatgtccagcagagctccgcttgcctgacccaggaggctgtggagcccgttgccctgaagcagatcggtgattcaataattgactcttcttcttttgccactcccgaataccctcccgattctactttccacatcacgggtaccaaagaccctaacctctctttctacctgatgctgctgctctgtatctctgtggtctcttccgcgctgatgttactggggatgttctgctgcctgatctgccgcagaaagagaaaagctcgctctcagggccaaccactgatgcccttcccctaccccccggattttgcagataacaagatatgagctcgctgctgacactaaccgctttactagcctgcgctctaacccttgtcgcttgcgactcgagattccacaatgtcacagctgtggcaggagaaaatgttactttcaactccacggccgatacccagtggtcgtggagtggctcaggtagctacttaactatctgcaatagctccacttcccccggcatatccccaaccaagtaccaatgcaatgccagcctgttcaccctcatcaacgcttccaccctggacaatggactctatgtaggctatgtaccctttggtgggcaaggaaagacccacgcttacaacctggaagttcgccagcccagaaccactacccaagcttctcccaccaccaccaccaccaccaccatcaccagcagcagcagcagcagcagccacagcagcagcagcagattattgactttggttttggccagctcatctgccgctacccaggccatctacagctctgtgcccgaaaccactcagatccaccgcccagaaacgaccaccgccaccaccctacacacctccagcgatcagatgccgaccaacatcacccccttggctcttcaaatgggacttacaagccccactccaaaaccagtggatgcggccgaggtctccgccctcgtcaatgactgggcggggctgggaatgtggtggttcgccataggcatgatggcgctctgcctgcttctgctctggctcatctgctgcctccaccgcaggcgagccagaccccccatctatagacccatcattgtcctgaaccccgataatgatgggatccatagattggatggcctgaaaaacctacttttttcttttacagtatgataaattgagacatgcctcgcattttcttgtacatgttccttctcccaccttttctggggtgttctacgctggccgctgtgtctcacctggaggtagactgcctctcacccttcactgtctacctgctttacggattggtcaccctcactctcatctgcagcctaatcacagtaatcatcgccttcatccagtgcattgattacatctgtgtgcgcctcgcatacttcagacaccacccgcagtaccgagacaggaacattgcccaacttctaagactgctctaatcatgcataagactgtgatctgccttctgatcctctgcatcctgcccaccctcacctcctgccagtacaccacaaaatctccgcgcaaaagacatgcctcctgccgcttcacccaactgtggaatatacccaaatgctacaacgaaaagagcgagctctccgaagcttggctgtatggggtcatctgtgtcttagttttctgcagcactgtctttgccctcataatctacccctactttgatttgggatggaacgcgatcgatgccatgaattaccccacctttcccgcacccgagataattccactgcgacaagttgtacccgttgtcgttaatcaacgccccccatcccctacgcccactgaaatcagctactttaacctaacaggcggagatgactgacgccctagatctagaaatggacggcatcagtaccgagcagcgtctcctagagaggcgcaggcaggcggctgagcaagagcgcctcaatcaggagctccgagatctcgttaacctgcaccagtgcaaaagaggcatcttttgtctggtaaagcaggccaaagtcacctacgagaagaccggcaacagccaccgcctcagttacaaattgcccacccagcgccagaagctggtgctcatggtgggtgagaatcccatcaccgtcacccagcactcggtagagaccgaggggtgtctgcactccccctgtcggggtccagaagacctctgcaccctggtaaagaccctgtgcggtctcagagatttagtcccctttaactaatcaaacactggaatcaataaaaagaatcacttacttaaaatcagacagcaggtctctgtccagtttattcagcagcacctccttcccctcctcccaactctggtactccaaacgccttctggcggcaaacttcctccacaccctgaagggaatgtcagattcttgctcctgtccctccgcacccactatcttcatgttgttgcagatgaagcgcaccaaaacgtctgacgagagcttcaaccccgtgtacccctatgacacggaaagcggccctccctccgtccctttcctcacccctcccttcgtgtctcccgatggattccaagaaagtccccccggggtcctgtctctgaacctggccgagcccctggtcacttcccacggcatgctcgccctgaaaatgggaagtggcctctccctggacgacgctggcaacctcacctctcaagatatcaccaccgctagccctcccctcaaaaaaaccaagaccaacctcagcctagaaacctcatcccccctaactgtgagcacctcaggcgccctcaccgtagcagccgccgctcccctggcggtggccggcacctccctcaccatgcaatcagaggcccccctgacagtacaggatgcaaaactcaccctggccaccaaaggccccctgaccgtgtctgaaggcaaactggccttgcaaacatcggccccgctgacggccgctgacagcagcaccctcacagtcagtgccacaccaccccttagcacaagcaatggcagcttgggtattgacatgcaagcccccatttacaccaccaatggaaaactaggacttaactttggcgctcccctgcatgtggtagacagcctaaatgcactgactgtagttactggccaaggtcttacgataaacggaacagccctacaaactagagtctcaggtgccctcaactatgacacatcaggaaacctagaattgagagctgcagggggtatgcgagttgatgcaaatggtcaacttatccttgatgtagcttacccatttgatgcacaaaacaatctcagccttaggcttggacagggacccctgtttgttaactctgcccacaacttggatgttaactacaacagaggcctctacctgttcacatctggaaataccaaaaagctagaagttaatatcaaaacagccaagggtctcatttatgatgacactgctatagcaatcaatgcgggtgatgggctacagtttgactcaggctcagatacaaatccattaaaaactaaacttggattaggactggattatgactccagcagagccataattgctaaactgggaactggcctaagctttgacaacacaggtgccatcacagtaggcaacaaaaatgatgacaagcttaccttgtggaccacaccagacccatcccctaactgtagaatctattcagagaaagatgctaaattcacacttgttttgactaaatgcggcagtcaggtgttggccagcgtttctgttttatctgtaaaaggtagccttgcgcccatcagtggcacagtaactagtgctcagattgtcctcagatttgatgaaaatggagttctactaagcaattcttcccttgaccctcaatactggaactacagaaaaggtgaccttacagagggcactgcatataccaacgcagtgggatttatgcccaacctcacagcatacccaaaaacacagagccaaactgctaaaagcaacattgtaagtcaggtttacttgaatggggacaaatccaaacccatgaccctcaccattaccctcaatggaactaatgaaacaggagatgccacagtaagcacttactccatgtcattctcatggaactggaatggaagtaattacattaatgaaacgttccaaaccaactccttcaccttctcctacatcgcccaagaataaaaagcatgacgctgttgatttgattcaatgtgtttctgttttattttcaagcacaacaaaatcattcaagtcattcttccatcttagcttaatagacacagtagcttaatagacccagtagtgcaaagccccattctagcttataactagtggagaagtactcgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagtcttagatctctcaacgcagcaccagcaccaacacttcgcagtgtaaaaggccaagtgccgagagagtatatataggaataaaaagtgacgtaaacgggcaaagtccaaaaaacgcccagaaaaaccgcacgcgaacctacgccccgaaacgaaagccaaaaaacactagacactcccttccggcgtcaacttccgctttcccacgctacgtcacttgccccagtcaaacaaactacatatcccgaacttccaagtcgccacgcccaaaacaccgcctacacctccccgcccgccggcccgcccccaaacccgcctcccgccccgcgccccgccccgcgccgcccatctcattatcatattggcttcaatccaaaataaggtatattattgatgatg

SEQ ID NO: 5 - protein, 1145 aa, synthetic construct
  REGION 1..1145: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLNDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNRKRRAPVKQTLNFDLLKLAGDVESNPGPMALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKYPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVELGGGGSGGGGMSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTVSDTNDHAKNNDTT

SEQ ID NO: 6 - DNA, 1187 bp, synthetic construct
  misc_feature 1..1187: note = Description of Artificial Sequence: Synthetic polynucleotide
  source 1..1187: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataaggcgaagcgctccctatcagtgatagagatctccctatcagtgatagagatcgtcgacgagctcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 7 - DNA, 2908 bp, synthetic construct
  misc_feature 1..2908: note = Description of Artificial Sequence: Synthetic polynucleotide
  source 1..2908: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgagatcttccgtttatctaggtaccagatatcgccaccatggccctgagcaaagtgaaactgaacgatacactgaacaaggaccagctgctgtccagcagcaagtacaccatccagcggagcaccggcgacagcatcgatacccccaactacgacgtgcagaagcacatcaacaagctgtgcggcatgctgctgatcacagaggacgccaaccacaagttcaccggcctgatcggcatgctgtacgccatgagccggctgggccgggaggacaccatcaagatcctgcgggacgccggctaccacgtgaaggccaatggcgtggacgtgaccacacaccggcaggacatcaacggcaaagaaatgaagttcgaggtgctgaccctggccagcctgaccaccgagatccagatcaatatcgagatcgagagccggaagtcctacaagaaaatgctgaaagaaatgggcgaggtggcccccgagtacagacacgacagccccgactgcggcatgatcatcctgtgtatcgccgccctggtgatcacaaagctggccgctggcgacagatctggcctgacagccgtgatcagacgggccaacaatgtgctgaagaacgagatgaagcggtacaagggcctgctgcccaaggacattgccaacagcttctacgaggtgttcgagaagtacccccacttcatcgacgtgttcgtgcacttcggcattgcccagagcagcaccagaggcggctccagagtggagggcatcttcgccggcctgttcatgaacgcctacggcgctggccaggtgatgctgagatggggcgtgctggccaagagcgtgaagaacatcatgctgggccacgccagcgtgcaggccgagatggaacaggtggtggaggtgtacgagtacgcccagaagctgggcggagaggccggcttctaccacatcctgaacaaccctaaggcctccctgctgtccctgacccagttcccccacttctccagcgtggtgctgggaaatgccgccggactgggcatcatgggcgagtaccggggcacccccagaaaccaggacctgtacgacgccgccaaggcctacgccgagcagctgaaagaaaacggcgtgatcaactacagcgtgctggacctgaccgctgaggaactggaagccatcaagcaccagctgaaccccaaggacaacgacgtggagctgggaggcggaggatctggcggcggaggcatgagcagacggaacccctgcaagttcgagatccggggccactgcctgaacggcaagcggtgccacttcagccacaactacttcgagtggccccctcatgctctgctggtgcggcagaacttcatgctgaaccggatcctgaagtccatggacaagagcatcgacaccctgagcgagatcagcggagccgccgagctggacagaaccgaggaatatgccctgggcgtggtgggagtgctggaaagctacatcggctccatcaacaacatcacaaagcagagcgcctgcgtggccatgagcaagctgctgacagagctgaacagcgacgacatcaagaagctgagggacaacgaggaactgaacagccccaagatccgggtgtacaacaccgtgatcagctacattgagagcaaccgcaagaacaacaagcagaccatccatctgctgaagcggctgcccgccgacgtgctgaaaaagaccatcaagaacaccctggacatccacaagtccatcaccatcaacaatcccaaagaaagcaccgtgtctgacaccaacgatcacgccaagaacaacgacaccacctgatgagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 8 - protein, 594 aa, synthetic construct
  REGION 1..594: note = Description of Artificial Sequence: Synthetic polypeptide
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKYPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVELGGGGSGGGGMSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTVSDTNDHAKNNDTT

SEQ ID NO: 9 - DNA, 3617 bp, synthetic construct
  misc_feature 1..3617: note = Description of Artificial Sequence: Synthetic polynucleotide
  source 1..3617: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataaggcgaagcgctccctatcagtgatagagatctccctatcagtgatagagatcgtcgacgagctcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggatatcgccaccatggaactgctgatcctgaaggccaacgccatcaccaccatcctgaccgccgtgaccttctgcttcgccagcggccagaacatcaccgaggaattctaccagagcacctgtagcgccgtgagcaagggctacctgagcgccctgagaaccggctggtacaccagcgtgatcaccatcgagctgagcaacatcaaagaaaacaagtgcaacggcaccgacgccaaagtgaagctgatcaagcaggaactggacaagtacaagaacgccgtgaccgagctgcagctgctgatgcagagcacccccgccaccaacaaccgggccagacgggagctgccccggttcatgaactacaccctgaacaacgccaaaaagaccaacgtgaccctgagcaagaagcggaagcggcggttcctgggctttctgctgggcgtgggcagcgccattgccagcggcgtggccgtgtctaaggtgctgcacctggaaggcgaagtgaacaagatcaagagcgccctgctgagcaccaacaaggccgtggtgtccctgagcaacggcgtgagcgtgctgaccagcaaggtgctggatctgaagaactacatcgacaagcagctgctgcccatcgtgaacaagcagagctgcagcatcagcaacatcgagacagtgatcgagttccagcagaagaacaaccggctgctggaaatcacccgggagttcagcgtgaacgccggcgtgaccacccctgtgtccacctacatgctgaccaacagcgagctgctgagcctgatcaacgacatgcccatcaccaacgaccagaaaaagctgatgagcaacaacgtgcagatcgtgcggcagcagagctactccatcatgtccatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgacagaggctggtactgcgacaacgccggcagcgtgtcattctttccacaggccgagacatgcaaggtgcagagcaaccgggtgttctgcgacaccatgaacagcctgaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgacgagttcgacgccagcatcagccaggtgaacgagaagatcaaccagagcctggccttcatccggaagtccgacgagctgctgcacaatgtgaatgccggcaagtccaccaccaactgatgagcggccatctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 10 - protein, 524 aa, synthetic construct
  REGION 1..524: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 11 - protein, 7 aa, synthetic construct
  REGION 1..7: note = Description of Artificial Sequence: Synthetic peptide
GGGSGGG

SEQ ID NO: 12 - protein, 9 aa, synthetic construct
  REGION 1..9: note = Description of Artificial Sequence: Synthetic peptide
GGGGSGGGG

(19) United States
(12) Patent Application Publication
(10) Pub. No.: US 2024/0093161 A1
(43) Pub. Date: Mar. 21, 2024

(54) SIMIAN ADENOVIRAL VECTORS WITH TWO EXPRESSION CASSETTES

(71) Applicant: GLAXOSMITHKLINE BIOLOGICALS SA, Rixensart (BE)

(72) Inventor: Stefano COLLOCA, Rome (IT)

(73) Assignee: GLAXOSMITHKLINE BIOLOGICALS SA, Rixensart (BE)

(21) Appl. No.: 18/453,078

(22) Filed: Aug. 21, 2023

Related U.S. Application Data
(63) Continuation of application No. 16/756,377, filed on Apr. 15, 2020, now abandoned.
(60) Provisional application No. 62/572,944, filed on Oct. 16, 2017.

(51) Int. Cl. C12N 7/00; A61K 39/235; C12N 15/86

(52) U.S. Cl. CPC C12N 7/00; A61K 39/235; C12N 15/86; A61K 2039/5256

(57) ABSTRACT

A simian adenoviral vector comprising two expression cassettes, wherein each expression cassette comprises a transgene and a promoter, and wherein the first expression cassette is inserted in the E1 region of the simian adenoviral vector, and the second expression cassette is inserted in a region of the adenoviral vector that is compatible with vector replication.

### CROSS-REFERENCE TO RELATED APPLICATIONS

This application is a Continuation of copending application Ser. No. 16/756,377 filed Apr. 15, 2020, which is the National Phase under 35 U.S.C. § 371 of International Application No. PCT/EP2018/078210, filed on Oct. 16, 2018, which claims the benefit under 35 U.S.C. § 119(e) to U.S. Provisional Application No. 62/572,944, filed on Oct. 16, 2017, all of which are hereby expressly incorporated by reference into the present application.

### REFERENCE TO ELECTRONIC SEQUENCE LISTING

This application contains a Sequence Listing which has been submitted electronically in .XML format and is hereby incorporated by reference in its entirety. Said .XML copy, created on Aug. 21, 2023, is named 2801-0326PUS2_SL.xml and is 138,799 bytes in size. The sequence listing contained in this .XML file is part of the specification and is hereby incorporated by reference herein in its entirety.

### FIELD OF THE INVENTION

This invention is in the field of recombinant adenoviral vectors. The invention relates to an adenoviral vector comprising two expression cassettes. In particular, the invention relates to a simian adenovirus such as a chimpanzee (chimp) adenovirus comprising two expression cassettes.

### BACKGROUND OF THE INVENTION

Recombinant adenoviruses are useful in gene therapy and as vaccines.

Human adenoviruses have been widely used for gene transfer applications due to their large transgene capacity and ability to achieve highly efficient gene transfer in a variety of target tissues.

However, most humans are exposed to and develop immunity to human adenoviruses. Therefore, there is a demand for vectors which effectively deliver molecules to a target and minimize the effect of pre-existing immunity to human adenovirus serotypes. Simian adenoviruses are effective in this regard; they are sufficiently closely related to human viruses to be effective in inducing immunity to delivered exogenous antigens to which humans have little or no pre-existing immunity. Therefore, viral vectors based on simian adenoviruses can provide an alternative to the use of human derived adenoviral vectors for the development of nucleic acid based vaccines.

Replication defective adenoviruses deliver their genome to the interior of a cell and, because they do not replicate, do not amplify the transgene payload. Typically, the E1 gene is replaced with a transgene cassette comprising a promoter of choice and a nucleic acid sequence corresponding to a gene or genes of interest, resulting in a replication defective recombinant virus.

There is a need in the art for improved recombinant adenoviruses.

### SUMMARY OF THE INVENTION

The invention relates to a simian adenoviral vector comprising two expression cassettes. In particular, the invention relates to a simian adenovirus such as a chimpanzee (chimp) adenovirus comprising two expression cassettes. Examples of suitable chimp adenoviruses include ChAd155 and ChAd83.

The adenoviral vectors of the invention are useful as components of immunogenic compositions for the induction of an immune response in a subject, methods for their use in treatment and processes for manufacture.

The term “vector” refers to an agent (such as a plasmid or virus) that contains or carries genetic material and can be used to introduce exogenous genes into an organism. The adenoviral vector of the present invention is derived from a non-human simian adenovirus, also referred to as a “simian adenovirus”. Preferably, the simian adenoviral vector of the present invention is a simian adenovirus.

Each expression cassette in the adenoviral vector of the invention comprises a transgene and a promoter. A “transgene” is a nucleic acid sequence, heterologous to the vector sequences flanking the transgene, which encodes a polypeptide of interest. The nucleic acid coding sequence is operatively linked to regulatory components in a manner which permits transgene transcription, translation, and/or expression in a host cell. A “promoter” is a nucleotide sequence that permits the binding of RNA polymerase and directs the transcription of a gene. Typically, a promoter is located in a non-coding region of a gene, proximal to the transcriptional start site.

In adenoviral vectors of the invention, the first expression cassette is inserted in the E1 region of the virus, and the second expression cassette is inserted into a second region of the adenoviral vector.

In a simian adenoviral vector comprising two expression cassettes of the invention, the first expression cassette is inserted in the E1 region of the simian adenoviral vector, and the second expression cassette is inserted in a region of the adenoviral vector that is compatible with vector replication. A region of the adenoviral vector genome is considered “compatible with vector replication” if disruption of this region would not affect the ability of the adenoviral vector to replicate.

Preferably, in adenoviral vectors of the invention, the first expression cassette is inserted in the E1 region of the virus, and the second expression cassette is inserted into the E3, HE1 or HE2 region of the adenoviral vector. As is well known in the art, the E3 genes are expressed in the early phase of transduction to prepare the host cell for viral replication. E3 is involved in immune modulation. The term “HE1” is used to describe a site located between the stop codons of L5 and E4. The term “HE2” has been used to define a site located between the end of the ITR and the cap site of E4 mRNA.

For example, in a ChAd155 adenovirus vector:
HE1 ChAd155: insertion site between bp 34611 and 34612 of SEQ ID NO: 1.
HE2 ChAd155: insertion site between bp 37662 and 37663 of SEQ ID NO: 1.

In another example, in a ChAd83 adenovirus vector:
HE1 ChAd83: insertion site between bp 33535 and 33536 of SEQ ID NO: 2.
HE2 ChAd83: insertion site between bp 36387 and 36388 of SEQ ID NO: 2.

As the first expression cassette is inserted in the E1 region of the adenoviral vector, the native E1 region is deleted. In order to increase the cloning capacity of the vector, the native E3 region can be removed from the adenoviral vector. The native E3 region can be deleted from the adenoviral vector in embodiments of the invention where the second expression cassette is inserted in the E3 region, or in embodiments where the second expression cassette is not inserted into the E3 region. The insertion in HE1 or HE2 site doesn't require deletion of any specific sequence of the vector backbone.

Preferably, the second expression cassette is inserted into the HE1 or HE2 region of the adenoviral vector. Most preferably, the second expression cassette is inserted in the HE2 region of the adenoviral vector. In one embodiment, the native E3 region is deleted from the adenoviral vector to increase the cloning capacity of the vector, and the second expression cassette is inserted in the HE1 or HE2 region of the adenoviral vector.

In embodiments of the invention, the first expression cassette of the adenoviral vector may comprise a human CMV or an enhanced human CMV promoter, and/or the second expression cassette may comprise a human CMV or an enhanced human CMV promoter.

In a preferred embodiment, the first and second expression cassettes comprise different promoters. For example, in one embodiment, the first expression cassette may comprise a human CMV promoter and the second expression cassette an enhanced human CMV promoter (or vice versa).

In one aspect of the invention, there is provided an adenoviral vector of the invention, wherein the first expression cassette is inserted in the E1 region of the virus, and the second expression cassette is inserted in a region of the adenoviral vector that is compatible with vector replication, wherein at least one of the first and second expression cassette comprises an enhanced CMV promoter. In some embodiments, the enhanced hCMV promoter can include a nucleic acid sequence having at least about 90%, at least about 95%, at least about 96%, at least about 97%, at least about 98%, at least about 99%, or more, sequence identity to SEQ ID NO: 6. In some embodiments, the promoter comprises or consists of a nucleic acid sequence of SEQ ID NO: 6.

Adenoviral vectors of the invention are derived from a simian adenoviral vector, for example, from chimpanzees (Pan troglodytes), bonobos (Pan paniscus), gorillas (Gorilla gorilla) and orangutans (Pongo abelii and Pongo pygnaeus). Chimpanzee adenoviruses include, but are not limited to AdY25, ChAd3, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155, ChAd15, SadV41, sAd4310A, sAd4312, SAdV31, SAdV-A1337, ChAdOx1, ChAdOx2 and ChAd157. Preferably, the simian adenoviral vector of the invention is a ChAd83 or ChAd155 adenovirus vector, most preferably a ChAd155 adenovirus vector.

Preferably, the adenoviral vector of the invention has a seroprevalence of less than 30%, preferably less than 10% in human subjects and, most preferably, no seroprevalence in human subjects.

In a preferred embodiment, the simian adenoviral vector of the invention is capable of infecting a mammalian cell.

In one embodiment, the first and second expression cassettes of the adenoviral vector of the invention comprise transgenes from respiratory syncytial virus (RSV). For example, in one embodiment, one of the expression cassettes comprises an RSV F antigen, and the other expression cassette comprises RSV M and N antigens. In such embodiments, the vector preferably encodes an RSV F0ΔTM antigen (fusion (F) protein deleted of the transmembrane and cytoplasmic regions), and RSV M2-1 (transcription anti-termination) and N (nucleocapsid) antigens.

The present invention also provides a composition comprising a simian adenoviral vector and a pharmaceutically acceptable excipient.

In addition, the present invention provides a simian adenoviral vector or composition comprising such an adenoviral vector for use as a medicament, a vaccine, and/or for the therapy or prophylaxis of a disease.

The invention also provides a method of inducing an immune response in a subject comprising administering the simian adenoviral vector or composition to the subject.

### DESCRIPTION OF THE FIGURES

FIG. 1: Simian adenoviral constructs with a single expression cassette. Inverted terminal repeats (ITR) flank the 3′ and 5′ ends; E1 is the early gene 1; CMV is the cytomegalovirus promoter; CASI is the CASI promoter, RG is a model antigen, WPRE is the Woodchuck Hepatitis Postranscriptional Regulatory Element, ΔE3 denotes that the early gene 3 is deleted; fiber denotes the adenoviral gene encoding the fiber protein and E4 is the early gene 4.

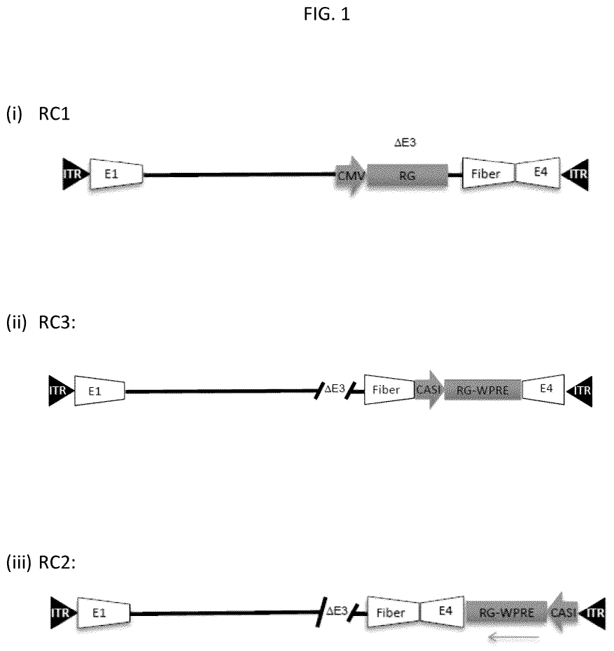

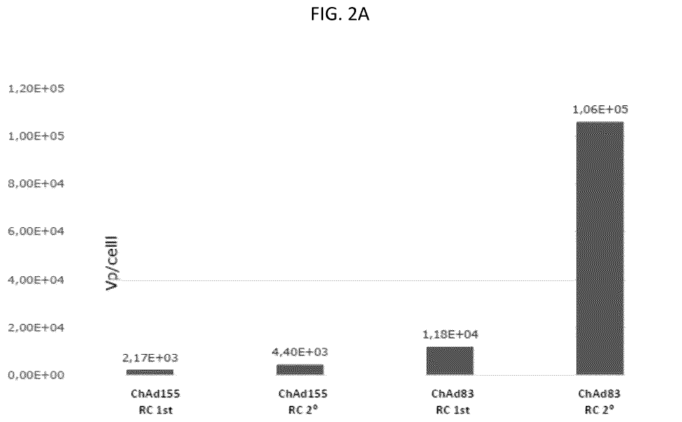

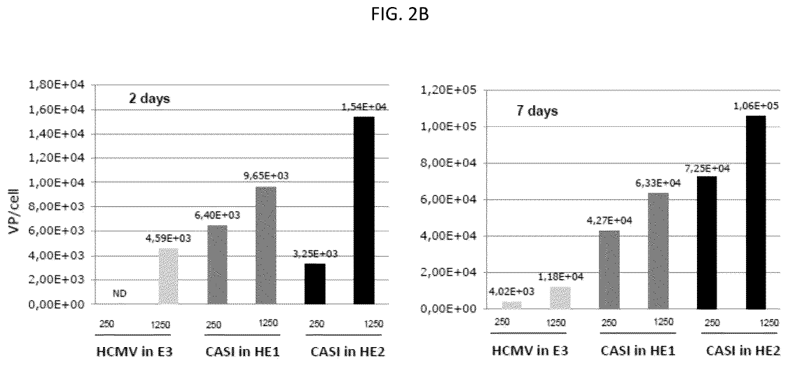

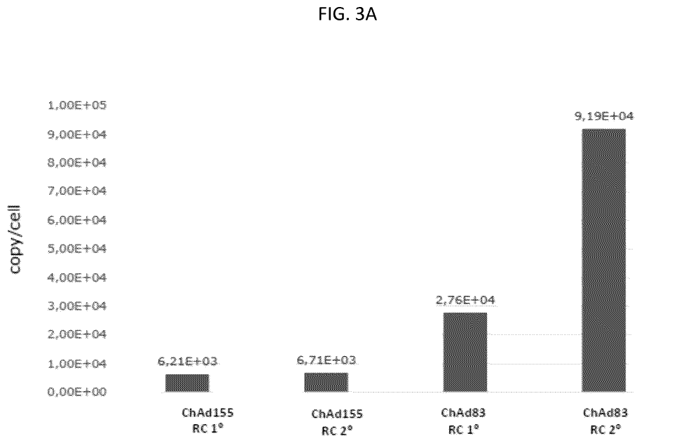

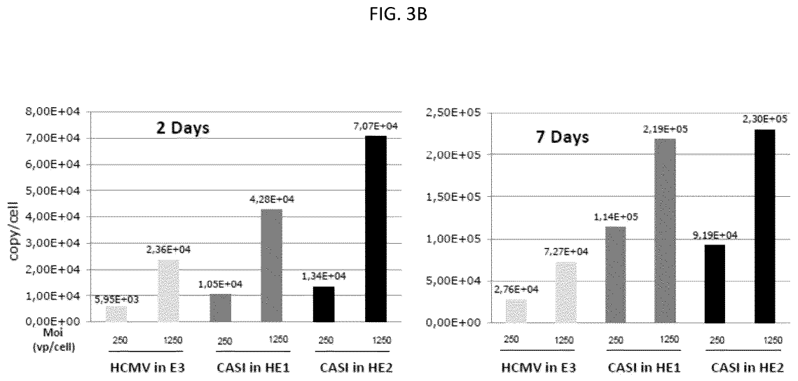

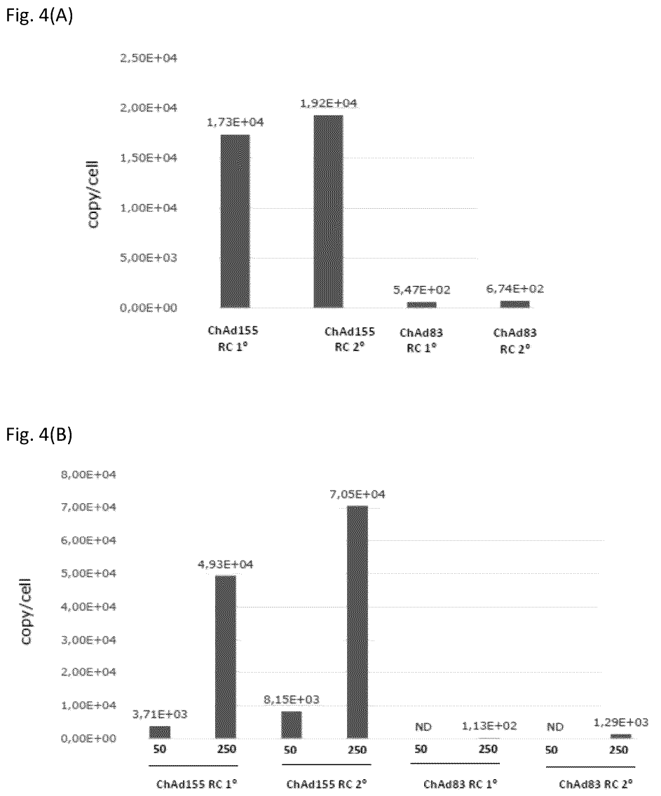

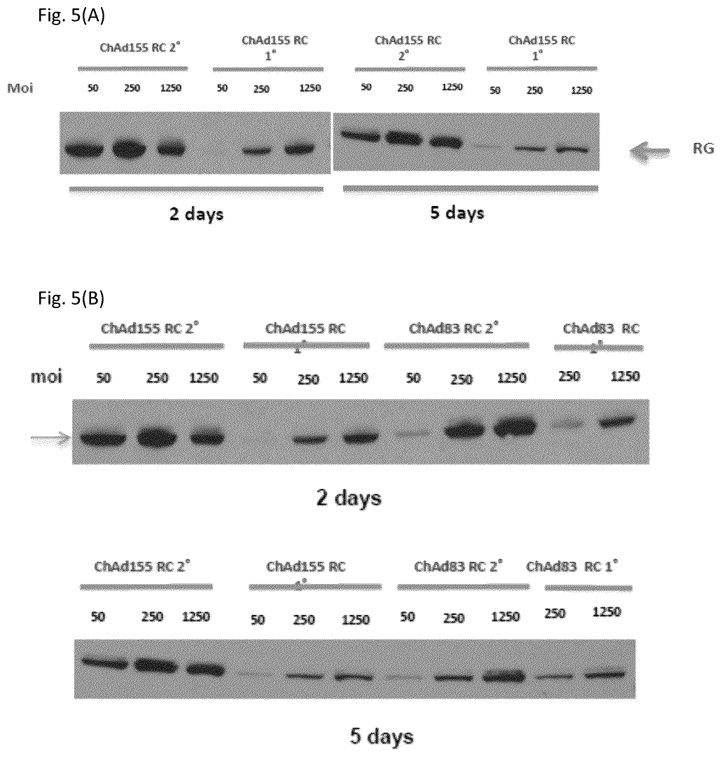

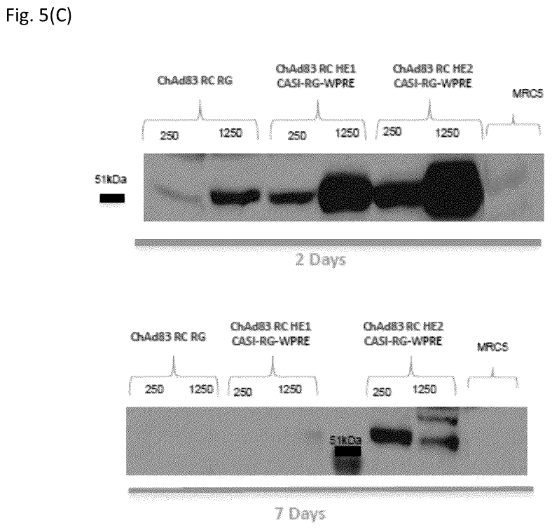

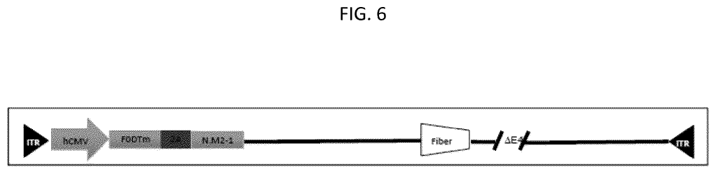

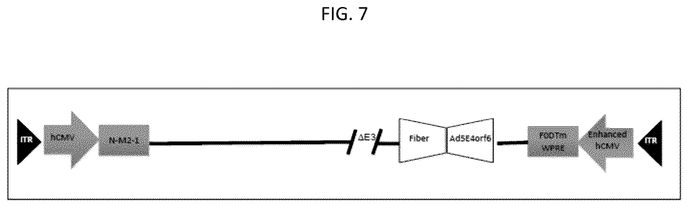

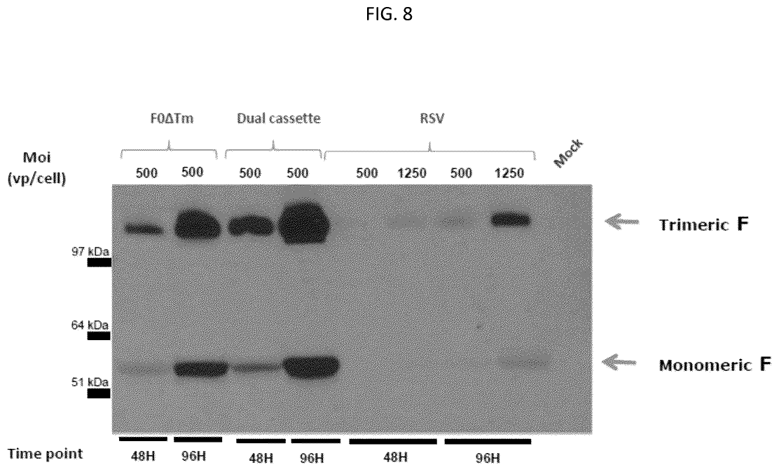

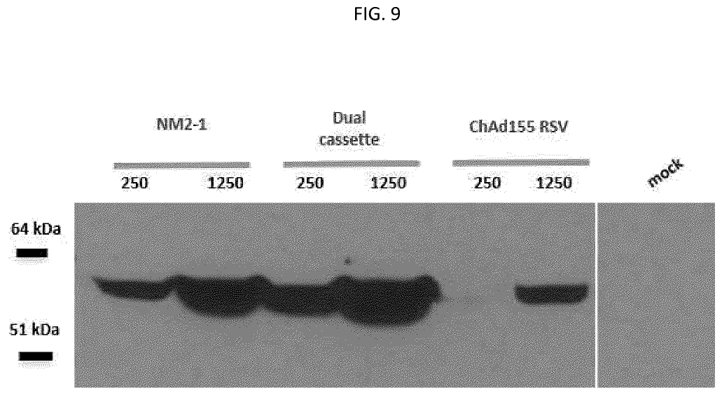

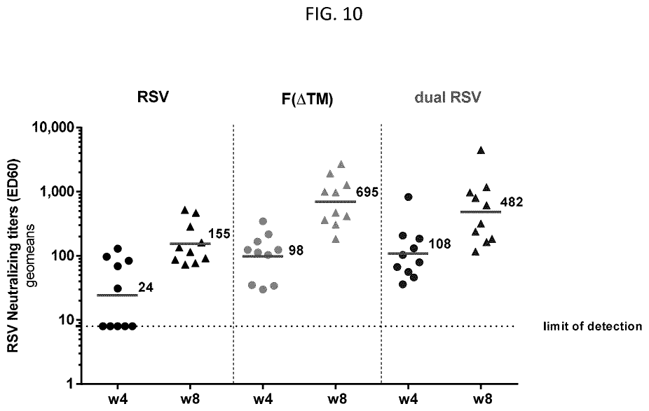

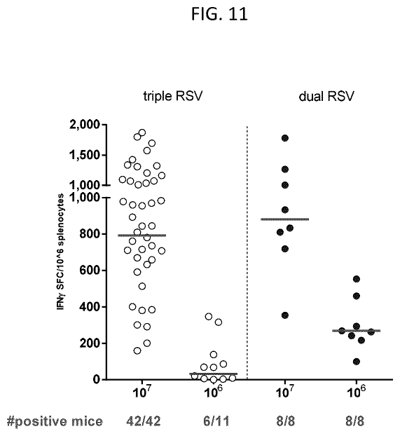

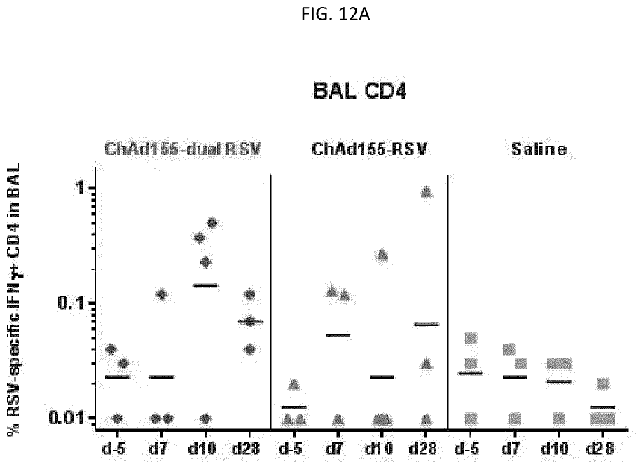

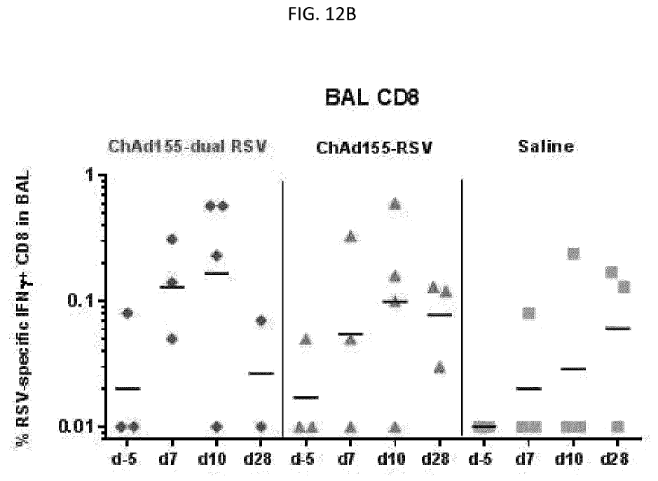

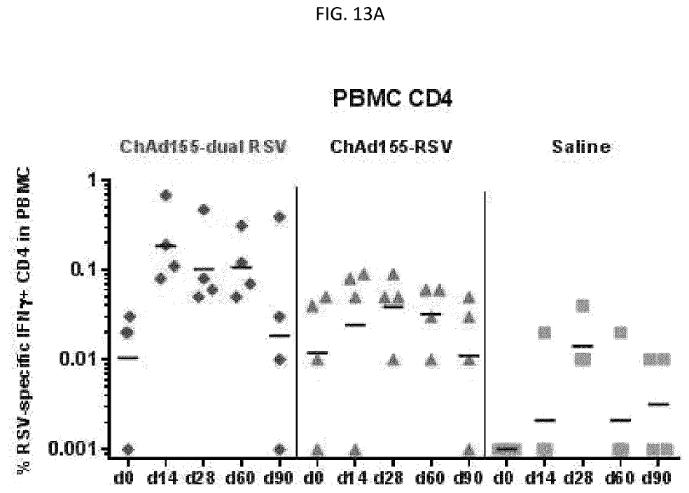

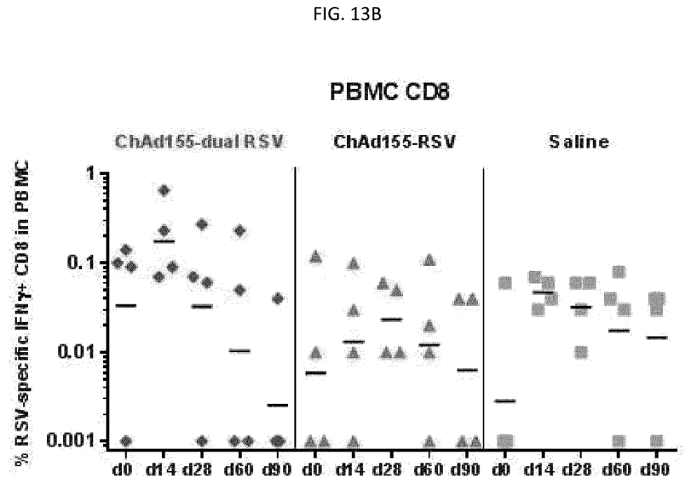

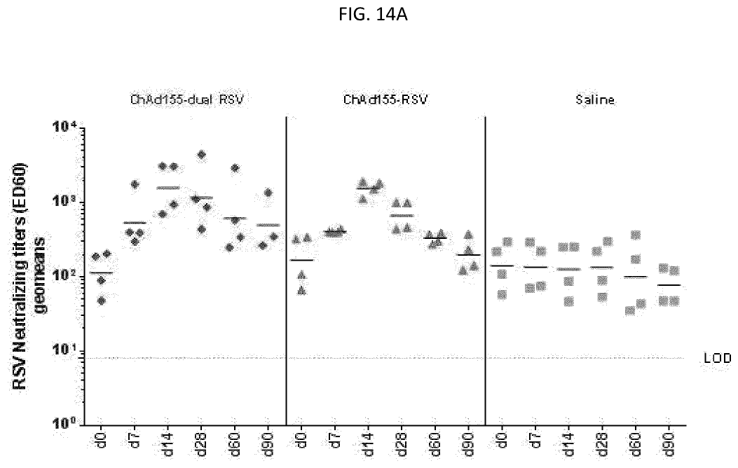

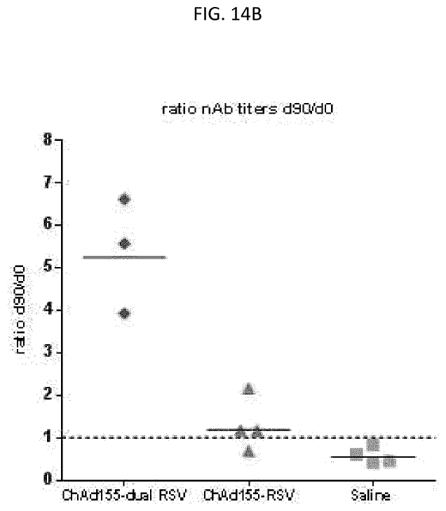

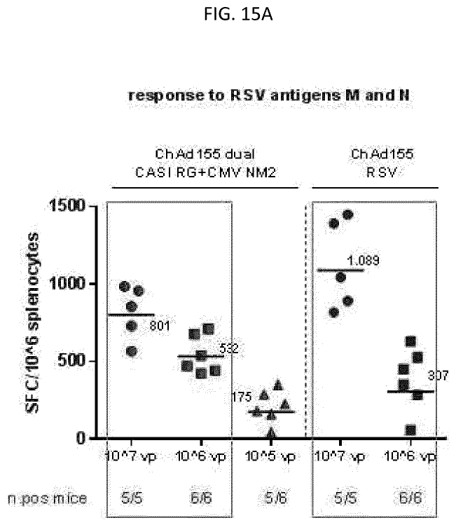

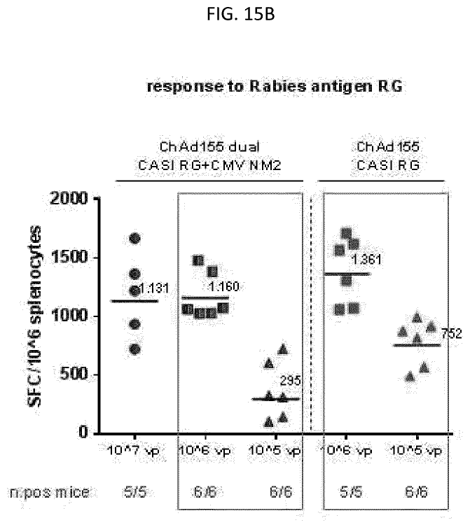

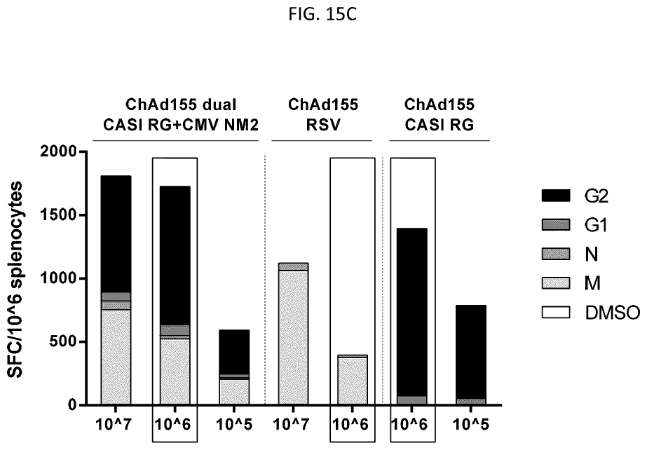

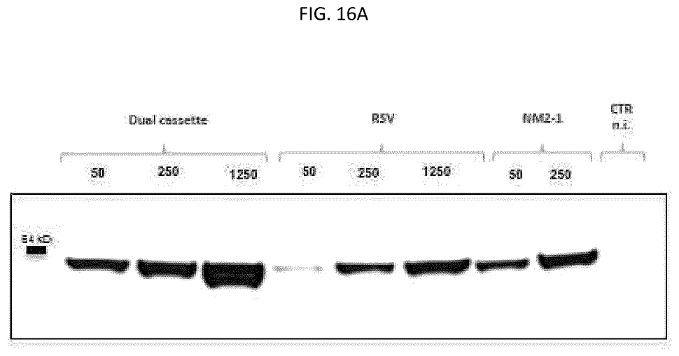

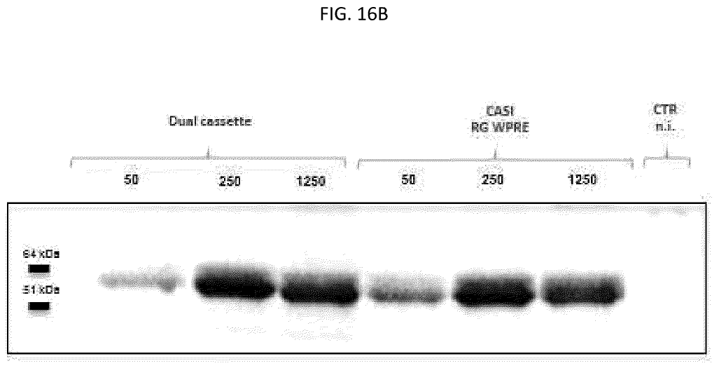

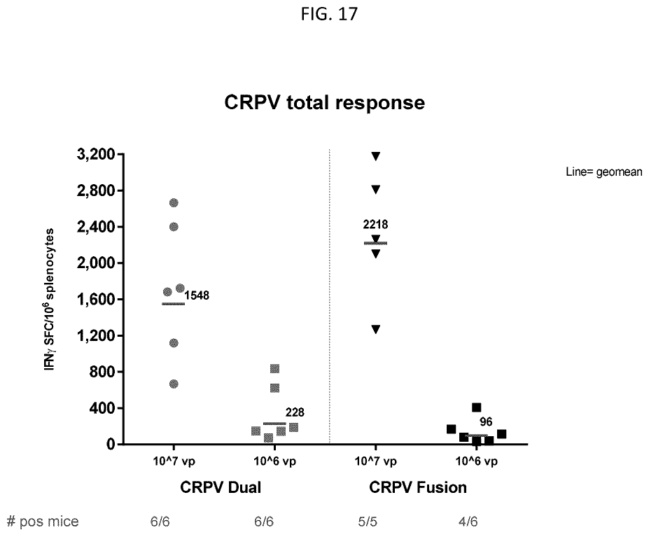

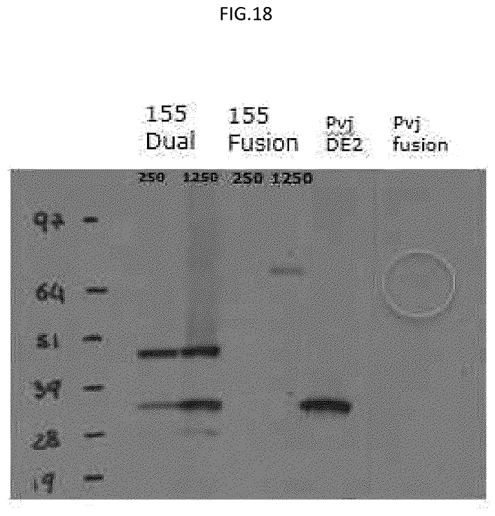

Three different simian adenoviral vectors are shown in FIG. 1. The vector of FIG. 1(i) was constructed by inserting a transgene expression cassette in place of the E3 region of the adenoviral genome (“RC1”) (top panel), the vector of FIG. 1(ii) was formed by inserting a transgene expression cassette in the HE1 region, i.e., between the stop codons of the fiber gene and the E4 region (“RC3”) (middle panel), and the vector of FIG. 1(iii) was made by inserting a transgene expression cassette in the HE2 region, i.e., between the end of the ITR and the cap site of E4 mRNA (“RC2”) (bottom panel).

FIG. 2A: Production of ChAd155 and ChAd83 with transgene cassette inserted in E3 and HE2 sites (RC1 and RC2 vectors of FIG. 1) in a primary human cell line.

FIG. 2B: Production of ChAd83 with transgene cassette inserted in E3, HE1 and HE2 (the RC1, RC2 and RC3 vectors of FIG. 1) in a human MRC5 cell line at two and seven days post-infection. Cells were infected at multiplicities of infection of 250 and 1250.

FIG. 3A: Total viral genome copy number of RC1 and RC2 vector (ChAd155 and ChAd83) of FIG. 1 in a primary human cell line.

FIG. 3B: Total viral genome copy number of RC1, RC2 and RC3 versions of ChAd83 vector of FIG. 1 in a human MRC5 cell line at two and seven days post-infection. Cells were infected at multiplicities of infection of 250 and 1250.

FIG. 4: Total viral genome copy number of ChAd155 RC1 and RC2 and ChAd83 RC1 and RC2 vectors of FIG. 1 in a murine cell line FIG. 4(A), top panel) and in a non-human primate cell line FIG. 4(B), bottom panel). Cells were infected at multiplicities of infection of 50 and 250.

FIG. 5: Comparison of the expression levels of ChAd155 RC1 and RC2 vectors expressing a model rabies glycoprotein (RG) transgene in a murine cell line, demonstrated by western blot at two and five days post-infection FIG. 5(A), top panel). Comparison of the expression levels of ChAd155 RC1 and RC2 vectors with ChAd83 RC1 and RC2 vectors expressing a model rabies glycoprotein (RG) transgene in a murine cell line, demonstrated by western blot at two and five days post-infection FIG. 5(B), bottom panel). Cells were infected at multiplicities of infection of 50, 250 and 1250.

FIG. 5(C): Comparison of the expression levels of ChAd83 RC1, RC2 and RC3 vectors expressing a model rabies glycoprotein (RG) transgene in a human MRC5 cell line, demonstrated by western blot at two and seven days post-infection. Cells were infected at multiplicities of infection of 250 and 1250.

FIG. 6: Another simian adenoviral construct of with a single expression cassette. Inverted terminal repeats (ITR) flank the 3′ and 5′ ends; human CMV (hCMV) is the cytomegalovirus promoter; FΔTM (F0DTM) and N.M2-1 are RSV antigens; 2A is a self-cleaving linking sequence; ΔE4 denotes that the early gene 4 is deleted; fiber denotes the adenoviral gene encoding the fiber protein. In the vector of FIG. 6, the transgene expression cassette is inserted in place of the E1 region of the adenoviral genome.

FIG. 7: A simian adenoviral construct according to the invention with a dual expression cassette. Inverted terminal repeats (ITR) flank the 3′ and 5′ ends; human CMV (hCMV) is the cytomegalovirus promoter; Enhanced hCMV is the enhanced cytomegalovirus promoter; N-M2-1 and FΔTM (F0DTM) are the RSV antigens; WPRE is the Woodchuck Hepatitis Postranscriptional Regulatory Element; ΔE3 denotes that the early gene 3 is deleted; fiber denotes the adenoviral gene encoding the fiber protein; and Ad5E4orf6 in a substitute in the early gene 4 (E4) region.

The vector of FIG. 7 was constructed by inserting a first transgene expression cassette in place of the E1 region of the adenoviral genome, and a second transgene expression cassette in the HE2 region, i.e., downstream of the right ITR.

FIG. 8: Comparison of the expression levels of vectors expressing F0ΔTM transgene in a MRC5 cell line, demonstrated by western blot at 48 hours and 96 hours post-infection under non-reducing conditions. Cells were infected at multiplicities of infection of 500 and 1250.

FIG. 9: Comparison of the expression levels of vectors expressing NM2-1 transgene in a MRC5 cell line, demonstrated by western blot at 48 hours post-infection under reducing conditions. Cells were infected at multiplicities of infection of 250 and 1250.

FIG. 10: Comparison of the immunogencity from ChAd155 vectors expressing the RSV antigen F0ΔTM (F×Tm). The data was collected at 4 weeks and 8 weeks after vaccination with a dose of 5×10⁸ virus particles.

FIG. 11: Comparison of the immunogencity from ChAd155 vectors expressing the M2 RSV antigen. The data was collected at 3 weeks after vaccination with a dose of either 10⁷ or 10⁶ virus particles.

FIG. 12A and 12B: Illustrate the results from the experiment of Example 9 to investigate the lung T cell responses from ChAd155 vectors. FIG. 12A shows the CD4+ response, and FIG. 12B shows the CD8+ response.

FIGS. 13A and 13B: Show the results from the experiment of Example 9 to investigate the peripheral T cell responses from ChAd155 vectors. FIG. 13A shows the PBMC CD4+ response, and FIG. 13B shows the PBMC CD8+ response.

FIG. 14A and 14B: Also show results from Example 9. FIG. 14A shows the RSV neutralising Ab titres, and FIG. 14B illustrates the ratio of the nAb from day D90 to D0.

FIG. 15A. 15B and 15C: Show the results of the immunogencity experiment of Example 10.

FIG. 16A and 16B: Western blots obtained using the expression in Hela cells of the vectors in Example 11.

FIG. 17: Illustrates the results of the CRPV experiment of Example 12.

FIG. 18: Shows the results of the HPV dual cassette vector characterisation of Example 13.

### ANNOTATION OF THE SEQUENCES

SEQ ID NO: 1—Polynucleotide sequence encoding wild type ChAd155

SEQ ID NO: 2—Polynucleotide sequence encoding wild type ChAd83

SEQ ID NO: 3—Polynucleotide sequence encoding the CASI promoter

SEQ ID NO: 4—Polynucleotide sequence encoding ChAd155/RSV

SEQ ID NO: 5—RSV F0ΔTM-N-M2-1 amino acid sequence

SEQ ID NO: 6—Polynucleotide sequence encoding the enhanced hCMV promoter

SEQ ID NO: 7—Polynucleotide sequence encoding the hCMV NM2 bghpolyA cassette

SEQ ID NO: 8—NM2 amino acid (protein) sequence

SEQ ID NO: 9—Polynucleotide sequence encoding the hCMV F0 WPRE bghpolyA cassette

SEQ ID NO: 10—F0 amino acid (protein) sequence

SEQ ID NO: 11—Amino acid sequence of a flexible linker

SEQ ID NO: 12—Amino acid sequence of a flexible linker

### DETAILED DESCRIPTION OF THE INVENTION

### Adenoviruses

Adenoviruses are nonenveloped icosahedral viruses with a linear double stranded DNA genome of approximately 36 kb. Adenoviruses can transduce numerous cell types of several mammalian species, including both dividing and nondividing cells, without integrating into the genome of the host cell. They have been widely used for gene transfer applications due to their proven safety, ability to achieve highly efficient gene transfer in a variety of target tissues, and large transgene capacity. Human adenoviral vectors are currently used in gene therapy and vaccines but have the drawback of a high worldwide prevalence of pre-existing immunity, following previous exposure to common human adenoviruses.

Adenoviruses have a characteristic morphology with an icosahedral capsid comprising three major proteins, hexon (II), penton base (III) and a knobbed fiber (IV), along with a number of other minor proteins, VI, VIII, IX, IIIa and IVa2. The hexon accounts for the majority of the structural components of the capsid, which consists of 240 trimeric hexon capsomeres and 12 penton bases. The hexon has three conserved double barrels and the top has three towers, each tower containing a loop from each subunit that forms most of the capsid. The base of the hexon is highly conserved between adenoviral serotypes, while the surface loops are variable. The penton is another adenoviral capsid protein; it forms a pentameric base to which the fiber attaches. The trimeric fiber protein protrudes from the penton base at each of the 12 vertices of the capsid and is a knobbed rod-like structure. The primary role of the fiber protein is to tether the viral capsid to the cell surface via the interaction of the knob region with a cellular receptor. Variations in the flexible shaft, as well as knob regions of fiber, are characteristic of the different adenovral serotypes.

The adenoviral genome has been well characterized. The linear, double-stranded DNA is associated with the highly basic protein VII and a small peptide pX (also termed mu). Another protein, V, is packaged with this DNA-protein complex and provides a structural link to the capsid via protein VI. There is general conservation in the overall organization of the adenoviral genome with respect to specific open reading frames being similarly positioned, e.g. the location of the E1A, E1B, E2A, E2B, E3, E4, L1, L2, L3, L4 and L5 genes of each virus. Each extremity of the adenoviral genome comprises a sequence known as an inverted terminal repeat (ITR), which is necessary for viral replication. The 5′ end of the adenoviral genome contains the 5′ cis-elements necessary for packaging and replication; i.e., the 5′ ITR sequences (which can function as origins of replication) and the native 5′ packaging enhancer domains, which contain sequences necessary for packaging linear adenoviral genomes and enhancer elements for the E1 promoter. The 3′ end of the adenoviral genome includes 3′ cis-elements, including the ITRs, necessary for packaging and encapsidation. The virus also comprises a virus-encoded protease, which is necessary for processing some of the structural proteins required to produce infectious virions.

The structure of the adenoviral genome is described on the basis of the order in which the viral genes are expressed following host cell transduction. More specifically, the viral genes are referred to as early (E) or late (L) genes according to whether transcription occurs prior to or after onset of DNA replication. In the early phase of transduction, the E1A, E1B, E2A, E2B, E3 and E4 genes of adenovirus are expressed to prepare the host cell for viral replication. The E1 gene is considered a master switch, it acts as a transcription activator and is involved in both early and late gene transcription. E2 is involved in DNA replication; E3 is involved in immune modulation and E4 regulates viral mRNA metabolism. During the late phase of infection, expression of the late genes L1-L5, which encode the structural components of the viral particles, is activated. Late genes are transcribed from the Major Late Promoter (MLP) with alternative splicing.

HE1 and HE2 sites were identified as potential insertion sites for a transgene since the insertion in these specific points does not interrupt the coding sequences or important regulatory sequences of a chimp adenovirus, such as a Type C or E chimp adenovirus, for example, ChAd155 and ChAd83. The HE1 and HE2 sites can be identified by sequence alignment in any chimp adenovirus. Therefore, cloning of expression cassettes in the HE1 and HE2 sites of the ChAd genomes doesn't impact the virus replication cycle.

### Adenoviral Replication

Historically, adenovirus vaccine development has focused on defective, non-replicating vectors. They are rendered replication defective by deletion of the E1 region genes, which are essential for replication. Typically, non-essential E3 region genes are also deleted to make room for exogenous transgenes. An expression cassette comprising the transgene under the control of an exogenous promoter is then inserted. These replication-defective viruses are then produced in E1-complementing cells.

The term “replication-defective ” or “replication-incompetent” adenovirus refers to an adenovirus that is incapable of replication because it has been engineered to comprise at least a functional deletion (or “loss-of-function” mutation), i.e. a deletion or mutation which impairs the function of a gene without removing it entirely, e.g. introduction of artificial stop codons, deletion or mutation of active sites or interaction domains, mutation or deletion of a regulatory sequence of a gene etc, or a complete removal of a gene encoding a gene product that is essential for viral replication, such as one or more of the adenoviral genes selected from E1A, E1B, E2A, E2B, E3 and E4 (such as E3 ORF1, E3 ORF2, E3 ORF3, E3 ORF4, E3 ORF5, E3 ORF6, E3 ORF7, E3 ORF8, E3 ORF9, E4 ORF7, E4 ORF6, E4 ORF4, E4 ORF3, E4 ORF2 and/or E4 ORF1). Suitably, E1 and optionally E3 and/or E4 are deleted. If deleted, the aforementioned deleted gene region will suitably not be considered in the alignment when determining percent identity with respect to another sequence.

### Vectors of the Invention

Viral vectors based on non-human simian adenovirus represent an alternative to the use of human derived vectors for gene therapy and genetic vaccines. Certain adenoviruses isolated from non-human simians are closely related to adenoviruses isolated from humans, as demonstrated by their efficient propagation in cells of human origin. As humans develop little or no immunity to simian adenoviruses, they promise to provide an improved alternative to human adenoviral uses.

“Low seroprevalence” may mean having a reduced pre-existing neutralizing antibody level as compared to human adenovirus 5 (Ad5). Similarly or alternatively, “low seroprevalence” may mean less than about 30% seroprevalence, less than about 20% seroprevalence, less than about 15% seroprevalence, less than about 10% seroprevalence, less than about 5% seroprevalence, less than about 4% seroprevalence, less than about 3% seroprevalence, less than about 2% seroprevalence, less than about 1% seroprevalence or no detectable seroprevalence. Seroprevalence can be measured as the percentage of individuals having a clinically relevant neutralizing titer (defined as a 50% neutralisation titer >200) using methods as described in Hum. Gene Ther. (2004) 15: 293.

The adenoviral vector of the present invention is derived from a nonhuman simian adenovirus, also referred to as a “simian adenovirus.” Numerous adenoviruses have been isolated from nonhuman simians such as chimpanzees, bonobos, rhesus macaques, orangutans and gorillas. Vectors derived from these adenoviruses can induce strong immune responses to transgenes encoded by these vectors. Certain advantages of vectors based on nonhuman simian adenoviruses include a relative lack of cross-neutralizing antibodies to these adenoviruses in the human target population, thus their use overcomes the pre-existing immunity to human adenoviruses. For example, some simian adenoviruses have no cross reactivity with preexisting human neutralizing antibodies and cross-reaction of certain chimpanzee adenoviruses with pre-existing human neutralizing antibodies is only present in 2% of the target population, compared with 35% in the case of certain candidate human adenovirus vectors (Sci. Transl. Med. (2012) 4: 1).

Adenoviral vectors of the invention are derived from a simian adenovirus, e.g., from chimpanzees (Pan troglodytes), bonobos (Pan paniscus), gorillas (Gorilla gorilla) and orangutans (Pongo abelii and Pongo pygnaeus). They include adenoviruses from Group B, Group C, Group D, Group E and Group G. Chimpanzee adenoviruses include, but are not limited to AdY25, ChAd3, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155, ChAd15, SadV41 and ChAd157 ChAd3, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155, ChAd15, SadV41, sAd4310A, sAd4312, SAdV31, SAdV-A1337, ChAdOx1, ChAdOx2 and ChAd157. Alternatively, adenoviral vectors may be derived from nonhuman simian adenoviruses isolated from bonobos, such as PanAd1, PanAd2, PanAd3, Pan 5, Pan 6, Pan 7 (also referred to as C7) and Pan 9. Vectors may include, in whole or in part, a nucleotide encoding the fiber, penton or hexon of a non-human adenovirus.

In an embodiment of the adenoviral vectors of the invention, the adenoviral vector has a seroprevalence of less than 30%, less than 20%, less than 10% or less than 5% in human subjects, preferably no seroprevalence in human subjects and more preferably no seroprevalence in human subjects that have not previously been in contact with a chimpanzee adenoviral vector.

In embodiments of the adenoviral vectors of the invention, the adenoviral DNA is capable of entering a mammalian target cell, i.e. it is infectious. An infectious recombinant adenoviral vector of the invention can be used as a prophylactic or therapeutic vaccine and for gene therapy. Thus, in an embodiment, the recombinant adenoviral vector comprises an endogenous molecule for delivery into a target cell. The target cell is a mammalian cell, e.g. a bovine cell, a canine cell, a caprine cell, a cervine cell, a chimpanzee cell, a chiroptera cell, an equine cell, a feline cell, a human cell, a lupine cell, an ovine cell, a porcine cell, a rodent cell, an ursine cell or a vulpine cell. The endogenous molecule for delivery into a target cell is an expression cassette.

In an embodiment of the invention, the vector comprises a left ITR region, a deleted E1 region, then a deleted E3 region, and, optionally, additional enhancer elements; these are followed by a fiber region, an E4 region and a right ITR. Translation occurs in the rightward and leftward directions. In this embodiment, the first expression cassette is inserted in the deleted E1 region, and the second expression cassette is insertion in the deleted E3 region. In a further embodiment, the promoters of the two expression cassettes are CMV promoters. In a yet further embodiment, the enhancer element is the Hepatitis B Postranslational Regulatory Element (HPRE) or the Woodchuck Hepatitis Postranslational Regulatory Element (WPRE).

In one embodiment of the invention, the vector comprises left and right ITR regions; a deleted E1 region; at least a partially deleted E3 region; a fiber region; an E4 region; two expression cassettes, each comprising: a promoter and at least one an antigen of interest and, optionally, one or more enhancer elements. The first expression cassette is inserted in the deleted E1 region, and the second expression cassette is inserted at the HE1 site, i.e., between the stop codons of the fiber gene and an E4 region (“the HE1 site”). The ChAd155 HE1 insertion site is between bp 34611 and 34612 of the wild type ChAd155 sequence. The ChAd83 HE1 insertion site is between bp 33535 and 33536 of the wild type ChAd83 sequence. Translation occurs in the rightward and leftward directions. In a further embodiment, the promoters are CMV promoters. In a preferred embodiment, one promoter is a CMV promoter and the other is a eCMV promoter. In a yet further embodiment, the enhancer element is HPRE or WPRE.

In a further embodiment, the vector comprises left and right ITR regions; a deleted E1 region; at least a partially deleted E3 region; a fiber region; an E4 region; two expression cassettes, each comprising: a promoter, at least one antigen of interest and, optionally, one or more enhancer elements. The first expression cassette is inserted in the deleted E1 region, and the second expression cassette is inserted at the HE2 site, i.e., between the end of the left ITR and the cap site of the E4 mRNA (“the HE2 site”). The ChAd155 HE2 insertion site is between bp 37662 and 37663 of the wild type ChAd155 sequence. The ChAd83 HE2 insertion site is between bp 36387 and 36388 of the wild type ChAd83 sequence. Translation occurs in the rightward and leftward directions. In a further embodiment, the promoters are CMV promoters. In a preferred embodiment, one promoter is a CMV promoter and the other is a eCMV promoter. In a yet further embodiment, the enhancer element is HPRE or WPRE (the enhancer element increases expression of the transgene).

The HE1 and HE2 sites were identified as insertion sites for a transgene, as the insertion in these specific points does not interrupt the coding sequences or regulatory sequences of

ChAd155 and ChAd83. Therefore, inserting expression cassettes in the HE1 or HE2 sites of the ChAd genome does not affect the viral replication cycle.

In an embodiment of the invention, the vector is a functional or an immunogenic derivative of an adenoviral vector. By “derivative of an adenoviral vector” is meant a modified version of the vector, e.g., one or more nucleotides of the vector are deleted, inserted, modified or substituted.

### Regulatory Elements

Regulatory elements, i.e., expression control sequences, include appropriate transcription initiation, termination, promoter and enhancer sequences; efficient RNA processing signals such as splicing and polyadenylation (poly A) signals including rabbit beta-globin polyA; tetracycline regulatable systems, microRNAs, posttranscriptional regulatory elements (e.g., WPRE, posttranscriptional regulatory element of woodchuck hepatitis virus); sequences that stabilize cytoplasmic mRNA; sequences that enhance translation efficiency (e.g., Kozak consensus sequence); sequences that enhance protein stability; and when desired, sequences that enhance secretion of an encoded product.

A “promoter” is a nucleotide sequence that permits the binding of RNA polymerase and directs the transcription of a gene. Typically, a promoter is located in a non-coding region of a gene, proximal to the transcriptional start site. Sequence elements within promoters that function in the initiation of transcription are often characterized by consensus nucleotide sequences. Examples of promoters include, but are not limited to, promoters from bacteria, yeast, plants, viruses, and mammals, including simians and humans. A great number of expression control sequences, including promoters which are internal, native, constitutive, inducible and/or tissue-specific, are known in the art and may be utilized.

Promoters of the invention will typically be heterologous promoters. Promoters of the invention can be constitutive.

Examples of promoters include, but are not limited to, promoters from bacteria, yeast, plants, viruses, and mammals (including humans).

Examples of promoters include, without limitation, the TBG promoter, the retroviral Rous sarcoma virus LTR promoter (optionally with the enhancer), the cytomegalovirus (CMV) promoter (optionally with the CMV enhancer, see, e.g., Boshart et al, Cell, 41: 521-530 (1985)), the CASI promoter, the SV40 promoter, the dihydrofolate reductase promoter, the B-actin promoter, the phosphoglycerol kinase (PGK) promoter, and the EF1a promoter (Invitrogen).

Suitable promoters include the cytomegalovirus (CMV) promoter and the CASI promoter. The CMV promoter is strong and ubiquitously active. It has the ability to drive high levels of transgene expression in many tissue types and is well known in the art. The CMV promoter can be used in vectors of the invention, either with or without a CMV enhancer.

The CASI promoter is a synthetic promoter described as a combination of the CMV enhancer, the chicken beta-actin promoter, and a splice donor and splice acceptor flanking the ubiquitin (UBC) enhancer (U.S. Pat. No. 8,865,881).

In some embodiments, the CASI promoter can include a nucleic acid sequence having at least about 90%, at least about 95%, at least about 96%, at least about 97%, at least about 98%, at least about 99%, or more, sequence identity to SEQ ID NO: 3. In some embodiments, the promoter comprises or consists of a nucleic acid sequence of SEQ ID NO: 3.

In some embodiments, the enhanced hCMV promoter can include a nucleic acid sequence having at least about 90%, at least about 95%, at least about 96%, at least about 97%, at least about 98%, at least about 99%, or more, sequence identity to SEQ ID NO: 6. In some embodiments, the promoter comprises or consists of a nucleic acid sequence of SEQ ID NO: 6.

Optionally, vectors carrying transgenes encoding therapeutically useful or immunogenic products may also include selectable markers or reporter genes. The reporter gene may be chosen from those known in the art. Suitable reporter genes include, but are not limited to enhanced green fluorescent protein, red fluorescent protein, luciferase and secreted embryonic alkaline phosphatase (seAP), which may include sequences encoding geneticin, hygromicin or purimycin resistance, among others. Such selectable reporters or marker genes (which may or may not be located outside the viral genome to be packaged into a viral particle) can be used to signal the presence of the plasmids in bacterial cells, such as ampicillin resistance. Other components of the vector may include an origin of replication.

A “posttranscriptional regulatory element,” as used herein, is a DNA sequence that, when transcribed, enhances the expression of the transgene(s) or fragments thereof that are delivered by viral vectors of the invention. Postranscriptional regulatory elements include, but are not limited to the Hepatitis B Virus Postranscriptional Regulatory Element (HPRE) and the Woodchuck Hepatitis Postranscriptional Regulatory Element (WPRE). The WPRE is a tripartite cis-acting element that has been demonstrated to enhance transgene expression driven by certain, but not all promoters.

In embodiments of the invention, a ChAd155 vector may comprise one or more of a promoter, an enhancer, and a reporter gene. For example, vectors of the invention may comprise ChAd155-enhanced hCMV-SeAP, ChAd155-CASI-seAP and ChAd155-hCMV-seAP, optionally with a tetracycline on/off transcriptional control and ChAd155-CMV-hFerL-chEF1-seAP with a tetracycline on/off transcriptional control.

In embodiments of the invention, a ChAd83 vector may comprise one or more of a promoter, an enhancer, and a reporter gene. For example, vectors of the invention may comprise ChAd155-enhanced hCMV-SeAP, ChAd83-enhanced hCMV-SeAP, ChAd155-CASI-seAP and ChAd83-hCMV-seAP, optionally with a tetracycline on/off transcriptional control and ChAd83-CMV-hFerL-chEF1-seAP with a tetracycline on/off transcriptional control.

Vectors of the invention are generated using techniques provided herein, in conjunction with techniques known to those of skill in the art. Such techniques include conventional cloning techniques of cDNA such as those described in texts, use of overlapping oligonucleotide sequences of the adenovirus genomes, polymerase chain reaction, and any suitable method which provides the desired nucleotide sequence.

### Transgenes

A “transgene” is a nucleic acid sequence, heterologous to the vector sequences flanking the transgene, which encodes a polypeptide of interest. The nucleic acid coding sequence is operatively linked to regulatory components in a manner which permits transgene transcription, translation, and/or expression in a host cell. In embodiments of the invention, the vectors express transgenes at a therapeutic or a prophylactic level. A “functional derivative” of a transgenic polypeptide is a modified version of a polypeptide, e.g., wherein one or more amino acids are deleted, inserted, modified or substituted.

The transgene may be used for prophylaxis or treatment, e.g., as a vaccine for inducing an immune response, to correct genetic deficiencies by correcting or replacing a defective or missing gene, or as a cancer therapeutic. As used herein, induction of an immune response refers to the ability of a protein to induce a T cell and/or a humoral antibody immune response to the protein.

The immune response elicited by the transgene may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing cytokines, e.g. interferon gamma (IFN gamma), tumor necrosis factor alpha (TNF alpha) and/or interleukin 2 (IL2). Alternatively, or additionally, the antigen specific T cell response comprises a CD8+ T cell response, such as a response involving CD8+ T cells expressing cytokines, e.g., IFN gamma, TNF alpha and/or IL2.

The composition of the transgene sequence will depend upon the use to which the resulting vector will be put. In an embodiment, the transgene is a sequence encoding a product which is useful in biology and medicine, such as a prophylactic transgene, a therapeutic transgene or an immunogenic transgene, e.g., protein or RNA. Protein transgenes include antigens. Antigenic transgenes of the invention induce an immunogenic response to a disease causing organism.

Transgenes of the invention include, but are not limited to, rabies virus antigens, e.g., rabies glycoprotein (RG), respiratory syncytial virus (RSV) antigens, human immunodeficiency virus (HIV) antigens, or fragments thereof.

As a result of the redundancy in the genetic code, a polypeptide can be encoded by a variety of different nucleic acid sequences. Coding is biased to use some synonymous codons, i.e., codons that encode the same amino acid, more than others. By “codon optimized,” it is meant that modifications in the codon composition of a recombinant nucleic acid are made without altering the amino acid sequence. Codon optimization has been used to improve mRNA expression in different organisms by using organism-specific codon-usage frequencies.

In addition to, and independently from, codon bias, some synonymous codon pairs are used more frequently than others. This codon pair bias means that some codon pairs are overrepresented and others are underrepresented. Codon pair deoptimization has been used to reduce viral virulence. For example, it has been reported that polioviruses modified to contain underrepresented codon pairs demonstrated decreased translation efficiency and were attenuated compared to wild type poliovirus (Science (2008) 320: 1784). Engineering a synthetic attenuated virus by codon pair deoptimization can produce viruses that encode the same amino acid sequences as wild type but use different pairwise arrangements of synonymous codons. Viruses attenuated by codon pair deoptimization generated up to 1000-fold fewer plaques compared to wild type, produced fewer viral particles and required about 100 times as many viral particles to form a plaque.

In contrast, polioviruses modified to contain codon pairs that are overrepresented in the human genome acted in a manner similar to wild type RNA and generated plaques identical in size to wild type RNA (Coleman et al. (2008) Science 320: 1784). This occurred despite the fact that the virus with overrepresented codon pairs contained a similar number of mutations as the virus with underrepresented codon pairs and demonstrated enhanced translation compared to wild type. This observation suggests that codon pair optimized constructs would be expected to act in a manner similar to their non-codon pair optimized counterparts and would not be expected to provide a functional advantage. Without wishing to be constrained by theory, this may be because natural evolution has optimized codon pairing.

A construct of the invention may comprise a codon optimized nucleic acid sequence. Alternatively or additionally, a vector of the invention comprises a codon optimized sequence of a transgene or an immunogenic derivative or fragment thereof. A construct of the invention may comprise a codon pair optimized nucleic acid sequence. Alternatively or additionally, a vector of the invention comprises or consists of a codon pair optimized sequence of a transgene or an immunogenic derivative or fragment thereof.

### Respiratory Syncytial Virus (RSV) Transgenes

In one embodiment, the present invention provides the use of a recombinant simian-derived adenoviral vector comprising two expression cassettes, wherein each expression cassette comprises an immunogenic transgene derived from human respiratory syncytial virus (RSV), in the treatment or prophylaxis of RSV infection. In one embodiment, the recombinant simian-derived adenoviral vector of the present invention comprises an RSV F antigen in one of the expression cassettes, and another RSV viral antigen in the other expression cassette. Suitable antigens are discussed further below. In one embodiment, the recombinant simian-derived adenoviral vector comprises RSV M and N antigens in the second expression cassette. In such embodiments, the vector preferably encodes an RSV F0ΔTM antigen (fusion (F) protein deleted of the transmembrane and cytoplasmic regions), and RSV M2-1 (transcription anti-termination) and N (nucleocapsid) antigens.

Infection with RSV does not confer full protective immunity. Infection in infancy is followed by symptomatic RSV re-infections which continue throughout adulthood. These re-infections generally go undiagnosed because they usually present as common acute upper respiratory tract infections. In more vulnerable persons (e.g., immunocompromised adults or elderly), re-infections can however also lead to severe disease. Both arms of the immune system (humoral and cellular immunity) are involved in protection from severe disease [Guvenel A K, Chiu C and Openshaw P J. Current concepts and progress in RSV vaccine development. Expert Rev Vaccines. 2014; 13(3): 333-44.].

The humoral immune response is capable of neutralizing the virus and inhibiting viral replication, thereby playing a major role in protection against lower respiratory RSV infection and severe disease [Piedra PA, Jewell AM, Cron SG, et al., Correlates of immunity to respiratory syncytial virus (RSV) associated-hospitalization: establishment of minimum protective threshold levels of serum neutralizing antibodies. Vaccine. 2003; 21(24): 3479-82.]. Passive immunization, in the form of Immunoglobulin G (lgG) RSV-neutralizing monoclonal antibodies (Synagis) given prophylactically, has been shown to prevent RSV disease to some extent in premature infants and newborns with bronchopulmonary dysplasia or underlying cardiopulmonary disease [Cardenas S, Auais A and Piedimonte G. Palivizumab in the prophylaxis of respiratory syncytial virus infection. Expert Rev Anti Infect Ther. 2005; 3(5): 719-26].

T cells are also involved in the control of RSV disease. Lethal RSV infections have been described in patients with low CD8 T cells counts, as in the case of severe combined immunodeficiency, bone marrow and lung transplant recipients [Hertz, 1989]. The histopathology of fatal cases of RSV infection of newborns shows that there is a relative paucity of CD8 T cells in the lung infiltrate [Welliver T P, Garofalo R P, Hosakote Y, et al., Severe human lower respiratory tract illness caused by respiratory syncytial virus and influenza virus is characterized by the absence of pulmonary cytotoxic lymphocyte responses. J Infect Dis. 2007. 195(8): 1126-36.]. Moreover, the presence of CD8 T cells producing Interferon-gamma (IFN-γ) has been associated with diminished Th2 responses and reduced eosinophilia in animal models of RSV [Castilow E M and Varga S M. Overcoming T cell-mediated immunopathology to achieve safe RSV vaccination. Future Virol. 2008; 3(5): 445-454; Stevens W W, Sun J, Castillo J P, et al., Pulmonary eosinophilia is attenuated by early responding CD8(+) memory T cells in a murine model of RSV vaccine-enhanced disease. Viral Immunol. 2009; 22(4): 243-51].

Suitable antigens of RSV which are useful as immunogens to immunize a human or non-human animal can be selected from: the fusion protein (F), the attachment protein (G), the matrix protein (M2) and the nucleoprotein (N). The term “F protein” or “fusion protein” or “F protein polypeptide” or “fusion protein polypeptide” refers to a polypeptide or protein having all or part of an amino acid sequence of an RSV Fusion protein polypeptide. Similarly, the term “G protein” or “G protein polypeptide” refers to a polypeptide or protein having all or part of an amino acid sequence of an RSV Attachment protein polypeptide. The term “M protein” or “matrix protein” or

“M protein polypeptide” refers to a polypeptide or protein having all or part of an amino acid sequence of an RSV Matrix protein and may include either or both of the M2-1 (which may be written herein as M2.1) and M2-2 gene products. Likewise, the term “N protein” or “Nucleocapsid protein” or “N protein polypeptide” refers to a polypeptide or protein having all or part of an amino acid sequence of an RSV Nucleoprotein.

Two groups of human RSV strains have been described, the A and B groups, based mainly on differences in the antigenicity of the G glycoprotein. Numerous strains of RSV have been isolated to date, any of which are suitable in the context of the antigens of the immunogenic combinations disclosed herein. Exemplary strains indicated by GenBank and/or EMBL Accession number can be found in US published application number 2010/0203071 (WO2008114149), which is incorporated herein by reference for the purpose of disclosing the nucleic acid and polypeptide sequences of RSV F and G proteins suitable for use in present invention. In an embodiment, the RSV F protein can be an ectodomain of an RSV F Protein (F0ΔTM).

Exemplary M and N protein nucleic acids and protein sequences can be found, e.g., in US published application number 2014/0141042 (WO2012/089833), which are incorporated herein for purpose of disclosing the nucleic acid and polypeptide sequences of RSV M and N proteins suitable for use in present invention.

Suitably, for use with in present invention, transgene nucleic acids encode an RSV F antigen and RSV, M and N antigens. More specifically, the nucleic acids encode an RSV F0ΔTM antigen (fusion (F) protein deleted of the transmembrane and cytoplasmic regions), and RSV M2-1 (transcription anti-termination) and N (nucleocapsid) antigens.

### Fusion (F) Protein Deleted of the Transmembrane and Cytoplasmic Regions (F0ΔTM)

The RSV F protein is a major surface antigen and mediates viral fusion to target cells. The F protein is an antigen which is highly conserved among RSV subgroups and strains. The F protein is a target for neutralizing antibodies, including the prophylactic RSV-neutralizing monoclonal antibody Synagis. Deletion of the transmembrane region and cytoplasmic tail permits secretion of the F0ΔTM protein. Neutralizing antibodies including Synagis, that recognize this soluble form of the F protein, inhibit RSV infectivity in vitro [Magro M, Andreu D, Gómez-Puertas P, et al., Neutralization of human respiratory syncytial virus infectivity by antibodies and low-molecular-weight compounds targeted against the fusion glycoprotein. J Virol. 2010; 84(16): 7970-82].

### Nucleocapsid (N) Protein

The N protein is an internal (non-exposed) antigen, highly conserved between RSV strains and known to be a source of many T cell epitopes. The N protein is essential for the replication and transcription of the RSV genome. The primary function of the N protein is to encapsulate the virus genome for the purposes of RNA transcription, replication and packaging and protects it from ribonucleases.

### Transcription Anti-Termination (M2-1) Protein

The M2-1 protein is a transcription anti-termination factor that is important for the efficient synthesis of full-length messenger RNAs (mRNAs) as well as for the synthesis of polycistronic readthrough mRNAs, which are characteristic of non-segmented negative-strand RNA viruses. M2-1 is an internal (non-exposed) antigen, which is highly conserved between RSV strains and known to be a source of many T cell epitopes.

### N-M2-1 Fusion Protein

A polynucleotide encoding a linker is positioned between the polynucleotide encoding an RSV N antigen, or fragment thereof, and the polynucleotide encoding an RSV M2.1 antigen, or fragment thereof. Thus, in certain preferred examples, an expression cassette contains a transgene which encodes a fused RSV viral protein N-linker-M2.1 It is preferred that the linker is a flexible linker, preferably a flexible linker comprising an amino acid sequence according to SEQ ID NO: 11 (Gly-Gly-Gly-Ser-Gly-Gly-Gly) or SEQ ID NO: 12 (Gly-Gly-Gly-Gly-Ser-Gly-Gly-Gly-Gly).

### Papilloma (PV) Transgenes

In one embodiment, the present invention provides the use of a recombinant simian-derived adenoviral vector comprising two expression cassettes, wherein each expression cassette comprises an immunogenic transgene derived from a papilloma virus (PV), in the treatment or prophylaxis of a papilloma virus induced disease. Suitably, the recombinant simian-derived adenoviral vector of the present invention comprises a modified papilloma virus E1 antigen in one of the expression cassettes, and a modified papilloma virus E2 antigen in the other expression cassette.

Human Papillomavirus (HPV) are small DNA viruses that infect mucosal and/or cutaneous skin and cause multiple disease conditions, including cervical neoplasia, cervical cancer, and other anogenital cancers. There are over 40 types of HPV known to infect the anogenital tract of humans and about 15 high-risk HPV genotypes are causally associated with human cervical cancers. A majority of HPV infections of the cervical epithelium are subclinal and self-resolving within a two years period. However, persistant infection with high risk HPV types may cause lesions and progress to invasive cancer.

Suitable antigens of HPV which are useful as immunogens are described in WO2018060288 and include in particular HPV E1 and E2proteins.

### Rabies (RG) Transgenes

Lyssavirus is an enveloped, single stranded RNA virus in the Rhabdoviridae family. Members of the Lyssavirus genus cause rabies and have the highest fatality rate of all known human viral pathogens. Rabies is transmitted via the saliva of infected mammals. A neurotropic virus, it enters the nervous system of its host, causing an encephalomyelitis that is almost invariably fatal. Currently there are about 60,000 rabies deaths worldwide yearly, mostly caused by dog bites in developing countries in Asia and Africa and by wildlife and bats in North America.

Rabies presents either in a furious or a paralytic form. The incubation period varies between about five days and several years but is typically between about 20 and 90 days. Clinical illness most often starts with prodromal complaints of malaise, anorexia, fatigue, headache and fever followed by pain or parathesia at the site of exposure. Anxiety, agitation or irritability may be prominent during this period, followed by hyperactivity, disorientation, seizures, hydrophobia, hypersalivation and, eventually, paralysis, coma and death.

Rabies antigens may be derived from the rabies viral glycoprotein (RG). For example, rabies glycoprotein may be used as a model antigen.

### Delivery of Adenoviral Vectors

In some embodiments, the recombinant adenoviral vector of the invention is administered to a subject by epicutaneous administration, intradermal administration, intramuscular injection, intraperitoneal injection, intravenous injection, nasal administration, oral administration, rectal administration, subcutaneous injection, transdermal administration or intravaginal administration.

In an embodiment of the invention, the vectors can be administered intramuscularly (IM), i.e., injection directly into muscle. Muscles are well vascularized and the uptake is typically rapid.

### Adjuvants

Approaches to establishing strong and lasting immunity to specific pathogens include addition of adjuvants to vaccines. By “adjuvant” is meant an agent that augments, stimulates, activates, potentiates or modulates the immune response to an active ingredient of the composition. The adjuvant effect may occur at the cellular or humoral level, or both. Adjuvants stimulate the response of the immune system to the actual antigen but have no immunological effect themselves. Alternatively or additionally, adjuvented compositions of the invention may comprise one or more immunostimulants. By “immunostimulant” it is meant an agent that induces a general, temporary increase in a subject's immune response, whether administered with the antigen or separately.

A composition of the invention may be administered with or without an adjuvant. Alternatively, or additionally, the composition may comprise, or be administered in conjunction with, one or more adjuvants (e.g. vaccine adjuvants), in particular the composition comprises an immunologically effective amount of a vector of the invention encoding a transgene.

### Methods of Use/Uses

Methods are provided for inducing an immune response against a disease caused by a pathogen in a subject in need thereof comprising a step of administering an immunologically effective amount of a construct or composition as disclosed herein. In some embodiments are provided the use of the constructs or compositions disclosed herein for inducing an immune response to a transgenic antigen in a subject in need thereof. Vectors of the invention may be applied for the prophylaxis, treatment or amelioration of diseases due to infection.

Methods of the invention include the use of a vector of the invention in medicine. They include the use of a vector of the invention for the treatment of a disease caused by a pathogen. A vector of the invention can be used in the manufacture of a medicament for treating a disease caused by a pathogen. A vector of the invention can be used in the manufacture of a medicament for the prevention or treatment of a disease, for example, a disease caused by respiratory syncytial virus (RSV).

Effective immunization with adenoviral vectors depends on the intrinsic immnomodulatory capability of the adenoviral vector backbone. Immunologically less potent adenoviruses induce less antigen expression. Effective immunization also depends on the ability of the promoter to drive strong and sustained transgene expression. For example, adenoviral vectors driven by the cytomegalovirus immediate-early (CMV-IE) promoter do not sustain long-term transgene expression because they induce cytokines that dampen expression.

By “subject” is intended a vertebrate, such as a mammal e.g. a human or a veterinary mammal. In some embodiments the subject is human.

### General

Vectors of the invention are generated using techniques and sequences provided herein, in conjunction with techniques known to those of skill in the art. Such techniques include conventional cloning techniques of cDNA such as those described in texts, use of overlapping oligonucleotide sequences of the adenovirus genomes, polymerase chain reaction, and any suitable method which provides the desired nucleotide sequence.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” used herein is intended to mean the amount ±10%.

The present invention will now be further described by means of the following non-limiting examples.

### EXAMPLES

### Example 1: Construction of Chimpanzee Adenoviruses with a Single Expression Cassette

Wild type chimpanzee adenoviruses type 155 (ChAd155) (WO 2016/198621) and type 83 (ChAd83) (WO 2010/086189) were isolated from healthy chimpanzees using standard procedures and were constructed as described in Sci Transl Med (2012) 4:1 and WO 2010/086189.

In Example 1, the ChAd155 and ChAd 83 vectors were each constructed by inserting a single transgene expression cassette. The expression cassette components used either the classical human CMV promoter or the CASI promoter, rabies glycoprotein as a model antigen and, optionally, a WPRE enhancer. Three different insertion sites were tested for the transgene cassette:
(i) replacing the E3 region with the transgene cassette,
(ii) inserting the transgene cassette between the fiber and the E4 region (site HE1), and
(iii) inserting the transgene cassette downstream of the right ITR (site HE2).

This numbering of these insertion sites corresponds to the illustrations of FIG. 1 where:
(i) the top panel illustrates the RC1 vector, in which a transgene cassette replaced the E3 region,
(ii) the middle panel illustrates the RC3 vector, in which a transgene cassette is inserted between the stop codons of the fiber gene and the E4 region (site HE1), and
(iii) the bottom panel illustrates the RC2 vector, in which a transgene cassette is inserted downstream of the right ITR (site HE2).

In the vectors shown in Example 1, the E1 region remains intact in all configurations.

The transgene was inserted by homologous recombination techniques in the following positions of the SEQ ID NO: 1 and of the SEQ ID NO: 2:

HE1 ChAd155: insertion site between bp 34611 and 34612 of SEQ ID NO: 1;

HE2 ChAd155: insertion site between bp 37662 and 37663 of SEQ ID NO: 1;

HE1 ChAd83: insertion site between bp 33535 and 33536 of SEQ ID NO: 2;

HE2 ChAd83: insertion site between bp 36387 and 36388 of SEQ ID NO: 2.

When the transgene cassette was inserted in site HE1, ChAd155 failed to replicate. However, insertion of a transgene cassette into the HE1 site of ChAd83 produced a viable vector.

### Example 2: Virus Production, Vector Titer and Expression of Vectors of Example 1

To identify an animal model in which to evaluate vector replication, a type C adenovirus ChAd155 RC2 and a type E adenovirus ChAd83 RC2 vectors of Example 1 were assessed for their ability to replicate, measured by vector titer and genome copy number, in cells of various animal origins. The results are shown in Table 1.

TABLE 1
Replication and Expression of RC2 ChAd155 and
RC2 ChAd83 of Example 1

| Cell line: | | Vector | Genome | Expression | |
|---|---|---|---|---|---|
| Species | Vector | Titer | Copy | Day 2 | Day 7 |
| MRC5: | ChAd155 | +++ | +++ | ++ | ++++ |
| Human | ChAd83 | +++++ | +++++ | +++ | +++++ |
| PK15: | ChAd155 | +++++ | +++++ | NA | NA |
| Swine | ChAd83 | +++ | ++++ | NA | NA |
| NMuLi: | ChAd155 | ++ | +++ | +++ | +++ |
| Mouse | ChAd83 | ND | + | ++ | ++ |
| Vero: | ChAd155 | ++ | ++++ | +++ | +++ |
| Non-human | ChAd83 | ND | + | + | + |
| primate | | | | | |

ND = not detected; NA = not available

As shown in Table 1, human MRC5 cells and swine PK15 cells produced high vector titers and high genome copy numbers of both ChAd155 and ChAd83. Murine NMuLi and non-human primate Vero cells also produced RC ChAd155 but to a lesser extent than the human or swine cells. RC ChAd83 failed to grow well in murine NMuLi cells and, surprisingly, in non-human primate Vero cells.

Human MRC5, mouse NMuLi and non-human primate Vero cells supported the expression of RC ChAd155 through day 7. Human MRC5 cells supported the expression of RC ChAd83 through day 7, as did mouse NMuLi and non-human primate Vero cells, but to a lesser extent than the human cells.

### Virus Production

FIG. 2A shows the amount of virus produced by human primary MRC5 cells infected with either ChAd155 or ChAd83, each comprising either the RC1 or RC2 vector construction of Example 1. The cells were harvested seven days post-infection and the vector titer was evaluated in cell lysates obtained following three freeze-thaw cycles. Vector titers were measured by quantitative PCR (QPCR) analysis with primers designed for the respective promoter regions. The multiplicity of infection (moi) was 1250 virus particles per cell. The virus production is indicated in the number of virus particles per cell (vp/cell) above the bars.

Human MRC5 cells supported production of ChAd155 comprising either RC1 (2.17×10³ vp/cell) or RC2 (4.4033 10³ vp/cell) and also supported production of ChAd83 comprising either RC1 (1.18×10⁴ vp/cell) or RC2 (1.06×10⁵ vp/cell). As shown in FIG. 2A, ChAd83 was produced at a higher level than ChAd155; the ChAd83 vector comprising RC2 was the most robust of the four viral/vector combinations.

FIG. 2B shows the amount of virus produced by human primary MRC5 cells infected with ChAd83 comprising the RC1, RC2 or RC3 vector construction of Example 1. The cells were harvested two and seven days post-infection. As with FIG. 2A, vector titers were measured by quantitative PCR (QPCR) analysis with primers designed for the respective promoter regions. The multiplicity of infection (moi) was 250 or 1250 virus particles per cell. The virus production is indicated in the number of virus particles per cell (vp/cell) above the bars.

Human MRC5 cells supported production of ChAd83 comprising RC1, RC2 or RC3. As shown in FIG. 2B, there was higher virus production for the RC2 and RC3 ChAd83 vectors than for the RC1 vector. There was also higher virus production for the ChAd83 RC2 HE2 vector than the RC3 HE1 vector.

### Vector Genome Copy Number

After infection, the vector is replicated in the cell and the vector genome copy number can be measured by QPCR. Vector DNA replication can occur even in cells not fully permissive for viral replication and propagation. QPCR of vector DNA provides a measure of vector replication within the infected cell, independently of the ability of the virus to complete the replication cycle and be released as mature viral progeny. Vector replication can thus be quantified in animal species, tissue types and cell types which are not permissive for ChAd virus replication or propagation.

Vector genome copy number was measured in parallel with vector titer and the results shown in FIG. 3A and FIG. 3B.

As with the virus production shown in FIG. 2A, Human MRC5 cells were infected with either ChAd155 or ChAd83, each comprising either the RC1 or RC2 vector construction of Example 1. The cells were harvested seven days post-infection, the total DNA extracted, the viral genome quantified by QPCR and the results expressed as vector genome copy per cell. The multiplicity of infection (moi) was 250 virus particles per cell and the numbers of virus particles per cell are indicated above the bars denoting viral genome copies per cell. The copy number is directly proportional to the level of transgene expression.

As shown in FIG. 3A, the amount of viral DNA replication of RC1 (6.21×10³ vp/cell) and RC2 (6.71×10³ vp/cell) by ChAd155 was similar. ChAd83 produced more RC1 (2.76×10⁴ vp/cell) and RC2 (9.19×10⁴ vp/cell) viral DNA than ChAd155. The highest level of viral DNA replication was observed by ChAd83 RC2.

As with the virus production shown in FIG. 2B, Human MRC5 cells were infected with ChAd83, comprising the RC1, RC2 or RC3 vector construction of Example 1. The cells were harvested at two and seven days post-infection, the total DNA extracted, the viral genome quantified by QPCR and the results expressed as vector genome copy per cell. The multiplicity of infection (moi) was 250 or 1250 virus particles per cell and the numbers of virus particles per cell are indicated above the bars denoting viral genome copies per cell. The copy number is directly proportional to the level of transgene expression.

As shown in FIG. 3B, the amount of viral DNA replication was higher for the RC2 and RC3 ChAd83 vectors than for the RC1 vector. There was comparable viral DNA replication between the RC2 and RC3 ChAd83 vectors.

### Example 3: Adenoviral Genome Copy Number of Vectors of Example 1

The efficiency of the replication competent adenoviral vectors with the constructs of Example 1, expressed as vector copies per cell, was evaluated in cell cultures derived from both mice and non-human primates.

FIG. 4(A) shows the genome copy number of replication competent vectors grown in murine hepatic NMuLi cells grown in monolayers and infected with ChAd155 RC1, ChAd155 RC2, ChAd83 RC1 or ChAd83 RC2 at a multiplicity of infection of 250 virus particles per cell. Total DNA was extracted at five days post-infection and the vector replication was measured by QPCR using primers annealing to the vector's promoter region.

The results, expressed as vector copies per cell, are shown in FIG. 4(A). ChAd155 amplified both the RC1 and RC2 vector with high efficiency in NMuLi cells. ChAd155 replicated the RC1 (1.73×10⁴) and RC2 (1.92×10⁴) vectors to approximately the same degree. ChAd83 was less efficient than ChAd155 in replicating the RC1 and RC2 vectors. ChAd83 replicated the vector DNA only in small amounts in the murine cells. RC1 vector replicated at a level of 5.47×10² copies per cell and the RC2 vector at a level of 6.74×10² copies per cell.

Non-human primate Vero cells were also grown in monolayers and infected with ChAd155 RC1, ChAd155 RC2, ChAd83 RC1 or ChAd83 RC2 (FIG. 4(B)). Two different multiplicities of infection were used: 50 and 250 virus particles per cell. Total DNA was extracted at five days post-infection and the vector replication was measured by QPCR using primers annealing to the vector's promoter region.

The results, expressed as vector copies per cell, are shown in (FIG. 4(B)). The Vero primate cell line was permissive for ChAd155 RC1 (3.71×10³ copies per cell at an moi of 50 and 4.93×10⁴ copies per cell at an moi of 250) and ChAd155 RC2 (8.15×10³ copies per cell at an moi of 50 and 7.05×10⁴ copies per cell at an moi of 250). The Vero primate cell line was poorly, if at all, permissive for ChAd83 RC1 or ChAd83 RC2. No ChAd83 RC1 or ChAd83 RC2 vectors were detected to be expressed from Vero cells at an moi of 50. At an moi of 250, ChAd83 replicated the RC1 vector at a level of 1.13×10² copies per cell and the RC2 vector at a level of 1.29×10³ copies per cell.

### Example 4: Transgene Expression From Murine and Non-human Primate Cells of Vectors of Example 1

Western blot analysis was performed to compare the level of transgene expression by ChAd155 RC1 and ChAd155 RC2 in murine NMuLi cells (FIG. 5(A)). The cells were infected with ChAd155 RC1 or ChAd155 RC2 at a multiplicity of infection of 50, 250 or 1250 viral particles per cell. The cells were harvested at two and five days post infection, extracts prepared using standard methods and an equivalent amount of total cell extract loaded onto SDS-PAGE gels. Following electrophoretic separation, the proteins were transferred onto nitrocellulose membranes, which were then probed with a commercially available monoclonal antibody to the rabies glycoprotein transgene.

FIG. 5(A) demonstrates that both ChAd155 RC1 and ChAd155 RC2 express a transgene in murine NMuLi cells. Expression was observed at both two and five days post infection, indicated by the band of about 51 kDa, which corresponds to the expected molecular weight of the rabies glycoprotein (RG). The ChAd155 RC2 vector produced a higher level of transgene expression than the ChAd155 RC1 vector at both two and five days post-infection.

Western blot analysis was then performed to compare the level of transgene expression by ChAd155 RC1, ChAd155 RC2, ChAd83 RC1 and ChAd83 RC2 in murine NMuLi cells FIG. 5(B)). The cells were infected with ChAd155 RC1, ChAd155 RC2, ChAd83 RC1 or ChAd83 RC2 at a multiplicity of infection of 50, 250 or 1250 viral particles per cell (250 and 1250 for ChAd83 RC1). The cells were processed for western blot. The cells were harvested at two and seven days post infection, extracts prepared using standard methods and an equivalent amount of extract loaded onto SDS-PAGE gels. Following electrophoretic separation, the proteins were transferred onto nitrocellulose membranes, which were then probed with a commercially available monoclonal antibody to the rabies glycoprotein transgene.

FIG. 5(B) demonstrates that ChAd155 RC1, ChAd155 RC2, ChAd83 RC1 and ChAd83 RC2 express a transgene in murine NMuLi cells. Expression was observed at both two and five days post infection, indicated by the band of about 51 kDa, which corresponds to the expected molecular weight of the rabies glycoprotein (RG). ChAd155 demonstrated more efficient expression of the transgene than ChAd83. At two days post-infection, robust transgene expression by ChAd155 RC2 was observed even at the low multiplicity of 50 vp/cell, whereas robust transgene expression by ChAd155 RC1 was first observed at higher mois. Also, RC2 demonstrated more efficient transgene expression than RC1 in both ChAd155 and ChAd83 viral serotypes. RC2 was more robustly expressed than RC1 in each of the direct comparisons.

Western blot analysis was performed to compare the level of transgene expression by ChAd83 RC1, RC2 and RC3 in MRC5 cells (FIG. 5(C)). The cells were infected with ChAd83 RC1, RC2 or RC3 at a multiplicity of infection of 250 or 1250 viral particles per cell. The cells were harvested at two and seven days post infection, extracts prepared using standard methods and an equivalent amount of total cell extract loaded onto SDS-PAGE gels. Following electrophoretic separation, the proteins were transferred onto nitrocellulose membranes, which were then probed with a commercially available monoclonal antibody to the rabies glycoprotein transgene.

(FIG. 5(C)) demonstrates that all of ChAd83 RC1, RC2 and RC3 express a transgene in MRC5 cells. Expression was observed at both two and seven days post infection, indicated by the band of about 51 kDa, which corresponds to the expected molecular weight of the rabies glycoprotein (RG). The ChAd83 RC2 vector produced a higher level of transgene expression than the ChAd83 RC1 and RC3 vectors at both two and seven days post-infection. There was no rabies glycoprotein detection for the RC1 and RC3 vectors at 7days.

### Example 5: Construction of Alternative Chimpanzee Adenoviruses With a Single Expression Cassette

As in Example 1, wild type chimpanzee adenoviruses type 155 (ChAd155) (WO 2016/198621) isolated from healthy chimpanzees using standard procedures were constructed as replication defective viruses as described in Sci Transl Med (2012) 4:1 and WO 2010/086189.

In Example 5, the ChAd155 is constructed by inserting a single transgene expression cassette. This expression cassette comprises the classical human CMV (hCMV) promoter, F0ΔTM, N and M2-1 RSV antigens and, optionally, a WPRE enhancer. This vector is shown in FIG. 6. The expression cassette is inserted into the E1 region of the adeno virus (after the E1 region has been deleted).

The ChAd155 shown in FIG. 6 comprises a transgene encoding all of the RSV F0ΔTM, M2-1 and N antigens, wherein a self-cleavage site (“2A”) is included between the RSV F0ΔTM antigen and the composite RSV N.M2-1 antigen, in which a flexible linker is included between the RSV M2-1 and N antigens.

The ChAd155 RSV vector of Example 5 comprises the polynucleotide of SEQ ID NO: 4 and encodes the polypeptide of SEQ ID NO: 5.

### Example 6: Construction of a Chimpanzee Adenoviruses With a Dual Expression Cassette

Again, wild type chimpanzee adenoviruses type 155 (ChAd155) (WO 2016/198621) isolated from healthy chimpanzees using standard procedures were constructed as replication defective viruses as described in Sci Transl Med (2012) 4:1 and WO 2010/086189.

The ChAd155 of Example 6 is constructed by inserting two transgene expression cassettes into two different locations in the adenovirus:
(1) The first expression cassette components comprise the classical human CMV (hCMV) promoter and N.M2-1 RSV composite antigen. This first expression cassette is inserted into the E1 region of the adenovirus (after the E1 region has been deleted).
(2) The second expression cassette comprises an enhanced classical human CMV (enhanced hCMV) promoter, the F0ΔTM RSV antigen and a WPRE enhancer. This first expression cassette is inserted into the HE2 region of the adenovirus (after the HE2 region has been deleted).

This vector comprising a dual expression cassette is shown in FIG. 7.

In the construct of FIG. 7, Ad5E4orf6 has been substituted into the early gene 4 (E4) region. The substitution is necessary to increase the productivity in HEK 293 cells.

### Example 7: Transgene Expression From the Dual Expression Cassette of Example 6

Western blot analysis was performed to compare the level of transgene expression in the ChAd155 vector of Example 6 (labelled “Dual” or “Dual cassette” in the figures) in MRC5 cells with:
(i) a vector comprising a single F expression cassette (ChAd155-F0ΔTM, labelled “F0ΔTm”),
(ii) a vector comprising a single NM2 expression cassette (ChAd155-NM2, labelled “NM2-1”), and
(iii) the vector of Example 5 comprising a single expression cassette containing the F and N.M2-1 RSV antigens (ChAd155-F0ΔTM.NM2, also labelled “RSV”)

The western blot analysis is shown in FIG. 8 and FIG. 9.

As shown in FIG. 8, the cells were infected with ChAd155-F0ΔTM, ChAd155-F0ΔTM.NM2 (“RSV”) or the ChAd155 dual cassette of Example 6 at a multiplicity of infection of 500 viral particles per cell. In addition, cells were infected with ChAd155-F0ΔTM.NM2 (“RSV”) at a multiplicity of infection of 500 or 1250 viral particles per cell. The cells were harvested at 48 hours and 96 hours post infection, extracts prepared using standard methods and an equivalent amount of total cell extract loaded onto SDS-PAGE gels.

FIG. 8 shows that the ChAd155 dual cassette provides an expression level of the F antigen which is comparable to ChAd155F0ΔTM and higher than ChAd155-FΔTM.NM2 in MRC5 cells.

As shown in FIG. 9, the cells were infected with ChAd155-NM2, ChAd155-F0ΔTM.NM2 (“RSV”) or the ChAd155 dual cassette of Example 6 at a multiplicity of infection of 250 and 1250 viral particles per cell. The cells were harvested at 48 hours post infection, extracts prepared using standard methods and an equivalent amount of total cell extract loaded onto SDS-PAGE gels.

In FIG. 9, the ChAd155 dual cassette provides NM2-1 expression level at least comparable to the ChAd155-NM2 single vector and higher than ChAd155-FΔTM.NM2 (“RSV”) in MRC5 cells.

### Example 8: Immunogencity of the Dual Expression Cassette of Example 6

The immunogenicity of the dual expression cassette of Example 6 was evaluated in CD1 outbred mice (10 per group). The experiment was performed by injecting 5×10⁸ viral particles intramuscularly into the mice. The B-cell response was measured at 4 and 8 weeks after the immunization by measuring the RSV neutralising titres. Each dot represents the response in a single mouse, and the line corresponds to the mean for each dose group. The results of this analysis are shown in FIG. 10.

FIG. 10 shows that the ChAd155 dual cassette provides a B-cell response comparable to ChAd155F0ΔTM and higher than that produced by ChAd155-F0ΔTM.NM2 (“RSV”).

The immunogenicity of the dual expression cassette of Example 6 was also evaluated in BALB/c inbred mice (48, 11 or 8 per group). The experiment was performed by injecting 10⁷ or 10⁶ viral particles intramuscularly. The T-cell response was measured 3 weeks after the immunization by ex vivo IFN-gamma enzyme-linked immunospot (ELISpot) using a M2 peptide T cell epitope mapped in BALB/c mice. The results are shown in FIG. 11, expressed as IFN-gamma Spot Forming Cells (SFC) per million splenocytes. Each dot represents the response in a single mouse, and the line corresponds to the mean for each dose group. Injected dose in number of virus particles are shown on the x axis. The results are shown in FIG. 11. FIG. 11 shows that the ChAd155 dual cassette provides a T-cell response higher than that produced by the single cassette ChAd155-F0ΔTM.NM2 (“triple RSV”, the results for which are obtained from historical data). This difference in response is greater for the 10⁶ vp dose.

FIG. 11 refers to “#positive mice”, i.e. the number of mice which responded to the vaccine.

### Example 9: Immunogencity of the Dual Expression Cassette of Example 6 in Cows

The study design is detailed in Table 2 below:

| | No. | | | | | End of |
|---|---|---|---|---|---|---|
| Group | Cows | Vaccine | Route | Dose | Immunization | Study |
| Gp1 | 4 | ChAd155 | Intramuscular (IM) | 1 × 10¹¹ | D0 | D90 |
| | | single RSV | | | | |
| Gp2 | 4 | ChAd155 | Intramuscular (IM) | 1 × 10¹¹ | D0 | D90 |
| | | dual RSV | | | | |
| Gp3 | 4 | Saline | Intramuscular (IM) | N/A | D0 | D90 |

The “ChAd155 single RSV” is the ChAd155 of Example 5, and the “ChAd155 dual RSV” is the ChAd155 of Example 6.

A total of 12 adult cows were enrolled in the study. The cows ranged in age from 2.7 years to 7.8 years and had a mean range of 4.8 years.

Before they were enrolled in the study, the cows were pre-screened for bovine RSV (BRSV) antibodies by ELISA. This allowed study groups to be established that had a similar distribution and mean BRSV Ab titer (so as to not bias any of the groups).

Samples were collected from the cows before vaccination (D-5 or D0) and after vaccination (D7, 10,14,28,60,90). In this study, the cows were vaccinated with 1\hat{ }10\hat{ }11 viral particles of one of the two vaccines or with saline on day zero (D0).

A Bronchoalveolar lavage (BAL) was performed at day -5, 7, 10 or 28 after vaccination to isolate T cells in the lungs of the cow. Then IFN-gamma cytokine production of the CD4+ and CD8+ T cells upon stimulation with RSV antigens (in the form of peptide pools) encoded in the vaccines was detected using intracellular cytokine staining (ICS) (i.e. IFNγ ICS was used to detect the lung T cell responses in the animals). The results of this experiment are shown in FIGS. 12A and 12B. It can be concluded from this experiment that the ChAd155-dual RSV induces consistent RSV-specific CD4+ and CD8+ responses in Bronchoalveolar lavage (BAL).

Blood samples were also taken from the cows on day 0, 14, 28, 60 and 90 after vaccination in order for IFN-gamma cytokine production of the RSV-specific CD4+ and CD8+ responses of the peripheral blood mononuclear cells (PBMC) to be detected using intracellular cytokine staining (ICS) (i.e. IFNγ ICS was used to detect the peripheral T cell responses). The results of this experiment are shown in FIGS. 13A and 13B. Based on these results, it can be concluded that the ChAd155-dual RSV consistently expand the pre-existing RSV-specific CD4+ and CD8+ responses in PBMC.

The blood samples were also used to detect neutralising antibodies (nAbs) for RSV in the serum (i.e. the peripheral humoral response was detected). The results of this experiment are shown in FIGS. 14A and 14B. These results show that the ChAd155-dual RSV boosts RSV nAbs in serum which are maintained at levels higher than baseline 3 months after vaccination.

### Example 10: Immunogenicity of ChAd155 Dual Encoding Rabies G and RSV NM2 Proteins

Three different ChAd155 vectors used constructed in this experiment:
ChAd155 encoding both rabies G (RG) and RSV NM2 proteins (called “ChAd155 dual” in this example, and ChAd155 dual hCMV NM 2-1—CASI RG WPRE);
ChAd155 encoding just the rabies G (RG) protein (called “ChAd155 RG” in this example, and ChAd155(ΔE4)CASI RG WPRE); and

The ChAd155 vector shown in FIG. 6, i.e. the vector with transgene encoding all of the RSV F0ΔTM, M2-1 and N antigens(called “ChAd155 RSV”).

Three different doses of the ChAd155 dual adenovirus were administered to mice: a highest dose of 10⁷ viral particles, and a middle dose of 10⁶ viral particles, and a lowest dose of 10⁵ viral particles.

Two different doses of the ChAd155 RG and ChAd155 RSV vectors were administered to mice. For the ChAd155 RSV, this was a higher dose of 10⁷ vaccine particles, and a lower dose of 10⁶ vaccine particles. For the ChAd155 RG, this was a higher dose of 10⁶ vaccine particles, and a lower dose of 10⁵ vaccine particles. Mice were sacrificed 3 weeks later and splenocytes tested by IFNγ ELISpot for T cell response to the vaccine encoded antigens.

The results of this experiment are shown in FIG. 15A, 15B and 15C. As can be seen from FIG. 15A, 15B and 15C, the ChAd155 dual RG-NM2 vector shows overall comparable immune responses to the vectors encoding each of the RG and NM2 antigens alone.

FIG. 15C compares the cumulative response to all encoded antigens at the common 10⁶ vp dosage used for all three different vectors. The rabies G protein is listed twice (G1 and G2) as two pools of overlapping peptides were used to cover the whole sequence of the protein

Therefore, placing the two antigens in the same vector still produces a comparable immune response while allowing antigens for different pathogens to be provided in the same vector.

### Example 11: Expression of ChAd155 Dual Encoding Rabies G and RSV NM2 Proteins in HeLa Cells

In the experiments of Example 11, HeLa cells were infected with the purified “ChAd155 dual”, “ChAd155 RG” and “ChAd155 RSV” used in Example 10.

Multiplicities of infection (MOI) of 50, 250 and 1250 were used in this experiment.

In order to obtain the Western Blot shown in FIG. 16A (obtained under reducing conditions), the cell lysate was harvested 48 hours post-infection. The estimated size of the NM2-1 is 65 kDa. FIG. 16A shows a comparable expression level for ChAd155 dual cassette and ChAd155 NM2-1. In addition, the NM2-1 expression level was higher for the ChAd155 dual cassette than the ChAd155 RSV vector.

To obtain the Western Blot shown in FIG. 16B, the supernatent was harvested 48 hours post-infection. The estimated size of the rabies glycoprotein is 57.6 kDa. FIG. 16B shows a comparable expression level for the ChAd155 dual and ChAd155 RG adenoviruses.

In addition, infectivity data was also collected using the four different vectors. The infectivity of purified virus was evaluated in adherent Procell 92 cells by Hexon Immunostaining. The results are given in Table 3 below (vp=virus particle, ifu=infectious unit, and R is the ratio between these two numbers). The infectivity results indicate that all of the vectors have similar infectivity. In addition, as all of the R values were below 300, the infectivity of all vectors was deemed to be within the range of acceptability.

TABLE 3

| | Vp/ml | Ifu/ml | R (vp/ifu) |
|---|---|---|---|
| ChAd155 hCMV NM 2-1 − | 5.51E+11 | 4.53E+09 | 122 |
| CASI RG WPRE | | | |
| ChAd155(ΔE4)hCMV-RSV | 1.12E+11 | 1.05E+09 | 107 |
| ChAd155(ΔE4)hCMV NM2-1 | 5.68E+11 | 4.26E+09 | 133 |
| ChAd155(ΔE4)CASI RG WPRE | 3.48E+11 | 3.35E+09 | 104 |

### Example 12: Immunogenicity of ChAd155 Dual Encoding CRPV E2 and E1 Proteins

Two different ChAd155 vectors were constructed in this experiment:
ChAd155 encoding a modified CRPV E2 protein in a first expression cassette, and a modified CRPV E1 protein in a second expression cassette (called “CRPV Dual”); and
ChAd155 encoding a fusion of the modified CRPV E2 and E1 proteins in a single expression cassette (called “CRPV Fusion”)

Two different doses of the two adeno vectors were administered to mice: a higher dose of 10⁷ viral particles, and a lower dose of 10⁶ viral particles. The results of this experiment are shown in FIG. 17. FIG. 17 is a IFNγ ELISpot on splenocytes 3 weeks post vaccination. A statistical analysis was performed on the results and the differences between the response from the different vectors was not deemed to be statistically significant. However, as can be seen from FIG. 17, the ChAd155 CRPV Dual vectors show increased frequency of responding mice at lowest dosage than the CRPV Fusion vectors ( 6/6 positive responding mice for the 10⁶ dose of the CRPV dual vector, but only 4/6 positive responding mice for the 10⁶ dose of the CRPV fusion vector.

### Example 13: Expression of ChAd155 Dual Encoding CRPV E2 and E1 Proteins

The two different ChAd155 vectors used in Example 12 were also used in Example 13.

Multiplicities of infection (MOI) of 250 and 1250 were used in this experiment. The cell lysate was harvested 48 hours post-infection. The estimated size of the modified E1 protein is 48 kDa, the modified E2 protein is 35 kDa, and the fusion protein containing both the modified E1 and E2 proteins is 88 kDa.

FIG. 18 shows a Western blot (obtained under reducing conditions) illustrating that there was better expression of the modified E1 and E2 proteins by the CRPV dual vector than the CRPV fusion vector. The “Pvj” columns shown in FIG. 18 are the controls used.

In addition, infectivity data was collected using the two different vectors. The infectivity of purified virus was evaluated in adherent Procell 92 cells by Hexon Immunostaining. The results are given in Table 4 below (vp=virus particle, ifu=infectious unit, and R is the ratio between these two numbers). The infectivity results indicate that the two vectors have similar infectivity. In addition, as all of the R values were below 300, the infectivity of all vectors was deemed to be within the range of acceptability.

TABLE 4

| | Vp/ml | Ifu/ml | R (vp/ifu) |
|---|---|---|---|
| ChAd155 hCMV CRPV | 4.52E+11 | 2.97E+09 | 153 |
| DE2DE1 Fusion HA WPRE | | | |
| “CRPV Fusion” | | | |
| ChAd155 hCMV CRPV DE2 | 6.20E+11 | 4.39E+09 | 143 |
| HA WPRE - Enh · CMV CRPV | | | |
| DE1 HA WPRE | | | |
| “CRPV Dual” | | | |

### Description of the Sequences

DESCRIPTION OF THE SEQUENCES
Polynucleotide sequence encoding wild type ChAd155
SEQ ID NO: 1
CATCATCAATAATATACCTTATTTTGGATTGAAGCCAATATGATAATGAGATGGGCGGCGCGGGGCGGGAG
GCGGGTCCGGGGGCGGGCCGGCGGGCGGGGCGGTGTGGCGGAAGTGGACTTTGTAAGTGTGGCGGATGTGACTTGCT
AGTGCCGGGCGCGGTAAAAGTGACGTTTTCCGTGCGCGACAACGCCCACGGGAAGTGACATTTTTCCCGCGGTTTTT
ACCGGATGTTGTAGTGAATTTGGGCGTAACCAAGTAAGATTTGGCCATTTTCGCGGGAAAACTGAAACGGGGAAGTG
AAATCTGATTAATTTCGCGTTAGTCATACCGCGTAATATTTGTCGAGGGCCGAGGGACTTTGGCCGATTACGTGGAG
GACTCGCCCAGGTGTTTTTTGAGGTGAATTTCCGCGTTCCGGGTCAAAGTCTCCGTTTTATTATTATAGTCAGCTGA
CGCGGAGTGTATTTATACCCTCTGATCTCGTCAAGTGGCCACTCTTGAGTGCCAGCGAGTAGAGTTTTCTCCTCTGC
CGCTCTCCGCTCCGCTCCGCTCGGCTCTGACACCGGGGAAAAAATGAGACATTTCACCTACGATGGCGGTGTGCTCA
CCGGCCAGCTGGCTGCTGAAGTCCTGGACACCCTGATCGAGGAGGTATTGGCCGATAATTATCCTCCCTCGACTCCT
TTTGAGCCACCTACACTTCACGAACTCTACGATCTGGATGTGGTGGGGCCCAGCGATCCGAACGAGCAGGCGGTTTC
CAGTTTTTTTCCAGAGTCCATGTTGTTGGCCAGCCAGGAGGGGGTCGAACTTGAGACCCCTCCTCCGATCGTGGATT
CCCCCGATCCGCCGCAGCTGACTAGGCAGCCCGAGCGCTGTGCGGGACCTGAGACTATGCCCCAGCTGCTACCTGAG
GTGATCGATCTCACCTGTAATGAGTCTGGTTTTCCACCCAGCGAGGATGAGGACGAAGAGGGTGAGCAGTTTGTGTT
AGATTCTGTGGAACAACCCGGGCGAGGATGCAGGTCTTGTCAATATCACCGGAAAAACACAGGAGACTCCCAGATTA
TGTGTTCTCTGTGTTATATGAAGATGACCTGTATGTTTATTTACAGTAAGTTTATCATCTGTGGGCAGGTGGGCTAT
AGTGTGGGTGGTGGTCTTTGGGGGGTTTTTTAATATATGTCAGGGGTTATGCTGAAGACTTTTTTATTGTGATTTTT
AAAGGTCCAGTGTCTGAGCCCGAGCAAGAACCTGAACCGGAGCCTGAGCCTTCTCGCCCCAGGAGAAAGCCTGTAAT
CTTAACTAGACCCAGCGCACCGGTAGCGAGAGGCCTCAGCAGCGCGGAGACCACCGACTCCGGTGCTTCCTCATCAC
CCCCGGAGATTCACCCCCTGGTGCCCCTGTGTCCCGTTAAGCCCGTTGCCGTGAGAGTCAGTGGGCGGCGGTCTGCT
GTGGAGTGCATTGAGGACTTGCTTTTTGATTCACAGGAACCTTTGGACTTGAGCTTGAAACGCCCCAGGCATTAAAC
CTGGTCACCTGGACTGAATGAGTTGACGCCTATGTTTGCTTTTGAATGACTTAATGTGTATAGATAATAAAGAGTGA
GATAATGTTTTAATTGCATGGTGTGTTTAACTTGGGCGGAGTCTGCTGGGTATATAAGCTTCCCTGGGCTAAACTTG
GTTACACTTGACCTCATGGAGGCCTGGGAGTGTTTGGAGAACTTTGCCGGAGTTCGTGCCTTGCTGGACGAGAGCTC
TAACAATACCTCTTGGTGGTGGAGGTATTTGTGGGGCTCTCCCCAGGGCAAGTTAGTTTGTAGAATCAAGGAGGATT
ACAAGTGGGAATTTGAAGAGCTTTTGAAATCCTGTGGTGAGCTATTGGATTCTTTGAATCTAGGCCACCAGGCTCTC
TTCCAGGAGAAGGTCATCAGGACTTTGGATTTTTCCACACCGGGGCGCATTGCAGCCGCGGTTGCTTTTCTAGCTTT
TTTGAAGGATAGATGGAGCGAAGAGACCCACTTGAGTTCGGGCTACGTCCTGGATTTTCTGGCCATGCAACTGTGGA
GAGCATGGATCAGACACAAGAACAGGCTGCAACTGTTGTCTTCCGTCCGCCCGTTGCTGATTCCGGCGGAGGAGCAA
CAGGCCGGGTCAGAGGACCGGGCCCGTCGGGATCCGGAGGAGAGGGCACCGAGGCCGGGCGAGAGGAGCGCGCTGAA
CCTGGGAACCGGGCTGAGCGGCCATCCACATCGGGAGTGAATGTCGGGCAGGTGGTGGATCTTTTTCCAGAACTGCG
GCGGATTTTGACTATTAGGGAGGATGGGCAATTTGTTAAGGGTCTTAAGAGGGAGAGGGGGGCTTCTGAGCATAACG
AGGAGGCCAGTAATTTAGCTTTTAGCTTGATGACCAGACACCGTCCAGAGTGCATCACTTTTCAGCAGATTAAGGAC
AATTGTGCCAATGAGTTGGATCTGTTGGGTCAGAAGTATAGCATAGAGCAGCTGACCACTTACTGGCTGCAGCCGGG
TGATGATCTGGAGGAAGCTATTAGGGTGTATGCTAAGGTGGCCCTGCGGCCCGATTGCAAGTACAAGCTCAAGGGGC
TGGTGAATATCAGGAATTGTTGCTACATTTCTGGCAACGGGGCGGAGGTGGAGATAGAGACCGAAGACAGGGTGGCT
TTCAGATGCAGCATGATGAATATGTGGCCGGGGGTGCTGGGCATGGACGGGGTGGTGATTATGAATGTGAGGTTCAC
GGGGCCCAACTTTAACGGCACGGTGTTTTTGGGGAACACCAACCTGGTCCTGCACGGGGTGAGCTTCTATGGGTTTA
ACAACACCTGTGTGGAGGCCTGGACCGATGTGAAGGTCCGCGGTTGCGCCTTTTATGGATGTTGGAAGGCCATAGTG
AGCCGCCCTAAGAGCAGGAGTTCCATTAAGAAATGCTTGTTTGAGAGGTGCACCTTGGGGATCCTGGCCGAGGGCAA
CTGCAGGGTGCGCCACAATGTGGCCTCCGAGTGCGGTTGCTTCATGCTAGTCAAGAGCGTGGCGGTAATCAAGCATA
ATATGGTGTGCGGCAACAGCGAGGACAAGGCCTCACAGATGCTGACCTGCACGGATGGCAACTGCCACTTGCTGAAG
ACCATCCATGTAACCAGCCACAGCCGGAAGGCCTGGCCCGTGTTCGAGCACAACTTGCTGACCCGCTGCTCCTTGCA
TCTGGGCAACAGGCGGGGGGTGTTCCTGCCCTATCAATGCAACTTTAGTCACACCAAGATCTTGCTAGAGCCCGAGA
GCATGTCCAAGGTGAACTTGAACGGGGTGTTTGACATGACCATGAAGATCTGGAAGGTGCTGAGGTACGACGAGACC
AGGTCCCGGTGCAGACCCTGCGAGTGCGGGGGCAAGCATATGAGGAACCAGCCCGTGATGCTGGATGTGACCGAGGA
GCTGAGGACAGACCACTTGGTTCTGGCCTGCACCAGGGCCGAGTTTGGTTCTAGCGATGAAGACACAGATTGAGGTG
GGTGAGTGGGCGTGGCCTGGGGTGGTCATGAAAATATATAAGTTGGGGGTCTTAGGGTCTCTTTATTTGTGTTGCAG
AGACCGCCGGAGCCATGAGCGGGAGCAGCAGCAGCAGCAGTAGCAGCAGCGCCTTGGATGGCAGCATCGTGAGCCCT
TATTTGACGACGCGGATGCCCCACTGGGCCGGGGTGCGTCAGAATGTGATGGGCTCCAGCATCGACGGCCGACCCGT
CCTGCCCGCAAATTCCGCCACGCTGACCTATGCGACCGTCGCGGGGACGCCGTTGGACGCCACCGCCGCCGCCGCCG
CCACCGCAGCCGCCTCGGCCGTGCGCAGCCTGGCCACGGACTTTGCATTCCTGGGACCACTGGCGACAGGGGCTACT
TCTCGGGCCGCTGCTGCCGCCGTTCGCGATGACAAGCTGACCGCCCTGCTGGCGCAGTTGGATGCGCTTACTCGGGA
ACTGGGTGACCTTTCTCAGCAGGTCATGGCCCTGCGCCAGCAGGTCTCCTCCCTGCAAGCTGGCGGGAATGCTTCTC
CCACAAATGCCGTTTAAGATAAATAAAACCAGACTCTGTTTGGATTAAAGAAAAGTAGCAAGTGCATTGCTCTCTTT
ATTTCATAATTTTCCGCGCGCGATAGGCCCTAGACCAGCGTTCTCGGTCGTTGAGGGTGCGGTGTATCTTCTCCAGG
ACGTGGTAGAGGTGGCTCTGGACGTTGAGATACATGGGCATGAGCCCGTCCCGGGGGTGGAGGTAGCACCACTGCAG
AGCTTCATGCTCCGGGGTGGTGTTGTAGATGATCCAGTCGTAGCAGGAGCGCTGGGCATGGTGCCTAAAAATGTCCT
TCAGCAGCAGGCCGATGGCCAGGGGGAGGCCCTTGGTGTAAGTGTTTACAAAACGGTTAAGTTGGGAAGGGTGCATT
CGGGGAGAGATGATGTGCATCTTGGACTGTATTTTTAGATTGGCGATGTTTCCGCCCAGATCCCTTCTGGGATTCAT
GTTGTGCAGGACCACCAGTACAGTGTATCCGGTGCACTTGGGGAATTTGTCATGCAGCTTAGAGGGAAAAGCGTGGA
AGAACTTGGAGACGCCTTTGTGGCCTCCCAGATTTTCCATGCATTCGTCCATGATGATGGCAATGGGCCCGCGGGAG
GCAGCTTGGGCAAAGATATTTCTGGGGTCGCTGACGTCGTAGTTGTGTTCCAGGGTGAGGTCGTCATAGGCCATTTT
TACAAAGCGCGGGCGGAGGGTGCCCGACTGGGGGATGATGGTCCCCTCTGGCCCTGGGGCGTAGTTGCCCTCGCAGA
TCTGCATTTCCCAGGCCTTAATCTCGGAGGGGGGAATCATATCCACCTGCGGGGCGATGAAGAAAACGGTTTCCGGA
GCCGGGGAGATTAACTGGGATGAGAGCAGGTTTCTAAGCAGCTGTGATTTTCCACAACCGGTGGGCCCATAAATAAC
ACCTATAACCGGTTGCAGCTGGTAGTTTAGAGAGCTGCAGCTGCCGTCGTCCCGGAGGAGGGGGGCCACCTCGTTGA
GCATGTCCCTGACGCGCATGTTCTCCCCGACCAGATCCGCCAGAAGGCGCTCGCCGCCCAGGGACAGCAGCTCTTGC
AAGGAAGCAAAGTTTTTCAGCGGCTTGAGGCCGTCCGCCGTGGGCATGTTTTTCAGGGTCTGGCTCAGCAGCTCCAG
GCGGTCCCAGAGCTCGGTGACGTGCTCTACGGCATCTCTATCCAGCATATCTCCTCGTTTCGCGGGTTGGGGCGACT
TTCGCTGTAGGGCACCAAGCGGTGGTCGTCCAGCGGGGCCAGAGTCATGTCCTTCCATGGGCGCAGGGTCCTCGTCA
GGGTGGTCTGGGTCACGGTGAAGGGGTGCGCTCCGGGCTGAGCGCTTGCCAAGGTGCGCTTGAGGCTGGTTCTGCTG
GTGCTGAAGCGCTGCCGGTCTTCGCCCTGCGCGTCGGCCAGGTAGCATTTGACCATGGTGTCATAGTCCAGCCCCTC
CGCGGCGTGTCCCTTGGCGCGCAGCTTGCCCTTGGAGGTGGCGCCGCACGAGGGGCAGAGCAGGCTCTTGAGCGCGT
AGAGCTTGGGGGCGAGGAAGACCGATTCGGGGGAGTAGGCGTCCGCGCCGCAGACCCCGCACACGGTCTCGCACTCC
ACCAGCCAGGTGAGCTCGGGGCGCGCCGGGTCAAAAACCAGGTTTCCCCCATGCTTTTTGATGCGTTTCTTACCTCG
GGTCTCCATGAGGTGGTGTCCCCGCTCGGTGACGAAGAGGCTGTCCGTGTCTCCGTAGACCGACTTGAGGGGTCTTT
TCTCCAGGGGGGTCCCTCGGTCTTCCTCGTAGAGGAACTCGGACCACTCTGAGACGAAGGCCCGCGTCCAGGCCAGG
ACGAAGGAGGCTATGTGGGAGGGGTAGCGGTCGTTGTCCACTAGGGGGTCCACCTTCTCCAAGGTGTGAAGACACAT
GTCGCCTTCCTCGGCGTCCAGGAAGGTGATTGGCTTGTAGGTGTAGGCCACGTGACCGGGGGTTCCTGACGGGGGGG
TATAAAAGGGGGTGGGGGCGCGCTCGTCGTCACTCTCTTCCGCATCGCTGTCTGCGAGGGCCAGCTGCTGGGGTGAG
TATTCCCTCTCGAAGGCGGGCATGACCTCCGCGCTGAGGTTGTCAGTTTCCAAAAACGAGGAGGATTTGATGTTCAC
CTGTCCCGAGGTGATACCTTTGAGGGTACCCGCGTCCATCTGGTCAGAAAACACGATCTTTTTATTGTCCAGCTTGG
TGGCGAACGACCCGTAGAGGGCGTTGGAGAGCAGCTTGGCGATGGAGCGCAGGGTCTGGTTCTTGTCCCTGTCGGCG
CGCTCCTTGGCCGCGATGTTGAGCTGCACGTACTCGCGCGCGACGCAGCGCCACTCGGGGAAGACGGTGGTGCGCTC
GTCGGGCACCAGGCGCACGCGCCAGCCGCGGTTGTGCAGGGTGACCAGGTCCACGCTGGTGGCGACCTCGCCGCGCA
GGCGCTCGTTGGTCCAGCAGAGACGGCCGCCCTTGCGCGAGCAGAAGGGGGGCAGGGGGTCGAGCTGGGTCTCGTCC
GGGGGGTCCGCGTCCACGGTGAAAACCCCGGGGCGCAGGCGCGCGTCGAAGTAGTCTATCTTGCAACCTTGCATGTC
CAGCGCCTGCTGCCAGTCGCGGGCGGCGAGCGCGCGCTCGTAGGGGTTGAGCGGCGGGCCCCAGGGCATGGGGTGGG
TGAGTGCGGAGGCGTACATGCCGCAGATGTCATAGACGTAGAGGGGCTCCCGCAGGACCCCGATGTAGGTGGGGTAG
CAGCGGCCGCCGCGGATGCTGGCGCGCACGTAGTCATACAGCTCGTGCGAGGGGGCGAGGAGGTCGGGGCCCAGGTT
GGTGCGGGCGGGGCGCTCCGCGCGGAAGACGATCTGCCTGAAGATGGCATGCGAGTTGGAAGAGATGGTGGGGCGCT
GGAAGACGTTGAAGCTGGCGTCCTGCAGGCCGACGGCGTCGCGCACGAAGGAGGCGTAGGAGTCGCGCAGCTTGTGT
ACCAGCTCGGCGGTGACCTGCACGTCGAGCGCGCAGTAGTCGAGGGTCTCGCGGATGATGTCATATTTAGCCTGCCC
CTTCTTTTTCCACAGCTCGCGGTTGAGGACAAACTCTTCGCGGTCTTTCCAGTACTCTTGGATCGGGAAACCGTCCG
GTTCCGAACGGTAAGAGCCTAGCATGTAGAACTGGTTGACGGCCTGGTAGGCGCAGCAGCCCTTCTCCACGGGGAGG
GCGTAGGCCTGCGCGGCCTTGCGGAGCGAGGTGTGGGTCAGGGCGAAGGTGTCCCTGACCATGACTTTGAGGTACTG
GTGCTTGAAGTCGGAGTCGTCGCAGCCGCCCCGCTCCCAGAGCGAGAAGTCGGTGCGCTTCTTGGAGCGGGGGTTGG
GCAGAGCGAAGGTGACATCGTTGAAGAGGATTTTGCCCGCGCGGGGCATGAAGTTGCGGGTGATGCGGAAGGGCCCC
GGCACTTCAGAGCGGTTGTTGATGACCTGGGCGGCGAGCACGATCTCGTCGAAGCCGTTGATGTTGTGGCCCACGAT
GTAGAGTTCCAGGAAGCGGGGCCGGCCCTTTACGGTGGGCAGCTTCTTTAGCTCTTCGTAGGTGAGCTCCTCGGGCG
AGGCGAGGCCGTGCTCGGCCAGGGCCCAGTCCGCGAGGTGCGGGTTGTCTCTGAGGAAGGACTTCCAGAGGTCGCGG
GCCAGGAGGGTCTGCAGGCGGTCTCTGAAGGTCCTGAACTGGCGGCCCACGGCCATTTTTTCGGGGGTGATGCAGTA
GAAGGTGAGGGGGTCTTGCTGCCAGCGGTCCCAGTCGAGCTGCAGGGCGAGGTCGCGCGCGGCGGTGACCAGGCGCT
CGTCGCCCCCGAATTTCATGACCAGCATGAAGGGCACGAGCTGCTTTCCGAAGGCCCCCATCCAAGTGTAGGTCTCT
ACATCGTAGGTGACAAAGAGGCGCTCCGTGCGAGGATGCGAGCCGATCGGGAAGAACTGGATCTCCCGCCACCAGTT
GGAGGAGTGGCTGTTGATGTGGTGGAAGTAGAAGTCCCGTCGCCGGGCCGAACACTCGTGCTGGCTTTTGTAAAAGC
GAGCGCAGTACTGGCAGCGCTGCACGGGCTGTACCTCATGCACGAGATGCACCTTTCGCCCGCGCACGAGGAAGCCG
AGGGGAAATCTGAGCCCCCCGCCTGGCTCGCGGCATGGCTGGTTCTCTTCTACTTTGGATGCGTGTCCGTCTCCGTC
TGGCTCCTCGAGGGGTGTTACGGTGGAGCGGACCACCACGCCGCGCGAGCCGCAGGTCCAGATATCGGCGCGCGGCG
GTCGGAGTTTGATGACGACATCGCGCAGCTGGGAGCTGTCCATGGTCTGGAGCTCCCGCGGCGGCGGCAGGTCAGCC
GGGAGTTCTTGCAGGTTCACCTCGCAGAGTCGGGCCAGGGCGCGGGGCAGGTCTAGGTGGTACCTGATCTCTAGGGG
CGTGTTGGTGGCGGCGTCGATGGCTTGCAGGAGCCCGCAGCCCCGGGGGGCGACGACGGTGCCCCGCGGGGTGGTGG
TGGTGGTGGCGGTGCAGCTCAGAAGCGGTGCCGCGGGCGGGCCCCCGGAGGTAGGGGGGGCTCCGGTCCCGCGGGCA
GGGGCGGCAGCGGCACGTCGGCGTGGAGCGCGGGCAGGAGTTGGTGCTGTGCCCGGAGGTTGCTGGCGAAGGCGACG
ACGCGGCGGTTGATCTCCTGGATCTGGCGCCTCTGCGTGAAGACGACGGGCCCGGTGAGCTTGAACCTGAAAGAGAG
TTCGACAGAATCAATCTCGGTGTCATTGACCGCGGCCTGGCGCAGGATCTCCTGCACGTCTCCCGAGTTGTCTTGGT
AGGCGATCTCGGCCATGAACTGCTCGATCTCTTCCTCCTGGAGGTCTCCGCGTCCGGCGCGTTCCACGGTGGCCGCC
AGGTCGTTGGAGATGCGCCCCATGAGCTGCGAGAAGGCGTTGAGTCCGCCCTCGTTCCAGACTCGGCTGTAGACCAC
GCCCCCCTGGTCATCGCGGGCGCGCATGACCACCTGCGCGAGGTTGAGCTCCACGTGCCGCGCGAAGACGGCGTAGT
TGCGCAGACGCTGGAAGAGGTAGTTGAGGGTGGTGGCGGTGTGCTCGGCCACGAAGAAGTTCATGACCCAGCGGCGC
AACGTGGATTCGTTGATGTCCCCCAAGGCCTCCAGCCGTTCCATGGCCTCGTAGAAGTCCACGGCGAAGTTGAAAAA
CTGGGAGTTGCGCGCCGACACGGTCAACTCCTCCTCCAGAAGACGGATGAGCTCGGCGACGGTGTCGCGCACCTCGC
GCTCGAAGGCTATGGGGATCTCTTCCTCCGCTAGCATCACCACCTCCTCCTCTTCCTCCTCTTCTGGCACTTCCATG
ATGGCTTCCTCCTCTTCGGGGGGTGGCGGCGGCGGCGGTGGGGGAGGGGGCGCTCTGCGCCGGCGGCGGCGCACCGG
GAGGCGGTCCACGAAGCGCGCGATCATCTCCCCGCGGCGGCGGCGCATGGTCTCGGTGACGGCGCGGCCGTTCTCCC
GGGGGCGCAGTTGGAAGACGCCGCCGGACATCTGGTGCTGGGGCGGGTGGCCGTGAGGCAGCGAGACGGCGCTGACG
ATGCATCTCAACAATTGCTGCGTAGGTACGCCGCCGAGGGACCTGAGGGAGTCCATATCCACCGGATCCGAAAACCT
TTCGAGGAAGGCGTCTAACCAGTCGCAGTCGCAAGGTAGGCTGAGCACCGTGGCGGGCGGCGGGGGGTGGGGGGAGT
GTCTGGCGGAGGTGCTGCTGATGATGTAATTGAAGTAGGCGGACTTGACACGGCGGATGGTCGACAGGAGCACCATG
TCCTTGGGTCCGGCCTGCTGGATGCGGAGGCGGTCGGCTATGCCCCAGGCTTCGTTCTGGCATCGGCGCAGGTCCTT
GTAGTAGTCTTGCATGAGCCTTTCCACCGGCACCTCTTCTCCTTCCTCTTCTGCTTCTTCCATGTCTGCTTCGGCCC
TGGGGCGGCGCCGCGCCCCCCTGCCCCCCATGCGCGTGACCCCGAACCCCCTGAGCGGTTGGAGCAGGGCCAGGTCG
GCGACGACGCGCTCGGCCAGGATGGCCTGCTGCACCTGCGTGAGGGTGGTTTGGAAGTCATCCAAGTCCACGAAGCG
GTGGTAGGCGCCCGTGTTGATGGTGTAGGTGCAGTTGGCCATGACGGACCAGTTGACGGTCTGGTGGCCCGGTTGCG
ACATCTCGGTGTACCTGAGTCGCGAGTAGGCGCGGGAGTCGAAGACGTAGTCGTTGCAAGTCCGCACCAGGTACTGG
TAGCCCACCAGGAAGTGCGGCGGCGGCTGGCGGTAGAGGGGCCAGCGCAGGGTGGCGGGGGCTCCGGGGGCCAGGTC
TTCCAGCATGAGGCGGTGGTAGGCGTAGATGTACCTGGACATCCAGGTGATACCCGCGGCGGTGGTGGAGGCGCGCG
GGAAGTCGCGCACCCGGTTCCAGATGTTGCGCAGGGGCAGAAAGTGCTCCATGGTAGGCGTGCTCTGTCCAGTCAGA
CGCGCGCAGTCGTTGATACTCTAGACCAGGGAAAACGAAAGCCGGTCAGCGGGCACTCTTCCGTGGTCTGGTGAATA
GATCGCAAGGGTATCATGGCGGAGGGCCTCGGTTCGAGCCCCGGGTCCGGGCCGGACGGTCCGCCATGATCCACGCG
GTTACCGCCCGCGTGTCGAACCCAGGTGTGCGACGTCAGACAACGGTGGAGTGTTCCTTTTGGCGTTTTTCTGGCCG
GGCGCCGGCGCCGCGTAAGAGACTAAGCCGCGAAAGCGAAAGCAGTAAGTGGCTCGCTCCCCGTAGCCGGAGGGATC
CTTGCTAAGGGTTGCGTTGCGGCGAACCCCGGTTCGAATCCCGTACTCGGGCCGGCCGGACCCGCGGCTAAGGTGTT
GGATTGGCCTCCCCCTCGTATAAAGACCCCGCTTGCGGATTGACTCCGGACACGGGGACGAGCCCCTTTTATTTTTG
CTTTCCCCAGATGCATCCGGTGCTGCGGCAGATGCGCCCCCCGCCCCAGCAGCAGCAACAACACCAGCAAGAGCGGC
AGCAACAGCAGCGGGAGTCATGCAGGGCCCCCTCACCCACCCTCGGCGGGCCGGCCACCTCGGCGTCCGCGGCCGTG
TCTGGCGCCTGCGGCGGCGGCGGGGGGCCGGCTGACGACCCCGAGGAGCCCCCGCGGCGCAGGGCCAGACACTACCT
GGACCTGGAGGAGGGCGAGGGCCTGGCGCGGCTGGGGGCGCCGTCTCCCGAGCGCCACCCGCGGGTGCAGCTGAAGC
GCGACTCGCGCGAGGCGTACGTGCCTCGGCAGAACCTGTTCAGGGACCGCGCGGGCGAGGAGCCCGAGGAGATGCGG
GACAGGAGGTTCAGCGCAGGGCGGGAGCTGCGGCAGGGGCTGAACCGCGAGCGGCTGCTGCGCGAGGAGGACTTTGA
GCCCGACGCGCGGACGGGGATCAGCCCCGCGCGCGCGCACGTGGCGGCCGCCGACCTGGTGACGGCGTACGAGCAGA
CGGTGAACCAGGAGATCAACTTCCAAAAGAGTTTCAACAACCACGTGCGCACGCTGGTGGCGCGCGAGGAGGTGACC
ATCGGGCTGATGCACCTGTGGGACTTTGTAAGCGCGCTGGTGCAGAACCCCAACAGCAAGCCTCTGACGGCGCAGCT
GTTCCTGATAGTGCAGCACAGCAGGGACAACGAGGCGTTTAGGGACGCGCTGCTGAACATCACCGAGCCCGAGGGTC
GGTGGCTGCTGGACCTGATTAACATCCTGCAGAGCATAGTGGTGCAGGAGCGCAGCCTGAGCCTGGCCGACAAGGTG
GCGGCCATCAACTACTCGATGCTGAGCCTGGGCAAGTTTTACGCGCGCAAGATCTACCAGACGCCGTACGTGCCCAT
AGACAAGGAGGTGAAGATCGACGGTTTTTACATGCGCATGGCGCTGAAGGTGCTCACCCTGAGCGACGACCTGGGCG
TGTACCGCAACGAGCGCATCCACAAGGCCGTGAGCGTGAGCCGGCGGCGCGAGCTGAGCGACCGCGAGCTGATGCAC
AGCCTGCAGCGGGCGCTGGCGGGCGCCGGCAGCGGCGACAGGGAGGCGGAGTCCTACTTCGATGCGGGGGCGGACCT
GCGCTGGGCGCCCAGCCGGCGGGCCCTGGAGGCCGCGGGGGTCCGCGAGGACTATGACGAGGACGGCGAGGAGGATG
AGGAGTACGAGCTAGAGGAGGGCGAGTACCTGGACTAAACCGCGGGTGGTGTTTCCGGTAGATGCAAGACCCGAACG
TGGTGGACCCGGCGCTGCGGGCGGCTCTGCAGAGCCAGCCGTCCGGCCTTAACTCCTCAGACGACTGGCGACAGGTC
ATGGACCGCATCATGTCGCTGACGGCGCGTAACCCGGACGCGTTCCGGCAGCAGCCGCAGGCCAACAGGCTCTCCGC
CATCCTGGAGGCGGTGGTGCCTGCGCGCTCGAACCCCACGCACGAGAAGGTGCTGGCCATAGTGAACGCGCTGGCCG
AGAACAGGGCCATCCGCCCGGACGAGGCCGGGCTGGTGTACGACGCGCTGCTGCAGCGCGTGGCCCGCTACAACAGC
GGCAACGTGCAGACCAACCTGGACCGGCTGGTGGGGGACGTGCGCGAGGCGGTGGCGCAGCGCGAGCGCGCGGATCG
GCAGGGCAACCTGGGCTCCATGGTGGCGCTGAATGCCTTCCTGAGCACGCAGCCGGCCAACGTGCCGCGGGGGCAGG
AAGACTACACCAACTTTGTGAGCGCGCTGCGGCTGATGGTGACCGAGACCCCCCAGAGCGAGGTGTACCAGTCGGGC
CCGGACTACTTCTTCCAGACCAGCAGACAGGGCCTGCAGACGGTGAACCTGAGCCAGGCTTTCAAGAACCTGCGGGG
GCTGTGGGGCGTGAAGGCGCCCACCGGCGACCGGGCGACGGTGTCCAGCCTGCTGACGCCCAACTCGCGCCTGCTGC
TGCTGCTGATCGCGCCGTTCACGGACAGCGGCAGCGTGTCCCGGGACACCTACCTGGGGCACCTGCTGACCCTGTAC
CGCGAGGCCATCGGGCAGGCGCAGGTGGACGAGCACACCTTCCAGGAGATCACCAGCGTGAGCCGCGCGCTGGGGCA
GGAGGACACGAGCAGCCTGGAGGCGACTCTGAACTACCTGCTGACCAACCGGCGGCAGAAGATTCCCTCGCTGCACA
GCCTGACCTCCGAGGAGGAGCGCATCTTGCGCTACGTGCAGCAGAGCGTGAGCCTGAACCTGATGCGCGACGGGGTG
ACGCCCAGCGTGGCGCTGGACATGACCGCGCGCAACATGGAACCGGGCATGTACGCCGCGCACCGGCCTTACATCAA
CCGCCTGATGGACTACCTGCATCGCGCGGCGGCCGTGAACCCCGAGTACTTTACCAACGCCATCCTGAACCCGCACT
GGCTCCCGCCGCCCGGGTTCTACAGCGGGGGCTTCGAGGTCCCGGAGACCAACGATGGCTTCCTGTGGGACGACATG
GACGACAGCGTGTTCTCCCCGCGGCCGCAGGCGCTGGCGGAAGCGTCCCTGCTGCGTCCCAAGAAGGAGGAGGAGGA
GGAGGCGAGTCGCCGCCGCGGCAGCAGCGGCGTGGCTTCTCTGTCCGAGCTGGGGGCGGCAGCCGCCGCGCGCCCCG
GGTCCCTGGGCGGCAGCCCCTTTCCGAGCCTGGTGGGGTCTCTGCACAGCGAGCGCACCACCCGCCCTCGGCTGCTG
GGCGAGGACGAGTACCTGAATAACTCCCTGCTGCAGCCGGTGCGGGAGAAAAACCTGCCTCCCGCCTTCCCCAACAA
CGGGATAGAGAGCCTGGTGGACAAGATGAGCAGATGGAAGACCTATGCGCAGGAGCACAGGGACGCGCCTGCGCTCC
GGCCGCCCACGCGGCGCCAGCGCCACGACCGGCAGCGGGGGCTGGTGTGGGATGACGAGGACTCCGCGGACGATAGC
AGCGTGCTGGACCTGGGAGGGAGCGGCAACCCGTTCGCGCACCTGCGCCCCCGCCTGGGGAGGATGTTTTAAAAAAA
AAAAAAAAAAGCAAGAAGCATGATGCAAAAATTAAATAAAACTCACCAAGGCCATGGCGACCGAGCGTTGGTTTCTT
GTGTTCCCTTCAGTATGCGGCGCGCGGCGATGTACCAGGAGGGACCTCCTCCCTCTTACGAGAGCGTGGTGGGCGCG
GCGGCGGCGGCGCCCTCTTCTCCCTTTGCGTCGCAGCTGCTGGAGCCGCCGTACGTGCCTCCGCGCTACCTGCGGCC
TACGGGGGGGAGAAACAGCATCCGTTACTCGGAGCTGGCGCCCCTGTTCGACACCACCCGGGTGTACCTGGTGGACA
ACAAGTCGGCGGACGTGGCCTCCCTGAACTACCAGAACGACCACAGCAATTTTTTGACCACGGTCATCCAGAACAAT
GACTACAGCCCGAGCGAGGCCAGCACCCAGACCATCAATCTGGATGACCGGTCGCACTGGGGCGGCGACCTGAAAAC
CATCCTGCACACCAACATGCCCAACGTGAACGAGTTCATGTTCACCAATAAGTTCAAGGCGCGGGTGATGGTGTCGC
GCTCGCACACCAAGGAAGACCGGGTGGAGCTGAAGTACGAGTGGGTGGAGTTCGAGCTGCCAGAGGGCAACTACTCC
GAGACCATGACCATTGACCTGATGAACAACGCGATCGTGGAGCACTATCTGAAAGTGGGCAGGCAGAACGGGGTCCT
GGAGAGCGACATCGGGGTCAAGTTCGACACCAGGAACTTCCGCCTGGGGCTGGACCCCGTGACCGGGCTGGTTATGC
CCGGGGTGTACACCAACGAGGCCTTCCATCCCGACATCATCCTGCTGCCCGGCTGCGGGGTGGACTTCACTTACAGC
CGCCTGAGCAACCTCCTGGGCATCCGCAAGCGGCAGCCCTTCCAGGAGGGCTTCAGGATCACCTACGAGGACCTGGA
GGGGGGCAACATCCCCGCGCTCCTCGATGTGGAGGCCTACCAGGATAGCTTGAAGGAAAATGAGGCGGGACAGGAGG
ATACCGCCCCCGCCGCCTCCGCCGCCGCCGAGCAGGGCGAGGATGCTGCTGACACCGCGGCCGCGGACGGGGCAGAG
GCCGACCCCGCTATGGTGGTGGAGGCTCCCGAGCAGGAGGAGGACATGAATGACAGTGCGGTGCGCGGAGACACCTT
CGTCACCCGGGGGGAGGAAAAGCAAGCGGAGGCCGAGGCCGCGGCCGAGGAAAAGCAACTGGCGGCAGCAGCGGCGG
CGGCGGCGTTGGCCGCGGCGGAGGCTGAGTCTGAGGGGACCAAGCCCGCCAAGGAGCCCGTGATTAAGCCCCTGACC
GAAGATAGCAAGAAGCGCAGTTACAACCTGCTCAAGGACAGCACCAACACCGCGTACCGCAGCTGGTACCTGGCCTA
CAACTACGGCGACCCGTCGACGGGGGTGCGCTCCTGGACCCTGCTGTGCACGCCGGACGTGACCTGCGGCTCGGAGC
AGGTGTACTGGTCGCTGCCCGACATGATGCAAGACCCCGTGACCTTCCGCTCCACGCGGCAGGTCAGCAACTTCCCG
GTGGTGGGCGCCGAGCTGCTGCCCGTGCACTCCAAGAGCTTCTACAACGACCAGGCCGTCTACTCCCAGCTCATCCG
CCAGTTCACCTCTCTGACCCACGTGTTCAATCGCTTTCCTGAGAACCAGATTCTGGCGCGCCCGCCCGCCCCCACCA
TCACCACCGTCAGTGAAAACGTTCCTGCTCTCACAGATCACGGGACGCTACCGCTGCGCAACAGCATCGGAGGAGTC
CAGCGAGTGACCGTTACTGACGCCAGACGCCGCACCTGCCCCTACGTTTACAAGGCCTTGGGCATAGTCTCGCCGCG
CGTCCTTTCCAGCCGCACTTTTTGAGCAACACCACCATCATGTCCATCCTGATCTCACCCAGCAATAACTCCGGCTG
GGGACTGCTGCGCGCGCCCAGCAAGATGTTCGGAGGGGCGAGGAAGCGTTCCGAGCAGCACCCCGTGCGCGTGCGCG
GGCACTTCCGCGCCCCCTGGGGAGCGCACAAACGCGGCCGCGCGGGGCGCACCACCGTGGACGACGCCATCGACTCG
GTGGTGGAGCAGGCGCGCAACTACAGGCCCGCGGTCTCTACCGTGGACGCGGCCATCCAGACCGTGGTGCGGGGCGC
GCGGCGGTACGCCAAGCTGAAGAGCCGCCGGAAGCGCGTGGCCCGCCGCCACCGCCGCCGACCCGGGGCCGCCGCCA
AACGCGCCGCCGCGGCCCTGCTTCGCCGGGCCAAGCGCACGGGCCGCCGCGCCGCCATGAGGGCCGCGCGCCGCTTG
GCCGCCGGCATCACCGCCGCCACCATGGCCCCCCGTACCCGAAGACGCGCGGCCGCCGCCGCCGCCGCCGCCATCAG
TGACATGGCCAGCAGGCGCCGGGGCAACGTGTACTGGGTGCGCGACTCGGTGACCGGCACGCGCGTGCCCGTGCGCT
TCCGCCCCCCGCGGACTTGAGATGATGTGAAAAAACAACACTGAGTCTCCTGCTGTTGTGTGTATCCCAGCGGCGGC
GGCGCGCGCAGCGTCATGTCCAAGCGCAAAATCAAAGAAGAGATGCTCCAGGTCGTCGCGCCGGAGATCTATGGGCC
CCCGAAGAAGGAAGAGCAGGATTCGAAGCCCCGCAAGATAAAGCGGGTCAAAAAGAAAAAGAAAGATGATGACGATG
CCGATGGGGAGGTGGAGTTCCTGCGCGCCACGGCGCCCAGGCGCCCGGTGCAGTGGAAGGGCCGGCGCGTAAAGCGC
GTCCTGCGCCCCGGCACCGCGGTGGTCTTCACGCCCGGCGAGCGCTCCACCCGGACTTTCAAGCGCGTCTATGACGA
GGTGTACGGCGACGAAGACCTGCTGGAGCAGGCCAACGAGCGCTTCGGAGAGTTTGCTTACGGGAAGCGTCAGCGGG
CGCTGGGGAAGGAGGACCTGCTGGCGCTGCCGCTGGACCAGGGCAACCCCACCCCCAGTCTGAAGCCCGTGACCCTG
CAGCAGGTGCTGCCGAGCAGCGCACCCTCCGAGGCGAAGCGGGGTCTGAAGCGCGAGGGCGGCGACCTGGCGCCCAC
CGTGCAGCTCATGGTGCCCAAGCGGCAGAGGCTGGAGGATGTGCTGGAGAAAATGAAAGTAGACCCCGGTCTGCAGC
CGGACATCAGGGTCCGCCCCATCAAGCAGGTGGCGCCGGGCCTCGGCGTGCAGACCGTGGACGTGGTCATCCCCACC
GGCAACTCCCCCGCCGCCGCCACCACTACCGCTGCCTCCACGGACATGGAGACACAGACCGATCCCGCCGCAGCCGC
AGCCGCAGCCGCCGCCGCGACCTCCTCGGCGGAGGTGCAGACGGACCCCTGGCTGCCGCCGGCGATGTCAGCTCCCC
GCGCGCGTCGCGGGCGCAGGAAGTACGGCGCCGCCAACGCGCTCCTGCCCGAGTACGCCTTGCATCCTTCCATCGCG
CCCACCCCCGGCTACCGAGGCTATACCTACCGCCCGCGAAGAGCCAAGGGTTCCACCCGCCGTCCCCGCCGACGCGC
CGCCGCCACCACCCGCCGCCGCCGCCGCAGACGCCAGCCCGCACTGGCTCCAGTCTCCGTGAGGAAAGTGGCGCGCG
ACGGACACACCCTGGTGCTGCCCAGGGCGCGCTACCACCCCAGCATCGTTTAAAAGCCTGTTGTGGTTCTTGCAGAT
ATGGCCCTCACTTGCCGCCTCCGTTTCCCGGTGCCGGGATACCGAGGAGGAAGATCGCGCCGCAGGAGGGGTCTGGC
CGGCCGCGGCCTGAGCGGAGGCAGCCGCCGCGCGCACCGGCGGCGACGCGCCACCAGCCGACGCATGCGCGGCGGGG
TGCTGCCCCTGTTAATCCCCCTGATCGCCGCGGCGATCGGCGCCGTGCCCGGGATCGCCTCCGTGGCCTTGCAAGCG
TCCCAGAGGCATTGACAGACTTGCAAACTTGCAAATATGGAAAAAAAAACCCCAATAAAAAAGTCTAGACTCTCACG
CTCGCTTGGTCCTGTGACTATTTTGTAGAATGGAAGACATCAACTTTGCGTCGCTGGCCCCGCGTCACGGCTCGCGC
CCGTTCCTGGGACACTGGAACGATATCGGCACCAGCAACATGAGCGGTGGCGCCTTCAGTTGGGGCTCTCTGTGGAG
CGGCATTAAAAGTATCGGGTCTGCCGTTAAAAATTACGGCTCCCGGGCCTGGAACAGCAGCACGGGCCAGATGTTGA
GAGACAAGTTGAAAGAGCAGAACTTCCAGCAGAAGGTGGTGGAGGGCCTGGCCTCCGGCATCAACGGGGTGGTGGAC
CTGGCCAACCAGGCCGTGCAGAATAAGATCAACAGCAGACTGGACCCCCGGCCGCCGGTGGAGGAGGTGCCGCCGGC
GCTGGAGACGGTGTCCCCCGATGGGCGTGGCGAGAAGCGCCCGCGGCCCGATAGGGAAGAGACCACTCTGGTCACGC
AGACCGATGAGCCGCCCCCGTATGAGGAGGCCCTGAAGCAAGGTCTGCCCACCACGCGGCCCATCGCGCCCATGGCC
ACCGGGGTGGTGGGCCGCCACACCCCCGCCACGCTGGACTTGCCTCCGCCCGCCGATGTGCCGCAGCAGCAGAAGGC
GGCACAGCCGGGCCCGCCCGCGACCGCCTCCCGTTCCTCCGCCGGTCCTCTGCGCCGCGCGGCCAGCGGCCCCCGCG
GGGGGGTCGCGAGGCACGGCAACTGGCAGAGCACGCTGAACAGCATCGTGGGTCTGGGGGTGCGGTCCGTGAAGCGC
CGCCGATGCTACTGAATAGCTTAGCTAACGTGTTGTATGTGTGTATGCGCCCTATGTCGCCGCCAGAGGAGCTGCTG
AGTCGCCGCCGTTCGCGCGCCCACCACCACCGCCACTCCGCCCCTCAAGATGGCGACCCCATCGATGATGCCGCAGT
GGTCGTACATGCACATCTCGGGCCAGGACGCCTCGGAGTACCTGAGCCCCGGGCTGGTGCAGTTCGCCCGCGCCACC
GAGAGCTACTTCAGCCTGAGTAACAAGTTTAGGAACCCCACGGTGGCGCCCACGCACGATGTGACCACCGACCGGTC
TCAGCGCCTGACGCTGCGGTTCATTCCCGTGGACCGCGAGGACACCGCGTACTCGTACAAGGCGCGGTTCACCCTGG
CCGTGGGCGACAACCGCGTGCTGGACATGGCCTCCACCTACTTTGACATCCGCGGGGTGCTGGACCGGGGTCCCACT
TTCAAGCCCTACTCTGGCACCGCCTACAACTCCCTGGCCCCCAAGGGCGCTCCCAACTCCTGCGAGTGGGAGCAAGA
GGAAACTCAGGCAGTTGAAGAAGCAGCAGAAGAGGAAGAAGAAGATGCTGACGGTCAAGCTGAGGAAGAGCAAGCAG
CTACCAAAAAGACTCATGTATATGCTCAGGCTCCCCTTTCTGGCGAAAAAATTAGTAAAGATGGTCTGCAAATAGGA
ACGGACGCTACAGCTACAGAACAAAAACCTATTTATGCAGACCCTACATTCCAGCCCGAACCCCAAATCGGGGAGTC
CCAGTGGAATGAGGCAGATGCTACAGTCGCCGGCGGTAGAGTGCTAAAGAAATCTACTCCCATGAAACCATGCTATG
GTTCCTATGCAAGACCCACAAATGCTAATGGAGGTCAGGGTGTACTAACGGCAAATGCCCAGGGACAGCTAGAATCT
CAGGTTGAAATGCAATTCTTTTCAACTTCTGAAAACGCCCGTAACGAGGCTAACAACATTCAGCCCAAATTGGTGCT
GTATAGTGAGGATGTGCACATGGAGACCCCGGATACGCACCTTTCTTACAAGCCCGCAAAAAGCGATGACAATTCAA
AAATCATGCTGGGTCAGCAGTCCATGCCCAACAGACCTAATTACATCGGCTTCAGAGACAACTTTATCGGCCTCATG
TATTACAATAGCACTGGCAACATGGGAGTGCTTGCAGGTCAGGCCTCTCAGTTGAATGCAGTGGTGGACTTGCAAGA
CAGAAACACAGAACTGTCCTACCAGCTCTTGCTTGATTCCATGGGTGACAGAACCAGATACTTTTCCATGTGGAATC
AGGCAGTGGACAGTTATGACCCAGATGTTAGAATTATTGAAAATCATGGAACTGAAGACGAGCTCCCCAACTATTGT
TTCCCTCTGGGTGGCATAGGGGTAACTGACACTTACCAGGCTGTTAAAACCAACAATGGCAATAACGGGGGCCAGGT
GACTTGGACAAAAGATGAAACTTTTGCAGATCGCAATGAAATAGGGGTGGGAAACAATTTCGCTATGGAGATCAACC
TCAGTGCCAACCTGTGGAGAAACTTCCTGTACTCCAACGTGGCGCTGTACCTACCAGACAAGCTTAAGTACAACCCC
TCCAATGTGGACATCTCTGACAACCCCAACACCTACGATTACATGAACAAGCGAGTGGTGGCCCCGGGGCTGGTGGA
CTGCTACATCAACCTGGGCGCGCGCTGGTCGCTGGACTACATGGACAACGTCAACCCCTTCAACCACCACCGCAATG
CGGGCCTGCGCTACCGCTCCATGCTCCTGGGCAACGGGCGCTACGTGCCCTTCCACATCCAGGTGCCCCAGAAGTTC
TTTGCCATCAAGAACCTCCTCCTCCTGCCGGGCTCCTACACCTACGAGTGGAACTTCAGGAAGGATGTCAACATGGT
CCTCCAGAGCTCTCTGGGTAACGATCTCAGGGTGGACGGGGCCAGCATCAAGTTCGAGAGCATCTGCCTCTACGCCA
CCTTCTTCCCCATGGCCCACAACACGGCCTCCACGCTCGAGGCCATGCTCAGGAACGACACCAACGACCAGTCCTTC
AATGACTACCTCTCCGCCGCCAACATGCTCTACCCCATACCCGCCAACGCCACCAACGTCCCCATCTCCATCCCCTC
GCGCAACTGGGCGGCCTTCCGCGGCTGGGCCTTCACCCGCCTCAAGACCAAGGAGACCCCCTCCCTGGGCTCGGGAT
TCGACCCCTACTACACCTACTCGGGCTCCATTCCCTACCTGGACGGCACCTTCTACCTCAACCACACTTTCAAGAAG
GTCTCGGTCACCTTCGACTCCTCGGTCAGCTGGCCGGGCAACGACCGTCTGCTCACCCCCAACGAGTTCGAGATCAA
GCGCTCGGTCGACGGGGAGGGCTACAACGTGGCCCAGTGCAACATGACCAAGGACTGGTTCCTGGTCCAGATGCTGG
CCAACTACAACATCGGCTACCAGGGCTTCTACATCCCAGAGAGCTACAAGGACAGGATGTACTCCTTCTTCAGGAAC
TTCCAGCCCATGAGCCGGCAGGTGGTGGACCAGACCAAGTACAAGGACTACCAGGAGGTGGGCATCATCCACCAGCA
CAACAACTCGGGCTTCGTGGGCTACCTCGCCCCCACCATGCGCGAGGGACAGGCCTACCCCGCCAACTTCCCCTATC
CGCTCATAGGCAAGACCGCGGTCGACAGCATCACCCAGAAAAAGTTCCTCTGCGACCGCACCCTCTGGCGCATCCCC
TTCTCCAGCAACTTCATGTCCATGGGTGCGCTCTCGGACCTGGGCCAGAACTTGCTCTACGCCAACTCCGCCCACGC
CCTCGACATGACCTTCGAGGTCGACCCCATGGACGAGCCCACCCTTCTCTATGTTCTGTTCGAAGTCTTTGACGTGG
TCCGGGTCCACCAGCCGCACCGCGGCGTCATCGAGACCGTGTACCTGCGTACGCCCTTCTCGGCCGGCAACGCCACC
ACCTAAAGAAGCAAGCCGCAGTCATCGCCGCCTGCATGCCGTCGGGTTCCACCGAGCAAGAGCTCAGGGCCATCGTC
AGAGACCTGGGATGCGGGCCCTATTTTTTGGGCACCTTCGACAAGCGCTTCCCTGGCTTTGTCTCCCCACACAAGCT
GGCCTGCGCCATCGTCAACACGGCCGGCCGCGAGACCGGGGGCGTGCACTGGCTGGCCTTCGCCTGGAACCCGCGCT
CCAAAACATGCTTCCTCTTTGACCCCTTCGGCTTTTCGGACCAGCGGCTCAAGCAAATCTACGAGTTCGAGTACGAG
GGCTTGCTGCGTCGCAGCGCCATCGCCTCCTCGCCCGACCGCTGCGTCACCCTCGAAAAGTCCACCCAGACCGTGCA
GGGGCCCGACTCGGCCGCCTGCGGTCTCTTCTGCTGCATGTTTCTGCACGCCTTTGTGCACTGGCCTCAGAGTCCCA
TGGACCGCAACCCCACCATGAACTTGCTGACGGGGGTGCCCAACTCCATGCTCCAGAGCCCCCAGGTCGAGCCCACC
CTGCGCCGCAACCAGGAGCAGCTCTACAGCTTCCTGGAGCGCCACTCGCCTTACTTCCGCCGCCACAGCGCACAGAT
CAGGAGGGCCACCTCCTTCTGCCACTTGCAAGAGATGCAAGAAGGGTAATAACGATGTACACACTTTTTTTCTCAAT
AAATGGCATCTTTTTATTTATACAAGCTCTCTGGGGTATTCATTTCCCACCACCACCCGCCGTTGTCGCCATCTGGC
TCTATTTAGAAATCGAAAGGGTTCTGCCGGGAGTCGCCGTGCGCCACGGGCAGGGACACGTTGCGATACTGGTAGCG
GGTGCCCCACTTGAACTCGGGCACCACCAGGCGAGGCAGCTCGGGGAAGTTTTCGCTCCACAGGCTGCGGGTCAGCA
CCAGCGCGTTCATCAGGTCGGGCGCCGAGATCTTGAAGTCGCAGTTGGGGCCGCCGCCCTGCGCGCGCGAGTTGCGG
TACACCGGGTTGCAGCACTGGAACACCAACAGCGCCGGGTGCTTCACGCTGGCCAGCACGCTGCGGTCGGAGATCAG
CTCGGCGTCCAGGTCCTCCGCGTTGCTCAGCGCGAACGGGGTCATCTTGGGCACTTGCCGCCCCAGGAAGGGCGCGT
GCCCCGGTTTCGAGTTGCAGTCGCAGCGCAGCGGGATCAGCAGGTGCCCGTGCCCGGACTCGGCGTTGGGGTACAGC
GCGCGCATGAAGGCCTGCATCTGGCGGAAGGCCATCTGGGCCTTGGCGCCCTCCGAGAAGAACATGCCGCAGGACTT
GCCCGAGAACTGGTTTGCGGGGCAGCTGGCGTCGTGCAGGCAGCAGCGCGCGTCGGTGTTGGCGATCTGCACCACGT
TGCGCCCCCACCGGTTCTTCACGATCTTGGCCTTGGACGATTGCTCCTTCAGCGCGCGCTGCCCGTTCTCGCTGGTC
ACATCCATCTCGATCACATGTTCCTTGTTCACCATGCTGCTGCCGTGCAGACACTTCAGCTCGCCCTCCGTCTCGGT
GCAGCGGTGCTGCCACAGCGCGCAGCCCGTGGGCTCGAAAGACTTGTAGGTCACCTCCGCGAAGGACTGCAGGTACC
CCTGCAAAAAGCGGCCCATCATGGTCACGAAGGTCTTGTTGCTGCTGAAGGTCAGCTGCAGCCCGCGGTGCTCCTCG
TTCAGCCAGGTCTTGCACACGGCCGCCAGCGCCTCCACCTGGTCGGGCAGCATCTTGAAGTTCACCTTCAGCTCATT
CTCCACGTGGTACTTGTCCATCAGCGTGCGCGCCGCCTCCATGCCCTTCTCCCAGGCCGACACCAGCGGCAGGCTCA
CGGGGTTCTTCACCATCACCGTGGCCGCCGCCTCCGCCGCGCTTTCGCTTTCCGCCCCGCTGTTCTCTTCCTCTTCC
TCCTCTTCCTCGCCGCCGCCCACTCGCAGCCCCCGCACCACGGGGTCGTCTTCCTGCAGGCGCTGCACCTTGCGCTT
GCCGTTGCGCCCCTGCTTGATGCGCACGGGCGGGTTGCTGAAGCCCACCATCACCAGCGCGGCCTCTTCTTGCTCGT
CCTCGCTGTCCAGAATGACCTCCGGGGAGGGGGGGTTGGTCATCCTCAGTACCGAGGCACGCTTCTTTTTCTTCCTG
GGGGCGTTCGCCAGCTCCGCGGCTGCGGCCGCTGCCGAGGTCGAAGGCCGAGGGCTGGGCGTGCGCGGCACCAGCGC
GTCCTGCGAGCCGTCCTCGTCCTCCTCGGACTCGAGACGGAGGCGGGCCCGCTTCTTCGGGGGCGCGCGGGGCGGCG
GAGGCGGCGGCGGCGACGGAGACGGGGACGAGACATCGTCCAGGGTGGGTGGACGGCGGGCCGCGCCGCGTCCGCGC
TCGGGGGTGGTCTCGCGCTGGTCCTCTTCCCGACTGGCCATCTCCCACTGCTCCTTCTCCTATAGGCAGAAAGAGAT
CATGGAGTCTCTCATGCGAGTCGAGAAGGAGGAGGACAGCCTAACCGCCCCCTCTGAGCCCTCCACCACCGCCGCCA
CCACCGCCAATGCCGCCGCGGACGACGCGCCCACCGAGACCACCGCCAGTACCACCCTCCCCAGCGACGCACCCCCG
CTCGAGAATGAAGTGCTGATCGAGCAGGACCCGGGTTTTGTGAGCGGAGAGGAGGATGAGGTGGATGAGAAGGAGAA
GGAGGAGGTCGCCGCCTCAGTGCCAAAAGAGGATAAAAAGCAAGACCAGGACGACGCAGATAAGGATGAGACAGCAG
TCGGGCGGGGGAACGGAAGCCATGATGCTGATGACGGCTACCTAGACGTGGGAGACGACGTGCTGCTTAAGCACCTG
CACCGCCAGTGCGTCATCGTCTGCGACGCGCTGCAGGAGCGCTGCGAAGTGCCCCTGGACGTGGCGGAGGTCAGCCG
CGCCTACGAGCGGCACCTCTTCGCGCCGCACGTGCCCCCCAAGCGCCGGGAGAACGGCACCTGCGAGCCCAACCCGC
GTCTCAACTTCTACCCGGTCTTCGCGGTACCCGAGGTGCTGGCCACCTACCACATCTTTTTCCAAAACTGCAAGATC
CCCCTCTCCTGCCGCGCCAACCGCACCCGCGCCGACAAAACCCTGACCCTGCGGCAGGGCGCCCACATACCTGATAT
CGCCTCTCTGGAGGAAGTGCCCAAGATCTTCGAGGGTCTCGGTCGCGACGAGAAACGGGCGGCGAACGCTCTGCACG
GAGACAGCGAAAACGAGAGTCACTCGGGGGTGCTGGTGGAGCTCGAGGGCGACAACGCGCGCCTGGCCGTACTCAAG
CGCAGCATAGAGGTCACCCACTTTGCCTACCCGGCGCTCAACCTGCCCCCCAAGGTCATGAGTGTGGTCATGGGCGA
GCTCATCATGCGCCGCGCCCAGCCCCTGGCCGCGGATGCAAACTTGCAAGAGTCCTCCGAGGAAGGCCTGCCCGCGG
TCAGCGACGAGCAGCTGGCGCGCTGGCTGGAGACCCGCGACCCCGCGCAGCTGGAGGAGCGGCGCAAGCTCATGATG
GCCGCGGTGCTGGTCACCGTGGAGCTCGAGTGTCTGCAGCGCTTCTTCGCGGACCCCGAGATGCAGCGCAAGCTCGA
GGAGACCCTGCACTACACCTTCCGCCAGGGCTACGTGCGCCAGGCCTGCAAGATCTCCAACGTGGAGCTCTGCAACC
TGGTCTCCTACCTGGGCATCCTGCACGAGAACCGCCTCGGGCAGAACGTCCTGCACTCCACCCTCAAAGGGGAGGCG
CGCCGCGACTACATCCGCGACTGCGCCTACCTCTTCCTCTGCTACACCTGGCAGACGGCCATGGGGGTCTGGCAGCA
GTGCCTGGAGGAGCGCAACCTCAAGGAGCTGGAAAAGCTCCTCAAGCGCACCCTCAGGGACCTCTGGACGGGCTTCA
ACGAGCGCTCGGTGGCCGCCGCGCTGGCGGACATCATCTTTCCCGAGCGCCTGCTCAAGACCCTGCAGCAGGGCCTG
CCCGACTTCACCAGCCAGAGCATGCTGCAGAACTTCAGGACTTTCATCCTGGAGCGCTCGGGCATCCTGCCGGCCAC
TTGCTGCGCGCTGCCCAGCGACTTCGTGCCCATCAAGTACAGGGAGTGCCCGCCGCCGCTCTGGGGCCACTGCTACC
TCTTCCAGCTGGCCAACTACCTCGCCTACCACTCGGACCTCATGGAAGACGTGAGCGGCGAGGGCCTGCTCGAGTGC
CACTGCCGCTGCAACCTCTGCACGCCCCACCGCTCTCTAGTCTGCAACCCGCAGCTGCTCAGCGAGAGTCAGATTAT
CGGTACCTTCGAGCTGCAGGGTCCCTCGCCTGACGAGAAGTCCGCGGCTCCAGGGCTGAAACTCACTCCGGGGCTGT
GGACTTCCGCCTACCTACGCAAATTTGTACCTGAGGACTACCACGCCCACGAGATCAGGTTCTACGAAGACCAATCC
CGCCCGCCCAAGGCGGAGCTCACCGCCTGCGTCATCACCCAGGGGCACATCCTGGGCCAATTGCAAGCCATCAACAA
AGCCCGCCGAGAGTTCTTGCTGAAAAAGGGTCGGGGGGTGTACCTGGACCCCCAGTCCGGCGAGGAGCTAAACCCGC
TACCCCCGCCGCCGCCCCAGCAGCGGGACCTTGCTTCCCAGGATGGCACCCAGAAAGAAGCAGCAGCCGCCGCCGCC
GCCGCAGCCATACATGCTTCTGGAGGAAGAGGAGGAGGACTGGGACAGTCAGGCAGAGGAGGTTTCGGACGAGGAGC
AGGAGGAGATGATGGAAGACTGGGAGGAGGACAGCAGCCTAGACGAGGAAGCTTCAGAGGCCGAAGAGGTGGCAGAC
GCAACACCATCGCCCTCGGTCGCAGCCCCCTCGCCGGGGCCCCTGAAATCCTCCGAACCCAGCACCAGCGCTATAAC
CTCCGCTCCTCCGGCGCCGGCGCCACCCGCCCGCAGACCCAACCGTAGATGGGACACCACAGGAACCGGGGTCGGTA
AGTCCAAGTGCCCGCCGCCGCCACCGCAGCAGCAGCAGCAGCAGCGCCAGGGCTACCGCTCGTGGCGCGGGCACAAG
AACGCCATAGTCGCCTGCTTGCAAGACTGCGGGGGCAACATCTCTTTCGCCCGCCGCTTCCTGCTATTCCACCACGG
GGTCGCCTTTCCCCGCAATGTCCTGCATTACTACCGTCATCTCTACAGCCCCTACTGCAGCGGCGACCCAGAGGCGG
CAGCGGCAGCCACAGCGGCGACCACCACCTAGGAAGATATCCTCCGCGGGCAAGACAGCGGCAGCAGCGGCCAGGAG
ACCCGCGGCAGCAGCGGCGGGAGCGGTGGGCGCACTGCGCCTCTCGCCCAACGAACCCCTCTCGACCCGGGAGCTCA
GACACAGGATCTTCCCCACTTTGTATGCCATCTTCCAACAGAGCAGAGGCCAGGAGCAGGAGCTGAAAATAAAAAAC
AGATCTCTGCGCTCCCTCACCCGCAGCTGTCTGTATCACAAAAGCGAAGATCAGCTTCGGCGCACGCTGGAGGACGC
GGAGGCACTCTTCAGCAAATACTGCGCGCTCACTCTTAAAGACTAGCTCCGCGCCCTTCTCGAATTTAGGCGGGAGA
AAACTACGTCATCGCCGGCCGCCGCCCAGCCCGCCCAGCCGAGATGAGCAAAGAGATTCCCACGCCATACATGTGGA
GCTACCAGCCGCAGATGGGACTCGCGGCGGGAGCGGCCCAGGACTACTCCACCCGCATGAACTACATGAGCGCGGGA
CCCCACATGATCTCACAGGTCAACGGGATCCGCGCCCAGCGAAACCAAATACTGCTGGAACAGGCGGCCATCACCGC
CACGCCCCGCCATAATCTCAACCCCCGAAATTGGCCCGCCGCCCTCGTGTACCAGGAAACCCCCTCCGCCACCACCG
TACTACTTCCGCGTGACGCCCAGGCCGAAGTCCAGATGACTAACTCAGGGGCGCAGCTCGCGGGCGGCTTTCGTCAC
GGGGCGCGGCCGCTCCGACCAGGTATAAGACACCTGATGATCAGAGGCCGAGGTATCCAGCTCAACGACGAGTCGGT
GAGCTCTTCGCTCGGTCTCCGTCCGGACGGAACTTTCCAGCTCGCCGGATCCGGCCGCTCTTCGTTCACGCCCCGCC
AGGCGTACCTGACTCTGCAGACCTCGTCCTCGGAGCCCCGCTCCGGCGGCATCGGAACCCTCCAGTTCGTGGAGGAG
TTCGTGCCCTCGGTCTACTTCAACCCCTTCTCGGGACCTCCCGGACGCTACCCCGACCAGTTCATTCCGAACTTTGA
CGCGGTGAAGGACTCGGCGGACGGCTACGACTGAATGTCAGGTGTCGAGGCAGAGCAGCTTCGCCTGAGACACCTCG
AGCACTGCCGCCGCCACAAGTGCTTCGCCCGCGGTTCTGGTGAGTTCTGCTACTTTCAGCTACCCGAGGAGCATACC
GAGGGGCCGGCGCACGGCGTCCGCCTGACCACCCAGGGCGAGGTTACCTGTTCCCTCATCCGGGAGTTTACCCTCCG
TCCCCTGCTAGTGGAGCGGGAGCGGGGTCCCTGTGTCCTAACTATCGCCTGCAACTGCCCTAACCCTGGATTACATC
AAGATCTTTGCTGTCATCTCTGTGCTGAGTTTAATAAACGCTGAGATCAGAATCTACTGGGGCTCCTGTCGCCATCC
TGTGAACGCCACCGTCTTCACCCACCCCGACCAGGCCCAGGCGAACCTCACCTGCGGTCTGCATCGGAGGGCCAAGA
AGTACCTCACCTGGTACTTCAACGGCACCCCCTTTGTGGTTTACAACAGCTTCGACGGGGACGGAGTCTCCCTGAAA
GACCAGCTCTCCGGTCTCAGCTACTCCATCCACAAGAACACCACCCTCCAACTCTTCCCTCCCTACCTGCCGGGAAC
CTACGAGTGCGTCACCGGCCGCTGCACCCACCTCACCCGCCTGATCGTAAACCAGAGCTTTCCGGGAACAGATAACT
CCCTCTTCCCCAGAACAGGAGGTGAGCTCAGGAAACTCCCCGGGGACCAGGGCGGAGACGTACCTTCGACCCTTGTG
GGGTTAGGATTTTTTATTACCGGGTTGCTGGCTCTTTTAATCAAAGTTTCCTTGAGATTTGTTCTTTCCTTCTACGT
GTATGAACACCTCAACCTCCAATAACTCTACCCTTTCTTCGGAATCAGGTGACTTCTCTGAAATCGGGCTTGGTGTG
CTGCTTACTCTGTTGATTTTTTTCCTTATCATACTCAGCCTTCTGTGCCTCAGGCTCGCCGCCTGCTGCGCACACAT
CTATATCTACTGCTGGTTGCTCAAGTGCAGGGGTCGCCACCCAAGATGAACAGGTACATGGTCCTATCGATCCTAGG
CCTGCTGGCCCTGGCGGCCTGCAGCGCCGCCAAAAAAGAGATTACCTTTGAGGAGCCCGCTTGCAATGTAACTTTCA
AGCCCGAGGGTGACCAATGCACCACCCTCGTCAAATGCGTTACCAATCATGAGAGGCTGCGCATCGACTACAAAAAC
AAAACTGGCCAGTTTGCGGTCTATAGTGTGTTTACGCCCGGAGACCCCTCTAACTACTCTGTCACCGTCTTCCAGGG
CGGACAGTCTAAGATATTCAATTACACTTTCCCTTTTTATGAGTTATGCGATGCGGTCATGTACATGTCAAAACAGT
ACAACCTGTGGCCTCCCTCTCCCCAGGCGTGTGTGGAAAATACTGGGTCTTACTGCTGTATGGCTTTCGCAATCACT
ACGCTCGCTCTAATCTGCACGGTGCTATACATAAAATTCAGGCAGAGGCGAATCTTTATCGATGAAAAGAAAATGCC
TTGATCGCTAACACCGGCTTTCTATCTGCAGAATGAATGCAATCACCTCCCTACTAATCACCACCACCCTCCTTGCG
ATTGCCCATGGGTTGACACGAATCGAAGTGCCAGTGGGGTCCAATGTCACCATGGTGGGCCCCGCCGGCAATTCCAC
CCTCATGTGGGAAAAATTTGTCCGCAATCAATGGGTTCATTTCTGCTCTAACCGAATCAGTATCAAGCCCAGAGCCA
TCTGCGATGGGCAAAATCTAACTCTGATCAATGTGCAAATGATGGATGCTGGGTACTATTACGGGCAGCGGGGAGAA
ATCATTAATTACTGGCGACCCCACAAGGACTACATGCTGCATGTAGTCGAGGCACTTCCCACTACCACCCCCACTAC
CACCTCTCCCACCACCACCACCACTACTACTACTACTACTACTACTACTACTACTACCACTACCGCTGCCCGCCATA
CCCGCAAAAGCACCATGATTAGCACAAAGCCCCCTCGTGCTCACTCCCACGCCGGCGGGCCCATCGGTGCGACCTCA
GAAACCACCGAGCTTTGCTTCTGCCAATGCACTAACGCCAGCGCTCATGAACTGTTCGACCTGGAGAATGAGGATGT
CCAGCAGAGCTCCGCTTGCCTGACCCAGGAGGCTGTGGAGCCCGTTGCCCTGAAGCAGATCGGTGATTCAATAATTG
ACTCTTCTTCTTTTGCCACTCCCGAATACCCTCCCGATTCTACTTTCCACATCACGGGTACCAAAGACCCTAACCTC
TCTTTCTACCTGATGCTGCTGCTCTGTATCTCTGTGGTCTCTTCCGCGCTGATGTTACTGGGGATGTTCTGCTGCCT
GATCTGCCGCAGAAAGAGAAAAGCTCGCTCTCAGGGCCAACCACTGATGCCCTTCCCCTACCCCCCGGATTTTGCAG
ATAACAAGATATGAGCTCGCTGCTGACACTAACCGCTTTACTAGCCTGCGCTCTAACCCTTGTCGCTTGCGACTCGA
GATTCCACAATGTCACAGCTGTGGCAGGAGAAAATGTTACTTTCAACTCCACGGCCGATACCCAGTGGTCGTGGAGT
GGCTCAGGTAGCTACTTAACTATCTGCAATAGCTCCACTTCCCCCGGCATATCCCCAACCAAGTACCAATGCAATGC
CAGCCTGTTCACCCTCATCAACGCTTCCACCCTGGACAATGGACTCTATGTAGGCTATGTACCCTTTGGTGGGCAAG
GAAAGACCCACGCTTACAACCTGGAAGTTCGCCAGCCCAGAACCACTACCCAAGCTTCTCCCACCACCACCACCACC
ACCACCATCACCAGCAGCAGCAGCAGCAGCAGCCACAGCAGCAGCAGCAGATTATTGACTTTGGTTTTGGCCAGCTC
ATCTGCCGCTACCCAGGCCATCTACAGCTCTGTGCCCGAAACCACTCAGATCCACCGCCCAGAAACGACCACCGCCA
CCACCCTACACACCTCCAGCGATCAGATGCCGACCAACATCACCCCCTTGGCTCTTCAAATGGGACTTACAAGCCCC
ACTCCAAAACCAGTGGATGCGGCCGAGGTCTCCGCCCTCGTCAATGACTGGGCGGGGCTGGGAATGTGGTGGTTCGC
CATAGGCATGATGGCGCTCTGCCTGCTTCTGCTCTGGCTCATCTGCTGCCTCCACCGCAGGCGAGCCAGACCCCCCA
TCTATAGACCCATCATTGTCCTGAACCCCGATAATGATGGGATCCATAGATTGGATGGCCTGAAAAACCTACTTTTT
TCTTTTACAGTATGATAAATTGAGACATGCCTCGCATTTTCTTGTACATGTTCCTTCTCCCACCTTTTCTGGGGTGT
TCTACGCTGGCCGCTGTGTCTCACCTGGAGGTAGACTGCCTCTCACCCTTCACTGTCTACCTGCTTTACGGATTGGT
CACCCTCACTCTCATCTGCAGCCTAATCACAGTAATCATCGCCTTCATCCAGTGCATTGATTACATCTGTGTGCGCC
TCGCATACTTCAGACACCACCCGCAGTACCGAGACAGGAACATTGCCCAACTTCTAAGACTGCTCTAATCATGCATA
AGACTGTGATCTGCCTTCTGATCCTCTGCATCCTGCCCACCCTCACCTCCTGCCAGTACACCACAAAATCTCCGCGC
AAAAGACATGCCTCCTGCCGCTTCACCCAACTGTGGAATATACCCAAATGCTACAACGAAAAGAGCGAGCTCTCCGA
AGCTTGGCTGTATGGGGTCATCTGTGTCTTAGTTTTCTGCAGCACTGTCTTTGCCCTCATAATCTACCCCTACTTTG
ATTTGGGATGGAACGCGATCGATGCCATGAATTACCCCACCTTTCCCGCACCCGAGATAATTCCACTGCGACAAGTT
GTACCCGTTGTCGTTAATCAACGCCCCCCATCCCCTACGCCCACTGAAATCAGCTACTTTAACCTAACAGGCGGAGA
TGACTGACGCCCTAGATCTAGAAATGGACGGCATCAGTACCGAGCAGCGTCTCCTAGAGAGGCGCAGGCAGGCGGCT
GAGCAAGAGCGCCTCAATCAGGAGCTCCGAGATCTCGTTAACCTGCACCAGTGCAAAAGAGGCATCTTTTGTCTGGT
AAAGCAGGCCAAAGTCACCTACGAGAAGACCGGCAACAGCCACCGCCTCAGTTACAAATTGCCCACCCAGCGCCAGA
AGCTGGTGCTCATGGTGGGTGAGAATCCCATCACCGTCACCCAGCACTCGGTAGAGACCGAGGGGTGTCTGCACTCC
CCCTGTCGGGGTCCAGAAGACCTCTGCACCCTGGTAAAGACCCTGTGCGGTCTCAGAGATTTAGTCCCCTTTAACTA
ATCAAACACTGGAATCAATAAAAAGAATCACTTACTTAAAATCAGACAGCAGGTCTCTGTCCAGTTTATTCAGCAGC
ACCTCCTTCCCCTCCTCCCAACTCTGGTACTCCAAACGCCTTCTGGCGGCAAACTTCCTCCACACCCTGAAGGGAAT
GTCAGATTCTTGCTCCTGTCCCTCCGCACCCACTATCTTCATGTTGTTGCAGATGAAGCGCACCAAAACGTCTGACG
AGAGCTTCAACCCCGTGTACCCCTATGACACGGAAAGCGGCCCTCCCTCCGTCCCTTTCCTCACCCCTCCCTTCGTG
TCTCCCGATGGATTCCAAGAAAGTCCCCCCGGGGTCCTGTCTCTGAACCTGGCCGAGCCCCTGGTCACTTCCCACGG
CATGCTCGCCCTGAAAATGGGAAGTGGCCTCTCCCTGGACGACGCTGGCAACCTCACCTCTCAAGATATCACCACCG
CTAGCCCTCCCCTCAAAAAAACCAAGACCAACCTCAGCCTAGAAACCTCATCCCCCCTAACTGTGAGCACCTCAGGC
GCCCTCACCGTAGCAGCCGCCGCTCCCCTGGCGGTGGCCGGCACCTCCCTCACCATGCAATCAGAGGCCCCCCTGAC
AGTACAGGATGCAAAACTCACCCTGGCCACCAAAGGCCCCCTGACCGTGTCTGAAGGCAAACTGGCCTTGCAAACAT
CGGCCCCGCTGACGGCCGCTGACAGCAGCACCCTCACAGTCAGTGCCACACCACCCCTTAGCACAAGCAATGGCAGC
TTGGGTATTGACATGCAAGCCCCCATTTACACCACCAATGGAAAACTAGGACTTAACTTTGGCGCTCCCCTGCATGT
GGTAGACAGCCTAAATGCACTGACTGTAGTTACTGGCCAAGGTCTTACGATAAACGGAACAGCCCTACAAACTAGAG
TCTCAGGTGCCCTCAACTATGACACATCAGGAAACCTAGAATTGAGAGCTGCAGGGGGTATGCGAGTTGATGCAAAT
GGTCAACTTATCCTTGATGTAGCTTACCCATTTGATGCACAAAACAATCTCAGCCTTAGGCTTGGACAGGGACCCCT
GTTTGTTAACTCTGCCCACAACTTGGATGTTAACTACAACAGAGGCCTCTACCTGTTCACATCTGGAAATACCAAAA
AGCTAGAAGTTAATATCAAAACAGCCAAGGGTCTCATTTATGATGACACTGCTATAGCAATCAATGCGGGTGATGGG
CTACAGTTTGACTCAGGCTCAGATACAAATCCATTAAAAACTAAACTTGGATTAGGACTGGATTATGACTCCAGCAG
AGCCATAATTGCTAAACTGGGAACTGGCCTAAGCTTTGACAACACAGGTGCCATCACAGTAGGCAACAAAAATGATG
ACAAGCTTACCTTGTGGACCACACCAGACCCATCCCCTAACTGTAGAATCTATTCAGAGAAAGATGCTAAATTCACA
CTTGTTTTGACTAAATGCGGCAGTCAGGTGTTGGCCAGCGTTTCTGTTTTATCTGTAAAAGGTAGCCTTGCGCCCAT
CAGTGGCACAGTAACTAGTGCTCAGATTGTCCTCAGATTTGATGAAAATGGAGTTCTACTAAGCAATTCTTCCCTTG
ACCCTCAATACTGGAACTACAGAAAAGGTGACCTTACAGAGGGCACTGCATATACCAACGCAGTGGGATTTATGCCC
AACCTCACAGCATACCCAAAAACACAGAGCCAAACTGCTAAAAGCAACATTGTAAGTCAGGTTTACTTGAATGGGGA
CAAATCCAAACCCATGACCCTCACCATTACCCTCAATGGAACTAATGAAACAGGAGATGCCACAGTAAGCACTTACT
CCATGTCATTCTCATGGAACTGGAATGGAAGTAATTACATTAATGAAACGTTCCAAACCAACTCCTTCACCTTCTCC
TACATCGCCCAAGAATAAAAAGCATGACGCTGTTGATTTGATTCAATGTGTTTCTGTTTTATTTTCAAGCACAACAA
AATCATTCAAGTCATTCTTCCATCTTAGCTTAATAGACACAGTAGCTTAATAGACCCAGTAGTGCAAAGCCCCATTC
TAGCTTATAGATCAGACAGTGATAATTAACCACCACCACCACCATACCTTTTGATTCAGGAAATCATGATCATCACA
GGATCCTAGTCTTCAGGCCGCCCCCTCCCTCCCAAGACACAGAATACACAGTCCTCTCCCCCCGACTGGCTTTAAAT
AACACCATCTGGTTGGTCACAGACATGTTCTTAGGGGTTATATTCCACACGGTCTCCTGCCGCGCCAGGCGCTCGTC
GGTGATGTTGATAAACTCTCCCGGCAGCTCGCTCAAGTTCACGTCGCTGTCCAGCGGCTGAACCTCCGGCTGACGCG
ATAACTGTGCGACCGGCTGCTGGACGAACGGAGGCCGCGCCTACAAGGGGGTAGAGTCATAATCCTCGGTCAGGATA
GGGCGGTGATGCAGCAGCAGCGAGCGAAACATCTGCTGCCGCCGCCGCTCCGTCCGGCAGGAAAACAACACGCCGGT
GGTCTCCTCCGCGATAATCCGCACCGCCCGCAGCATCAGCTTCCTCGTTCTCCGCGCGCAGCACCTCACCCTTATCT
CGCTCAAATCGGCGCAGTAGGTACAGCACAGCACCACGATGTTATTCATGATCCCACAGTGCAGGGCGCTGTATCCA
AAGCTCATGCCGGGAACCACCGCCCCCACGTGGCCATCGTACCACAAGCGCACGTAAATCAAGTGTCGACCCCTCAT
GAACGCGCTGGACACAAACATTACTTCCTTGGGCATGTTGTAATTCACCACCTCCCGGTACCAGATAAACCTCTGGT
TGAACAGGGCACCTTCCACCACCATCCTGAACCAAGAGGCCAGAACCTGCCCACCGGCTATGCACTGCAGGGAACCC
GGGTTGGAACAATGACAATGCAGACTCCAAGGCTCGTAACCGTGGATCATCCGGCTGCTGAAGGCATCGATGTTGGC
ACAACACAGACACACGTGCATGCACTTTCTCATGATTAGCAGCTCTTCCCTCGTCAGGATCATATCCCAAGGAATAA
CCCATTCTTGAATCAACGTAAAACCCACACAGCAGGGAAGGCCTCGCACATAACTCACGTTGTGCATGGTCAGCGTG
TTGCATTCCGGAAACAGCGGATGATCCTCCAGTATCGAGGCGCGGGTCTCCTTCTCACAGGGAGGTAAAGGGTCCCT
GCTGTACGGACTGCGCCGGGACGACCGAGATCGTGTTGAGCGTAGTGTCATGGAAAAGGGAACGCCGGACGTGGTCA
TACTTCTTGAAGCAGAACCAGGTTCGCGCGTGGCAGGCCTCCTTGCGTCTGCGGTCTCGCCGTCTAGCTCGCTCCGT
GTGATAGTTGTAGTACAGCCACTCCCGCAGAGCGTCGAGGCGCACCCTGGCTTCCGGATCTATGTAGACTCCGTCTT
GCACCGCGGCCCTGATAATATCCACCACCGTAGAATAAGCAACACCCAGCCAAGCAATACACTCGCTCTGCGAGCGG
CAGACAGGAGGAGCGGGCAGAGATGGGAGAACCATGATAAAAAACTTTTTTTAAAGAATATTTTCCAATTCTTCGAA
AGTAAGATCTATCAAGTGGCAGCGCTCCCCTCCACTGGCGCGGTCAAACTCTACGGCCAAAGCACAGACAACGGCAT
TTCTAAGATGTTCCTTAATGGCGTCCAAAAGACACACCGCTCTCAAGTTGCAGTAAACTATGAATGAAAACCCATCC
GGCTGATTTTCCAATATAGACGCGCCGGCAGCGTCCACCAAACCCAGATAATTTTCTTCTCTCCAGCGGTTTACGAT
CTGTCTAAGCAAATCCCTTATATCAAGTCCGACCATGCCAAAAATCTGCTCAAGAGCGCCCTCCACCTTCATGTACA
AGCAGCGCATCATGATTGCAAAAATTCAGGTTCTTCAGAGACCTGTATAAGATTCAAAATGGGAACATTAACAAAAA
TTCCTCTGTCGCGCAGATCCCTTCGCAGGGCAAGCTGAACATAATCAGACAGGTCCGAACGGACCAGTGAGGCCAAA
TCCCCACCAGGAACCAGATCCAGAGACCCTATACTGATTATGACGCGCATACTCGGGGCTATGCTGACCAGCGTAGC
GCCGATGTAGGCGTGCTGCATGGGCGGCGAGATAAAATGCAAAGTGCTGGTTAAAAAATCAGGCAAAGCCTCGCGCA
AAAAAGCTAACACATCATAATCATGCTCATGCAGGTAGTTGCAGGTAAGCTCAGGAACCAAAACGGAATAACACACG
ATTTTCCTCTCAAACATGACTTCGCGGATACTGCGTAAAACAAAAAATTATAAATAAAAAATTAATTAAATAACTTA
AACATTGGAAGCCTGTCTCACAACAGGAAAAACCACTTTAATCAACATAAGACGGGCCACGGGCATGCCGGCATAGC
CGTAAAAAAATTGGTCCCCGTGATTAACAAGTACCACAGACAGCTCCCCGGTCATGTCGGGGGTCATCATGTGAGAC
TCTGTATACACGTCTGGATTGTGAACATCAGACAAACAAAGAAATCGAGCCACGTAGCCCGGAGGTATAATCACCCG
CAGGCGGAGGTACAGCAAAACGACCCCCATAGGAGGAATCACAAAATTAGTAGGAGAAAAAAATACATAAACACCAG
AAAAACCCTGTTGCTGAGGCAAAATAGCGCCCTCCCGATCCAAAACAACATAAAGCGCTTCCACAGGAGCAGCCATA
ACAAAGACCCGAGTCTTACCAGTAAAAGAAAAAAGATCTCTCAACGCAGCACCAGCACCAACACTTCGCAGTGTAAA
AGGCCAAGTGCCGAGAGAGTATATATAGGAATAAAAAGTGACGTAAACGGGCAAAGTCCAAAAAACGCCCAGAAAAA
CCGCACGCGAACCTACGCCCCGAAACGAAAGCCAAAAAACACTAGACACTCCCTTCCGGCGTCAACTTCCGCTTTCC
CACGCTACGTCACTTCCCCCGGTCAAACAAACTACATATCCCGAACTTCCAAGTCGCCACGCCCAAAACACCGCCTA
CACCTCCCCGCCCGCCGGCCCGCCCCCGGACCCGCCTCCCGCCCCGCGCCGCCCATCTCATTATCATATTGGCTTCA
ATCCAAAATAAGGTATATTATTGATGATG
Polynucleotide sequence encoding wild type ChAd83
SEQ ID NO: 2
CATCATCAATAATATACCTCAAACTTTTGGTGCGCGTTAATATGCAAATGAGCTGTTTGAATTTGGGGAT
GCGGGGCGCTGATTGGCTGCGGGAGCGGCGACCGTTAGGGGCGGGGCGGGTGACGTTTTGATGACGTGGC
CGTGAGGCGGAGCCGGTTTGCAAGTTCTCGTGGGAAAAGTGACGTCAAACGAGGTGTGGTTTGAACACGG
AAATACTCAATTTTCCCGCGCTCTCTGACAGGAAATGAGGTGTTTCTGGGCGGATGCAAGTGAAAACGGG
CCATTTTCGCGCGAAAACTGAATGAGGAAGTGAAAATCTGAGTAATTTCGCGTTTATGGCAGGGAGGAGT
ATTTGCCGAGGGCCGAGTAGACTTTGACCGATTACGTGGGGGTTTCGATTACCGTATTTTTCACCTAAAT
TTCCGCGTACGGTGTCAAAGTCCGGTGTTTTTACGTAGGCGTCAGCTGATCGCCAGGGTATTTAAACCTG
CGCTCACTAGTCAAGAGGCCACTCTTGAGTGCCAGCGAGTAGAGTTTTCTCCTCCGCGCCGCGAGTCAGA
TCTACACTTTGAAAGATGAGGCACTTGAGAGACCTGCCCGGTAATGTTTTCCTGGCTACTGGGAACGAGA
TTCTGGAATTGGTGGTGGACGCCATGATGGGTGACGACCCTCCCGAGCCCCCTACCCCATTTGAGGCGCC
TTCGCTGTACGATTTGTATGATCTGGAGGTGGATGTGCCCGAGAACGACCCCAACGAGGAGGCGGTGAAT
GATTTGTTTAGCGATGCCGCGCTGCTGGCTGCCGAGCAGGCTAATACGGACTTTGGCTCAGACAGCGATT
CTTCTCTCCATACCCCGAGACCCGGCAGAGGTGAGAAAAAGATCCCCGAGCTTAAAGGGGAAGAGCTCGA
CCTGCGCTGCTATGAGGAATGCTTGCCTCCGAGCGATGATGAGGAGGACGAGGAGGCGATTCGAGCTGCA
GCGAACCAGGGAGTGAAAGCTGCGGGCGAAAGCTTTAGCCTGGACTGTCCTACTCTGCCCGGACACGGCT
GTAAGTCTTGTGAATTTCATCGCATGAATACTGGAGATAAGAATGTGATGTGTGCCCTGTGCTATATGAG
AGCTTACAACCATTGTGTTTACAGTAAGTGTGATTAACTTTAGTTGGGAAGGCAGAGGGTGACTGGGTGC
TGACTGGTTTATTTATGTATATGTTTTTTATGTGTAGGTCCCGTCTCTGACGCAGATGAGACCCCCACTT
CAGAGTGCATTTCATCACCCCCAGAAATTGGCGAGGAACCGCCCGAAGATATTATTCATAGACCAGTTGC
AGTGAGAGTCACCGGGCGGAGAGCAGCTGTGGAGAGTTTGGATGACTTGCTACAGGGTGGGGATGAACCT
TTGGACTTGTGTACCCGGAAACGCCCCAGGCACTAAGTGCCACACATGTGTGTTTACTTAAGGTGATGTC
AGTATTTATAGGGTGTGGAGTGCAATAAAATCCGTGTTGACTTTAAGTGCGTGGTTTATGACTCAGGGGT
GGGGACTGTGGGTATATAAGCAGGTGCAGACCTGTGTGGTCAGTTCAGAGCAGGACTCATGGAGATCTGG
ACGGTCTTGGAAGACTTTCACCAGACTAGACAGCTGCTAGAGAACTCATCGGAGGGAGTCTCTTACCTGT
GGAGATTCTGCTTCGGTGGGCCTCTAGCTAAGCTAGTCTATAGGGCCAAGCAGGATTATAAGGATCAATT
TGAGGATATTTTGAGAGAGTGTCCTGGTATTTTTGACTCTCTCAACTTGGGCCATCAGTCTCACTTTAAC
CAGAGTATTCTGAGAGCCCTTGACTTTTCCACTCCTGGCAGAACTACCGCCGCGGTAGCCTTTTTTGCCT
TTATCCTTGACAAATGGAGTCAAGAAACCCATTTCAGCAGGGATTACCGTCTGGACTGCTTAGCAGTAGC
TTTGTGGAGAACATGGAGGTGCCAGCGCCTGAATGCAATCTCCGGCTACTTGCCAGTACAGCCGGTAGAC
ACGCTGAGGATCCTGAGTCTCCAGTCACCCCAGGAACACCAACGCCGCCAGCAGCCGCAGCAGGAGCAGC
AGCAAGAGGAGGACCGAGAAGAGAACCCGAGAGCCGGTCTGGACCCTCCGGTGGCGGAGGAGGAGGAGTA
GCTGACTTGTTTCCCGAGCTGCGCCGGGTGCTGACTAGGTCTTCCAGTGGACGGGAGAGGGGGATTAAGC
GGGAGAGGCATGAGGAGACTAGTCACAGAACTGAACTGACTGTCAGTCTGATGAGCCGCAGGCGCCCAGA
ATCGGTGTGGTGGCATGAGGTGCAGTCGCAGGGGATAGATGAGGTCTCGGTGATGCATGAGAAATATTCC
CTAGAACAAGTCAAGACTTGTTGGTTGGAGCCTGAGGATGATTGGGAGGTAGCCATCAGGAATTATGCCA
AGCTAGCTCTGAAGCCAGACAAGAAGTACAAGATTACCAAACTGATTAATATCAGAAATTCCTGCTACAT
TTCAGGGAATGGGGCCGAGGTGGAGATCAGTACCCAGGAGAGGGTGGCCTTCAGATGCTGCATGATGAAT
ATGTACCCGGGGGTGGTGGGCATGGAGGGAGTCACCTTTATGAACGCGAGGTTCAGGGGCGATGGGTATA
ATGGGGTGGTCTTTATGGCCAACACCAAGCTGACAGTGCACGGATGCTCCTTCTTTGGCTTCAATAACAT
GTGCATCGAGGCCTGGGGCAGTGTTTCAGTGAGGGGATGCAGTTTTTCAGCCAACTGGATGGGGGTCGTG
GGCAGAACCAAGAGCAAGGTGTCAGTGAAGAAATGCCTGTTCGAGAGGTGCCACCTGGGGGTGATGAGCG
AGGGCGAAGCCAAAGTCAAACACTGCGCCTCTACTGAGACGGGCTGCTTTGTGCTGATCAAGGGCAATGC
CCAAGTCAAGCATAACATGATCTGTGGGGCCTCGGATGAGCGCGGCTACCAGATGCTGACCTGCGCCGGT
GGGAACAGCCATATGCTGGCCACCGTGCATGTGACCTCGCACCCCCGCAAGACATGGCCCGAGTTCGAGC
ACAACGTCATGACCCGCTGCAATGTGCACCTGGGCTCCCGCCGAGGCATGTTCATGCCCTACCAGTGCAA
CATGCAATTTGTGAAGGTGCTGCTGGAGCCCGATGCCATGTCCAGAGTGAGCCTGACGGGGGTGTTTGAC
ATGAATGTGGAGATGTGGAAAATTCTGAGATATGATGAATCCAAGACCAGGTGCCGGGCCTGCGAATGCG
GAGGCAAGCACGCCAGGCTTCAGCCCGTGTGTGTGGAGGTGACGGAGGACCTGCGACCCGATCATTTGGT
GTTGTCCTGCAACGGGACGGAGTTCGGCTCCAGCGGGGAAGAATCTGACTAGAGTGAGTAGTGTTTGGGG
GAGGTGGAGGGCCTGGATGAGGGGCAGAATGACTAAAATCTGTGTTTTTCTGCGCAGCAGCATGAGCGGA
AGCGCCTCCTTTGAGGGAGGGGTATTCAGCCCTTATCTGACGGGGCGTCTCCCCTCCTGGGCGGGAGTGC
GTCAGAATGTGATGGGATCCACGGTGGACGGCCGGCCCGTGCAGCCCGCGAACTCTTCAACCCTGACCTA
CGCGACCCTGAGCTCCTCGTCCGTGGACGCAGCTGCCGCCGCAGCTGCTGCTTCCGCCGCCAGCGCCGTG
CGCGGAATGGCCCTGGGCGCCGGCTACTACAGCTCTCTGGTGGCCAACTCGAGTTCCACCAATAATCCCG
CCAGCCTGAACGAGGAGAAGCTGCTGCTGCTGATGGCCCAGCTCGAGGCCCTGACCCAGCGCCTGGGCGA
GCTGACCCAGCAGGTTGCTCAGCTGCAGGCGGAGACGCGGGCCGCGGTTGCCACGGTGAAAACCAAATAA
AAAATGAATCAATAAATAAACGGAGACGGTTGTTGATTTTAACACAGAGTCTTGAATCTTTATTTGATTT
TTCGCGCGCGGTAGGCCCTGGACCACCGGTCTCGATCATTGAGCACCCGGTGGATCTTTTCCAGGACCCG
GTAGAGGTGGGCTTGGATGTTGAGGTACATGGGCATGAGCCCGTCCCGGGGGTGGAGGTAGCTCCATTGC
AGGGCCTCGTGCTCGGGGGTGGTGTTGTAAATCACCCAGTCATAGCAGGGGCGCAGGGCGTGGTGCTGCA
CGATGTCCTTGAGGAGGAGACTGATGGCCACGGGCAGCCCCTTGGTGTAGGTGTTGACGAACCTGTTGAG
CTGGGAGGGATGCATGCGGGGGGAGATGAGATGCATCTTGGCCTGGATCTTGAGATTGGCGATGTTCCCG
CCCAGATCCCGCCGGGGGTTCATGTTGTGCAGGACCACCAGCACGGTGTATCCGGTGCACTTGGGGAATT
TGTCATGCAACTTGGAAGGGAAGGCGTGAAAGAATTTGGAGACGCCCTTGTGGCCGCCCAGGTTTTCCAT
GCACTCATCCATGATGATGGCGATGGGCCCGTGGGCGGCGGCCTGGGCAAAGACGTTTCGGGGGTCGGAC
ACATCGTAGTTGTGGTCCTGGGTGAGCTCGTCATAGGCCATTTTAATGAATTTGGGGCGGAGGGTGCCCG
ACTGGGGGACGAAGGTGCCCTCGATCCCGGGGGCGTAGTTGCCCTCGCAGATCTGCATCTCCCAGGCCTT
GAGCTCGGAGGGGGGGATCATGTCCACCTGCGGGGCGATGAAAAAAACGGTTTCCGGGGCGGGGGAGATG
AGCTGCGCCGAAAGCAGGTTCCGGAGCAGCTGGGACTTGCCGCAGCCGGTGGGGCCGTAGATGACCCCGA
TGACCGGCTGCAGGTGGTAGTTGAGGGAGAGACAGCTGCCGTCCTCGCGGAGGAGGGGGGCCACCTCGTT
CATCATCTCGCGCACATGCATGTTCTCGCGCACGAGTTCCGCCAGGAGGCGCTCGCCCCCCAGCGAGAGG
AGCTCTTGCAGCGAGGCGAAGTTTTTCAGCGGCTTGAGCCCGTCGGCCATGGGCATTTTGGAGAGGGTCT
GTTGCAAGAGTTCCAGACGGTCCCAGAGCTCGGTGATGTGCTCTAGGGCATCTCGATCCAGCAGACCTCC
TCGTTTCGCGGGTTGGGGCGACTGCGGGAGTAGGGCACCAGGCGATGGGCGTCCAGCGAGGCCAGGGTCC
GGTCCTTCCAGGGTCGCAGGGTCCGCGTCAGCGTGGTCTCCGTCACGGTGAAGGGGTGCGCGCCGGGCTG
GGCGCTTGCGAGGGTGCGCTTCAGGCTCATCCGGCTGGTCGAGAACCGCTCCCGGTCGGCGCCCTGTGCG
TCGGCCAGGTAGCAATTGAGCATGAGTTCGTAGTTGAGCGCCTCGGCCGCGTGGCCCTTGGCGCGGAGCT
TACCTTTGGAAGTGTGTCCGCAGACGGGACAGAGGAGGGACTTGAGGGCGTAGAGCTTGGGGGCGAGGAA
GACGGACTCGGGGGCGTAGGCGTCCGCGCCGCAGCTGGCGCAGACGGTCTCGCACTCCACGAGCCAGGTG
AGGTCGGGGCGGTCGGGGTCAAAAACGAGGTTTCCTCCGTGCTTTTTGATGCGTTTCTTACCTCTGGTCT
CCATGAGCTCGTGTCCCCGCTGGGTGACAAAGAGGCTGTCCGTGTCCCCGTAGACCGACTTTATGGGCCG
GTCCTCGAGCGGGGTGCCGCGGTCCTCGTCGTAGAGGAACCCCGCCCACTCCGAGACGAAGGCCCGGGTC
CAGGCCAGCACGAAGGAGGCCACGTGGGAGGGGTAGCGGTCGTTGTCCACCAGCGGGTCCACCTTCTCCA
GGGTATGCAAGCACATGTCCCCCTCGTCCACATCCAGGAAGGTGATTGGCTTGTAAGTGTAGGCCACGTG
ACCGGGGGTCCCGGCCGGGGGGGTATAAAAGGGGGCGGGCCCCTGCTCGTCCTCACTGTCTTCCGGATCG
CTGTCCAGGAGCGCCAGCTGTTGGGGTAGGTATTCCCTCTCGAAGGCGGGCATGACCTCGGCACTCAGGT
TGTCAGTTTCTAGAAACGAGGAGGATTTGATATTGACGGTGCCGTTGGAGACGCCTTTCATGAGCCCCTC
GTCCATCTGGTCAGAAAAGACGATCTTTTTGTTGTCGAGCTTGGTGGCGAAGGAGCCGTAGAGGGCGTTG
GAGAGCAGCTTGGCGATGGAGCGCATGGTCTGGTTCTTTTCCTTGTCGGCGCGCTCCTTGGCGGCGATGT
TGAGCTGCACGTACTCGCGCGCCACGCACTTCCATTCGGGGAAGACGGTGGTGAGCTCGTCGGGCACGAT
TCTGACCCGCCAGCCGCGGTTGTGCAGGGTGATGAGGTCCACGCTGGTGGCCACCTCGCCGCGCAGGGGC
TCGTTGGTCCAGCAGAGGCGCCCGCCCTTGCGCGAGCAGAAGGGGGGCAGCGGGTCCAGCATGAGCTCGT
CGGGGGGGTCGGCGTCCACGGTGAAGATGCCGGGCAGGAGCTCGGGGTCGAAGTAGCTGATGCAGGTGCC
CAGATCGTCCAGCGCCGCTTGCCAGTCGCGCACGGCCAGCGCGCGCTCGTAGGGGCTGAGGGGCGTGCCC
CAGGGCATGGGGTGCGTGAGCGCGGAGGCGTACATGCCGCAGATGTCGTAGACGTAGAGGGGCTCCTCGA
GGACGCCGATGTAGGTGGGGTAGCAGCGCCCCCCGCGGATGCTGGCGCGCACGTAGTCGTACAGCTCGTG
CGAGGGCGCGAGGAGCCCCGTGCCGAGGTTGGAGCGTTGCGGCTTTTCGGCGCGGTAGACGATCTGGCGG
AAGATGGCGTGGGAGTTGGAGGAGATGGTGGGCCTCTGGAAGATGTTGAAGTGGGCGTGGGGCAGGCCGA
CCGAGTCCCTGATGAAGTGGGCGTAGGAGTCCTGCAGCTTGGCGACGAGCTCGGCGGTGACGAGGACGTC
CAGGGCGCAGTAGTCGAGGGTCTCTTGGATGATGTCGTACTTGAGCTGGCCCTTCTGCTTCCACAGCTCG
CGGTTGAGAAGGAACTCTTCGCGGTCCTTCCAGTACTCTTCGAGGGGGAACCCGTCCTGATCGGCACGGT
AAGAGCCCACCATGTAGAACTGGTTGACGGCCTTGTAGGCGCAGCAGCCCTTCTCCACGGGGAGGGCGTA
AGCTTGCGCGGCCTTGCGCAGGGAGGTGTGGGTGAGGGCGAAGGTGTCGCGCACCATGACTTTGAGGAAC
TGGTGCTTGAAGTCGAGGTCGTCGCAGCCGCCCTGCTCCCAGAGTTGGAAGTCCGTGCGCTTCTTGTAGG
CGGGGTTGGGCAAAGCGAAAGTAACATCGTTGAAGAGGATCTTGCCCGCGCGGGGCATGAAGTTGCGAGT
GATGCGGAAAGGCTGGGGCACCTCGGCCCGGTTGTTGATGACCTGGGCGGCGAGGACGATCTCGTCGAAG
CCGTTGATGTTGTGCCCGACGATGTAGAGTTCCACGAATCGCGGGCAGCCCTTGACGTGGGGCAGCTTCT
TGAGCTCGTCGTAGGTGAGCTCGGCGGGGTCGCTGAGCCCGTGCTGCTCGAGGGCCCAGTCGGCGACGTG
GGGGTTGGCGCTGAGGAAGGAAGTCCAGAGATCCACGGCCAGGGCGGTCTGCAAGCGGTCCCGGTACTGA
CGGAACTGCTGGCCCACGGCCATTTTTTCGGGGGTGACGCAGTAGAAGGTGCGGGGGTCGCCGTGCCAGC
GGTCCCACTTGAGTTGGAGGGCGAGGTCGTGGGCGAGCTCGACGAGCGGCGGGTCCCCGGAGAGTTTCAT
GACCAGCATGAAGGGGACGAGCTGCTTGCCGAAGGACCCCATCCAGGTGTAGGTTTCCACATCGTAGGTG
AGGAAGAGCCTTTCGGTGCGAGGATGCGAGCCGATGGGGAAGAACTGGATCTCCTGCCACCAGTTGGAGG
AATGGCTGTTGATGTGATGGAAGTAGAAATGCCGACGGCGCGCCGAGCACTCGTGCTTGTGTTTATACAA
GCGTCCGCAGTGCTCGCAACGCTGCACGGGATGCACGTGCTGCACGAGCTGTACCTGGGTTCCTTTGACG
AGGAATTTCAGTGGGCAGTGGAGCGCTGGCGGCTGCATCTGGTGCTGTACTACGTCCTGGCCATCGGCGT
GGCCATCGTCTGCCTCGATGGTGGTCATGCTGACGAGCCCGCGCGGGAGGCAGGTCCAGACCTCGGCTCG
GACGGGTCGGAGAGCGAGGACGAGGGCGCGCAGGCCGGAGCTGTCCAGGGTCCTGAGACGCTGCGGAGTC
AGGTCAGTGGGCAGCGGCGGCGCGCGGTTGACTTGCAGGAGCTTTTCCAGGGCGCGCGGGAGGTCCAGAT
GGTACTTGATCTCCACGGCGCCGTTGGTGGCGACGTCCACGGCTTGCAGGGTCCCGTGCCCCTGGGGCGC
CACCACCGTGCCCCGTTTCTTCTTGGGCGGCGGCGGCTCCATGCTTAGAAGCGGCGGCGAGGACGCGCGC
CGGGCGGCAGGGGCGGCTCGGGGCCCGGAGGCAGGGGCGGCAGGGGCACGTCGGCGCCGCGCGCGGGCAG
GTTCTGGTACTGCGCCCGGAGAAGACTGGCGTGAGCGACGACGCGACGGTTGACGTCCTGGATCTGACGC
CTCTGGGTGAAGGCCACGGGACCCGTGAGTTTGAACCTGAAAGAGAGTTCGACAGAATCAATTTCGGTAT
CGTTGACGGCGGCCTGCCGCAGGATCTCTTGCACGTCGCCCGAGTTGTCCTGGTAGGCGATCTCGGTCAT
GAACTGCTCGATCTCCTCCTCCTGAAGGTCTCCGCGGCCGGCGCGCTCGACGGTGGCCGCGAGGTCGTTG
GAGATGCGGCCCATGAGCTGCGAGAAGGCGTTCATGCCGGCCTCGTTCCAGACGCGGCTGTAGACCACGG
CTCCGTTGGGGTCGCGCGCGCGCATGACCACCTGGGCGAGGTTAAGCTCGACGTGGCGCGTGAAGACCGC
GTAGTTGCAGAGGCGCTGGTAGAGGTAGTTGAGCGTGGTGGCGATGTGCTCGGTGACGAAGAAGTACATG
ATCCAGCGGCGGAGCGGCATCTCGCTGACGTCGCCCAGGGCTTCCAAGCGCTCCATGGTCTCGTAGAAGT
CCACGGCGAAGTTGAAAAACTGGGAGTTGCGCGCCGAGACGGTCAACTCCTCCTCCAGAAGACGGATGAG
CTCGGCGATGGTGGCGCGCACCTCGCGCTCGAAGGCCCCGGGGGGCTCCTCTTCTTCCATCTCCTCCTCC
TCTTCCTCCTCCACTAACATCTCTTCTACTTCCTCCTCAGGAGGCGGCGGCGGGGGAGGGGCCCTGCGTC
GCCGGCGGCGCACGGGCAGACGGTCGATGAAGCGCTCGATGGTCTCCCCGCGCCGGCGACGCATGGTCTC
GGTGACGGCGCGCCCGTCCTCGCGGGGCCGCAGCGTGAAGACGCCGCCGCGCATCTCCAGGTGGCCGCCG
GGGGGGTCTCCGTTGGGCAGGGAGAGGGCGCTGACGATGCATCTTATCAATTGGCCCGTAGGGACTCCGC
GCAAGGACCTGAGCGTCTCGAGATCCACGGGATCCGAAAACCGCTGAACGAAGGCTTCGAGCCAGTCGCA
GTCGCAAGGTAGGCTGAGCCCGGTTTCTTGTTCTTCGGGTATTTGGTCGGGAGGCGGGCGGGCGATGCTG
CTGGTGATGAAGTTGAAGTAGGCGGTCCTGAGACGGCGGATGGTGGCGAGGAGCACCAGGTCCTTGGGCC
CGGCTTGCTGGATGCGCAGACGGTCGGCCATGCCCCAGGCGTGGTCCTGACACCTGGCGAGGTCCTTGTA
GTAGTCCTGCATGAGCCGCTCTACGGGCACGTCCTCCTCGCCCGCGCGGCCGTGCATGCGCGTGAGCCCG
AACCCGCGCTGCGGCTGGACGAGCGCCAGGTCGGCGACGACGCGCTCGGCGAGGATGGCCTGCTGGATCT
GGGTGAGGGTGGTCTGGAAGTCGTCGAAGTCGACGAAGCGGTGGTAGGCTCCGGTGTTGATGGTGTAGGA
GCAGTTGGCCATGACGGACCAGTTGACGGTCTGGTGGCCGGGGCGCACGAGCTCGTGGTACTTGAGGCGC
GAGTAGGCGCGCGTGTCGAAGATGTAGTCGTTGCAGGTGCGCACGAGGTACTGGTATCCGACGAGGAAGT
GCGGCGGCGGCTGGCGGTAGAGCGGCCATCGCTCGGTGGCGGGGGCGCCGGGCGCGAGGTCCTCGAGCAT
GAGGCGGTGGTAGCCGTAGATGTACCTGGACATCCAGGTGATGCCGGCGGCGGTGGTGGAGGCGCGCGGG
AACTCGCGGACGCGGTTCCAGATGTTGCGCAGCGGCAGGAAGTAGTTCATGGTGGCCGCGGTCTGGCCCG
TGAGGCGCGCGCAGTCGTGGATGCTCTAGACATACGGGCAAAAACGAAAGCGGTCAGCGGCTCGACTCCG
TGGCCTGGAGGCTAAGCGAACGGGTTGGGCTGCGCGTGTACCCCGGTTCGAATCTCGAATCAGGCTGGAG
CCGCAGCTAACGTGGTACTGGCACTCCCGTCTCGACCCAAGCCTGCTAACGAAACCTCCAGGATACGGAG
GCGGGTCGTTTTTTGGCCTTGGTCGCTGGTCATGAAAAACTAGTAAGCGCGGAAAGCGGCCGCCCGCGAT
GGCTCGCTGCCGTAGTCTGGAGAAAGAATCGCCAGGGTTGCGTTGCGGTGTGCCCCGGTTCGAGCCTCAG
CGCTCGGTGCCGGCCGGATTCCGCGGCTAACGTGGGCGTGGCTGCCCCGTCGTTTCCAAGACCCCTTAGC
CAGCCGACTTCTCCAGTTACGGAGCGAGCCCCTCTTTTTCTTGTGTTTTTGCCAGATGCATCCCGTACTG
CGGCAGATGCGCCCCCACCCTCCACCACAACCGCCCCTACCGCAGCAGCAGCAACAGCCGGCGCTTCTGC
CCCCGCCCCAGCAGCAGCAGCCAGCCACTACCGCGGCGGCCGCCGTGAGCGGAGCCGGCGTTCAGTATGA
CCTGGCCTTGGAAGAGGGCGAGGGGCTGGCGCGGCTGGGGGCGTCGTCGCCGGAGCGGCACCCGCGCGTG
CAGATGAAAAGGGACGCTCGCGAGGCCTACGTGCCCAAGCAGAACCTGTTCAGAGACAGGAGCGGCGAGG
AGCCCGAGGAGATGCGCGCCTCCCGCTTCCACGCGGGGCGGGAGCTGCGGCGCGGCCTGGACCGAAAGCG
GGTGCTGAGGGACGAGGATTTCGAGGCGGACGAGCTGACGGGGATCAGCCCCGCGCGCGCGCACGTGGCC
GCGGCCAACCTGGTCACGGCGTACGAGCAGACCGTGAAGGAGGAGAGCAACTTTCAAAAATCCTTCAACA
ACCACGTGCGCACGCTGATCGCGCGCGAGGAGGTGACCCTGGGCCTGATGCACCTGTGGGACCTGCTGGA
GGCCATCGTGCAGAACCCCACGAGCAAGCCGCTGACGGCGCAGCTGTTTCTGGTGGTGCAGCACAGTCGG
GACAACGAGACGTTCAGGGAGGCGCTGCTGAATATCACCGAGCCCGAGGGCCGCTGGCTCCTGGACCTGG
TGAACATTCTGCAGAGCATCGTGGTGCAGGAGCGCGGGCTGCCGCTGTCCGAGAAGCTGGCGGCCATCAA
CTTCTCGGTGCTGAGCCTGGGCAAGTACTACGCTAGGAAGATCTACAAGACCCCGTACGTGCCCATAGAC
AAGGAGGTGAAGATCGACGGGTTTTACATGCGCATGACCCTGAAAGTGCTGACCCTGAGCGACGATCTGG
GGGTGTACCGCAACGACAGGATGCACCGCGCGGTGAGCGCCAGCCGCCGGCGCGAGCTGAGCGACCAGGA
GCTGATGCACAGCCTGCAGCGGGCCCTGACCGGGGCCGGGACCGAGGGGGAGAGCTACTTTGACATGGGC
GCGGACCTGCGCTGGCAGCCCAGCCGCCGGGCCTTGGAAGCTGCCGGCGGCGTGCCCTACGTGGAGGAGG
TGGACGATGAGGAGGAGGAGGGCGAGTACCTGGAAGACTGATGGCGCGACCGTATTTTTGCTAGATGCAG
CAACAGCCACCGCCGCCTCCTGATCCCGCGATGCGGGCGGCGCTGCAGAGCCAGCCGTCCGGCATTAACT
CCTCGGACGATTGGACCCAGGCCATGCAACGCATCATGGCGCTGACGACCCGCAATCCCGAAGCCTTTAG
ACAGCAGCCTCAGGCCAACCGGCTCTCGGCCATCCTGGAGGCCGTGGTGCCCTCGCGCTCGAACCCCACG
CACGAGAAGGTGCTGGCCATCGTGAACGCGCTGGTGGAGAACAAGGCCATCCGCGGCGACGAGGCCGGGC
TGGTGTACAACGCGCTGCTGGAGCGCGTGGCCCGCTACAACAGCACCAACGTGCAGACGAACCTGGACCG
CATGGTGACCGACGTGCGCGAGGCGGTGTCGCAGCGCGAGCGGTTCCACCGCGAGTCGAACCTGGGCTCC
ATGGTGGCGCTGAACGCCTTCCTGAGCACGCAGCCCGCCAACGTGCCCCGGGGCCAGGAGGACTACACCA
ACTTCATCAGCGCGCTGCGGCTGATGGTGGCCGAGGTGCCCCAGAGCGAGGTGTACCAGTCGGGGCCGGA
CTACTTCTTCCAGACCAGTCGCCAGGGCTTGCAGACCGTGAACCTGAGCCAGGCTTTCAAGAACTTGCAG
GGACTGTGGGGCGTGCAGGCCCCGGTCGGGGACCGCGCGACGGTGTCGAGCCTGCTGACGCCGAACTCGC
GCCTGCTGCTGCTGCTGGTGGCGCCCTTCACGGACAGCGGCAGCGTGAGCCGCGACTCGTACCTGGGCTA
CCTGCTTAACCTGTACCGCGAGGCCATCGGGCAGGCGCACGTGGACGAGCAGACCTACCAGGAGATCACC
CACGTGAGCCGCGCGCTGGGCCAGGAGGACCCGGGCAACCTGGAGGCCACCCTGAACTTCCTGCTGACCA
ACCGGTCGCAGAAGATCCCGCCCCAGTACGCGCTGAGCACCGAGGAGGAGCGCATCCTGCGCTACGTGCA
GCAGAGCGTGGGGCTGTTCCTGATGCAGGAGGGGGCCACGCCCAGCGCCGCGCTCGACATGACCGCGCGC
AACATGGAGCCCAGCATGTACGCCCGCAACCGCCCGTTCATCAATAAGCTGATGGACTACTTGCATCGGG
CGGCCGCCATGAACTCGGACTACTTTACCAACGCCATCTTGAACCCGCACTGGCTCCCGCCGCCCGGGTT
CTACACGGGCGAGTACGACATGCCCGACCCCAACGACGGGTTCCTGTGGGATGACGTGGACAGCAGCGTG
TTCTCGCCGCGTCCCACCACCACCGTGTGGAAGAAAGAGGGCGGGGACCGGCGGCCGTCCTCGGCGCTGT
CCGGTCGCGCGGGTGCTGCCGCGGCGGTGCCCGAGGCCGCCAGCCCCTTTCCGAGCCTGCCCTTTTCGCT
GAACAGCGTGCGCAGCAGCGAGCTGGGTCGGCTGACGCGGCCGCGCCTGCTGGGCGAGGAGGAGTACCTG
AACGACTCCTTGTTGAGGCCCGAGCGCGAAAAGAACTTCCCCAATAACGGGATAGAGAGCCTGGTGGACA
AGATGAGCCGCTGGAAGACGTACGCGCACGAGCACAGGGACGAGCCCCGAGCTAGCAGCGCAGGCACCCG
TAGACGCCAGCGGCACGACAGGCAGCGGGGTCTGGTGTGGGACGATGAGGATTCCGCCGACGACAGCAGC
GTGTTGGACTTGGGTGGGAGTGGTGGTGGTAACCCGTTCGCTCACTTGCGCCCCCGTATCGGGCGCCTGA
TGTAAGAATCTGAAAAATAAAAAACGGTACTCACCAAGGCCATGGCGACCAGCGTGCGTTCTTCTCTGTT
GTTTGTAGTAGTATGATGAGGCGCGTGTACCCGGAGGGTCCTCCTCCCTCGTACGAGAGCGTGATGCAGC
AGGCGGTGGCGGCGGCGATGCAGCCCCCGCTGGAGGCGCCTTACGTGCCCCCGCGGTACCTGGCGCCTAC
GGAGGGGCGGAACAGCATTCGTTACTCGGAGCTGGCACCCTTGTACGATACCACCCGGTTGTACCTGGTG
GACAACAAGTCGGCGGACATCGCCTCGCTGAACTACCAGAACGACCACAGCAACTTCCTGACCACCGTGG
TGCAGAACAACGATTTCACCCCCACGGAGGCCAGCACCCAGACCATCAACTTTGACGAGCGCTCGCGGTG
GGGCGGCCAGCTGAAAACCATCATGCACACCAACATGCCCAACGTGAACGAGTTCATGTACAGCAACAAG
TTCAAGGCGCGGGTGATGGTCTCGCGCAAGACCCCCAACGGGGTCACAGTAACAGATGGTAGTCAGGACG
AGCTGACCTACGAGTGGGTGGAGTTTGAGCTGCCCGAGGGCAACTTCTCGGTGACCATGACCATCGATCT
GATGAACAACGCCATCATCGACAACTACTTGGCGGTGGGGCGGCAGAACGGGGTGCTGGAGAGCGACATC
GGCGTGAAGTTCGACACGCGCAACTTCCGGCTGGGCTGGGACCCCGTGACCGAGCTGGTGATGCCGGGCG
TGTACACCAACGAGGCCTTCCACCCCGACATCGTCCTGCTGCCCGGCTGCGGCGTGGACTTCACCGAGAG
CCGCCTCAGCAACCTGCTGGGCATCCGCAAGCGGCAGCCCTTCCAGGAGGGCTTCCAGATCCTGTACGAG
GACCTGGAGGGGGGCAACATCCCCGCGCTCTTGGATGTCGAAGCCTACGAGAAAAGCAAGGAGGATAGCA
CCGCCGTGGCTACCGCCGCGACTGTGGCAGATGCCACTGTCACCAGGGGCGATACATTCGCCACCCAGGC
GGAGGAAGCAGCCGCCCTAGCGGCGACCGATGATAGTGAAAGTAAGATAGTTATCAAGCCGGTGGAGAAG
GACAGCAAGGACAGGAGCTACAACGTTCTATCGGATGGAAAGAACACCGCCTACCGCAGCTGGTACCTGG
CCTACAACTACGGCGACCCCGAGAAGGGCGTGCGCTCCTGGACGCTGCTCACCACCTCGGACGTCACCTG
CGGCGTGGAGCAAGTCTACTGGTCGCTGCCCGACATGATGCAAGACCCGGTCACCTTCCGCTCCACGCGT
CAAGTTAGCAACTACCCGGTGGTGGGCGCCGAGCTCCTGCCCGTCTACTCCAAGAGCTTCTTCAACGAGC
AGGCCGTCTACTCGCAGCAGCTGCGCGCCTTCACCTCGCTCACGCACGTCTTCAACCGCTTCCCCGAGAA
CCAGATCCTCGTCCGCCCGCCCGCGCCCACCATTACCACCGTCAGTGAAAACGTTCCTGCTCTCACAGAT
CACGGGACCCTGCCGCTGCGCAGCAGTATCCGGGGAGTCCAGCGCGTGACCGTCACTGACGCCAGACGCC
GCACCTGCCCCTACGTCTACAAGGCCCTGGGCGTAGTCGCGCCGCGCGTCCTCTCGAGCCGCACCTTCTA
AAAAATGTCCATTCTCATCTCGCCCAGTAATAACACCGGTTGGGGCCTGCGCGCGCCCAGCAAGATGTAC
GGAGGCGCTCGCCAACGCTCCACGCAACACCCCGTGCGCGTGCGCGGGCACTTCCGCGCTCCCTGGGGCG
CCCTCAAGGGTCGCGTGCGCTCGCGCACCACCGTCGACGACGTGATCGACCAGGTGGTGGCCGACGCGCG
CAACTACACGCCCGCCGCCGCGCCCGCCTCCACCGTGGACGCCGTCATCGACAGCGTGGTGGCCGACGCG
CGCCGGTACGCCCGCGCCAAGAGCCGGCGGCGGCGCATCGCCCGGCGGCACCGGAGCACCCCCGCCATGC
GCGCGGCGCGAGCCTTGCTGCGCAGGGCCAGGCGCACGGGACGCAGGGCCATGCTCAGGGCGGCCAGACG
CGCGGCCTCCGGCAGCAGCAGCGCCGGCAGGACCCGCAGACGCGCGGCCACGGCGGCGGCGGCGGCCATC
GCCAGCATGTCCCGCCCGCGGCGCGGCAACGTGTACTGGGTGCGCGACGCCGCCACCGGTGTGCGCGTGC
CCGTGCGCACCCGCCCCCCTCGCACTTGAAGATGCTGACTTCGCGATGTTGATGTGTCCCAGCGGCGAGG
AGGATGTCCAAGCGCAAATACAAGGAAGAGATGCTCCAGGTCATCGCGCCTGAGATCTACGGCCCCGCGG
CGGCGGTGAAGGAGGAAAGAAAGCCCCGCAAACTGAAGCGGGTCAAAAAGGACAAAAAGGAGGAGGAAGA
TGTGGACGGACTGGTGGAGTTTGTGCGCGAGTTCGCCCCCCGGCGGCGCGTGCAGTGGCGCGGGCGGAAA
GTGAAACCGGTGCTGCGGCCCGGCACCACGGTGGTCTTCACGCCCGGCGAGCGTTCCGGCTCCGCCTCCA
AGCGCTCCTACGACGAGGTGTACGGGGACGAGGACATCCTCGAGCAGGCGGCCGAGCGTCTGGGCGAGTT
TGCTTACGGCAAGCGCAGCCGCCCCGCGCCCTTGAAAGAGGAGGCGGTGTCCATCCCGCTGGACCACGGC
AACCCCACGCCGAGCCTGAAGCCGGTGACCCTGCAGCAGGTGCTGCCGAGCGCGGCGCCGCGCCGGGGCT
TCAAGCGCGAGGGCGGCGAGGATCTGTACCCGACCATGCAGCTGATGGTGCCCAAGCGCCAGAAGCTGGA
GGACGTGCTGGAGCACATGAAGGTGGACCCCGAGGTGCAGCCCGAGGTCAAGGTGCGGCCCATCAAGCAG
GTGGCCCCGGGCCTGGGCGTGCAGACCGTGGACATCAAGATCCCCACGGAGCCCATGGAAACGCAGACCG
AGCCCGTGAAGCCCAGCACCAGCACCATGGAGGTGCAGACGGATCCCTGGATGCCGGCGCCGGCTTCCAC
CACCACTCGCCGAAGACGCAAGTACGGCGCGGCCAGCCTGCTGATGCCCAACTACGCGCTGCATCCTTCC
ATCATCCCCACGCCGGGCTACCGCGGCACGCGCTTCTACCGCGGCTACAGCAGCCGCCGCAAGACCACCA
CCCGCCGCCGCCGTCGCCGCACCCGCCGCAGCACCACCGCGACTTCCGCCGCCGCCTTGGTGCGGAGAGT
GTACCGCAGCGGGCGTGAGCCTCTGACCCTGCCGCGCGCGCGCTACCACCCGAGCATCGCCATTTAACTC
TGCCGTCGCCTCCTTGCAGATATGGCCCTCACATGCCGCCTCCGCGTCCCCATTACGGGCTACCGAGGAA
GAAAGCCGCGCCGTAGAAGGCTGACGGGGAACGGGCTGCGTCGCCATCACCACCGGCGGCGGCGCGCCAT
CAGCAAGCGGTTGGGGGGAGGCTTCCTGCCCGCGCTGATCCCCATCATCGCCGCGGCGATCGGGGCGATC
CCCGGCATAGCTTCCGTGGCGGTGCAGGCCTCTCAGCGCCACTGAGACACAGCTTGGAAAATTTGTAATA
AAAAAATGGACTGACGCTCCTGGTCCTGTGATGTGTGTTTTTAGATGGAAGACATCAATTTTTCGTCCCT
GGCACCGCGACACGGCACGCGGCCGTTTATGGGCACCTGGAGCGACATCGGCAACAGCCAACTGAACGGG
GGCGCCTTCAATTGGAGCAGTCTCTGGAGCGGGCTTAAGAATTTCGGGTCCACGCTCAAAACCTATGGCA
ACAAGGCGTGGAACAGCAGCACAGGGCAGGCGCTGAGGGAAAAGCTGAAAGAGCAGAACTTCCAGCAGAA
GGTGGTCGATGGCCTGGCCTCGGGCATCAACGGGGTGGTGGACCTGGCCAACCAGGCCGTGCAGAAACAG
ATCAACAGCCGCCTGGACGCGGTCCCGCCCGCGGGGTCCGTGGAGATGCCCCAGGTGGAGGAGGAGCTGC
CTCCCCTGGACAAGCGCGGCGACAAGCGACCGCGTCCCGACGCGGAGGAGACGCTGCTGACGCACACGGA
CGAGCCGCCCCCGTACGAGGAGGCGGTGAAACTGGGTCTGCCCACCACGCGGCCCGTGGCGCCTCTGGCC
ACCGGGGTGCTGAAACCCAGCAGCAGCAGCAGCCAGCCCGCGACCCTGGACTTGCCTCCACCTCGCCCCT
CCACAGTGGCTAAGCCCCTGCCGCCGGTGGCCGTCGCGTCGCGCGCCCCCCGAGGCCGCCCCCAGGCGAA
CTGGCAGAGCACTCTGAACAGCATCGTGGGTCTGGGAGTGCAGAGTGTGAAGCGCCGCCGCTGCTATTAA
AAGACACTGTAGCGCTTAACTTGCTTGTCTGTGTGTATATGTATGTCCGCCGACCAGAAGGAGGAGGAAG
AGGCGCGTCGCCGAGTTGCAAGATGGCCACCCCATCGATGCTGCCCCAGTGGGCGTACATGCACATCGCC
GGACAGGACGCTTCGGAGTACCTGAGTCCGGGTCTGGTGCAGTTCGCCCGCGCCACAGACACCTACTTCA
GTCTGGGGAACAAGTTTAGGAACCCCACGGTGGCACCCACGCACGATGTGACCACCGACCGCAGCCAGCG
GCTGACGCTGCGCTTCGTGCCCGTGGACCGCGAGGACAACACCTACTCGTACAAAGTGCGCTACACGCTG
GCCGTGGGCGACAACCGCGTGCTGGACATGGCCAGCACCTACTTTGACATCCGCGGCGTGCTGGATCGGG
GCCCCAGCTTCAAACCCTACTCCGGCACCGCCTACAACAGCCTGGCTCCCAAGGGAGCGCCCAACACCTC
ACAGTGGATAACCAAAGACAATGGAACTGATAAGACATACAGTTTTGGAAATGCTCCAGTCAGAGGATTG
GACATTACAGAAGAGGGTCTCCAAATAGGAACCGATGAGTCAGGGGGTGAAAGCAAGAAAATTTTTGCAG
ACAAAACCTATCAGCCTGAACCTCAGCTTGGAGATGAGGAATGGCATGATACTATTGGAGCTGAAGACAA
GTATGGAGGCAGAGCGCTTAAACCTGCCACCAACATGAAACCCTGCTATGGGTCTTTCGCCAAGCCAACT
AATGCTAAGGGAGGTCAGGCTAAAAGCAGAACCAAGGACGATGGCACTACTGAGCCTGATATTGACATGG
CCTTCTTTGACGATCGCAGTCAGCAAGCTAGTTTCAGTCCAGAACTTGTTTTGTATACTGAGAATGTCGA
TCTGGACACCCCGGATACCCACATTATTTACAAACCTGGCACTGATGAAACAAGTTCTTCTTTCAACTTG
GGTCAGCAGTCCATGCCCAACAGACCCAACTACATTGGCTTCAGAGACAACTTTATCGGGCTCATGTACT
ACAACAGCACTGGCAATATGGGTGTACTGGCCGGTCAGGCCTCCCAGCTGAATGCTGTGGTGGACTTGCA
GGACAGAAACACTGAACTGTCCTACCAGCTCTTGCTTGACTCTCTGGGTGACAGAACCAGGTATTTCAGT
ATGTGGAATCAGGCGGTGGACAGCTATGACCCCGATGTGCGCATTATTGAAAATCACGGTGTGGAGGATG
AACTCCCCAACTATTGCTTCCCTTTGAATGGTGTGGGCTTTACAGATACATTCCAGGGAATTAAGGTTAA
AACTACAAATAACGGAACAGCAAATGCTACAGAGTGGGAATCTGATACCTCTGTCAATAATGCTAATGAG
ATTGCCAAGGGCAATCCTTTCGCCATGGAGATCAACATCCAGGCCAACCTGTGGCGGAACTTCCTCTACG
CGAACGTGGCGCTGTACCTGCCCGACTCCTACAAGTACACGCCGGCCAACATCACGCTGCCCACCAACAC
CAACACCTACGATTACATGAACGGCCGCGTGGTGGCGCCCTCGCTGGTGGACGCCTACATCAACATCGGG
GCGCGCTGGTCGCTGGACCCCATGGACAACGTCAACCCCTTCAACCACCACCGCAACGCGGGCCTGCGCT
ACCGCTCCATGCTCCTGGGCAACGGGCGCTACGTGCCCTTCCACATCCAGGTGCCCCAAAAGTTTTTCGC
CATCAAGAGCCTCCTGCTCCTGCCCGGGTCCTACACCTACGAGTGGAACTTCCGCAAGGACGTCAACATG
ATCCTGCAGAGCTCCCTCGGCAACGACCTGCGCACGGACGGGGCCTCCATCGCCTTCACCAGCATCAACC
TCTACGCCACCTTCTTCCCCATGGCGCACAACACCGCCTCCACGCTCGAGGCCATGCTGCGCAACGACAC
CAACGACCAGTCCTTCAACGACTACCTCTCGGCGGCCAACATGCTCTACCCCATCCCGGCCAACGCCACC
AACGTGCCCATCTCCATCCCCTCGCGCAACTGGGCCGCCTTCCGCGGATGGTCCTTCACGCGCCTCAAGA
CCCGCGAGACGCCCTCGCTCGGCTCCGGGTTCGACCCCTACTTCGTCTACTCGGGCTCCATCCCCTACCT
CGACGGCACCTTCTACCTCAACCACACCTTCAAGAAGGTCTCCATCACCTTCGACTCCTCCGTCAGCTGG
CCCGGCAACGACCGCCTCCTGACGCCCAACGAGTTCGAAATCAAGCGCACCGTCGACGGAGAGGGGTACA
ACGTGGCCCAGTGCAACATGACCAAGGACTGGTTCCTGGTCCAGATGCTGGCCCACTACAACATCGGCTA
CCAGGGCTTCTACGTGCCCGAGGGCTACAAGGACCGCATGTACTCCTTCTTCCGCAACTTCCAGCCCATG
AGCCGCCAGGTCGTGGACGAGGTCAACTACAAGGACTACCAGGCCGTCACCCTGGCCTACCAGCACAACA
ACTCGGGCTTCGTCGGCTACCTCGCGCCCACCATGCGCCAGGGCCAGCCCTACCCCGCCAACTACCCCTA
CCCGCTCATCGGCAAGAGCGCCGTCGCCAGCGTCACCCAGAAAAAGTTCCTCTGCGACCGGGTCATGTGG
CGCATCCCCTTCTCCAGCAACTTCATGTCCATGGGCGCGCTCACCGACCTCGGCCAGAACATGCTCTACG
CCAACTCCGCCCACGCGCTAGACATGAATTTCGAAGTCGACCCCATGGATGAGTCCACCCTTCTCTATGT
TGTCTTCGAAGTCTTCGACGTCGTCCGAGTGCACCAGCCCCACCGCGGCGTCATCGAGGCCGTCTACCTG
CGCACGCCCTTCTCGGCCGGCAACGCCACCACCTAAGCCTCTTGCTTCTTGCAAGATGACGGCCTGTGGC
TCCGGCGAGCAGGAGCTCAGGGCCATCCTCCGCGACCTGGGCTGCGGGCCCTACTTCCTGGGCACCTTCG
ACAAGCGCTTCCCGGGATTCATGGCCCCGCACAAGCTGGCCTGCGCCATCGTCAACACGGCCGGCCGCGA
GACCGGGGGCGAGCACTGGCTGGCCTTCGCCTGGAACCCGCGCACCCACACCTGCTACCTCTTCGACCCC
TTCGGGTTCTCGGACGAGCGCCTCAAGCAGATCTACCAGTTCGAGTACGAGGGCCTGCTGCGCCGCAGCG
CCCTGGCCACCGAGGACCGCTGCGTCACCCTGGAAAAGTCCACCCAGACCGTGCAGGGTCCGCGCTCGGC
CGCCTGCGGGCTCTTCTGCTGCATGTTCCTGCACGCCTTCGTGCACTGGCCCGACCGCCCCATGGACAAG
AACCCCACCATGAACTTGCTGACGGGGGTGCCCAACGGCATGCTCCAGTCGCCCCAGGTGGAACCCACCC
TGCGCCGCAACCAGGAGGCGCTCTACCGCTTCCTCAACGCCCACTCCGCCTACTTTCGCTCCCACCGCGC
GCGCATCGAGAAGGCCACCGCCTTCGACCGCATGAATCAAGACATGTAAACTGTGTGTATGTGAATGCTT
TATTCATAATAAACAGCACATGTTTATGCCACCTTCTCTGAGGCTCTGACTTTATTTAGAAATCGAAGGG
GTTCTGCCGGCTCTCGGCGTGCCCCGCGGGCAGGGATACGTTGCGGAACTGGTACTTGGGCAGCCACTTG
AACTCGGGGATCAGCAGCTTCGGCACGGGGAGGTCGGGGAACGAGTCGCTCCACAGCTTGCGCGTGAGTT
GCAGGGCGCCCAGCAGGTCGGGCGCGGATATCTTGAAATCACAGTTGGGACCCGCGTTCTGCGCGCGAGA
GTTGCGGTACACGGGGTTGCAGCACTGGAACACCATCAGGGCCGGGTGCTTCACGCTCGCCAGCACCGTC
GCGTCGGTGATGCCCTCCACGTCCAGATCCTCGGCGTTGGCCATCCCGAAGGGGGTCATCTTGCAGGTCT
GCCGCCCCATGCTGGGCACGCAGCCGGGCTTGTGGTTGCAATCGCAGTGCAGGGGGATCAGCATCATCTG
GGCCTGCTCGGAGCTCATGCCCGGGTACATGGCCTTCATGAAAGCCTCCAGCTGGCGGAAGGCCTGCTGC
GCCTTGCCGCCCTCGGTGAAGAAGACCCCGCAGGACTTGCTAGAGAACTGGTTGGTGGCGCAGCCGGCGT
CGTGCACGCAGCAGCGCGCGTCGTTGTTGGCCAGCTGCACCACGCTGCGCCCCCAGCGGTTCTGGGTGAT
CTTGGCCCGGTCGGGGTTCTCCTTCAGCGCGCGCTGCCCGTTCTCGCTCGCCACATCCATCTCGATCGTG
TGCTCCTTCTGGATCATCACGGTCCCGTGCAGGCACCGCAGCTTGCCCTCGGCTTCGGTGCATCCGTGCA
GCCACAGCGCGCAGCCGGTGCACTCCCAGTTCTTGTGGGCGATCTGGGAGTGCGAGTGCACGAAGCCCTG
CAGGAAGCGGCCCATCATCGCGGTCAGGGTCTTGTTGCTGGTGAAGGTCAGCGGGATGCCGCGGTGCTCC
TCGTTCACATACAGGTGGCAGATGCGGCGGTACACCTCGCCCTGCTCGGGCATCAGCTGGAAGGCGGACT
TCAGGTCGCTCTCCACGCGGTACCGCTCCATCAGCAGCGTCATGACTTCCATGCCCTTCTCCCAGGCCGA
AACGATCGGCAGGCTCAGGGGGTTCTTCACCGTTGTCATCTTAGTCGCCGCCGCCGAGGTCAGGGGGTCG
TTCTCGTCCAGGGTCTCAAACACTCGCTTGCCGTCCTTCTCGGTGATGCGCACGGGGGGAAAGCTGAAGC
CCACGGCCGCCAGCTCCTCCTCGGCCTGCCTTTCGTCCTCGCTGTCCTGGCTGATGTCTTGCAAAGGCAC
ATGCTTGGTCTTGCGGGGTTTCTTTTTGGGCGGCAGAGGCGGCGGCGGAGACGTGCTGGGCGAGCGCGAG
TTCTCGCTCACCACGACTATTTCTTCTTCTTGGCCGTCGTCCGAGACCACGCGGCGGTAGGCATGCCTCT
TCTGGGGCAGAGGCGGAGGCGACGGGCTCTCGCGGTTCGGCGGGCGGCTGGCAGAGCCCCTTCCGCGTTC
GGGGGTGCGCTCCTGGCGGCGCTGCTCTGACTGACTTCCTCCGCGGCCGGCCATTGTGTTCTCCTAGGGA
GCAAGCATGGAGACTCAGCCATCGTCGCCAACATCGCCATCTGCCCCCGCCGCCGCCGACGAGAACCAGC
AGCAGCAGAATGAAAGCTTAACCGCCCCGCCGCCCAGCCCCACCTCCGACGCCGCGGCCCCAGACATGCA
AGAGATGGAGGAATCCATCGAGATTGACCTGGGCTACGTGACGCCCGCGGAGCACGAGGAGGAGCTGGCA
GCGCGCTTTTCAGCCCCGGAAGAGAACCACCAAGAGCAGCCAGAGCAGGAAGCAGAGAGCGAGCAGAGCC
AGGCTGGGCTCGAGCATGGCGACTACCTGAGCGGGGCAGAGGACGTGCTCATCAAGCATCTGGCCCGCCA
ATGCATCATCGTCAAGGATGCGCTGCTCGACCGCGCCGAGGTGCCCCTCAGCGTGGCGGAGCTCAGCCGC
GCCTACGAGCGCAACCTCTTCTCGCCGCGCGTGCCCCCCAAGCGCCAGCCCAACGGCACCTGCGAGCCCA
ACCCGCGCCTCAACTTCTACCCGGTCTTCGCGGTGCCCGAGGCCCTGGCCACCTACCACCTCTTTTTCAA
GAACCAAAGGATCCCCGTCTCCTGCCGCGCCAACCGCACCCGCGCCGACGCCCTGCTCAACCTGGGCCCC
GGCGCCCGCCTACCTGATATCGCCTCCTTGGAAGAGGTTCCCAAGATCTTCGAGGGTCTGGGCAGCGACG
AGACTCGGGCCGCGAACGCTCTGCAAGGAAGCGGAGAGGAGCATGAGCACCACAGCGCCCTGGTGGAGTT
GGAAGGCGACAACGCGCGCCTGGCGGTCCTCAAGCGCACGGTCGAGCTGACCCACTTCGCCTACCCGGCG
CTCAACCTGCCCCCCAAGGTCATGAGCGCCGTCATGGACCAGGTGCTCATCAAGCGCGCCTCGCCCCTCT
CGGAGGAGGAGATGCAGGACCCCGAGAGCTCGGACGAGGGCAAGCCCGTGGTCAGCGACGAGCAGCTGGC
GCGCTGGCTGGGAGCGAGTAGCACCCCCCAGAGCCTGGAAGAGCGGCGCAAGCTCATGATGGCCGTGGTC
CTGGTGACCGTGGAGCTGGAGTGTCTGCGCCGCTTCTTCGCCGACGCGGAGACCCTGCGCAAGGTCGAGG
AGAACCTGCACTACCTCTTCAGGCACGGGTTCGTGCGCCAGGCCTGCAAGATCTCCAACGTGGAGCTGAC
CAACCTGGTCTCCTACATGGGCATCCTGCACGAGAACCGCCTGGGGCAGAACGTGCTGCACACCACCCTG
CGCGGGGAGGCCCGCCGCGACTACATCCGCGACTGCGTCTACCTGTACCTCTGCCACACCTGGCAGACGG
GCATGGGCGTGTGGCAGCAGTGCCTGGAGGAGCAGAACCTGAAAGAGCTCTGCAAGCTCCTGCAGAAGAA
CCTGAAGGCCCTGTGGACCGGGTTCGACGAGCGCACCACCGCCTCGGACCTGGCCGACCTCATCTTCCCC
GAGCGCCTGCGGCTGACGCTGCGCAACGGGCTGCCCGACTTTATGAGCCAAAGCATGTTGCAAAACTTTC
GCTCTTTCATCCTCGAACGCTCCGGGATCCTGCCCGCCACCTGCTCCGCGCTGCCCTCGGACTTCGTGCC
GCTGACCTTCCGCGAGTGCCCCCCGCCGCTCTGGAGCCACTGCTACCTGCTGCGTCTGGCCAACTACCTG
GCCTACCACTCGGACGTGATCGAGGACGTCAGCGGCGAGGGTCTGCTCGAGTGCCACTGCCGCTGCAACC
TCTGCACGCCGCACCGCTCCCTGGCCTGCAACCCCCAGCTGCTGAGCGAGACCCAGATCATCGGCACCTT
CGAGTTGCAAGGCCCCGGCGAGGAGGGCAAGGGGGGTCTGAAACTCACCCCGGGGCTGTGGACCTCGGCC
TACTTGCGCAAGTTCGTGCCCGAGGACTACCATCCCTTCGAGATCAGGTTCTACGAGGACCAATCCCAGC
CGCCCAAGGCCGAGCTGTCGGCCTGCGTCATCACCCAGGGGGCCATCCTGGCCCAATTGCAAGCCATCCA
GAAATCCCGCCAAGAATTTCTGCTGAAAAAGGGCCACGGGGTCTACTTGGACCCCCAGACCGGAGAGGAG
CTCAACCCCAGCTTCCCCCAGGATGCCCAGAGGAAGCAGCAAGAAGCTGAAAGTGGAGCTGCCGCTGCCG
CCGGAGGATTTGGAGGAAGACTGGGAGAGCAGTCAGGCAGAGGAGGAGGAGATGGAAGACTGGGACAGCA
CTCAGGCAGAGGAGGACAGCCTGCAAGACAGTCTGGAAGACGAGGTGGAGGAGGAGGCAGAGGAAGAAGC
AGCCGCCGCCAGACCGTCGTCCTCGGCGGAGAAAGCAAGCAGCACGGATACCATCTCCGCTCCGGGTCGG
GGTCTCGGCGGCCGGGCCCACAGTAGGTGGGACGAGACCGGGCGCTTCCCGAACCCCACCACCCAGACCG
GTAAGAAGGAGCGGCAGGGATACAAGTCCTGGCGGGGGCACAAAAACGCCATCGTCTCCTGCTTGCAAGC
CTGCGGGGGCAACATCTCCTTCACCCGGCGCTACCTGCTCTTCCACCGCGGGGTGAACTTCCCCCGCAAC
ATCTTGCATTACTACCGTCACCTCCACAGCCCCTACTACTGTTTCCAAGAAGAGGCAGAAACCCAGCAGC
AGCAGAAAACCAGCAGCAGCTAGAAAATCCACAGCGGCGGCGGCGGCAGGTGGACTGAGGATCGCGGCGA
ACGAGCCGGCGCAGACCCGGGAGCTGAGGAACCGGATCTTTCCCACCCTCTATGCCATCTTCCAGCAGAG
TCGGGGGCAGGAGCAGGAACTGAAAGTCAAGAACCGTTCTCTGCGCTCGCTCACCCGCAGTTGTCTGTAT
CACAAGAGCGAAGACCAACTTCAGCGCACTCTCGAGGACGCCGAGGCTCTCTTCAACAAGTACTGCGCGC
TCACTCTTAAAGAGTAGCCCGCGCCCGCCCACACACGGAAAAAGGCGGGAATTACGTCACCACCTGCGCC
CTTCGCCCGACCATCATCATGAGCAAAGAGATTCCCACGCCTTACATGTGGAGCTACCAGCCCCAGATGG
GCCTGGCCGCCGGCGCCGCCCAGGACTACTCCACCCGCATGAACTGGCTCAGTGCCGGGCCCGCGATGAT
CTCACGGGTGAATGACATCCGCGCCCGCCGAAACCAGATACTCCTAGAACAGTCAGCGATCACCGCCACG
CCCCGCCATCACCTTAATCCGCGTAATTGGCCCGCCGCCCTGGTGTACCAGGAAATTCCCCAGCCCACGA
CCGTACTACTTCCGCGAGACGCCCAGGCCGAAGTCCAGCTGACTAACTCAGGTGTCCAGCTGGCCGGCGG
CGCCGCCCTGTGTCGTCACCGCCCCGCTCAGGGTATAAAGCGGCTGGTGATCCGAGGCAGAGGCACACAG
CTCAACGACGAGGTGGTGAGCTCTTCGCTGGGTCTGCGACCTGACGGAGTCTTCCAACTCGCCGGATCGG
GGAGATCTTCCTTCACGCCTCGTCAGGCCGTCCTGACTTTGGAGAGTTCGTCCTCGCAGCCCCGCTCGGG
TGGCATCGGCACTCTCCAGTTCGTGGAGGAGTTCACTCCCTCGGTCTACTTCAACCCCTTCTCCGGCTCC
CCCGGCCACTACCCGGACGAGTTCATCCCGAACTTCGACGCCATCAGCGAGTCGGTGGACGGCTACGATT
GAATGTCCCATGGTGGCGCGGCTGACCTAGCTCGGCTTCGACACCTGGACCACTGCCGCCGCTTCCGCTG
CTTCGCTCGGGATCTCGCCGAGTTTGCCTACTTTGAGCTGCCCGAGGAGCACCCTCAGGGCCCGGCCCAC
GGAGTGCGGATCATCGTCGAAGGGGGCCTCGACTCCCACCTGCTTCGGATCTTCAGCCAGCGTCCGATCC
TGGTCGAGCGCGAGCAAGGACAGACCCGTCTGACCCTGTACTGCATCTGCAACCACCCCGGCCTGCATGA
AAGTCTTTGTTGTCTGCTGTGTACTGAGTATAATAAAAGCTGAGATCAGCGACTACTCCGGACTTCCGTG
TGTTCCTGAATCCATCAACCAGTCCCTGTTCTTCACCGGGAACGAGACCGAGCTCCAGCTCCAGTGTAAG
CCCCACAAGAAGTACCTCACCTGGCTGTTCCAGGGCTCCCCGATCGCCGTTGTCAACCACTGCGACAACG
ACGGAGTCCTGCTGAGCGGCCCTGCCAACCTTACTTTTTCCACCCGCAGAAGCAAGCTCCAGCTCTTCCA
ACCCTTCCTCCCCGGGACCTATCAGTGCGTCTCGGGACCCTGCCATCACACCTTCCACCTGATCCCGAAT
ACCACAGCGTCGCTCCCCGCTACTAACAACCAAACTACCCACCAACGCCACCGTCGCGACCTTTCCTCTG
AATCTAATACCACTACCGGAGGTGAGCTCCGAGGTCGACCAACCTCTGGGATTTACTACGGCCCCTGGGA
GGTGGTGGGGTTAATAGCGCTAGGCCTAGTTGTGGGTGGGCTTTTGGCTCTCTGCTACCTATACCTCCCT
TGCTGTTCGTACTTAGTGGTGCTGTGTTGCTGGTTTAAGAAATGGGGCAGATCACCCTAGTGAGCTGCGG
TGTGCTGGTGGCGGTGGTGCTTTCGATTGTGGGACTGGGCGGCGCGGCTGTAGTGAAGGAGAAGGCCGAT
CCCTGCTTGCATTTCAATCCCGACAAATGCCAGCTGAGTTTTCAGCCCGATGGCAATCGGTGCGCGGTGC
TGATCAAGTGCGGATGGGAATGCGAGAACGTGAGAATCGAGTACAATAACAAGACTCGGAACAATACTCT
CGCGTCCGTGTGGCAGCCCGGGGACCCCGAGTGGTACACCGTCTCTGTCCCCGGTGCTGACGGCTCCCCG
CGCACCGTGAATAATACTTTCATTTTTGCGCACATGTGCGACACGGTCATGTGGATGAGCAAGCAGTACG
ATATGTGGCCCCCCACGAAGGAGAACATCGTGGTCTTCTCCATCGCTTACAGCCTGTGCACGGTGCTAAT
CACCGCTATCGTGTGCCTGAGCATTCACATGCTCATCGCTATTCGCCCCAGAAATAATGCCGAAAAAGAG
AAACAGCCATAACACGTTTTTTCACACACCTTGTTTTTACAGACAATGCGTCTGTTAAATTTTTTAAACA
TTGTGCTCAGTATTGCTTATGCCTCTGGCTATGCAAACATACAGAAAACCCTCTATGTAGGATCTGATGA
TACACTAGAGGGTACCCAATCACAAGCTAGGGTTTCATGGTATTTTTATAAAAGCTCAGATAATCCTATT
ACTCTTTGCAAAGGTGATCAGGGGCGGACAACAAAGCCGCCTATCACATTTAGCTGTACCAGAACAAATC
TCACGCTTTTCTCAATTACAAAACAATATGCTGGTATTTATTACAGTACAAACTTTCATAGTGGGCAAGA
TAAATATTATACTGTTAAGGTAGAAAATCCTACCACTCCTAGAACTACCACCACCACCACCACCACCACC
ACTACTGCGAAGCCCACTAAACCTAAAACTACCAAGAAAACCACTGTGAAAACTACAACTAGAACCACCA
CAACTACAGAAACCACCACCAGCACAACACTTGCTGCAACTACACACACACACACTGAGCTAACCTTACA
GACCACTAATGATTTGATAGCCCTGTTGCAAAAGGGGGATAACAGCACCACTTCCAATGAGGAGATACCC
AAATCCATGATTGGCATTATTGTTGCTGTAGTGGTGTGCATGTTGATCATCGCCTTGTGCATGGTGTACT
ATGCCTTCTGCTACAGAAAGCACAGACTGAACGACAAGCTGGAACACTTACTAAGTGTTGAATTTTAATT
TTTTAGAACCATGAAGATCCTAGGCCTTTTAGTTTTTTCTATCATTACCTCTGCTCTATGCAATTCTGAC
AATGAGGACGTTACTGTCGTTGTCGGATCAAATTATACACTGAAAGGTCCAGCGAAGGGTATGCTTTCGT
GGTATTGCTGGTTTGGAACTGACACTGATCAAACTGAGCTTTGCAATGCAATGAAAGGTCAAATACCAAC
CTCAAAAATTAAACATAAATGCAATGGTACTGACTTAGTACTACTCAATATCACGAAATCATATGCTGGC
AGCTATTCATGCCCTGGAGATGATGCTGAGAACATGATTTTTTACAAAGTAACTGTTGTTGATCCCACTA
CTCCACCACCCACCACCACAACTACTCACACCACACACACAGAACAAACACCAGAGGCAGCAGAAGCAGA
GTTGGCCTTCCAGGTTCACGGAGATTCCTTTGCTGTCAATACCCCTACACCCGATCATCGGTGTCCGGGG
CTGCTAGTCAGCGGCATTGTCGGTGTGCTTTCGGGATTAGCAGTCATAATCATCTGCATGTTCATTTTTG
CTTGCTGCTATAGAAGGCTTTACCGACAAAAATCAGACCCACTGCTGAACCTCTATGTTTAATTTTTTCC
AGAGCCATGAAGGCAGTTAGCGCTCTAGTTTTTTGTTCTTTGATTGGCATTGTTTTTTGCAATCCTATTA
CTAGAGTTAGCTTTATTAAAGATGTGAATGTTACTGAGGGGGGCAATGTGACACTGGTAGGTGTAGAGGG
TGCTAAAAACACCACCTGGACAAAATACCACCTTGGGTGGAAAGATATTTGCAATTGGAGTGTCACTGTG
TACACATGTGAGGGAGTTAATCTTACCATTGTCAATGCCACCTCAGCTCAAAATGGTAGAATTCAAGGAC
AAAGTGTTAGTGTGACCAGTGATGGGTATTTTACCCAACATACTTTTATCTATGACGTTAAAGTCATACC
ACTGCCTACGCCTAGCCCACCTAGCACCACTACACAAACAACCCACACTACACAGACAACCACATACAGT
ACATCAAATCAGCCTACCACCACTACAGCAGCAGAGGTTGCCAGCTCGTCTGGAGTTCAAGTGGCATTTT
TGTTGTTGCCCCCATCTAGCAGTCCCACTGCTATTACCAATGAGCAGACTACTGCATTTTTGTCCACTGT
CGAGAGCCACACCACAGCTACCTCCAGTGCCTTCTCTAGCACCGCCAATCTCTCCTCGCTTTCCTCTACA
CCAATCAGTCCCGCTACTACTACTACCCCCGCTATTCTTCCCACTCCCCTGAAGCAAACAGACGGCGGCA
TGCAATGGCAGATCACCCTGCTCATTGTGATCGGGTTGGTCATCCTAGCCGTGTTGCTCTACTACATCTT
CTGCCGCCGCATTCCCAACGCGCACCGCAAGCCGGTCTACAAGCCCATCATTGTCGGGCAGCCGGAGCCG
CTTCAGGTGGAAGGGGGTCTAAGGAATCTTCTCTTCTCTTTTACAGTATGGTGATTGAACTATGATTCCT
AGACAATTCTTGATCACTATTCTTATCTGCCTCCTCCAAGTCTGTGCCACCCTCGCTCTGGTGGCCAACG
CCAGTCCAGACTGTATTGGGCCCTTCGCCTCCTACGTGCTCTTTGCCTTCATCACCTGCATCTGCTGCTG
TAGCATAGTCTGCCTGCTTATCACCTTCTTCCAGTTCATTGACTGGATCTTTGTGCGCATCGCCTACCTG
CGCCACCACCCCCAGTACCGCGACCAGCGAGTGGCGCAGCTGCTCAGGCTCCTCTGATAAGCATGCGGGC
TCTGCTACTTCTCGCGCTTCTGCTGTTAGTGCTCCCCCGTCCCGTTGACCCCCGGCCCCCCACTCAGTCC
CCCGAGGAGGTCCGCAAATGCAAATTCCAAGAACCCTGGAAATTCCTCAAATGCTACCGCCAAAAATCAG
ACATGCATCCCAGCTGGATCATGATCATTGGGATCGTGAACATTCTGGCCTGCACCCTCATCTCCTTTGT
GATTTACCCCTGCTTTGACTTTGGTTGGAACTCGCCAGAGGCGCTCTATCTCCCGCCTGAACCTGACACA
CCACCACAGCAACCTCAGGCACACGCACTACCACCACCACAGCCTAGGCCACAATACATGCCCATATTAG
ACTATGAGGCCGAGCCACAGCGACCCATGCTCCCCGCTATTAGTTACTTCAATCTAACCGGCGGAGATGA
CTGACCCACTGGCCAACAACAACGTCAACGACCTTCTCCTGGACATGGACGGCCGCGCCTCGGAGCAGCG
ACTCGCCCAACTTCGCATTCGCCAGCAGCAGGAGAGAGCCGTCAAGGAGCTGCAGGACGGCATAGCCATC
CACCAGTGCAAGAAAGGCATCTTCTGCCTGGTGAAACAGGCCAAGATCTCCTACGAGGTCACCCAGACCG
ACCATCGCCTCTCCTACGAGCTCCTGCAGCAGCGCCAGAAGTTCACCTGCCTGGTCGGAGTCAACCCCAT
CGTCATCACCCAGCAGTCGGGCGATACCAAGGGGTGCATCCACTGCTCCTGCGACTCCCCCGACTGCGTC
CACACTCTGATCAAGACCCTCTGCGGCCTCCGCGACCTCCTCCCCATGAACTAATCACCCACTTATCCAG
TGAAATAAAAAAATAATCATTTGATTTGAAATAAAGATACAATCATATTGATGATTTGAGTTTAACAAAA
ATAAAGAATCACTTACTTGAAATCTGATACCAGGTCTCTGTCCATATTTTCTGCCAACACCACCTCACTC
CCCTCTTCCCAGCTCTGGTACTGCAGGCCCCGGCGGGCTGCAAACTTCCTCCACACGCTGAAGGGGATGT
CAAATTCCTCCTGCCCCTCAATCTTCATTTTATCTTCTATCAGATGTCCAAAAAGCGCGTCCGGGTGGAT
GATGACTTCGACCCCGTCTACCCCTACGATGCAGACAACGCACCGACCGTGCCCTTCATCAACCCCCCCT
TCGTCTCTTCAGATGGATTCCAAGAGAAGCCCCTGGGGGTGTTGTCCCTGCGACTGGCCGACCCCGTCAC
CACCAAGAACGGGGAAATCACCCTCAAGCTGGGAGAGGGGGTGGACCTCGACTCCTCGGGAAAACTCATC
TCCAACACGGCCACCAAGGCCGCTGCCCCTCTCAGTTTTTCCAACAACACCATTTCCCTTAACATGGATC
ACCCCTTTTACACTAAAGATGGAAAATTAGCCTTACAAGTTTCTCCACCATTAAATATACTGAGAACAAG
CATTCTAAACACACTAGCTTTAGGTTTTGGATCAGGTTTAGGACTCCGTGGCTCTGCCTTGGCAGTACAG
TTAGTCTCTCCACTTACATTTGATACTGATGGAAACATAAAGCTTACCTTAGACAGAGGTTTGCATGTTA
CAACAGGAGATGCAATTGAAAGCAACATAAGCTGGGCTAAAGGTTTAAAATTTGAAGATGGAGCCATAGC
AACCAACATTGGAAATGGGTTAGAGTTTGGAAGCAGTAGTACAGAAACAGGTGTCGATGATGCTTACCCA
ATCCAAGTTAAACTTGGATCTGGCCTTAGCTTTGACAGTACAGGAGCCATAATGGCTGGTAACAAAGAAG
ACGATAAACTCACTTTGTGGACAACACCTGATCCATCACCAAACTGTCAAATACTCGCAGAAAATGATGC
AAAACTAACACTTTGCTTGACTAAATGTGGTAGTCAAATACTGGCCACTGTGTCAGTCTTAGTTGTAGGA
AGTGGAAACCTAAACCCCATTACTGGCACCGTAAGCAGTGCTCAGGTGTTTCTACGTTTTGATGCAAACG
GTGTTCTTTTAACAGAACATTCTACACTAAAAAAATACTGGGGGTATAGGCAGGGAGATAGCATAGATGG
CACTCCATATGTCAATGCTGTAGGATTCATGCCCAATTTAAAAGCTTATCCAAAGTCACAAAGTTCTACT
ACTAAAAATAATATAGTAGGGCAAGTATACATGAATGGAGATGTTTCAAAACCTATGCTTCTCACTATAA
CCCTCAATGGTACTGATGACAGCAACAGTACATATTCAATGTCATTTTCATACACCTGGACTAATGGAAG
CTATGTTGGAGCAACATTTGGAGCTAACTCTTATACCTTCTCCTACATCGCCCAAGAATGAATACTGTAT
CCCACCCTGCATGCCCAACCCTCCCCCACCTCTGTCTATATGGAAAACTCTGAAACACAAAATAAAATAA
AGTTCAAGTGTTTTATTGATTCAACAGTTTTACAGGATTCGAGCAGTTATTTTTCCTCCACCCTCCCAGG
ACATGGAATACACCACCCTCTCCCCCCGCACAGCCTTGAACATCTGAATGCCATTGGTGATGGACATGCT
TTTGGTCTCCACGTTCCACACAGTTTCAGAGCGAGCCAGTCTCGGGTCGGTCAGGGAGATGAAACCCTCC
GGGCACTCCCGCATCTGCACCTCACAGCTCAACAGCTGAGGATTGTCCTCGGTGGTCGGGATCACGGTTA
TCTGGAAGAAGCAGAAGAGCGGCGGTGGGAATCATAGTCCGCGAACGGGATCGGCCGGTGGTGTCGCATC
AGGCCCCGCAGCAGTCGCTGCCGCCGCCGCTCCGTCAAGCTGCTGCTCAGGGGGTCCGGGTCCAGGGACT
CCCTCAGCATGATGCCCACGGCCCTCAGCATCAGTCGTCTGGTGCGGCGGGCGCAGCAGCGCATGCGGAT
CTCGCTCAGGTCGCTGCAGTACGTGCAACACAGGACCACCAGGTTGTTCAACAGTCCATAGTTCAACACG
CTCCAGCCGAAACTCATCGCGGGAAGGATGCTACCCACGTGGCCGTCGTACCAGATCCTCAGGTAAATCA
AGTGGCGCCCCCTCCAGAACACGCTGCCCATGTACATGATCTCCTTGGGCATGTGGCGGTTCACCACCTC
CCGGTACCACATCACCCTCTGGTTGAACATGCAGCCCCGGATGATCCTGCGGAACCACAGGGCCAGCACC
GCCCCGCCCGCCATGCAGCGAAGAGACCCCGGGTCCCGGCAATGGCAATGGAGGACCCACCGCTCGTACC
CGTGGATCATCTGGGAGCTGAACAAGTCTATGTTGGCACAGCACAGGCACACGCTCATGCATCTCTTCAG
CACTCTCAGCTCCTCGGGGGTCAAAACCATATCCCAGGGCACGGGAAACTCTTGCAGGACAGCGAAGCCC
GCAGAACAGGGCAATCCTCGCACATAACTTACATTGTGCATGGACAGGGTATCGCAATCAGGCAGCACCG
GGTGATCCTCCACCAGAGAAGCGCGGGTCTCGGTCTCCTCACAGCGTGGTAAGGGGGCCGGCCGATACGG
GTGATGGCGGGACGCGGCTGATCGTGTTCGCGACCGTGTCATGATGCAGTTGCTTTCGGACATTTTCGTA
CTTGCTGAAGCAGAACCTGGTCCGGGCGCTGCACACCGATCGCCGGCGGCGGTCTCGGCGCTTGGAACGC
TCGGTGTTGAAGTTGTAAAACAGCCACTCTCTCAGACCGTGCAGCAGATCTAGGGCCTCAGGAGTGATGA
AGATCCCATCATGCCTGATGGCTCTGATCACATCGACCACCGTGGAATGGGCCAGACCCAGCCAGATGAT
GCAATTTTGTTGGGTTTCGGTGACGGCGGGGGAGGGAAGAACAGGAAGAACCATGATTAACTTTTAATCC
AAACGGTCTCGGAGCACTTCAAAATGAAGGTCGCGGAGATGGCACCTCTCGCCCCCGCTGTGTTGGTGGA
AAATAACAGCCAGGTCAAAGGTGATACGGTTCTCGAGATGTTCCACGGTGGCTTCCAGCAAAGCCTCCAC
GCGCACATCCAGAAACAAGACAATAGCGAAAGCGGGAGGGTTCTCTAATTCCTCAATCATCATGTTACAC
TCCTGCACCATCCCCAGATAATTTTCATTTTTCCAGCCTTGAATGATTCGAACTAGTTCCTGAGGTAAAT
CCAAGCCAGCCATGATAAAGAGCTCGCGCAGAGCGCCCTCCACCGGCATTCTTAAGCACACCCTCATAAT
TCCAAGATATTCTGCTCCTGGTTCACCTGCAGCAGATTGACAAGCGGGATATCAAAATCTCTGCCGCGAT
CCCTGAGCTCCTCCCTCAGCAATAACTGTAAGTACTCTTTCATATCCTCTCCGAAATTTTTAGCCATAGG
ACCCCCAGGAATAAGAGAAGGGCAAGCCACATTACAGATAAACCGAAGTCCCCCCCAGTGAGCATTGCCA
AATGTAAGATTGAAATAAGCATGCTGGCTAGACCCGGTGATATCTTCCAGATAACTGGACAGAAAATCGG
GCAAGCAATTTTTAAGAAAATCAACAAAAGAAAAATCTTCCAGGTGCACGTTTAGGGCCTCGGGAACAAC
GATGGAGTAAGTGCAAGGGGTGCGTTCCAGCATGGTTAGTTAGCTGATCTGTAAAAAAACAAAAAATAAA
ACATTAAACCATGCTAGCCTGGCGAACAGGTGGGTAAATCGTTCTCTCCAGCACCAGGCAGGCCACGGGG
TCTCCGGCGCGACCCTCGTAAAAATTGTCGCTATGATTGAAAACCATCACAGAGAGACGTTCCCGGTGGC
CGGCGTGAATGATTCGAGAAGAAGCATACACCCCCGGAACATTGGAGTCCGTGAGTGAAAAAAAGCGGCC
GAGGAAGCAATGAGGCACTACAACGCTCACTCTCAAGTCCAGCAAAGCGATGCCATGCGGATGAAGCACA
AAATTTTCAGGTGCGTAAAAAATGTAATTACTCCCCTCCTGCACAGGCAGCGAAGCTCCCGATCCCTCCA
GATACACATACAAAGCCTCAGCGTCCATAGCTTACCGAGCGGCAGCAGCAGCGGCACACAACAGGCGCAA
GAGTCAGAGAAAAGACTGAGCTCTAACCTGTCCGCCCGCTCTCTGCTCAATATATAGCCCCAGATCTACA
CTGACGTAAAGGCCAAAGTCTAAAAATACCCGCCAAATAATCACACACGCCCAGCACACGCCCAGAAACC
GGTGACACACTCAAAAAAATACGCGCACTTCCTCAAACGCCCAAACTGCCGTCATTTCCGGGTTCCCACG
CTACGTCATCAAAACACGACTTTCAAATTCCGTCGACCGTTAAAAACGTCACCCGCCCCGCCCCTAACGG
TCGCCGCTCCCGCAGCCAATCAGCGCCCCGCATCCCCAAATTCAAACAGCTCATTTGCATATTAACGCGC
ACCAAAAGTTTGAGGTATATTATTGATGATG
Polynucleotide sequence encoding the CASI promoter
SEQ ID NO: 3
GGAGTTCCGCGTTACATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTCAATGACGGTAAATGGCCCGC
CTGGCATTATGCCCAGTACATGACCTTATGGGACTTTCCTACTTGGCAGTACATCTACGTATTAGTCATCGCTATTA
CCATGGTCGAGGTGAGCCCCACGTTCTGCTTCACTCTCCCCATCTCCCCCCCCTCCCCACCCCCAATTTTGTATTTA
TTTATTTTTTAATTATTTTGTGCAGCGATGGGGGCGGGGGGGGGGGGGGGCGCGCGCCAGGCGGGGCGGGGCGGGGC
GAGGGGCGGGGCGGGGCGAGGCGGAGAGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCTCCCTATCAGTGATAGAGATCTCCCTATCAGTGAT
AGAGATCGTCGACGAGCTCGCGGCGGGCGGGAGTCGCTGCGCGCTGCCTTCGCCCCGTGCCCCGCTCCGCCGCCGCC
TCGCGCCGCCCGCCCCGGCTCTGACTGACCGCGTTACTAAAACAGGTAAGTCCGGCCTCCGCGCCGGGTTTTGGCGC
CTCCCGCGGGCGCCCCCCTCCTCACGGCGAGCGCTGCCACGTCAGACGAAGGGCGCAGCGAGCGTCCTGATCCTTCC
GCCCGGACGCTCAGGACAGCGGCCCGCTGCTCATAAGACTCGGCCTTAGAACCCCAGTATCAGCAGAAGGACATTTT
AGGACGGGACTTGGGTGACTCTAGGGCACTGGTTTTCTTTCCAGAGAGCGGAACAGGCGAGGAAAAGTAGTCCCTTC
TCGGCGATTCTGCGGAGGGATCTCCGTGGGGCGGTGAACGCCGATGATGCCTCTACTAACCATGTTCATGTTTTCTT
TTTTTTTCTACAGGTCCTGGGTGACGAACAG
Polynucleotide sequence encoding ChAd155/RSV
SEQ ID NO: 4
CATCATCAATAATATACCTTATTTTGGATTGAAGCCAATATGATAATGAGATGGGCGGCGCGGGGCGG
GGCGCGGGGCGGGAGGCGGGTTTGGGGGCGGGCCGGCGGGCGGGGCGGTGTGGCGGAAGTGGACTTT
GTAAGTGTGGCGGATGTGACTTGCTAGTGCCGGGCGCGGTAAAAGTGACGTTTTCCGTGCGCGACAAC
GCCCCCGGGAAGTGACATTTTTCCCGCGGTTTTTACCGGATGTTGTAGTGAATTTGGGCGTAACCAAGT
AAGATTTGGCCATTTTCGCGGGAAAACTGAAACGGGGAAGTGAAATCTGATTAATTTTGCGTTAGTCA
TACCGCGTAATATTTGTCTAGGGCCGAGGGACTTTGGCCGATTACGTGGAGGACTCGCCCAGGTGTTTT
TTGAGGTGAATTTCCGCGTTCCGGGTCAAAGTCTGCGTTTTATTATTATAGGATATCCCATTGCATACG
TTGTATCCATATCATAATATGTACATTTATATTGGCTCATGTCCAACATTACCGCCATGTTGACATTGAT
TATTGACTAGTTATTAATAGTAATCAATTACGGGGTCATTAGTTCATAGCCCATATATGGAGTTCCGCG
TTACATAACTTACGGTAAATGGCCCGCCTGGCTGACCGCCCAACGACCCCCGCCCATTGACGTCAATA
ATGACGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGGCAGTACA
TCTACGTATTAGTCATCGCTATTACCATGGTGATGCGGTTTTGGCAGTACATCAATGGGCGTGGATAGC
GGTTTGACTCACGGGGATTTCCAAGTCTCCACCCCATTGACGTCAATGGGAGTTTGTTTTGGCACCAAA
ATCAACGGGACTTTCCAAAATGTCGTAACAACTCCGCCCCATTGACGCAAATGGGCGGTAGGCGTGTA
CGGTGGGAGGTCTATATAAGCAGAGCTCTCCCTATCAGTGATAGAGATCTCCCTATCAGTGATAGAGA
TCGTCGACGAGCTCGTTTAGTGAACCGTCAGATCGCCTGGAGACGCCATCCACGCTGTTTTGACCTCCA
TAGAAGACACCGGGACCGATCCAGCCTCCGCGGCCGGGAACGGTGCATTGGAACGCGGATTCCCCGT
GCCAAGAGTGAGATCTTCCGTTTATCTAGGTACCAGATATCGCCACCATGGAACTGCTGATCCTGAAG
GCCAACGCCATCACCACCATCCTGACCGCCGTGACCTTCTGCTTCGCCAGCGGCCAGAACATCACCGA
GGAATTCTACCAGAGCACCTGTAGCGCCGTGAGCAAGGGCTACCTGAGCGCCCTGAGAACCGGCTGGT
ACACCAGCGTGATCACCATCGAGCTGAGCAACATCAAAGAAAACAAGTGCAACGGCACCGACGCCAA
AGTGAAGCTGATCAAGCAGGAACTGGACAAGTACAAGAACGCCGTGACCGAGCTGCAGCTGCTGATG
CAGAGCACCCCCGCCACCAACAACCGGGCCAGACGGGAGCTGCCCCGGTTCATGAACTACACCCTGA
ACAACGCCAAAAAGACCAACGTGACCCTGAGCAAGAAGCGGAAGCGGCGGTTCCTGGGCTTTCTGCT
GGGCGTGGGCAGCGCCATTGCCAGCGGCGTGGCCGTGTCTAAGGTGCTGCACCTGGAAGGCGAAGTG
AACAAGATCAAGAGCGCCCTGCTGAGCACCAACAAGGCCGTGGTGTCCCTGAGCAACGGCGTGAGCG
TGCTGACCAGCAAGGTGCTGGATCTGAAGAACTACATCGACAAGCAGCTGCTGCCCATCGTGAACAAG
CAGAGCTGCAGCATCAGCAACATCGAGACAGTGATCGAGTTCCAGCAGAAGAACAACCGGCTGCTGG
AAATCACCCGGGAGTTCAGCGTGAACGCCGGCGTGACCACCCCTGTGTCCACCTACATGCTGACCAAC
AGCGAGCTGCTGAGCCTGATCAACGACATGCCCATCACCAACGACCAGAAAAAGCTGATGAGCAACA
ACGTGCAGATCGTGCGGCAGCAGAGCTACTCCATCATGTCCATCATCAAAGAAGAGGTGCTGGCCTAC
GTGGTGCAGCTGCCCCTGTACGGCGTGATCGACACCCCCTGCTGGAAGCTGCACACCAGCCCCCTGTG
CACCACCAACACCAAAGAGGGCAGCAACATCTGCCTGACCCGGACCGACAGAGGCTGGTACTGCGAC
AACGCCGGCAGCGTGTCATTCTTTCCACAGGCCGAGACATGCAAGGTGCAGAGCAACCGGGTGTTCTG
CGACACCATGAACAGCCTGACCCTGCCCTCCGAAGTGAACCTGTGCAACGTGGACATCTTCAACCCCA
AGTACGACTGCAAGATCATGACCTCCAAGACCGACGTGTCCAGCTCCGTGATCACCTCCCTGGGCGCC
ATCGTGTCCTGCTACGGCAAGACCAAGTGCACCGCCAGCAACAAGAACCGGGGCATCATCAAGACCTT
CAGCAACGGCTGCGACTACGTGTCCAACAAGGGGGTGGACACCGTGTCCGTGGGCAACACCCTGTACT
ACGTGAACAAACAGGAAGGCAAGAGCCTGTACGTGAAGGGCGAGCCCATCATCAACTTCTACGACCC
CCTGGTGTTCCCCAGCGACGAGTTCGACGCCAGCATCAGCCAGGTGAACGAGAAGATCAACCAGAGC
CTGGCCTTCATCCGGAAGTCCGACGAGCTGCTGCACAATGTGAATGCCGGCAAGTCCACCACCAACCG
GAAGCGGAGAGCCCCTGTGAAGCAGACCCTGAACTTCGACCTGCTGAAGCTGGCCGGCGACGTGGAG
AGCAATCCCGGCCCTATGGCCCTGAGCAAAGTGAAACTGAACGATACACTGAACAAGGACCAGCTGC
TGTCCAGCAGCAAGTACACCATCCAGCGGAGCACCGGCGACAGCATCGATACCCCCAACTACGACGT
GCAGAAGCACATCAACAAGCTGTGCGGCATGCTGCTGATCACAGAGGACGCCAACCACAAGTTCACC
GGCCTGATCGGCATGCTGTACGCCATGAGCCGGCTGGGCCGGGAGGACACCATCAAGATCCTGCGGG
ACGCCGGCTACCACGTGAAGGCCAATGGCGTGGACGTGACCACACACCGGCAGGACATCAACGGCAA
AGAAATGAAGTTCGAGGTGCTGACCCTGGCCAGCCTGACCACCGAGATCCAGATCAATATCGAGATCG
AGAGCCGGAAGTCCTACAAGAAAATGCTGAAAGAAATGGGCGAGGTGGCCCCCGAGTACAGACACGA
CAGCCCCGACTGCGGCATGATCATCCTGTGTATCGCCGCCCTGGTGATCACAAAGCTGGCCGCTGGCG
ACAGATCTGGCCTGACAGCCGTGATCAGACGGGCCAACAATGTGCTGAAGAACGAGATGAAGCGGTA
CAAGGGCCTGCTGCCCAAGGACATTGCCAACAGCTTCTACGAGGTGTTCGAGAAGTACCCCCACTTCA
TCGACGTGTTCGTGCACTTCGGCATTGCCCAGAGCAGCACCAGAGGCGGCTCCAGAGTGGAGGGCATC
TTCGCCGGCCTGTTCATGAACGCCTACGGCGCTGGCCAGGTGATGCTGAGATGGGGCGTGCTGGCCAA
GAGCGTGAAGAACATCATGCTGGGCCACGCCAGCGTGCAGGCCGAGATGGAACAGGTGGTGGAGGTG
TACGAGTACGCCCAGAAGCTGGGCGGAGAGGCCGGCTTCTACCACATCCTGAACAACCCTAAGGCCTC
CCTGCTGTCCCTGACCCAGTTCCCCCACTTCTCCAGCGTGGTGCTGGGAAATGCCGCCGGACTGGGCAT
CATGGGCGAGTACCGGGGCACCCCCAGAAACCAGGACCTGTACGACGCCGCCAAGGCCTACGCCGAG
CAGCTGAAAGAAAACGGCGTGATCAACTACAGCGTGCTGGACCTGACCGCTGAGGAACTGGAAGCCA
TCAAGCACCAGCTGAACCCCAAGGACAACGACGTGGAGCTGGGAGGCGGAGGATCTGGCGGCGGAGG
CATGAGCAGACGGAACCCCTGCAAGTTCGAGATCCGGGGCCACTGCCTGAACGGCAAGCGGTGCCAC
TTCAGCCACAACTACTTCGAGTGGCCCCCTCATGCTCTGCTGGTGCGGCAGAACTTCATGCTGAACCGG
ATCCTGAAGTCCATGGACAAGAGCATCGACACCCTGAGCGAGATCAGCGGAGCCGCCGAGCTGGACA
GAACCGAGGAATATGCCCTGGGCGTGGTGGGAGTGCTGGAAAGCTACATCGGCTCCATCAACAACAT
CACAAAGCAGAGCGCCTGCGTGGCCATGAGCAAGCTGCTGACAGAGCTGAACAGCGACGACATCAAG
AAGCTGAGGGACAACGAGGAACTGAACAGCCCCAAGATCCGGGTGTACAACACCGTGATCAGCTACA
TTGAGAGCAACCGCAAGAACAACAAGCAGACCATCCATCTGCTGAAGCGGCTGCCCGCCGACGTGCT
GAAAAAGACCATCAAGAACACCCTGGACATCCACAAGTCCATCACCATCAACAATCCCAAAGAAAGC
ACCGTGTCTGACACCAACGATCACGCCAAGAACAACGACACCACCTGATGAGCGGCCGCGATCTGCTG
TGCCTTCTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCTATTCT
GGGGGGTGGGGTGGGGCAGGACAGCAAGGGGGAGGATTGGGAAGACAATAGCAGGCATGCTGGGGA
TGCGGTGGGCTCTATGGCCGATCAGCGATCGCTGAGGTGGGTGAGTGGGCGTGGCCTGGGGTGGTCAT
GAAAATATATAAGTTGGGGGTCTTAGGGTCTCTTTATTTGTGTTGCAGAGACCGCCGGAGCCATGAGC
GGGAGCAGCAGCAGCAGCAGTAGCAGCAGCGCCTTGGATGGCAGCATCGTGAGCCCTTATTTGACGA
CGCGGATGCCCCACTGGGCCGGGGTGCGTCAGAATGTGATGGGCTCCAGCATCGACGGCCGACCCGTC
CTGCCCGCAAATTCCGCCACGCTGACCTATGCGACCGTCGCGGGGACGCCGTTGGACGCCACCGCCGC
CGCCGCCGCCACCGCAGCCGCCTCGGCCGTGCGCAGCCTGGCCACGGACTTTGCATTCCTGGGACCAC
TGGCGACAGGGGCTACTTCTCGGGCCGCTGCTGCCGCCGTTCGCGATGACAAGCTGACCGCCCTGCTG
GCGCAGTTGGATGCGCTTACTCGGGAACTGGGTGACCTTTCTCAGCAGGTCATGGCCCTGCGCCAGCA
GGTCTCCTCCCTGCAAGCTGGCGGGAATGCTTCTCCCACAAATGCCGTTTAAGATAAATAAAACCAGA
CTCTGTTTGGATTAAAGAAAAGTAGCAAGTGCATTGCTCTCTTTATTTCATAATTTTCCGCGCGCGATA
GGCCCTAGACCAGCGTTCTCGGTCGTTGAGGGTGCGGTGTATCTTCTCCAGGACGTGGTAGAGGTGGC
TCTGGACGTTGAGATACATGGGCATGAGCCCGTCCCGGGGGTGGAGGTAGCACCACTGCAGAGCTTCA
TGCTCCGGGGTGGTGTTGTAGATGATCCAGTCGTAGCAGGAGCGCTGGGCATGGTGCCTAAAAATGTC
CTTCAGCAGCAGGCCGATGGCCAGGGGGAGGCCCTTGGTGTAAGTGTTTACAAAACGGTTAAGTTGGG
AAGGGTGCATTCGGGGAGAGATGATGTGCATCTTGGACTGTATTTTTAGATTGGCGATGTTTCCGCCCA
GATCCCTTCTGGGATTCATGTTGTGCAGGACCACCAGTACAGTGTATCCGGTGCACTTGGGGAATTTGT
CATGCAGCTTAGAGGGAAAAGCGTGGAAGAACTTGGAGACGCCTTTGTGGCCTCCCAGATTTTCCATG
CATTCGTCCATGATGATGGCAATGGGCCCGCGGGAGGCAGCTTGGGCAAAGATATTTCTGGGGTCGCT
GACGTCGTAGTTGTGTTCCAGGGTGAGGTCGTCATAGGCCATTTTTACAAAGCGCGGGCGGAGGGTGC
CCGACTGGGGGATGATGGTCCCCTCTGGCCCTGGGGCGTAGTTGCCCTCGCAGATCTGCATTTCCCAG
GCCTTAATCTCGGAGGGGGGAATCATATCCACCTGCGGGGCGATGAAGAAAACGGTTTCCGGAGCCG
GGGAGATTAACTGGGATGAGAGCAGGTTTCTAAGCAGCTGTGATTTTCCACAACCGGTGGGCCCATAA
ATAACACCTATAACCGGTTGCAGCTGGTAGTTTAGAGAGCTGCAGCTGCCGTCGTCCCGGAGGAGGGG
GGCCACCTCGTTGAGCATGTCCCTGACGCGCATGTTCTCCCCGACCAGATCCGCCAGAAGGCGCTCGC
CGCCCAGGGACAGCAGCTCTTGCAAGGAAGCAAAGTTTTTCAGCGGCTTGAGGCCGTCCGCCGTGGGC
ATGTTTTTCAGGGTCTGGCTCAGCAGCTCCAGGCGGTCCCAGAGCTCGGTGACGTGCTCTACGGCATCT
CTATCCAGCATATCTCCTCGTTTCGCGGGTTGGGGCGACTTTCGCTGTAGGGCACCAAGCGGTGGTCGT
CCAGCGGGGCCAGAGTCATGTCCTTCCATGGGCGCAGGGTCCTCGTCAGGGTGGTCTGGGTCACGGTG
AAGGGGTGCGCTCCGGGCTGAGCGCTTGCCAAGGTGCGCTTGAGGCTGGTTCTGCTGGTGCTGAAGCG
CTGCCGGTCTTCGCCCTGCGCGTCGGCCAGGTAGCATTTGACCATGGTGTCATAGTCCAGCCCCTCCGC
GGCGTGTCCCTTGGCGCGCAGCTTGCCCTTGGAGGTGGCGCCGCACGAGGGGCAGAGCAGGCTCTTGA
GCGCGTAGAGCTTGGGGGCGAGGAAGACCGATTCGGGGGAGTAGGCGTCCGCGCCGCAGACCCCGCA
CACGGTCTCGCACTCCACCAGCCAGGTGAGCTCGGGGCGCGCCGGGTCAAAAACCAGGTTTCCCCCAT
GCTTTTTGATGCGTTTCTTACCTCGGGTCTCCATGAGGTGGTGTCCCCGCTCGGTGACGAAGAGGCTGT
CCGTGTCTCCGTAGACCGACTTGAGGGGTCTTTTCTCCAGGGGGGTCCCTCGGTCTTCCTCGTAGAGGA
ACTCGGACCACTCTGAGACGAAGGCCCGCGTCCAGGCCAGGACGAAGGAGGCTATGTGGGAGGGGTA
GCGGTCGTTGTCCACTAGGGGGTCCACCTTCTCCAAGGTGTGAAGACACATGTCGCCTTCCTCGGCGTC
CAGGAAGGTGATTGGCTTGTAGGTGTAGGCCACGTGACCGGGGGTTCCTGACGGGGGGGTATAAAAG
GGGGTGGGGGCGCGCTCGTCGTCACTCTCTTCCGCATCGCTGTCTGCGAGGGCCAGCTGCTGGGGTGA
GTATTCCCTCTCGAAGGCGGGCATGACCTCCGCGCTGAGGTTGTCAGTTTCCAAAAACGAGGAGGATT
TGATGTTCACCTGTCCCGAGGTGATACCTTTGAGGGTACCCGCGTCCATCTGGTCAGAAAACACGATCT
TTTTATTGTCCAGCTTGGTGGCGAACGACCCGTAGAGGGCGTTGGAGAGCAGCTTGGCGATGGAGCGC
AGGGTCTGGTTCTTGTCCCTGTCGGCGCGCTCCTTGGCCGCGATGTTGAGCTGCACGTACTCGCGCGCG
ACGCAGCGCCACTCGGGGAAGACGGTGGTGCGCTCGTCGGGCACCAGGCGCACGCGCCAGCCGCGGT
TGTGCAGGGTGACCAGGTCCACGCTGGTGGCGACCTCGCCGCGCAGGCGCTCGTTGGTCCAGCAGAGA
CGGCCGCCCTTGCGCGAGCAGAAGGGGGGCAGGGGGTCGAGCTGGGTCTCGTCCGGGGGGTCCGCGT
CCACGGTGAAAACCCCGGGGCGCAGGCGCGCGTCGAAGTAGTCTATCTTGCAACCTTGCATGTCCAGC
GCCTGCTGCCAGTCGCGGGCGGCGAGCGCGCGCTCGTAGGGGTTGAGCGGCGGGCCCCAGGGCATGG
GGTGGGTGAGTGCGGAGGCGTACATGCCGCAGATGTCATAGACGTAGAGGGGCTCCCGCAGGACCCC
GATGTAGGTGGGGTAGCAGCGGCCGCCGCGGATGCTGGCGCGCACGTAGTCATACAGCTCGTGCGAG
GGGGCGAGGAGGTCGGGGCCCAGGTTGGTGCGGGCGGGGCGCTCCGCGCGGAAGACGATCTGCCTGA
AGATGGCATGCGAGTTGGAAGAGATGGTGGGGCGCTGGAAGACGTTGAAGCTGGCGTCCTGCAGGCC
GACGGCGTCGCGCACGAAGGAGGCGTAGGAGTCGCGCAGCTTGTGTACCAGCTCGGCGGTGACCTGC
ACGTCGAGCGCGCAGTAGTCGAGGGTCTCGCGGATGATGTCATATTTAGCCTGCCCCTTCTTTTTCCAC
AGCTCGCGGTTGAGGACAAACTCTTCGCGGTCTTTCCAGTACTCTTGGATCGGGAAACCGTCCGGTTCC
GAACGGTAAGAGCCTAGCATGTAGAACTGGTTGACGGCCTGGTAGGCGCAGCAGCCCTTCTCCACGGG
GAGGGCGTAGGCCTGCGCGGCCTTGCGGAGCGAGGTGTGGGTCAGGGCGAAGGTGTCCCTGACCATG
ACTTTGAGGTACTGGTGCTTGAAGTCGGAGTCGTCGCAGCCGCCCCGCTCCCAGAGCGAGAAGTCGGT
GCGCTTCTTGGAGCGGGGGTTGGGCAGAGCGAAGGTGACATCGTTGAAGAGGATTTTGCCCGCGCGG
GGCATGAAGTTGCGGGTGATGCGGAAGGGCCCCGGCACTTCAGAGCGGTTGTTGATGACCTGGGCGG
CGAGCACGATCTCGTCGAAGCCGTTGATGTTGTGGCCCACGATGTAGAGTTCCAGGAAGCGGGGCCGG
CCCTTTACGGTGGGCAGCTTCTTTAGCTCTTCGTAGGTGAGCTCCTCGGGCGAGGCGAGGCCGTGCTCG
GCCAGGGCCCAGTCCGCGAGGTGCGGGTTGTCTCTGAGGAAGGACTTCCAGAGGTCGCGGGCCAGGA
GGGTCTGCAGGCGGTCTCTGAAGGTCCTGAACTGGCGGCCCACGGCCATTTTTTCGGGGGTGATGCAG
TAGAAGGTGAGGGGGTCTTGCTGCCAGCGGTCCCAGTCGAGCTGCAGGGCGAGGTCGCGCGCGGCGG
TGACCAGGCGCTCGTCGCCCCCGAATTTCATGACCAGCATGAAGGGCACGAGCTGCTTTCCGAAGGCC
CCCATCCAAGTGTAGGTCTCTACATCGTAGGTGACAAAGAGGCGCTCCGTGCGAGGATGCGAGCCGAT
CGGGAAGAACTGGATCTCCCGCCACCAGTTGGAGGAGTGGCTGTTGATGTGGTGGAAGTAGAAGTCCC
GTCGCCGGGCCGAACACTCGTGCTGGCTTTTGTAAAAGCGAGCGCAGTACTGGCAGCGCTGCACGGGC
TGTACCTCATGCACGAGATGCACCTTTCGCCCGCGCACGAGGAAGCCGAGGGGAAATCTGAGCCCCCC
GCCTGGCTCGCGGCATGGCTGGTTCTCTTCTACTTTGGATGCGTGTCCGTCTCCGTCTGGCTCCTCGAG
GGGTGTTACGGTGGAGCGGACCACCACGCCGCGCGAGCCGCAGGTCCAGATATCGGCGCGCGGCGGT
CGGAGTTTGATGACGACATCGCGCAGCTGGGAGCTGTCCATGGTCTGGAGCTCCCGCGGCGGCGGCAG
GTCAGCCGGGAGTTCTTGCAGGTTCACCTCGCAGAGTCGGGCCAGGGCGCGGGGCAGGTCTAGGTGGT
ACCTGATCTCTAGGGGCGTGTTGGTGGCGGCGTCGATGGCTTGCAGGAGCCCGCAGCCCCGGGGGGCG
ACGACGGTGCCCCGCGGGGTGGTGGTGGTGGTGGCGGTGCAGCTCAGAAGCGGTGCCGCGGGCGGGC
CCCCGGAGGTAGGGGGGGCTCCGGTCCCGCGGGCAGGGGCGGCAGCGGCACGTCGGCGTGGAGCGCG
GGCAGGAGTTGGTGCTGTGCCCGGAGGTTGCTGGCGAAGGCGACGACGCGGCGGTTGATCTCCTGGAT
CTGGCGCCTCTGCGTGAAGACGACGGGCCCGGTGAGCTTGAACCTGAAAGAGAGTTCGACAGAATCA
ATCTCGGTGTCATTGACCGCGGCCTGGCGCAGGATCTCCTGCACGTCTCCCGAGTTGTCTTGGTAGGCG
ATCTCGGCCATGAACTGCTCGATCTCTTCCTCCTGGAGGTCTCCGCGTCCGGCGCGTTCCACGGTGGCC
GCCAGGTCGTTGGAGATGCGCCCCATGAGCTGCGAGAAGGCGTTGAGTCCGCCCTCGTTCCAGACTCG
GCTGTAGACCACGCCCCCCTGGTCATCGCGGGCGCGCATGACCACCTGCGCGAGGTTGAGCTCCACGT
GCCGCGCGAAGACGGCGTAGTTGCGCAGACGCTGGAAGAGGTAGTTGAGGGTGGTGGCGGTGTGCTC
GGCCACGAAGAAGTTCATGACCCAGCGGCGCAACGTGGATTCGTTGATGTCCCCCAAGGCCTCCAGCC
GTTCCATGGCCTCGTAGAAGTCCACGGCGAAGTTGAAAAACTGGGAGTTGCGCGCCGACACGGTCAAC
TCCTCCTCCAGAAGACGGATGAGCTCGGCGACGGTGTCGCGCACCTCGCGCTCGAAGGCTATGGGGAT
CTCTTCCTCCGCTAGCATCACCACCTCCTCCTCTTCCTCCTCTTCTGGCACTTCCATGATGGCTTCCTCCT
CTTCGGGGGGTGGCGGCGGCGGCGGTGGGGGAGGGGGCGCTCTGCGCCGGCGGCGGCGCACCGGGAG
GCGGTCCACGAAGCGCGCGATCATCTCCCCGCGGCGGCGGCGCATGGTCTCGGTGACGGCGCGGCCGT
TCTCCCGGGGGCGCAGTTGGAAGACGCCGCCGGACATCTGGTGCTGGGGCGGGTGGCCGTGAGGCAG
CGAGACGGCGCTGACGATGCATCTCAACAATTGCTGCGTAGGTACGCCGCCGAGGGACCTGAGGGAG
TCCATATCCACCGGATCCGAAAACCTTTCGAGGAAGGCGTCTAACCAGTCGCAGTCGCAAGGTAGGCT
GAGCACCGTGGCGGGCGGCGGGGGGTGGGGGGAGTGTCTGGCGGAGGTGCTGCTGATGATGTAATTG
AAGTAGGCGGACTTGACACGGCGGATGGTCGACAGGAGCACCATGTCCTTGGGTCCGGCCTGCTGGAT
GCGGAGGCGGTCGGCTATGCCCCAGGCTTCGTTCTGGCATCGGCGCAGGTCCTTGTAGTAGTCTTGCAT
GAGCCTTTCCACCGGCACCTCTTCTCCTTCCTCTTCTGCTTCTTCCATGTCTGCTTCGGCCCTGGGGCGG
CGCCGCGCCCCCCTGCCCCCCATGCGCGTGACCCCGAACCCCCTGAGCGGTTGGAGCAGGGCCAGGTC
GGCGACGACGCGCTCGGCCAGGATGGCCTGCTGCACCTGCGTGAGGGTGGTTTGGAAGTCATCCAAGT
CCACGAAGCGGTGGTAGGCGCCCGTGTTGATGGTGTAGGTGCAGTTGGCCATGACGGACCAGTTGACG
GTCTGGTGGCCCGGTTGCGACATCTCGGTGTACCTGAGTCGCGAGTAGGCGCGGGAGTCGAAGACGTA
GTCGTTGCAAGTCCGCACCAGGTACTGGTAGCCCACCAGGAAGTGCGGCGGCGGCTGGCGGTAGAGG
GGCCAGCGCAGGGTGGCGGGGGCTCCGGGGGCCAGGTCTTCCAGCATGAGGCGGTGGTAGGCGTAGA
TGTACCTGGACATCCAGGTGATACCCGCGGCGGTGGTGGAGGCGCGCGGGAAGTCGCGCACCCGGTTC
CAGATGTTGCGCAGGGGCAGAAAGTGCTCCATGGTAGGCGTGCTCTGTCCAGTCAGACGCGCGCAGTC
GTTGATACTCTAGACCAGGGAAAACGAAAGCCGGTCAGCGGGCACTCTTCCGTGGTCTGGTGAATAGA
TCGCAAGGGTATCATGGCGGAGGGCCTCGGTTCGAGCCCCGGGTCCGGGCCGGACGGTCCGCCATGAT
CCACGCGGTTACCGCCCGCGTGTCGAACCCAGGTGTGCGACGTCAGACAACGGTGGAGTGTTCCTTTT
GGCGTTTTTCTGGCCGGGCGCCGGCGCCGCGTAAGAGACTAAGCCGCGAAAGCGAAAGCAGTAAGTG
GCTCGCTCCCCGTAGCCGGAGGGATCCTTGCTAAGGGTTGCGTTGCGGCGAACCCCGGTTCGAATCCC
GTACTCGGGCCGGCCGGACCCGCGGCTAAGGTGTTGGATTGGCCTCCCCCTCGTATAAAGACCCCGCT
TGCGGATTGACTCCGGACACGGGGACGAGCCCCTTTTATTTTTGCTTTCCCCAGATGCATCCGGTGCTG
CGGCAGATGCGCCCCCCGCCCCAGCAGCAGCAACAACACCAGCAAGAGCGGCAGCAACAGCAGCGGG
AGTCATGCAGGGCCCCCTCACCCACCCTCGGCGGGCCGGCCACCTCGGCGTCCGCGGCCGTGTCTGGC
GCCTGCGGCGGCGGCGGGGGGCCGGCTGACGACCCCGAGGAGCCCCCGCGGCGCAGGGCCAGACACT
ACCTGGACCTGGAGGAGGGCGAGGGCCTGGCGCGGCTGGGGGCGCCGTCTCCCGAGCGCCACCCGCG
GGTGCAGCTGAAGCGCGACTCGCGCGAGGCGTACGTGCCTCGGCAGAACCTGTTCAGGGACCGCGCG
GGCGAGGAGCCCGAGGAGATGCGGGACAGGAGGTTCAGCGCAGGGCGGGAGCTGCGGCAGGGGCTG
AACCGCGAGCGGCTGCTGCGCGAGGAGGACTTTGAGCCCGACGCGCGGACGGGGATCAGCCCCGCGC
GCGCGCACGTGGCGGCCGCCGACCTGGTGACGGCGTACGAGCAGACGGTGAACCAGGAGATCAACTT
CCAAAAGAGTTTCAACAACCACGTGCGCACGCTGGTGGCGCGCGAGGAGGTGACCATCGGGCTGATG
CACCTGTGGGACTTTGTAAGCGCGCTGGTGCAGAACCCCAACAGCAAGCCTCTGACGGCGCAGCTGTT
CCTGATAGTGCAGCACAGCAGGGACAACGAGGCGTTTAGGGACGCGCTGCTGAACATCACCGAGCCC
GAGGGTCGGTGGCTGCTGGACCTGATTAACATCCTGCAGAGCATAGTGGTGCAGGAGCGCAGCCTGA
GCCTGGCCGACAAGGTGGCGGCCATCAACTACTCGATGCTGAGCCTGGGCAAGTTTTACGCGCGCAAG
ATCTACCAGACGCCGTACGTGCCCATAGACAAGGAGGTGAAGATCGACGGTTTTTACATGCGCATGGC
GCTGAAGGTGCTCACCCTGAGCGACGACCTGGGCGTGTACCGCAACGAGCGCATCCACAAGGCCGTG
AGCGTGAGCCGGCGGCGCGAGCTGAGCGACCGCGAGCTGATGCACAGCCTGCAGCGGGCGCTGGCGG
GCGCCGGCAGCGGCGACAGGGAGGCGGAGTCCTACTTCGATGCGGGGGCGGACCTGCGCTGGGCGCC
CAGCCGGCGGGCCCTGGAGGCCGCGGGGGTCCGCGAGGACTATGACGAGGACGGCGAGGAGGATGA
GGAGTACGAGCTAGAGGAGGGCGAGTACCTGGACTAAACCGCGGGTGGTGTTTCCGGTAGATGCAAG
ACCCGAACGTGGTGGACCCGGCGCTGCGGGCGGCTCTGCAGAGCCAGCCGTCCGGCCTTAACTCCTCA
GACGACTGGCGACAGGTCATGGACCGCATCATGTCGCTGACGGCGCGTAACCCGGACGCGTTCCGGCA
GCAGCCGCAGGCCAACAGGCTCTCCGCCATCCTGGAGGCGGTGGTGCCTGCGCGCTCGAACCCCACGC
ACGAGAAGGTGCTGGCCATAGTGAACGCGCTGGCCGAGAACAGGGCCATCCGCCCGGACGAGGCCGG
GCTGGTGTACGACGCGCTGCTGCAGCGCGTGGCCCGCTACAACAGCGGCAACGTGCAGACCAACCTG
GACCGGCTGGTGGGGGACGTGCGCGAGGCGGTGGCGCAGCGCGAGCGCGCGGATCGGCAGGGCAACC
TGGGCTCCATGGTGGCGCTGAATGCCTTCCTGAGCACGCAGCCGGCCAACGTGCCGCGGGGGCAGGA
AGACTACACCAACTTTGTGAGCGCGCTGCGGCTGATGGTGACCGAGACCCCCCAGAGCGAGGTGTACC
AGTCGGGCCCGGACTACTTCTTCCAGACCAGCAGACAGGGCCTGCAGACGGTGAACCTGAGCCAGGCT
TTCAAGAACCTGCGGGGGCTGTGGGGCGTGAAGGCGCCCACCGGCGACCGGGCGACGGTGTCCAGCC
TGCTGACGCCCAACTCGCGCCTGCTGCTGCTGCTGATCGCGCCGTTCACGGACAGCGGCAGCGTGTCC
CGGGACACCTACCTGGGGCACCTGCTGACCCTGTACCGCGAGGCCATCGGGCAGGCGCAGGTGGACG
AGCACACCTTCCAGGAGATCACCAGCGTGAGCCGCGCGCTGGGGCAGGAGGACACGAGCAGCCTGGA
GGCGACTCTGAACTACCTGCTGACCAACCGGCGGCAGAAGATTCCCTCGCTGCACAGCCTGACCTCCG
AGGAGGAGCGCATCTTGCGCTACGTGCAGCAGAGCGTGAGCCTGAACCTGATGCGCGACGGGGTGAC
GCCCAGCGTGGCGCTGGACATGACCGCGCGCAACATGGAACCGGGCATGTACGCCGCGCACCGGCCT
TACATCAACCGCCTGATGGACTACCTGCATCGCGCGGCGGCCGTGAACCCCGAGTACTTTACCAACGC
CATCCTGAACCCGCACTGGCTCCCGCCGCCCGGGTTCTACAGCGGGGGCTTCGAGGTCCCGGAGACCA
ACGATGGCTTCCTGTGGGACGACATGGACGACAGCGTGTTCTCCCCGCGGCCGCAGGCGCTGGCGGAA
GCGTCCCTGCTGCGTCCCAAGAAGGAGGAGGAGGAGGAGGCGAGTCGCCGCCGCGGCAGCAGCGGCG
TGGCTTCTCTGTCCGAGCTGGGGGCGGCAGCCGCCGCGCGCCCCGGGTCCCTGGGCGGCAGCCCCTTT
CCGAGCCTGGTGGGGTCTCTGCACAGCGAGCGCACCACCCGCCCTCGGCTGCTGGGCGAGGACGAGTA
CCTGAATAACTCCCTGCTGCAGCCGGTGCGGGAGAAAAACCTGCCTCCCGCCTTCCCCAACAACGGGA
TAGAGAGCCTGGTGGACAAGATGAGCAGATGGAAGACCTATGCGCAGGAGCACAGGGACGCGCCTGC
GCTCCGGCCGCCCACGCGGCGCCAGCGCCACGACCGGCAGCGGGGGCTGGTGTGGGATGACGAGGAC
TCCGCGGACGATAGCAGCGTGCTGGACCTGGGAGGGAGCGGCAACCCGTTCGCGCACCTGCGCCCCC
GCCTGGGGAGGATGTTTTAAAAAAAAAAAAAAAAAGCAAGAAGCATGATGCAAAAATTAAATAAAA
CTCACCAAGGCCATGGCGACCGAGCGTTGGTTTCTTGTGTTCCCTTCAGTATGCGGCGCGCGGCGATGT
ACCAGGAGGGACCTCCTCCCTCTTACGAGAGCGTGGTGGGCGCGGCGGCGGCGGCGCCCTCTTCTCCC
TTTGCGTCGCAGCTGCTGGAGCCGCCGTACGTGCCTCCGCGCTACCTGCGGCCTACGGGGGGGAGAAA
CAGCATCCGTTACTCGGAGCTGGCGCCCCTGTTCGACACCACCCGGGTGTACCTGGTGGACAACAAGT
CGGCGGACGTGGCCTCCCTGAACTACCAGAACGACCACAGCAATTTTTTGACCACGGTCATCCAGAAC
AATGACTACAGCCCGAGCGAGGCCAGCACCCAGACCATCAATCTGGATGACCGGTCGCACTGGGGCG
GCGACCTGAAAACCATCCTGCACACCAACATGCCCAACGTGAACGAGTTCATGTTCACCAATAAGTTC
AAGGCGCGGGTGATGGTGTCGCGCTCGCACACCAAGGAAGACCGGGTGGAGCTGAAGTACGAGTGGG
TGGAGTTCGAGCTGCCAGAGGGCAACTACTCCGAGACCATGACCATTGACCTGATGAACAACGCGATC
GTGGAGCACTATCTGAAAGTGGGCAGGCAGAACGGGGTCCTGGAGAGCGACATCGGGGTCAAGTTCG
ACACCAGGAACTTCCGCCTGGGGCTGGACCCCGTGACCGGGCTGGTTATGCCCGGGGTGTACACCAAC
GAGGCCTTCCATCCCGACATCATCCTGCTGCCCGGCTGCGGGGTGGACTTCACTTACAGCCGCCTGAG
CAACCTCCTGGGCATCCGCAAGCGGCAGCCCTTCCAGGAGGGCTTCAGGATCACCTACGAGGACCTGG
AGGGGGGCAACATCCCCGCGCTCCTCGATGTGGAGGCCTACCAGGATAGCTTGAAGGAAAATGAGGC
GGGACAGGAGGATACCGCCCCCGCCGCCTCCGCCGCCGCCGAGCAGGGCGAGGATGCTGCTGACACC
GCGGCCGCGGACGGGGCAGAGGCCGACCCCGCTATGGTGGTGGAGGCTCCCGAGCAGGAGGAGGACA
TGAATGACAGTGCGGTGCGCGGAGACACCTTCGTCACCCGGGGGGAGGAAAAGCAAGCGGAGGCCGA
GGCCGCGGCCGAGGAAAAGCAACTGGCGGCAGCAGCGGCGGCGGCGGCGTTGGCCGCGGCGGAGGC
TGAGTCTGAGGGGACCAAGCCCGCCAAGGAGCCCGTGATTAAGCCCCTGACCGAAGATAGCAAGAAG
CGCAGTTACAACCTGCTCAAGGACAGCACCAACACCGCGTACCGCAGCTGGTACCTGGCCTACAACTA
CGGCGACCCGTCGACGGGGGTGCGCTCCTGGACCCTGCTGTGCACGCCGGACGTGACCTGCGGCTCGG
AGCAGGTGTACTGGTCGCTGCCCGACATGATGCAAGACCCCGTGACCTTCCGCTCCACGCGGCAGGTC
AGCAACTTCCCGGTGGTGGGCGCCGAGCTGCTGCCCGTGCACTCCAAGAGCTTCTACAACGACCAGGC
CGTCTACTCCCAGCTCATCCGCCAGTTCACCTCTCTGACCCACGTGTTCAATCGCTTTCCTGAGAACCA
GATTCTGGCGCGCCCGCCCGCCCCCACCATCACCACCGTCAGTGAAAACGTTCCTGCTCTCACAGATC
ACGGGACGCTACCGCTGCGCAACAGCATCGGAGGAGTCCAGCGAGTGACCGTTACTGACGCCAGACG
CCGCACCTGCCCCTACGTTTACAAGGCCTTGGGCATAGTCTCGCCGCGCGTCCTTTCCAGCCGCACTTT
TTGAGCAACACCACCATCATGTCCATCCTGATCTCACCCAGCAATAACTCCGGCTGGGGACTGCTGCG
CGCGCCCAGCAAGATGTTCGGAGGGGCGAGGAAGCGTTCCGAGCAGCACCCCGTGCGCGTGCGCGGG
CACTTCCGCGCCCCCTGGGGAGCGCACAAACGCGGCCGCGCGGGGCGCACCACCGTGGACGACGCCA
TCGACTCGGTGGTGGAGCAGGCGCGCAACTACAGGCCCGCGGTCTCTACCGTGGACGCGGCCATCCAG
ACCGTGGTGCGGGGCGCGCGGCGGTACGCCAAGCTGAAGAGCCGCCGGAAGCGCGTGGCCCGCCGCC
ACCGCCGCCGACCCGGGGCCGCCGCCAAACGCGCCGCCGCGGCCCTGCTTCGCCGGGCCAAGCGCAC
GGGCCGCCGCGCCGCCATGAGGGCCGCGCGCCGCTTGGCCGCCGGCATCACCGCCGCCACCATGGCCC
CCCGTACCCGAAGACGCGCGGCCGCCGCCGCCGCCGCCGCCATCAGTGACATGGCCAGCAGGCGCCG
GGGCAACGTGTACTGGGTGCGCGACTCGGTGACCGGCACGCGCGTGCCCGTGCGCTTCCGCCCCCCGC
GGACTTGAGATGATGTGAAAAAACAACACTGAGTCTCCTGCTGTTGTGTGTATCCCAGCGGCGGCGGC
GCGCGCAGCGTCATGTCCAAGCGCAAAATCAAAGAAGAGATGCTCCAGGTCGTCGCGCCGGAGATCT
ATGGGCCCCCGAAGAAGGAAGAGCAGGATTCGAAGCCCCGCAAGATAAAGCGGGTCAAAAAGAAAA
AGAAAGATGATGACGATGCCGATGGGGAGGTGGAGTTCCTGCGCGCCACGGCGCCCAGGCGCCCGGT
GCAGTGGAAGGGCCGGCGCGTAAAGCGCGTCCTGCGCCCCGGCACCGCGGTGGTCTTCACGCCCGGC
GAGCGCTCCACCCGGACTTTCAAGCGCGTCTATGACGAGGTGTACGGCGACGAAGACCTGCTGGAGCA
GGCCAACGAGCGCTTCGGAGAGTTTGCTTACGGGAAGCGTCAGCGGGCGCTGGGGAAGGAGGACCTG
CTGGCGCTGCCGCTGGACCAGGGCAACCCCACCCCCAGTCTGAAGCCCGTGACCCTGCAGCAGGTGCT
GCCGAGCAGCGCACCCTCCGAGGCGAAGCGGGGTCTGAAGCGCGAGGGCGGCGACCTGGCGCCCACC
GTGCAGCTCATGGTGCCCAAGCGGCAGAGGCTGGAGGATGTGCTGGAGAAAATGAAAGTAGACCCCG
GTCTGCAGCCGGACATCAGGGTCCGCCCCATCAAGCAGGTGGCGCCGGGCCTCGGCGTGCAGACCGTG
GACGTGGTCATCCCCACCGGCAACTCCCCCGCCGCCGCCACCACTACCGCTGCCTCCACGGACATGGA
GACACAGACCGATCCCGCCGCAGCCGCAGCCGCAGCCGCCGCCGCGACCTCCTCGGCGGAGGTGCAG
ACGGACCCCTGGCTGCCGCCGGCGATGTCAGCTCCCCGCGCGCGTCGCGGGCGCAGGAAGTACGGCG
CCGCCAACGCGCTCCTGCCCGAGTACGCCTTGCATCCTTCCATCGCGCCCACCCCCGGCTACCGAGGCT
ATACCTACCGCCCGCGAAGAGCCAAGGGTTCCACCCGCCGTCCCCGCCGACGCGCCGCCGCCACCACC
CGCCGCCGCCGCCGCAGACGCCAGCCCGCACTGGCTCCAGTCTCCGTGAGGAAAGTGGCGCGCGACG
GACACACCCTGGTGCTGCCCAGGGCGCGCTACCACCCCAGCATCGTTTAAAAGCCTGTTGTGGTTCTTG
CAGATATGGCCCTCACTTGCCGCCTCCGTTTCCCGGTGCCGGGATACCGAGGAGGAAGATCGCGCCGC
AGGAGGGGTCTGGCCGGCCGCGGCCTGAGCGGAGGCAGCCGCCGCGCGCACCGGCGGCGACGCGCCA
CCAGCCGACGCATGCGCGGCGGGGTGCTGCCCCTGTTAATCCCCCTGATCGCCGCGGCGATCGGCGCC
GTGCCCGGGATCGCCTCCGTGGCCTTGCAAGCGTCCCAGAGGCATTGACAGACTTGCAAACTTGCAAA
TATGGAAAAAAAAACCCCAATAAAAAAGTCTAGACTCTCACGCTCGCTTGGTCCTGTGACTATTTTGT
AGAATGGAAGACATCAACTTTGCGTCGCTGGCCCCGCGTCACGGCTCGCGCCCGTTCCTGGGACACTG
GAACGATATCGGCACCAGCAACATGAGCGGTGGCGCCTTCAGTTGGGGCTCTCTGTGGAGCGGCATTA
AAAGTATCGGGTCTGCCGTTAAAAATTACGGCTCCCGGGCCTGGAACAGCAGCACGGGCCAGATGTTG
AGAGACAAGTTGAAAGAGCAGAACTTCCAGCAGAAGGTGGTGGAGGGCCTGGCCTCCGGCATCAACG
GGGTGGTGGACCTGGCCAACCAGGCCGTGCAGAATAAGATCAACAGCAGACTGGACCCCCGGCCGCC
GGTGGAGGAGGTGCCGCCGGCGCTGGAGACGGTGTCCCCCGATGGGCGTGGCGAGAAGCGCCCGCGG
CCCGATAGGGAAGAGACCACTCTGGTCACGCAGACCGATGAGCCGCCCCCGTATGAGGAGGCCCTGA
AGCAAGGTCTGCCCACCACGCGGCCCATCGCGCCCATGGCCACCGGGGTGGTGGGCCGCCACACCCCC
GCCACGCTGGACTTGCCTCCGCCCGCCGATGTGCCGCAGCAGCAGAAGGCGGCACAGCCGGGCCCGC
CCGCGACCGCCTCCCGTTCCTCCGCCGGTCCTCTGCGCCGCGCGGCCAGCGGCCCCCGCGGGGGGGTC
GCGAGGCACGGCAACTGGCAGAGCACGCTGAACAGCATCGTGGGTCTGGGGGTGCGGTCCGTGAAGC
GCCGCCGATGCTACTGAATAGCTTAGCTAACGTGTTGTATGTGTGTATGCGCCCTATGTCGCCGCCAGA
GGAGCTGCTGAGTCGCCGCCGTTCGCGCGCCCACCACCACCGCCACTCCGCCCCTCAAGATGGCGACC
CCATCGATGATGCCGCAGTGGTCGTACATGCACATCTCGGGCCAGGACGCCTCGGAGTACCTGAGCCC
CGGGCTGGTGCAGTTCGCCCGCGCCACCGAGAGCTACTTCAGCCTGAGTAACAAGTTTAGGAACCCCA
CGGTGGCGCCCACGCACGATGTGACCACCGACCGGTCTCAGCGCCTGACGCTGCGGTTCATTCCCGTG
GACCGCGAGGACACCGCGTACTCGTACAAGGCGCGGTTCACCCTGGCCGTGGGCGACAACCGCGTGCT
GGACATGGCCTCCACCTACTTTGACATCCGCGGGGTGCTGGACCGGGGTCCCACTTTCAAGCCCTACTC
TGGCACCGCCTACAACTCCCTGGCCCCCAAGGGCGCTCCCAACTCCTGCGAGTGGGAGCAAGAGGAA
ACTCAGGCAGTTGAAGAAGCAGCAGAAGAGGAAGAAGAAGATGCTGACGGTCAAGCTGAGGAAGAG
CAAGCAGCTACCAAAAAGACTCATGTATATGCTCAGGCTCCCCTTTCTGGCGAAAAAATTAGTAAAGA
TGGTCTGCAAATAGGAACGGACGCTACAGCTACAGAACAAAAACCTATTTATGCAGACCCTACATTCC
AGCCCGAACCCCAAATCGGGGAGTCCCAGTGGAATGAGGCAGATGCTACAGTCGCCGGCGGTAGAGT
GCTAAAGAAATCTACTCCCATGAAACCATGCTATGGTTCCTATGCAAGACCCACAAATGCTAATGGAG
GTCAGGGTGTACTAACGGCAAATGCCCAGGGACAGCTAGAATCTCAGGTTGAAATGCAATTCTTTTCA
ACTTCTGAAAACGCCCGTAACGAGGCTAACAACATTCAGCCCAAATTGGTGCTGTATAGTGAGGATGT
GCACATGGAGACCCCGGATACGCACCTTTCTTACAAGCCCGCAAAAAGCGATGACAATTCAAAAATCA
TGCTGGGTCAGCAGTCCATGCCCAACAGACCTAATTACATCGGCTTCAGAGACAACTTTATCGGCCTC
ATGTATTACAATAGCACTGGCAACATGGGAGTGCTTGCAGGTCAGGCCTCTCAGTTGAATGCAGTGGT
GGACTTGCAAGACAGAAACACAGAACTGTCCTACCAGCTCTTGCTTGATTCCATGGGTGACAGAACCA
GATACTTTTCCATGTGGAATCAGGCAGTGGACAGTTATGACCCAGATGTTAGAATTATTGAAAATCAT
GGAACTGAAGACGAGCTCCCCAACTATTGTTTCCCTCTGGGTGGCATAGGGGTAACTGACACTTACCA
GGCTGTTAAAACCAACAATGGCAATAACGGGGGCCAGGTGACTTGGACAAAAGATGAAACTTTTGCA
GATCGCAATGAAATAGGGGTGGGAAACAATTTCGCTATGGAGATCAACCTCAGTGCCAACCTGTGGA
GAAACTTCCTGTACTCCAACGTGGCGCTGTACCTACCAGACAAGCTTAAGTACAACCCCTCCAATGTG
GACATCTCTGACAACCCCAACACCTACGATTACATGAACAAGCGAGTGGTGGCCCCGGGGCTGGTGGA
CTGCTACATCAACCTGGGCGCGCGCTGGTCGCTGGACTACATGGACAACGTCAACCCCTTCAACCACC
ACCGCAATGCGGGCCTGCGCTACCGCTCCATGCTCCTGGGCAACGGGCGCTACGTGCCCTTCCACATC
CAGGTGCCCCAGAAGTTCTTTGCCATCAAGAACCTCCTCCTCCTGCCGGGCTCCTACACCTACGAGTGG
AACTTCAGGAAGGATGTCAACATGGTCCTCCAGAGCTCTCTGGGTAACGATCTCAGGGTGGACGGGGC
CAGCATCAAGTTCGAGAGCATCTGCCTCTACGCCACCTTCTTCCCCATGGCCCACAACACGGCCTCCAC
GCTCGAGGCCATGCTCAGGAACGACACCAACGACCAGTCCTTCAATGACTACCTCTCCGCCGCCAACA
TGCTCTACCCCATACCCGCCAACGCCACCAACGTCCCCATCTCCATCCCCTCGCGCAACTGGGCGGCCT
TCCGCGGCTGGGCCTTCACCCGCCTCAAGACCAAGGAGACCCCCTCCCTGGGCTCGGGATTCGACCCC
TACTACACCTACTCGGGCTCCATTCCCTACCTGGACGGCACCTTCTACCTCAACCACACTTTCAAGAAG
GTCTCGGTCACCTTCGACTCCTCGGTCAGCTGGCCGGGCAACGACCGTCTGCTCACCCCCAACGAGTTC
GAGATCAAGCGCTCGGTCGACGGGGAGGGCTACAACGTGGCCCAGTGCAACATGACCAAGGACTGGT
TCCTGGTCCAGATGCTGGCCAACTACAACATCGGCTACCAGGGCTTCTACATCCCAGAGAGCTACAAG
GACAGGATGTACTCCTTCTTCAGGAACTTCCAGCCCATGAGCCGGCAGGTGGTGGACCAGACCAAGTA
CAAGGACTACCAGGAGGTGGGCATCATCCACCAGCACAACAACTCGGGCTTCGTGGGCTACCTCGCCC
CCACCATGCGCGAGGGACAGGCCTACCCCGCCAACTTCCCCTATCCGCTCATAGGCAAGACCGCGGTC
GACAGCATCACCCAGAAAAAGTTCCTCTGCGACCGCACCCTCTGGCGCATCCCCTTCTCCAGCAACTTC
ATGTCCATGGGTGCGCTCTCGGACCTGGGCCAGAACTTGCTCTACGCCAACTCCGCCCACGCCCTCGA
CATGACCTTCGAGGTCGACCCCATGGACGAGCCCACCCTTCTCTATGTTCTGTTCGAAGTCTTTGACGT
GGTCCGGGTCCACCAGCCGCACCGCGGCGTCATCGAGACCGTGTACCTGCGTACGCCCTTCTCGGCCG
GCAACGCCACCACCTAAAGAAGCAAGCCGCAGTCATCGCCGCCTGCATGCCGTCGGGTTCCACCGAGC
AAGAGCTCAGGGCCATCGTCAGAGACCTGGGATGCGGGCCCTATTTTTTGGGCACCTTCGACAAGCGC
TTCCCTGGCTTTGTCTCCCCACACAAGCTGGCCTGCGCCATCGTCAACACGGCCGGCCGCGAGACCGG
GGGCGTGCACTGGCTGGCCTTCGCCTGGAACCCGCGCTCCAAAACATGCTTCCTCTTTGACCCCTTCGG
CTTTTCGGACCAGCGGCTCAAGCAAATCTACGAGTTCGAGTACGAGGGCTTGCTGCGTCGCAGCGCCA
TCGCCTCCTCGCCCGACCGCTGCGTCACCCTCGAAAAGTCCACCCAGACCGTGCAGGGGCCCGACTCG
GCCGCCTGCGGTCTCTTCTGCTGCATGTTTCTGCACGCCTTTGTGCACTGGCCTCAGAGTCCCATGGAC
CGCAACCCCACCATGAACTTGCTGACGGGGGTGCCCAACTCCATGCTCCAGAGCCCCCAGGTCGAGCC
CACCCTGCGCCGCAACCAGGAGCAGCTCTACAGCTTCCTGGAGCGCCACTCGCCTTACTTCCGCCGCC
ACAGCGCACAGATCAGGAGGGCCACCTCCTTCTGCCACTTGCAAGAGATGCAAGAAGGGTAATAACG
ATGTACACACTTTTTTTCTCAATAAATGGCATCTTTTTATTTATACAAGCTCTCTGGGGTATTCATTTCC
CACCACCACCCGCCGTTGTCGCCATCTGGCTCTATTTAGAAATCGAAAGGGTTCTGCCGGGAGTCGCC
GTGCGCCACGGGCAGGGACACGTTGCGATACTGGTAGCGGGTGCCCCACTTGAACTCGGGCACCACCA
GGCGAGGCAGCTCGGGGAAGTTTTCGCTCCACAGGCTGCGGGTCAGCACCAGCGCGTTCATCAGGTCG
GGCGCCGAGATCTTGAAGTCGCAGTTGGGGCCGCCGCCCTGCGCGCGCGAGTTGCGGTACACCGGGTT
GCAGCACTGGAACACCAACAGCGCCGGGTGCTTCACGCTGGCCAGCACGCTGCGGTCGGAGATCAGC
TCGGCGTCCAGGTCCTCCGCGTTGCTCAGCGCGAACGGGGTCATCTTGGGCACTTGCCGCCCCAGGAA
GGGCGCGTGCCCCGGTTTCGAGTTGCAGTCGCAGCGCAGCGGGATCAGCAGGTGCCCGTGCCCGGACT
CGGCGTTGGGGTACAGCGCGCGCATGAAGGCCTGCATCTGGCGGAAGGCCATCTGGGCCTTGGCGCCC
TCCGAGAAGAACATGCCGCAGGACTTGCCCGAGAACTGGTTTGCGGGGCAGCTGGCGTCGTGCAGGC
AGCAGCGCGCGTCGGTGTTGGCGATCTGCACCACGTTGCGCCCCCACCGGTTCTTCACGATCTTGGCCT
TGGACGATTGCTCCTTCAGCGCGCGCTGCCCGTTCTCGCTGGTCACATCCATCTCGATCACATGTTCCT
TGTTCACCATGCTGCTGCCGTGCAGACACTTCAGCTCGCCCTCCGTCTCGGTGCAGCGGTGCTGCCACA
GCGCGCAGCCCGTGGGCTCGAAAGACTTGTAGGTCACCTCCGCGAAGGACTGCAGGTACCCCTGCAAA
AAGCGGCCCATCATGGTCACGAAGGTCTTGTTGCTGCTGAAGGTCAGCTGCAGCCCGCGGTGCTCCTC
GTTCAGCCAGGTCTTGCACACGGCCGCCAGCGCCTCCACCTGGTCGGGCAGCATCTTGAAGTTCACCTT
CAGCTCATTCTCCACGTGGTACTTGTCCATCAGCGTGCGCGCCGCCTCCATGCCCTTCTCCCAGGCCGA
CACCAGCGGCAGGCTCACGGGGTTCTTCACCATCACCGTGGCCGCCGCCTCCGCCGCGCTTTCGCTTTC
CGCCCCGCTGTTCTCTTCCTCTTCCTCCTCTTCCTCGCCGCCGCCCACTCGCAGCCCCCGCACCACGGGG
TCGTCTTCCTGCAGGCGCTGCACCTTGCGCTTGCCGTTGCGCCCCTGCTTGATGCGCACGGGCGGGTTG
CTGAAGCCCACCATCACCAGCGCGGCCTCTTCTTGCTCGTCCTCGCTGTCCAGAATGACCTCCGGGGAG
GGGGGGTTGGTCATCCTCAGTACCGAGGCACGCTTCTTTTTCTTCCTGGGGGCGTTCGCCAGCTCCGCG
GCTGCGGCCGCTGCCGAGGTCGAAGGCCGAGGGCTGGGCGTGCGCGGCACCAGCGCGTCCTGCGAGC
CGTCCTCGTCCTCCTCGGACTCGAGACGGAGGCGGGCCCGCTTCTTCGGGGGCGCGCGGGGCGGCGGA
GGCGGCGGCGGCGACGGAGACGGGGACGAGACATCGTCCAGGGTGGGTGGACGGCGGGCCGCGCCG
CGTCCGCGCTCGGGGGTGGTCTCGCGCTGGTCCTCTTCCCGACTGGCCATCTCCCACTGCTCCTTCTCCT
ATAGGCAGAAAGAGATCATGGAGTCTCTCATGCGAGTCGAGAAGGAGGAGGACAGCCTAACCGCCCC
CTCTGAGCCCTCCACCACCGCCGCCACCACCGCCAATGCCGCCGCGGACGACGCGCCCACCGAGACCA
CCGCCAGTACCACCCTCCCCAGCGACGCACCCCCGCTCGAGAATGAAGTGCTGATCGAGCAGGACCCG
GGTTTTGTGAGCGGAGAGGAGGATGAGGTGGATGAGAAGGAGAAGGAGGAGGTCGCCGCCTCAGTGC
CAAAAGAGGATAAAAAGCAAGACCAGGACGACGCAGATAAGGATGAGACAGCAGTCGGGCGGGGGA
ACGGAAGCCATGATGCTGATGACGGCTACCTAGACGTGGGAGACGACGTGCTGCTTAAGCACCTGCAC
CGCCAGTGCGTCATCGTCTGCGACGCGCTGCAGGAGCGCTGCGAAGTGCCCCTGGACGTGGCGGAGGT
CAGCCGCGCCTACGAGCGGCACCTCTTCGCGCCGCACGTGCCCCCCAAGCGCCGGGAGAACGGCACCT
GCGAGCCCAACCCGCGTCTCAACTTCTACCCGGTCTTCGCGGTACCCGAGGTGCTGGCCACCTACCAC
ATCTTTTTCCAAAACTGCAAGATCCCCCTCTCCTGCCGCGCCAACCGCACCCGCGCCGACAAAACCCTG
ACCCTGCGGCAGGGCGCCCACATACCTGATATCGCCTCTCTGGAGGAAGTGCCCAAGATCTTCGAGGG
TCTCGGTCGCGACGAGAAACGGGCGGCGAACGCTCTGCACGGAGACAGCGAAAACGAGAGTCACTCG
GGGGTGCTGGTGGAGCTCGAGGGCGACAACGCGCGCCTGGCCGTACTCAAGCGCAGCATAGAGGTCA
CCCACTTTGCCTACCCGGCGCTCAACCTGCCCCCCAAGGTCATGAGTGTGGTCATGGGCGAGCTCATC
ATGCGCCGCGCCCAGCCCCTGGCCGCGGATGCAAACTTGCAAGAGTCCTCCGAGGAAGGCCTGCCCGC
GGTCAGCGACGAGCAGCTGGCGCGCTGGCTGGAGACCCGCGACCCCGCGCAGCTGGAGGAGCGGCGC
AAGCTCATGATGGCCGCGGTGCTGGTCACCGTGGAGCTCGAGTGTCTGCAGCGCTTCTTCGCGGACCC
CGAGATGCAGCGCAAGCTCGAGGAGACCCTGCACTACACCTTCCGCCAGGGCTACGTGCGCCAGGCCT
GCAAGATCTCCAACGTGGAGCTCTGCAACCTGGTCTCCTACCTGGGCATCCTGCACGAGAACCGCCTC
GGGCAGAACGTCCTGCACTCCACCCTCAAAGGGGAGGCGCGCCGCGACTACATCCGCGACTGCGCCTA
CCTCTTCCTCTGCTACACCTGGCAGACGGCCATGGGGGTCTGGCAGCAGTGCCTGGAGGAGCGCAACC
TCAAGGAGCTGGAAAAGCTCCTCAAGCGCACCCTCAGGGACCTCTGGACGGGCTTCAACGAGCGCTCG
GTGGCCGCCGCGCTGGCGGACATCATCTTTCCCGAGCGCCTGCTCAAGACCCTGCAGCAGGGCCTGCC
CGACTTCACCAGCCAGAGCATGCTGCAGAACTTCAGGACTTTCATCCTGGAGCGCTCGGGCATCCTGC
CGGCCACTTGCTGCGCGCTGCCCAGCGACTTCGTGCCCATCAAGTACAGGGAGTGCCCGCCGCCGCTC
TGGGGCCACTGCTACCTCTTCCAGCTGGCCAACTACCTCGCCTACCACTCGGACCTCATGGAAGACGT
GAGCGGCGAGGGCCTGCTCGAGTGCCACTGCCGCTGCAACCTCTGCACGCCCCACCGCTCTCTAGTCT
GCAACCCGCAGCTGCTCAGCGAGAGTCAGATTATCGGTACCTTCGAGCTGCAGGGTCCCTCGCCTGAC
GAGAAGTCCGCGGCTCCAGGGCTGAAACTCACTCCGGGGCTGTGGACTTCCGCCTACCTACGCAAATT
TGTACCTGAGGACTACCACGCCCACGAGATCAGGTTCTACGAAGACCAATCCCGCCCGCCCAAGGCGG
AGCTCACCGCCTGCGTCATCACCCAGGGGCACATCCTGGGCCAATTGCAAGCCATCAACAAAGCCCGC
CGAGAGTTCTTGCTGAAAAAGGGTCGGGGGGTGTACCTGGACCCCCAGTCCGGCGAGGAGCTAAACC
CGCTACCCCCGCCGCCGCCCCAGCAGCGGGACCTTGCTTCCCAGGATGGCACCCAGAAAGAAGCAGC
AGCCGCCGCCGCCGCCGCAGCCATACATGCTTCTGGAGGAAGAGGAGGAGGACTGGGACAGTCAGGC
AGAGGAGGTTTCGGACGAGGAGCAGGAGGAGATGATGGAAGACTGGGAGGAGGACAGCAGCCTAGA
CGAGGAAGCTTCAGAGGCCGAAGAGGTGGCAGACGCAACACCATCGCCCTCGGTCGCAGCCCCCTCG
CCGGGGCCCCTGAAATCCTCCGAACCCAGCACCAGCGCTATAACCTCCGCTCCTCCGGCGCCGGCGCC
ACCCGCCCGCAGACCCAACCGTAGATGGGACACCACAGGAACCGGGGTCGGTAAGTCCAAGTGCCCG
CCGCCGCCACCGCAGCAGCAGCAGCAGCAGCGCCAGGGCTACCGCTCGTGGCGCGGGCACAAGAACG
CCATAGTCGCCTGCTTGCAAGACTGCGGGGGCAACATCTCTTTCGCCCGCCGCTTCCTGCTATTCCACC
ACGGGGTCGCCTTTCCCCGCAATGTCCTGCATTACTACCGTCATCTCTACAGCCCCTACTGCAGCGGCG
ACCCAGAGGCGGCAGCGGCAGCCACAGCGGCGACCACCACCTAGGAAGATATCCTCCGCGGGCAAGA
CAGCGGCAGCAGCGGCCAGGAGACCCGCGGCAGCAGCGGCGGGAGCGGTGGGCGCACTGCGCCTCTC
GCCCAACGAACCCCTCTCGACCCGGGAGCTCAGACACAGGATCTTCCCCACTTTGTATGCCATCTTCCA
ACAGAGCAGAGGCCAGGAGCAGGAGCTGAAAATAAAAAACAGATCTCTGCGCTCCCTCACCCGCAGC
TGTCTGTATCACAAAAGCGAAGATCAGCTTCGGCGCACGCTGGAGGACGCGGAGGCACTCTTCAGCAA
ATACTGCGCGCTCACTCTTAAAGACTAGCTCCGCGCCCTTCTCGAATTTAGGCGGGAGAAAACTACGT
CATCGCCGGCCGCCGCCCAGCCCGCCCAGCCGAGATGAGCAAAGAGATTCCCACGCCATACATGTGG
AGCTACCAGCCGCAGATGGGACTCGCGGCGGGAGCGGCCCAGGACTACTCCACCCGCATGAACTACA
TGAGCGCGGGACCCCACATGATCTCACAGGTCAACGGGATCCGCGCCCAGCGAAACCAAATACTGCT
GGAACAGGCGGCCATCACCGCCACGCCCCGCCATAATCTCAACCCCCGAAATTGGCCCGCCGCCCTCG
TGTACCAGGAAACCCCCTCCGCCACCACCGTACTACTTCCGCGTGACGCCCAGGCCGAAGTCCAGATG
ACTAACTCAGGGGCGCAGCTCGCGGGCGGCTTTCGTCACGGGGCGCGGCCGCTCCGACCAGGTATAAG
ACACCTGATGATCAGAGGCCGAGGTATCCAGCTCAACGACGAGTCGGTGAGCTCTTCGCTCGGTCTCC
GTCCGGACGGAACTTTCCAGCTCGCCGGATCCGGCCGCTCTTCGTTCACGCCCCGCCAGGCGTACCTG
ACTCTGCAGACCTCGTCCTCGGAGCCCCGCTCCGGCGGCATCGGAACCCTCCAGTTCGTGGAGGAGTT
CGTGCCCTCGGTCTACTTCAACCCCTTCTCGGGACCTCCCGGACGCTACCCCGACCAGTTCATTCCGAA
CTTTGACGCGGTGAAGGACTCGGCGGACGGCTACGACTGAATGTCAGGTGTCGAGGCAGAGCAGCTTC
GCCTGAGACACCTCGAGCACTGCCGCCGCCACAAGTGCTTCGCCCGCGGTTCTGGTGAGTTCTGCTACT
TTCAGCTACCCGAGGAGCATACCGAGGGGCCGGCGCACGGCGTCCGCCTGACCACCCAGGGCGAGGT
TACCTGTTCCCTCATCCGGGAGTTTACCCTCCGTCCCCTGCTAGTGGAGCGGGAGCGGGGTCCCTGTGT
CCTAACTATCGCCTGCAACTGCCCTAACCCTGGATTACATCAAGATCTTTGCTGTCATCTCTGTGCTGA
GTTTAATAAACGCTGAGATCAGAATCTACTGGGGCTCCTGTCGCCATCCTGTGAACGCCACCGTCTTCA
CCCACCCCGACCAGGCCCAGGCGAACCTCACCTGCGGTCTGCATCGGAGGGCCAAGAAGTACCTCACC
TGGTACTTCAACGGCACCCCCTTTGTGGTTTACAACAGCTTCGACGGGGACGGAGTCTCCCTGAAAGA
CCAGCTCTCCGGTCTCAGCTACTCCATCCACAAGAACACCACCCTCCAACTCTTCCCTCCCTACCTGCC
GGGAACCTACGAGTGCGTCACCGGCCGCTGCACCCACCTCACCCGCCTGATCGTAAACCAGAGCTTTC
CGGGAACAGATAACTCCCTCTTCCCCAGAACAGGAGGTGAGCTCAGGAAACTCCCCGGGGACCAGGG
CGGAGACGTACCTTCGACCCTTGTGGGGTTAGGATTTTTTATTACCGGGTTGCTGGCTCTTTTAATCAA
AGTTTCCTTGAGATTTGTTCTTTCCTTCTACGTGTATGAACACCTCAACCTCCAATAACTCTACCCTTTC
TTCGGAATCAGGTGACTTCTCTGAAATCGGGCTTGGTGTGCTGCTTACTCTGTTGATTTTTTTCCTTATC
ATACTCAGCCTTCTGTGCCTCAGGCTCGCCGCCTGCTGCGCACACATCTATATCTACTGCTGGTTGCTC
AAGTGCAGGGGTCGCCACCCAAGATGAACAGGTACATGGTCCTATCGATCCTAGGCCTGCTGGCCCTG
GCGGCCTGCAGCGCCGCCAAAAAAGAGATTACCTTTGAGGAGCCCGCTTGCAATGTAACTTTCAAGCC
CGAGGGTGACCAATGCACCACCCTCGTCAAATGCGTTACCAATCATGAGAGGCTGCGCATCGACTACA
AAAACAAAACTGGCCAGTTTGCGGTCTATAGTGTGTTTACGCCCGGAGACCCCTCTAACTACTCTGTCA
CCGTCTTCCAGGGCGGACAGTCTAAGATATTCAATTACACTTTCCCTTTTTATGAGTTATGCGATGCGG
TCATGTACATGTCAAAACAGTACAACCTGTGGCCTCCCTCTCCCCAGGCGTGTGTGGAAAATACTGGG
TCTTACTGCTGTATGGCTTTCGCAATCACTACGCTCGCTCTAATCTGCACGGTGCTATACATAAAATTC
AGGCAGAGGCGAATCTTTATCGATGAAAAGAAAATGCCTTGATCGCTAACACCGGCTTTCTATCTGCA
GAATGAATGCAATCACCTCCCTACTAATCACCACCACCCTCCTTGCGATTGCCCATGGGTTGACACGA
ATCGAAGTGCCAGTGGGGTCCAATGTCACCATGGTGGGCCCCGCCGGCAATTCCACCCTCATGTGGGA
AAAATTTGTCCGCAATCAATGGGTTCATTTCTGCTCTAACCGAATCAGTATCAAGCCCAGAGCCATCTG
CGATGGGCAAAATCTAACTCTGATCAATGTGCAAATGATGGATGCTGGGTACTATTACGGGCAGCGGG
GAGAAATCATTAATTACTGGCGACCCCACAAGGACTACATGCTGCATGTAGTCGAGGCACTTCCCACT
ACCACCCCCACTACCACCTCTCCCACCACCACCACCACTACTACTACTACTACTACTACTACTACTACT
ACCACTACCGCTGCCCGCCATACCCGCAAAAGCACCATGATTAGCACAAAGCCCCCTCGTGCTCACTC
CCACGCCGGCGGGCCCATCGGTGCGACCTCAGAAACCACCGAGCTTTGCTTCTGCCAATGCACTAACG
CCAGCGCTCATGAACTGTTCGACCTGGAGAATGAGGATGTCCAGCAGAGCTCCGCTTGCCTGACCCAG
GAGGCTGTGGAGCCCGTTGCCCTGAAGCAGATCGGTGATTCAATAATTGACTCTTCTTCTTTTGCCACT
CCCGAATACCCTCCCGATTCTACTTTCCACATCACGGGTACCAAAGACCCTAACCTCTCTTTCTACCTG
ATGCTGCTGCTCTGTATCTCTGTGGTCTCTTCCGCGCTGATGTTACTGGGGATGTTCTGCTGCCTGATCT
GCCGCAGAAAGAGAAAAGCTCGCTCTCAGGGCCAACCACTGATGCCCTTCCCCTACCCCCCGGATTTT
GCAGATAACAAGATATGAGCTCGCTGCTGACACTAACCGCTTTACTAGCCTGCGCTCTAACCCTTGTCG
CTTGCGACTCGAGATTCCACAATGTCACAGCTGTGGCAGGAGAAAATGTTACTTTCAACTCCACGGCC
GATACCCAGTGGTCGTGGAGTGGCTCAGGTAGCTACTTAACTATCTGCAATAGCTCCACTTCCCCCGGC
ATATCCCCAACCAAGTACCAATGCAATGCCAGCCTGTTCACCCTCATCAACGCTTCCACCCTGGACAAT
GGACTCTATGTAGGCTATGTACCCTTTGGTGGGCAAGGAAAGACCCACGCTTACAACCTGGAAGTTCG
CCAGCCCAGAACCACTACCCAAGCTTCTCCCACCACCACCACCACCACCACCATCACCAGCAGCAGCA
GCAGCAGCAGCCACAGCAGCAGCAGCAGATTATTGACTTTGGTTTTGGCCAGCTCATCTGCCGCTACC
CAGGCCATCTACAGCTCTGTGCCCGAAACCACTCAGATCCACCGCCCAGAAACGACCACCGCCACCAC
CCTACACACCTCCAGCGATCAGATGCCGACCAACATCACCCCCTTGGCTCTTCAAATGGGACTTACAA
GCCCCACTCCAAAACCAGTGGATGCGGCCGAGGTCTCCGCCCTCGTCAATGACTGGGCGGGGCTGGGA
ATGTGGTGGTTCGCCATAGGCATGATGGCGCTCTGCCTGCTTCTGCTCTGGCTCATCTGCTGCCTCCAC
CGCAGGCGAGCCAGACCCCCCATCTATAGACCCATCATTGTCCTGAACCCCGATAATGATGGGATCCA
TAGATTGGATGGCCTGAAAAACCTACTTTTTTCTTTTACAGTATGATAAATTGAGACATGCCTCGCATT
TTCTTGTACATGTTCCTTCTCCCACCTTTTCTGGGGTGTTCTACGCTGGCCGCTGTGTCTCACCTGGAGG
TAGACTGCCTCTCACCCTTCACTGTCTACCTGCTTTACGGATTGGTCACCCTCACTCTCATCTGCAGCCT
AATCACAGTAATCATCGCCTTCATCCAGTGCATTGATTACATCTGTGTGCGCCTCGCATACTTCAGACA
CCACCCGCAGTACCGAGACAGGAACATTGCCCAACTTCTAAGACTGCTCTAATCATGCATAAGACTGT
GATCTGCCTTCTGATCCTCTGCATCCTGCCCACCCTCACCTCCTGCCAGTACACCACAAAATCTCCGCG
CAAAAGACATGCCTCCTGCCGCTTCACCCAACTGTGGAATATACCCAAATGCTACAACGAAAAGAGCG
AGCTCTCCGAAGCTTGGCTGTATGGGGTCATCTGTGTCTTAGTTTTCTGCAGCACTGTCTTTGCCCTCAT
AATCTACCCCTACTTTGATTTGGGATGGAACGCGATCGATGCCATGAATTACCCCACCTTTCCCGCACC
CGAGATAATTCCACTGCGACAAGTTGTACCCGTTGTCGTTAATCAACGCCCCCCATCCCCTACGCCCAC
TGAAATCAGCTACTTTAACCTAACAGGCGGAGATGACTGACGCCCTAGATCTAGAAATGGACGGCATC
AGTACCGAGCAGCGTCTCCTAGAGAGGCGCAGGCAGGCGGCTGAGCAAGAGCGCCTCAATCAGGAGC
TCCGAGATCTCGTTAACCTGCACCAGTGCAAAAGAGGCATCTTTTGTCTGGTAAAGCAGGCCAAAGTC
ACCTACGAGAAGACCGGCAACAGCCACCGCCTCAGTTACAAATTGCCCACCCAGCGCCAGAAGCTGG
TGCTCATGGTGGGTGAGAATCCCATCACCGTCACCCAGCACTCGGTAGAGACCGAGGGGTGTCTGCAC
TCCCCCTGTCGGGGTCCAGAAGACCTCTGCACCCTGGTAAAGACCCTGTGCGGTCTCAGAGATTTAGT
CCCCTTTAACTAATCAAACACTGGAATCAATAAAAAGAATCACTTACTTAAAATCAGACAGCAGGTCT
CTGTCCAGTTTATTCAGCAGCACCTCCTTCCCCTCCTCCCAACTCTGGTACTCCAAACGCCTTCTGGCG
GCAAACTTCCTCCACACCCTGAAGGGAATGTCAGATTCTTGCTCCTGTCCCTCCGCACCCACTATCTTC
ATGTTGTTGCAGATGAAGCGCACCAAAACGTCTGACGAGAGCTTCAACCCCGTGTACCCCTATGACAC
GGAAAGCGGCCCTCCCTCCGTCCCTTTCCTCACCCCTCCCTTCGTGTCTCCCGATGGATTCCAAGAAAG
TCCCCCCGGGGTCCTGTCTCTGAACCTGGCCGAGCCCCTGGTCACTTCCCACGGCATGCTCGCCCTGAA
AATGGGAAGTGGCCTCTCCCTGGACGACGCTGGCAACCTCACCTCTCAAGATATCACCACCGCTAGCC
CTCCCCTCAAAAAAACCAAGACCAACCTCAGCCTAGAAACCTCATCCCCCCTAACTGTGAGCACCTCA
GGCGCCCTCACCGTAGCAGCCGCCGCTCCCCTGGCGGTGGCCGGCACCTCCCTCACCATGCAATCAGA
GGCCCCCCTGACAGTACAGGATGCAAAACTCACCCTGGCCACCAAAGGCCCCCTGACCGTGTCTGAAG
GCAAACTGGCCTTGCAAACATCGGCCCCGCTGACGGCCGCTGACAGCAGCACCCTCACAGTCAGTGCC
ACACCACCCCTTAGCACAAGCAATGGCAGCTTGGGTATTGACATGCAAGCCCCCATTTACACCACCAA
TGGAAAACTAGGACTTAACTTTGGCGCTCCCCTGCATGTGGTAGACAGCCTAAATGCACTGACTGTAG
TTACTGGCCAAGGTCTTACGATAAACGGAACAGCCCTACAAACTAGAGTCTCAGGTGCCCTCAACTAT
GACACATCAGGAAACCTAGAATTGAGAGCTGCAGGGGGTATGCGAGTTGATGCAAATGGTCAACTTA
TCCTTGATGTAGCTTACCCATTTGATGCACAAAACAATCTCAGCCTTAGGCTTGGACAGGGACCCCTGT
TTGTTAACTCTGCCCACAACTTGGATGTTAACTACAACAGAGGCCTCTACCTGTTCACATCTGGAAATA
CCAAAAAGCTAGAAGTTAATATCAAAACAGCCAAGGGTCTCATTTATGATGACACTGCTATAGCAATC
AATGCGGGTGATGGGCTACAGTTTGACTCAGGCTCAGATACAAATCCATTAAAAACTAAACTTGGATT
AGGACTGGATTATGACTCCAGCAGAGCCATAATTGCTAAACTGGGAACTGGCCTAAGCTTTGACAACA
CAGGTGCCATCACAGTAGGCAACAAAAATGATGACAAGCTTACCTTGTGGACCACACCAGACCCATCC
CCTAACTGTAGAATCTATTCAGAGAAAGATGCTAAATTCACACTTGTTTTGACTAAATGCGGCAGTCA
GGTGTTGGCCAGCGTTTCTGTTTTATCTGTAAAAGGTAGCCTTGCGCCCATCAGTGGCACAGTAACTAG
TGCTCAGATTGTCCTCAGATTTGATGAAAATGGAGTTCTACTAAGCAATTCTTCCCTTGACCCTCAATA
CTGGAACTACAGAAAAGGTGACCTTACAGAGGGCACTGCATATACCAACGCAGTGGGATTTATGCCCA
ACCTCACAGCATACCCAAAAACACAGAGCCAAACTGCTAAAAGCAACATTGTAAGTCAGGTTTACTTG
AATGGGGACAAATCCAAACCCATGACCCTCACCATTACCCTCAATGGAACTAATGAAACAGGAGATG
CCACAGTAAGCACTTACTCCATGTCATTCTCATGGAACTGGAATGGAAGTAATTACATTAATGAAACG
TTCCAAACCAACTCCTTCACCTTCTCCTACATCGCCCAAGAATAAAAAGCATGACGCTGTTGATTTGAT
TCAATGTGTTTCTGTTTTATTTTCAAGCACAACAAAATCATTCAAGTCATTCTTCCATCTTAGCTTAATA
GACACAGTAGCTTAATAGACCCAGTAGTGCAAAGCCCCATTCTAGCTTATAACTAGTGGAGAAGTACT
CGCCTACATGGGGGTAGAGTCATAATCGTGCATCAGGATAGGGCGGTGGTGCTGCAGCAGCGCGCGA
ATAAACTGCTGCCGCCGCCGCTCCGTCCTGCAGGAATACAACATGGCAGTGGTCTCCTCAGCGATGAT
TCGCACCGCCCGCAGCATAAGGCGCCTTGTCCTCCGGGCACAGCAGCGCACCCTGATCTCACTTAAAT
CAGCACAGTAACTGCAGCACAGCACCACAATATTGTTCAAAATCCCACAGTGCAAGGCGCTGTATCCA
AAGCTCATGGCGGGGACCACAGAACCCACGTGGCCATCATACCACAAGCGCAGGTAGATTAAGTGGC
GACCCCTCATAAACACGCTGGACATAAACATTACCTCTTTTGGCATGTTGTAATTCACCACCTCCCGGT
ACCATATAAACCTCTGATTAAACATGGCGCCATCCACCACCATCCTAAACCAGCTGGCCAAAACCTGC
CCGCCGGCTATACACTGCAGGGAACCGGGACTGGAACAATGACAGTGGAGAGCCCAGGACTCGTAAC
CATGGATCATCATGCTCGTCATGATATCAATGTTGGCACAACACAGGCACACGTGCATACACTTCCTC
AGGATTACAAGCTCCTCCCGCGTTAGAACCATATCCCAGGGAACAACCCATTCCTGAATCAGCGTAAA
TCCCACACTGCAGGGAAGACCTCGCACGTAACTCACGTTGTGCATTGTCAAAGTGTTACATTCGGGCA
GCAGCGGATGATCCTCCAGTATGGTAGCGCGGGTTTCTGTCTCAAAAGGAGGTAGACGATCCCTACTG
TACGGAGTGCGCCGAGACAACCGAGATCGTGTTGGTCGTAGTGTCATGCCAAATGGAACGCCGGACGT
AGTCATATTTCCTGAAGTCTTAGATCTCTCAACGCAGCACCAGCACCAACACTTCGCAGTGTAAAAGG
CCAAGTGCCGAGAGAGTATATATAGGAATAAAAAGTGACGTAAACGGGCAAAGTCCAAAAAACGCCC
AGAAAAACCGCACGCGAACCTACGCCCCGAAACGAAAGCCAAAAAACACTAGACACTCCCTTCCGGC
GTCAACTTCCGCTTTCCCACGCTACGTCACTTGCCCCAGTCAAACAAACTACATATCCCGAACTTCCAA
GTCGCCACGCCCAAAACACCGCCTACACCTCCCCGCCCGCCGGCCCGCCCCCAAACCCGCCTCCCGCC
CCGCGCCCCGCCCCGCGCCGCCCATCTCATTATCATATTGGCTTCAATCCAAAATAAGGTATATTATTG
ATGATG
RSV F0ΔTM-N-M2-1 amino acid sequence
SEQ ID NO: 5
MELLILKANAITTILTAVTFCFASGQNITEEFYQSTCSAVSKGYLSALRTGWYTSVITIELSNIKENKCNGTDA
KVKLIKQELDKYKNAVTELQLLMQSTPATNNRARRELPRFMNYTLNNAKKTNVTLSKKRKRRFLGFLLGV
GSAIASGVAVSKVLHLEGEVNKIKSALLSTNKAVVSLSNGVSVLTSKVLDLKNYIDKQLLPIVNKQSCSISNI
ETVIEFQQKNNRLLEITREFSVNAGVTTPVSTYMLTNSELLSLNDMPITNDQKKLMSNNVQIVRQQSYSIMSI
IKEEVLAYVVQLPLYGVIDTPCWKLHTSPLCTTNTKEGSNICLTRTDRGWYCDNAGSVSFFPQAETCKVQS
NRVECDTMNSLTLPSEVNLCNVDIFNPKYDCKIMTSKTDVSSSVITSLGAIVSCYGKTKCTASNKNRGIIKTF
SNGCDYVSNKGVDTVSVGNTLYYVNKQEGKSLYVKGEPIINFYDPLVFPSDEFDASISQVNEKINQSLAFIR
KSDELLHNVNAGKSTTNRKRRAPVKQTLNFDLLKLAGDVESNPGPMALSKVKLNDTLNKDQLLSSSKYTI
QRSTGDSIDTPNYDVQKHINKLCGMLLITEDANHKFTGLIGMLYAMSRLGREDTIKILRDAGYHVKANGVD
VTTHRQDINGKEMKFEVLTLASLTTEIQINIEIESRKSYKKMLKEMGEVAPEYRHDSPDCGMIILCIAALVIT
KLAAGDRSGLTAVIRRANNVLKNEMKRYKGLLPKDIANS
FYEVFEKYPHFIDVFVHFGIAQSSTRGGSRVEGIFAGLEMNAYGAGQVMLRWGVLAKSVKNIMLGHASVQ
AEMEQVVEVYLYAQKLGGEAGFYHILNNPKASLLSLTQFPHFSSVVLGNAAGLGIMGEYRGTPRNQDLYD
AAKAYAEQLKENGVINYSVLDLTAEELEAIKHQLNPKDNDVELGGGGSGGGGMSRRNPCKFEIRGHCLNG
KRCHFSHNYFEWPPHALLVRQNFMLNRILKSMDKSIDTLSEISGAAELDRTEEYALGVVGVLESYIGSINNIT
KQSACVAMSKLLTELNSDDIKKLRDNEELNSPKIRVYNTVISYIESNRKNNKQTIHLLKRLPADVLKKTIKN
TLDIHKSITINNPKESTVSDTNDHAKNNDTT
Polynucleotide sequence encoding the enhanced hCMV promoter
SEQ ID NO: 6
CCATTGCATACGTTGTATCCATATCATAATATGTACATTTATATTGGCTCATGTCCAACATTACCGCCATGTTG
ACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATG
ACGTATGTTCCCATAGTAACGCCAATAGGGACTTTCCATTGACGTCAATGGGTGGAGTATTTACGGTAAACTGC
CCACTTGGCAGTACATCAAGTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACATCTACGTATTAGTCATCGCT
ATTACCATGGTGATGCGGTTTTGGCAGTACATCAATGGGCGTGGATAGCGGTTTGACTCACGGGGATTTCCAAG
TCTCCACCCCATTGACGTCAATGGGAGTTTGTTTTGGCACCAAAATCAACGGGACTTTCCAAAATGTCGTAACA
ACTCCGCCCCATTGACGCAAATGGGCGGTAGGCGTGTACGGTGGGAGGTCTATATAAGGCGAAGCGCTCCCTAT
CAGTGATAGAGATCTCCCTATCAGTGATAGAGATCGTCGACGAGCTCGCGGCGGGCGGGAGTCGCTGCGCGCTG
CCTTCGCCCCGTGCCCCGCTCCGCCGCCGCCTCGCGCCGCCCGCCCCGGCTCTGACTGACCGCGTTACTAAAAC
AGGTAAGTCCGGCCTCCGCGCCGGGTTTTGGCGCCTCCCGCGGGCGCCCCCCTCCTCACGGCGAGCGCTGCCAC
GTCAGACGAAGGGCGCAGCGAGCGTCCTGATCCTTCCGCCCGGACGCTCAGGACAGCGGCCCGCTGCTCATAAG
ACTCGGCCTTAGAACCCCAGTATCAGCAGAAGGACATTTTAGGACGGGACTTGGGTGACTCTAGGGCACTGGTT
TTCTTTCCAGAGAGCGGAACAGGCGAGGAAAAGTAGTCCCTTCTCGGCGATTCTGCGGAGGGATCTCCGTGGGG
CGGTGAACGCCGATGATGCCTCTACTAACCATGTTCATGTTTTCTTTTTTTTTCTACAGGTCCTGGGTGACGAA
CAG
Polynucleotide sequence encoding the hCMV NM2 bghpolyA cassette
SEQ ID NO: 7
CCATTGCATACGTTGTATCCATATCATAATATGTACATTTATATTGGCTCATGTCCAACATTACCGCCATGTTG
ACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATG
ACGTATGTTCCCATAGTAACGCCAATAGGGACTTTCCATTGACGTCAATGGGTGGAGTATTTACGGTAAACTGC
CCACTTGGCAGTACATCAAGTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACATCTACGTATTAGTCATCGCT
ATTACCATGGTGATGCGGTTTTGGCAGTACATCAATGGGCGTGGATAGCGGTTTGACTCACGGGGATTTCCAAG
TCTCCACCCCATTGACGTCAATGGGAGTTTGTTTTGGCACCAAAATCAACGGGACTTTCCAAAATGTCGTAACA
ACTCCGCCCCATTGACGCAAATGGGCGGTAGGCGTGTACGGTGGGAGGTCTATATAAGCAGAGCTCTCCCTATC
AGTGATAGAGATCTCCCTATCAGTGATAGAGATCGTCGACGAGCTCGTTTAGTGAACCGTCAGATCGCCTGGAG
ACGCCATCCACGCTGTTTTGACCTCCATAGAAGACACCGGGACCGATCCAGCCTCCGCGGCCGGGAACGGTGCA
TTGGAACGCGGATTCCCCGTGCCAAGAGTGAGATCTTCCGTTTATCTAGGTACCAGATATCGCCACCATGGCCC
TGAGCAAAGTGAAACTGAACGATACACTGAACAAGGACCAGCTGCTGTCCAGCAGCAAGTACACCATCCAGCGG
AGCACCGGCGACAGCATCGATACCCCCAACTACGACGTGCAGAAGCACATCAACAAGCTGTGCGGCATGCTGCT
GATCACAGAGGACGCCAACCACAAGTTCACCGGCCTGATCGGCATGCTGTACGCCATGAGCCGGCTGGGCCGGG
AGGACACCATCAAGATCCTGCGGGACGCCGGCTACCACGTGAAGGCCAATGGCGTGGACGTGACCACACACCGG
CAGGACATCAACGGCAAAGAAATGAAGTTCGAGGTGCTGACCCTGGCCAGCCTGACCACCGAGATCCAGATCAA
TATCGAGATCGAGAGCCGGAAGTCCTACAAGAAAATGCTGAAAGAAATGGGCGAGGTGGCCCCCGAGTACAGAC
ACGACAGCCCCGACTGCGGCATGATCATCCTGTGTATCGCCGCCCTGGTGATCACAAAGCTGGCCGCTGGCGAC
AGATCTGGCCTGACAGCCGTGATCAGACGGGCCAACAATGTGCTGAAGAACGAGATGAAGCGGTACAAGGGCCT
GCTGCCCAAGGACATTGCCAACAGCTTCTACGAGGTGTTCGAGAAGTACCCCCACTTCATCGACGTGTTCGTGC
ACTTCGGCATTGCCCAGAGCAGCACCAGAGGCGGCTCCAGAGTGGAGGGCATCTTCGCCGGCCTGTTCATGAAC
GCCTACGGCGCTGGCCAGGTGATGCTGAGATGGGGCGTGCTGGCCAAGAGCGTGAAGAACATCATGCTGGGCCA
CGCCAGCGTGCAGGCCGAGATGGAACAGGTGGTGGAGGTGTACGAGTACGCCCAGAAGCTGGGCGGAGAGGCCG
GCTTCTACCACATCCTGAACAACCCTAAGGCCTCCCTGCTGTCCCTGACCCAGTTCCCCCACTTCTCCAGCGTG
GTGCTGGGAAATGCCGCCGGACTGGGCATCATGGGCGAGTACCGGGGCACCCCCAGAAACCAGGACCTGTACGA
CGCCGCCAAGGCCTACGCCGAGCAGCTGAAAGAAAACGGCGTGATCAACTACAGCGTGCTGGACCTGACCGCTG
AGGAACTGGAAGCCATCAAGCACCAGCTGAACCCCAAGGACAACGACGTGGAGCTGGGAGGCGGAGGATCTGGC
GGCGGAGGCATGAGCAGACGGAACCCCTGCAAGTTCGAGATCCGGGGCCACTGCCTGAACGGCAAGCGGTGCCA
CTTCAGCCACAACTACTTCGAGTGGCCCCCTCATGCTCTGCTGGTGCGGCAGAACTTCATGCTGAACCGGATCC
TGAAGTCCATGGACAAGAGCATCGACACCCTGAGCGAGATCAGCGGAGCCGCCGAGCTGGACAGAACCGAGGAA
TATGCCCTGGGCGTGGTGGGAGTGCTGGAAAGCTACATCGGCTCCATCAACAACATCACAAAGCAGAGCGCCTG
CGTGGCCATGAGCAAGCTGCTGACAGAGCTGAACAGCGACGACATCAAGAAGCTGAGGGACAACGAGGAACTGA
ACAGCCCCAAGATCCGGGTGTACAACACCGTGATCAGCTACATTGAGAGCAACCGCAAGAACAACAAGCAGACC
ATCCATCTGCTGAAGCGGCTGCCCGCCGACGTGCTGAAAAAGACCATCAAGAACACCCTGGACATCCACAAGTC
CATCACCATCAACAATCCCAAAGAAAGCACCGTGTCTGACACCAACGATCACGCCAAGAACAACGACACCACCT
GATGAGCGGCCGCGATCTGCTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATCGCATTGTCTGAGTAGGT
GTCATTCTATTCTGGGGGGTGGGGTGGGGCAGGACAGCAAGGGGGAGGATTGGGAAGACAATAGCAGGCATGCT
GGGGATGCGGTGGGCTCTATGG
CMV Promoter sequence: bold
Transgene sequence NM2: Italic
bghpolyA PolyA signal: italic + underline
NM2 protein sequence
SEQ ID NO: 8
MALSKVKLNDTLNKDQLLSSSKYTIQRSTGDSIDTPNYDVQKHINKLCGMLLITEDANHKFTGLIGMLYAMSRL
GREDTIKILRDAGYHVKANGVDVTTHRQDINGKEMKFEVLTLASLTTEIQINIEIESRKSYKKMLKEMGEVAPE
YRHDSPDCGMIILCIAALVITKLAAGDRSGLTAVIRRANNVLKNEMKRYKGLLPKDIANSFYEVFEKYPHFIDV
FVHFGIAQSSTRGGSRVEGIFAGLFMNAYGAGQVMLRWGVLAKSVKNIMLGHASVQAEMEQVVEVYEYAQKLGG
EAGFYHILNNPKASLLSLTQFPHFSSVVLGNAAGLGIMGEYRGTPRNQDLYDAAKAYAEQLKENGVINYSVLDL
TAEELEAIKHQLNPKDNDVELGGGGSGGGGMSRRNPCKFEIRGHCLNGKRCHFSHNYFEWPPHALLVRQNFMLN
RILKSMDKSIDTLSEISGAAELDRTEEYALGVVGVLESYIGSINNITKQSACVAMSKLLTELNSDDIKKLRDNE
ELNSPKIRVYNTVISYIESNRKNNKQTIHLLKRLPADVLKKTIKNTLDIHKSITINNPKESTVSDTNDHAKNND
TT
Polynucleotide sequence encoding the hCMV F0 WPRE bghpolyA cassette
SEQ ID NO: 9
CCATTGCATACGTTGTATCCATATCATAATATGTACATTTATATTGGCTCATGTCCAACATTACCGCCATGTTG
ACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATG
ACGTATGTTCCCATAGTAACGCCAATAGGGACTTTCCATTGACGTCAATGGGTGGAGTATTTACGGTAAACTGC
CCACTTGGCAGTACATCAAGTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACATCTACGTATTAGTCATCGCT
ATTACCATGGTGATGCGGTTTTGGCAGTACATCAATGGGCGTGGATAGCGGTTTGACTCACGGGGATTTCCAAG
TCTCCACCCCATTGACGTCAATGGGAGTTTGTTTTGGCACCAAAATCAACGGGACTTTCCAAAATGTCGTAACA
ACTCCGCCCCATTGACGCAAATGGGCGGTAGGCGTGTACGGTGGGAGGTCTATATAAGGCGAAGCGCTCCCTAT
CAGTGATAGAGATCTCCCTATCAGTGATAGAGATCGTCGACGAGCTCGCGGCGGGCGGGAGTCGCTGCGCGCTG
CCTTCGCCCCGTGCCCCGCTCCGCCGCCGCCTCGCGCCGCCCGCCCCGGCTCTGACTGACCGCGTTACTAAAAC
AGGTAAGTCCGGCCTCCGCGCCGGGTTTTGGCGCCTCCCGCGGGCGCCCCCCTCCTCACGGCGAGCGCTGCCAC
GTCAGACGAAGGGCGCAGCGAGCGTCCTGATCCTTCCGCCCGGACGCTCAGGACAGCGGCCCGCTGCTCATAAG
ACTCGGCCTTAGAACCCCAGTATCAGCAGAAGGACATTTTAGGACGGGACTTGGGTGACTCTAGGGCACTGGTT
TTCTTTCCAGAGAGCGGAACAGGCGAGGAAAAGTAGTCCCTTCTCGGCGATTCTGCGGAGGGATCTCCGTGGGG
CGGTGAACGCCGATGATGCCTCTACTAACCATGTTCATGTTTTCTTTTTTTTTCTACAGGTCCTGGGTGACGAA
CAGGATATCGCCACCATGGAACTGCTGATCCTGAAGGCCAACGCCATCACCACCATCCTGACCGCCGTGACCTT
CTGCTTCGCCAGCGGCCAGAACATCACCGAGGAATTCTACCAGAGCACCTGTAGCGCCGTGAGCAAGGGCTACC
TGAGCGCCCTGAGAACCGGCTGGTACACCAGCGTGATCACCATCGAGCTGAGCAACATCAAAGAAAACAAGTGC
AACGGCACCGACGCCAAAGTGAAGCTGATCAAGCAGGAACTGGACAAGTACAAGAACGCCGTGACCGAGCTGCA
GCTGCTGATGCAGAGCACCCCCGCCACCAACAACCGGGCCAGACGGGAGCTGCCCCGGTTCATGAACTACACCC
TGAACAACGCCAAAAAGACCAACGTGACCCTGAGCAAGAAGCGGAAGCGGCGGTTCCTGGGCTTTCTGCTGGGC
GTGGGCAGCGCCATTGCCAGCGGCGTGGCCGTGTCTAAGGTGCTGCACCTGGAAGGCGAAGTGAACAAGATCAA
GAGCGCCCTGCTGAGCACCAACAAGGCCGTGGTGTCCCTGAGCAACGGCGTGAGCGTGCTGACCAGCAAGGTGC
TGGATCTGAAGAACTACATCGACAAGCAGCTGCTGCCCATCGTGAACAAGCAGAGCTGCAGCATCAGCAACATC
GAGACAGTGATCGAGTTCCAGCAGAAGAACAACCGGCTGCTGGAAATCACCCGGGAGTTCAGCGTGAACGCCGG
CGTGACCACCCCTGTGTCCACCTACATGCTGACCAACAGCGAGCTGCTGAGCCTGATCAACGACATGCCCATCA
CCAACGACCAGAAAAAGCTGATGAGCAACAACGTGCAGATCGTGCGGCAGCAGAGCTACTCCATCATGTCCATC
ATCAAAGAAGAGGTGCTGGCCTACGTGGTGCAGCTGCCCCTGTACGGCGTGATCGACACCCCCTGCTGGAAGCT
GCACACCAGCCCCCTGTGCACCACCAACACCAAAGAGGGCAGCAACATCTGCCTGACCCGGACCGACAGAGGCT
GGTACTGCGACAACGCCGGCAGCGTGTCATTCTTTCCACAGGCCGAGACATGCAAGGTGCAGAGCAACCGGGTG
TTCTGCGACACCATGAACAGCCTGACCCTGCCCTCCGAAGTGAACCTGTGCAACGTGGACATCTTCAACCCCAA
GTACGACTGCAAGATCATGACCTCCAAGACCGACGTGTCCAGCTCCGTGATCACCTCCCTGGGCGCCATCGTGT
CCTGCTACGGCAAGACCAAGTGCACCGCCAGCAACAAGAACCGGGGCATCATCAAGACCTTCAGCAACGGCTGC
GACTACGTGTCCAACAAGGGGGTGGACACCGTGTCCGTGGGCAACACCCTGTACTACGTGAACAAACAGGAAGG
CAAGAGCCTGTACGTGAAGGGCGAGCCCATCATCAACTTCTACGACCCCCTGGTGTTCCCCAGCGACGAGTTCG
ACGCCAGCATCAGCCAGGTGAACGAGAAGATCAACCAGAGCCTGGCCTTCATCCGGAAGTCCGACGAGCTGCTG
CACAATGTGAATGCCGGCAAGTCCACCACCAACTGATGAGCGGCCATCTAATCAACCTCTGGATTACAAAATTT
GTGAAAGATTGACTGGTATTCTTAACTATGTTGCTCCTTTTACGCTATGTGGATACGCTGCTTTAATGCCTTTG
TATCATGCTATTGCTTCCCGTATGGCTTTCATTTTCTCCTCCTTGTATAAATCCTGGTTGCTGTCTCTTTATGA
GGAGTTGTGGCCCGTTGTCAGGCAACGTGGCGTGGTGTGCACTGTGTTTGCTGACGCAACCCCCACTGGTTGGG
GCATTGCCACCACCTGTCAGCTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACAATTCCGTGGTGTTGTCGGGGAA
ATCATCGTCCTTTCCTTGGCTGCTCGCCTGTGTTGCCACCTGGATTCTGCGCGGGACGTCCTTCTGCTACGTCC
CTTCGGCCCTCAATCCAGCGGACCTTCCTTCCCGCGGCCTGCTGCCGGCTCTGCGGCCTCTTCCGCGTCTTCGC
CTTCGCCCTCAGACGAGTCGGATCTCCCTTTGGGCCGCCTCCCCGCCTGCGGCCGCGATCTGCTGTGCCTTCTA
GTTGCCAGCCATCTGTTGTTTGCCCCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTCTATTCTGGGGGGTGGGGTGGGGCA
GGACAGCAAGGGGGAGGATTGGGAAGACAATAGCAGGCATGCTGGGGATGCGGTGGGCTCTATGG
Enhanced CMV Promoter sequence: bold
Transgene sequence F0: Italic
WPRE sequence: underlined bold
bghpolyA PolyA signal: italic + underline
F0 protein sequence
SEQ ID NO: 10
MELLILKANAITTILTAVTFCFASGQNITEEFYQSTCSAVSKGYLSALRTGWYTSVITIELSNIKENKCNGTDA
KVKLIKQELDKYKNAVTELQLLMQSTPATNNRARRELPRFMNYTLNNAKKTNVTLSKKRKRRFLGFLLGVGSAI
ASGVAVSKVLHLEGEVNKIKSALLSTNKAVVSLSNGVSVLTSKVLDLKNYIDKQLLPIVNKQSCSISNIETVIE
FQQKNNRLLEITREFSVNAGVTTPVSTYMLTNSELLSLINDMPITNDQKKLMSNNVQIVRQQSYSIMSIIKEEV
LAYVVQLPLYGVIDTPCWKLHTSPLCTTNTKEGSNICLTRTDRGWYCDNAGSVSFFPQAETCKVQSNRVFCDTM
NSLTLPSEVNLCNVDIFNPKYDCKIMTSKTDVSSSVITSLGAIVSCYGKTKCTASNKNRGIIKTFSNGCDYVSN
KGVDTVSVGNTLYYVNKQEGKSLYVKGEPIINFYDPLVFPSDEFDASISQVNEKINQSLAFIRKSDELLHNVNA
GKSTTN
Amino acid sequence of a flexible linker
SEQ ID NO: 11
Gly-Gly-Gly-Ser-Gly-Gly-Gly
Amino acid sequence of a flexible linker
SEQ ID NO: 12
Gly-Gly-Gly-Gly-Ser-Gly-Gly-Gly-Gly

1. A simian adenoviral vector comprising two expression cassettes, wherein each expression cassette comprises a transgene and a promoter, and wherein the first expression cassette is inserted in the E1 region of the simian adenoviral vector, and the second expression cassette is inserted in a region of the adenoviral vector that is compatible with vector replication; the transgene of the first and second expression cassettes encode an antigen; and the first expression cassette and/or the second expression cassette comprises an enhanced human CMV promoter having a nucleic acid sequence having at least about 90% sequence identity to SEQ ID NO: 6.

2. A simian adenoviral vector comprising two expression cassettes, wherein each expression cassette comprises a transgene and a promoter, and wherein the first expression cassette is inserted in the E1 region of the simian adenoviral vector, and the second expression cassette is inserted in the E3 region, between the stop codons of L5 and E4 (the “HE1” region) or between the end of the ITR and the cap site of E4 mRNA (the “HE2” region) of the simian adenoviral vector.

3. The simian adenoviral vector of claim 2, wherein the second expression cassette is inserted in the E3 region of the simian adenoviral vector.

4. The simian adenoviral vector of claim 2, wherein the second expression cassette is inserted in the HEI region of the simian adenoviral vector.

5. The simian adenoviral vector of claim 2, wherein the second expression cassette is inserted in the HE2 region of the simian adenoviral vector.

6. The simian adenoviral vector of claim 1, wherein the vector is a chimpanzee adenoviral vector.

7. The simian adenoviral vector of claim 1, wherein the vector is an adenovirus.

8. The simian adenoviral vector of claim 7, wherein the vector is ChAd155.

9. The simian adenoviral vector of claim 7, wherein the vector is ChAd83.

10. (canceled)

11. (canceled)

12. The simian adenoviral vector of claim 1, wherein the enhanced hCMV promoter has a nucleic acid sequence having at least about 95%, at least about 96%, at least about 97%, at least about 98%, or at least about 99% sequence identity to SEQ ID NO: 6.

13. The simian adenoviral vector of claim 1, wherein, the promoter comprises or consists of a nucleic acid sequence of SEQ ID NO: 6.

14. The simian adenoviral vector of claim 1, wherein the first and second expression cassettes comprise different promoters.

15. The simian adenoviral vector of claim 1, wherein the adenoviral vector is capable of infecting a mammalian cell.

16. The simian adenoviral vector of claim 1, wherein the first and/or second expression cassette further comprises a posttranscriptional regulatory element.

17. The simian adenoviral vector of claim 16, wherein the posttranscriptional regulatory element is a Woodchuck Hepatitis Postranscriptional Regulatory Element.

18. A composition comprising a simian adenoviral vector of claim 1 and a pharmaceutically acceptable excipient.

19. (canceled)

20. (canceled)

21. A simian adenoviral vector according to claim 1 for the therapy or prophylaxis of a disease.

22. A method of inducing an immune response in a subject comprising administering the simian adenoviral vector according to claim 1 to the subject.

23. A method of inducing an immune response in a subject comprising administering the composition according to claim 18 to the subject.
